(19) United States
(12) Patent Application Publication
(10) Pub. No.: US 2024/0287081 A1
(43) Pub. Date: Aug. 29, 2024

(54) PERFLUOROALKANE SUBSTITUTED PYRAZOLO[3,4-D]PYRIMIDIN AND PYRROLO[2,3-D]PYRIMIDIN COMPOUNDS AND USES THEREOF

(71) Applicant: ICAHN SCHOOL OF MEDICINE AT MOUNT SINAI, New York, NY (US)

(72) Inventors: Alexander P. SCOPTON, New York, NY (US); Ross L. CAGAN, New York, NY (US); Arvin C. DAR, New York, NY (US); Ernesto GUCCIONE, New York, NY (US); Alexander RIALDI, New York, NY (US); Slim MZOUGHI, New York, NY (US)

(21) Appl. No.: 18/568,814

(22) PCT Filed: Jun. 9, 2022
(86) PCT No.: PCT/US2022/032872

§ 371 Date: Dec. 9, 2023

Related U.S. Application Data
(60) Provisional application No. 63/208,623, filed on Jun. 9, 2021.

(51) Int. Cl. C07D 487/04; A61K 31/519; A61P 35/00

(52) U.S. Cl. CPC C07D 487/04; A61K 31/519; A61P 35/00

(57) ABSTRACT

The present disclosure relates to compounds of formula (I) having the following structure; and to compounds of formula (II) having the structure: or stereoisomers, pharmaceutically acceptable salts, oxides, or solvates thereof, where R¹, R², R³, R⁴, and R⁵ are as described herein. The present disclosure also relates to compositions containing the compounds having the structure of formula (I) and/or formula (II), and treatment methods in a subject using the compounds and/or compositions.

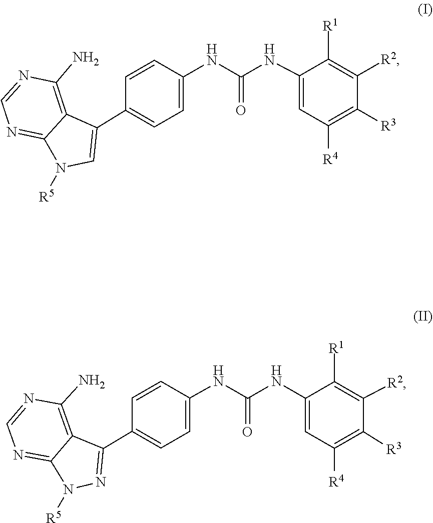

This application claims the priority benefit of U.S. Provisional Patent Application Ser. No. 63/208,623, filed Jun. 9, 2021, which is hereby incorporated by reference in its entirety.

This invention was made with government support under Grant No. U54OD020353 awarded by the National Institutes of Health. The government has certain rights in the invention

### FIELD

The present disclosure relates to perfluoro substituted pyrazolo[3,4-d]pyrimidin and pyrrolo[2,3-d]pyrimidin kinase inhibitor compounds, compositions containing the compounds, and methods of their use.

### BACKGROUND

The kinase domain is found in ˜2% of human genes, and the full complement of 538 human kinases—the kinome—represent one of the largest super families of enzymes (Manning et al., “The Protein Kinase Complement of the Human Genome,” Science 298:1912-1934 (2002)). Kinases regulate signal transduction networks through phosphorylation, but can also play roles as scaffolds and conformational switches to modulate signaling via catalytically-independent mechanisms (Kung & Jura, “Prospects for Pharmacological Targeting of Pseudokinases,” Nat. Rev. Drug Discov. 18(7):501-526 (2019)). Defects in kinase function are linked to a broad array of diseases including diabetes (Delepine et al., “EIF2AK3, Encoding Translation Initiation factor 2-alpha Kinase 3, is Mutated in Patients with Wolcott-Rallison Syndrome,” Nat. Genet. 25:406-409 (2000); Kim et al., “Normal Insulin-dependent Activation of Akt/protein Kinase B, with Diminished Activation of Phosphoinositide 3-kinase, in Muscle in Type 2 Diabetes,” J. Clin. Invest. 104:733-741 (1999)), neurodegeneration (Moore, D. J., “The Biology and Pathobiology of LRRK2: Implications for Parkinson's Disease,” Parkinsonism Relat. Disord. 14 (Suppl 2):S92-8 (2008)), and cancer (Hanahan & Weinberg, “Hallmarks of Cancer: The Next Generation,” Cell 144:646-674 (2011)). For example, within human cancer, thousands of somatic mutations have been found within hundreds of different kinases (Greenman et al., “Patterns of Somatic Mutation in Human Cancer Genomes,” Nature 446:153-158 (2007); Greenman et al., “Statistical Analysis of Pathogenicity of Somatic Mutations in Cancer,” [symbol] 187-2198 (2006); Stephens et al., “Lung Cancer: Intragenic ERBB2 Kinase Mutations in Tumours,” Nature 431:525-526 (2004); Davies et al., “Mutations of the BRAF Gene in Human Cancer,” Nature 417:949-954 (2002)). Over the past two decades, kinases have emerged as one of the most successfully drugged classes of therapeutic targets (Zhang et al., “Targeting Cancer with Small Molecule Kinase Inhibitors,” Nat. Rev. Cancer 9:28-39 (2009); Wu et al., “FDA-approved Small-molecule Kinase Inhibitors,” Trends Pharmacol. Sci. 36:422-439 (2015)). Yet, only a small percentage of the human kinome is identified as the functional targets of clinical drugs (Fleuren et al., “The Kinome ‘At Large’ in Cancer,” Nat. Rev. Cancer 16:83-98 (2016)). Accordingly, novel modalities, including new small molecule approaches, are required to fully harness the therapeutic potential of kinases, and for the further expansion of this target class as a major source of new drugs.

The kinase domain is composed of a smaller N-terminal lobe that is hinged by a single polypeptide strand to a larger C-terminal lobe (De Bondt et al., “Crystal Structure of Cyclin-dependent Kinase 2,” Nature 363:595-602 (1993); Madhusudan et al., “cAMP-dependent Protein Kinase: Crystallographic Insights into Substrate Recognition and Phosphotransfer,” Protein Sci. 3:176-187 (1994); Hubbard et al., “Crystal Structure of the Tyrosine Kinase Domain of the Human Insulin Receptor,” Nature 372:746-754 (1994)). The central ATP binding pocket serves as a bridge between the N- and C-terminal regions. Conformational switching within the kinase domain involves concerted movements of structural elements between and within both sub-domains, which can form the basis for enzyme activation and also for regulating interactions with accessory or effector proteins within higher-order assemblies (Sicheri et al., “Crystal Structure of the Src Family Tyrosine Kinase Hck,” Nature 385:602-609 (1997); Nagar et al., “Structural Basis for the Autoinhibition of c-Abl Tyrosine Kinase,” Cell 112:859-871 (2003); Xu et al., “Three-dimensional Structure of the Tyrosine Kinase c-Src,” Nature 385:595-602 (1997); Russo et al., “Crystal Structure of the p27Kip1 Cyclin-dependent-kinase Inhibitor Bound to the Cyclin A-Cdk2 Complex,” Nature 382:325-331 (1996); Jeffrey et al., “Mechanism of CDK Activation Revealed by the Structure of a CyclinA-CDK2 Complex,” Nature 376:313-320 (1995); Hubbard, S. R., “Crystal Structure of the Activated Insulin Receptor Tyrosine kinase in Complex with Peptide Substrate and ATP Analog,” EMBO J. 16:5572-5581 (1997); Mohammadi et al., “Structure of the FGF Receptor Tyrosine Kinase Domain Reveals a Novel Autoinhibitory Mechanism,” Cell 86:577-587 (1996)). There is an increasing appreciation for small molecule ligands that bias specific kinase conformations as a means to modulate important pharmacodynamic parameters, such as drug residence time (Copeland, R. A., “The Drug-target Residence Time Model: A 10-year Retrospective,” Nat. Rev. Drug Discov. 15:87 (2015)), and thereby kinase signaling output and therapeutic pathways.

A conserved structural element within the kinase domain is the activation segment, which starts at the N-terminal end with the highly conserved DFG tripeptide motif. In the active ‘DFG-IN’ state, the activation segment serves as a platform for substrate binding, whereas in the inactive ‘DFG-OUT’ state, this same region can be unfolded to prevent ATP-substrate complexes (Plenker et al., “Drugging the Catalytically Inactive State of RET Kinase in RET-rearranged Tumors,” Sci. Transl. Med. 9 (2017)). The DFG flip is thought to be critical for the binding and release of ATP, ADP, or products during phosphorylation cycles (Shan et al., “A Conserved Protonation-dependent Switch Controls Drug Binding in the Abl Kinase,” Proceedings of the National Academy of Sciences 106:139-144 (2009)), and therefore this conformational switch is likely pervasive across the kinome (Ung et al., “Redefining the Protein Kinase Conformational Space with Machine Learning,” Cell Chemical Biology 25:916-924.e2 (2018)). Small molecule inhibitors that bind to the inactive DFG-OUT state configuration, in which the DFG-flip enables access to a dynamic allosteric pocket, are referred to as type II inhibitors (Sonoshita et al., “A Whole-animal Platform to Advance a Clinical Kinase Inhibitor into New Disease Space,” Nat. Chem. Biol. 14(3):291-298 (2018); Zhao et al., “Exploration of Type II Binding Mode: A Privileged Approach for Kinase Inhibitor Focused Drug Discovery?” ACS Chem. Biol. 9:1230-1241 (2014)). Several clinical kinase inhibitors (KIs) are type II drugs; examples include imatinib, sorafenib (1), regorafenib (2), and cabozantinib.

The present disclosure is directed to overcoming deficiencies in the art.

### SUMMARY

One aspect of the present disclosure relates to a compound of formula (I) having the following structure:

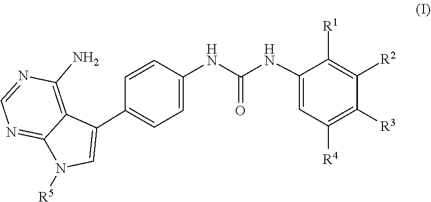

or a stereoisomer, pharmaceutically acceptable salt, oxide, or solvate thereof, wherein
R¹, R², R³, and R⁴ are independently selected from the group consisting of H, F, CF₃, C₂F₅, C₃F₇, C₄F₉, C₅F₁₁, C₆F₁₃, C₇F₁₅, and C₈F₁₇; and
R⁵ is a C₁-C₆ alkyl or a C₃-C₇ cycloalkyl.

Another aspect of the present disclosure relates to a compound of formula (II) having the following structure:

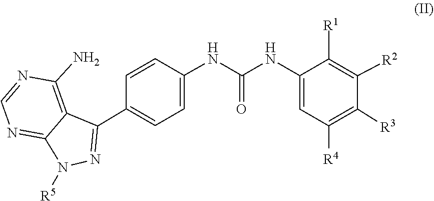

or a stereoisomer, pharmaceutically acceptable salt, oxide, or solvate thereof, wherein
R¹, R², and R³ are independently selected from the group consisting of H, F, CF₃, C₂F₅, C₃F₇, C₄F₉, C₅F₁₁, C₆F₁₃, C₇F₁₅, and C₈F₁₇;
R⁴ is selected from the group consisting of H, F, C₂F₅, C₃F₇, C₄F₉, C₅F₁₁, C₆F₁₃, C₇F₁₅, and C₈F₁₇; and
R⁵ is a C₁-C₆ alkyl or a C₃-C₇ cycloalkyl.

A further aspect of the present disclosure relates to a composition comprising a compound of formula (I) or formula (II) described herein and a carrier.

Another aspect of the present disclosure relates to a method of treating cancer in a subject. This method involves administering to a subject in need thereof a compound of formula (I) having the following structure:

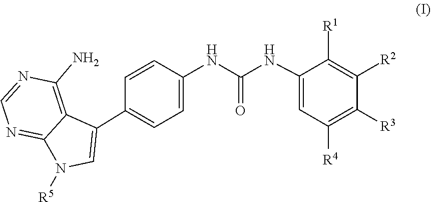

or a stereoisomer, pharmaceutically acceptable salt, oxide, or solvate thereof, wherein
R¹, R², R³, and R⁴ are independently selected from the group consisting of H, F, CF₃, C₂F₅, C₃F₇, C₄F₉, C₅F₁₁, C₆F₁₃, C₇F₁₅, and C₈F₁₇; and
R⁵ is a C₁-C₆ alkyl or a C₃-C₇ cycloalkyl.

A further aspect of the present disclosure relates to a method of treating cancer in a subject. This method involves administering to a subject in need thereof a compound of formula (II) having the following structure:

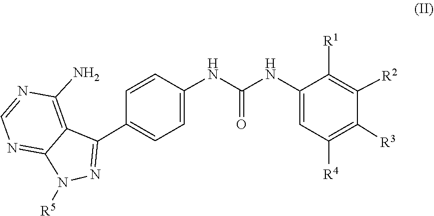

or a stereoisomer, pharmaceutically acceptable salt, oxide, or solvate thereof, wherein
R¹, R², and R³ are independently selected from the group consisting of H, F, CF₃, C₂F₅, C₃F₇, C₄F₉, C₅F₁₁, C₆F₁₃, C₇F₁₅, and C₈F₁₇;
R⁴ is selected from the group consisting of H, F, C₂F₅, C₃F₇, C₄F₉, C₅F₁₁, C₆F₁₃, C₇F₁₅, and C₈F₁₇; and
R⁵ is a C₁-C₆ alkyl or a C₃-C₇ cycloalkyl.

The prototypical type II Kinase Inhibitor (“KI”) format is outlined in FIG. 1A, and is comprised of an H-bond accepting hinge binder, an H-bond donating linker, and a CAP-group subunit, which occupies the allosteric DFG-pocket. In previous work, it was demonstrated that the DFG-pocket is highly variable in size and composition across the kinome, and modifications to the CAP can be utilized to refine unique polypharmacological KI activities (Sonoshita et al., “A Whole-animal Platform to Advance a Clinical Kinase Inhibitor into New Disease Space,” Nat. Chem. Biol. 14(3):291-298 (2018); Zhao et al., “Exploration of Type II Binding Mode: A Privileged Approach for Kinase Inhibitor Focused Drug Discovery?,” ACS Chem. Biol. 9:1230-1241 (2014), which are hereby incorporated by reference in their entirety). Important to such refinement are decorations on the phenyl CAP-group, which is often meta-CF₃ substituted and is sometimes found to have concomitant 2- or 4-position substituents, with 3- and 6-position substituents not being prevalent. Through previous work, APS-6-45 (3)

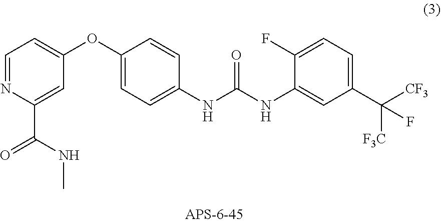 [SMILES: CNC(=O)c1cc(Oc2ccc(NC(=O)Nc3cc(C(F)(C(F)(F)F)C(F)(F)F)ccc3F)cc2)ccn1]

was identified as a strong lead compound in a Drosophila model for RET-mutated MTC, which performed well in a mouse xenograft study using the human MTC cell line TT (FIG. 1B) (Sonoshita et al., “A Whole-animal Platform to Advance a Clinical Kinase Inhibitor into New Disease Space,” Nat. Chem. Biol. 14(3):291-298 (2018); Zhao et al., “Exploration of Type II Binding Mode: A Privileged Approach for Kinase Inhibitor Focused Drug Discovery?,” ACS Chem. Biol. 9:1230-1241 (2014); Yamamura et al. “Tiny Drosophila Makes Giant Strides in Cancer Research,” Cancer Sci. 12(2):505-514, which are hereby incorporated by reference in their entirety).

In the present disclosure, the evolution of CAP-group modifications was explored using an expanded library of perfluoroalkyl-groups (—R^{F}). While the role of fluorine in drug design/development continues to expand and is appreciated (Bohm et al., “Fluorine in Medicinal Chemistry,” Chembiochem 5:637-643 (2004); Müller et al., “Fluorine in Pharmaceuticals: Looking Beyond Intuition,” Science 317:1881-1886 (2007), which are hereby incorporated by reference in their entirety), the use of perfluoroalkanes in KI development is not well explored (Pertusati et al., “3-Polyfluorinated Scaffolds in Drug Discovery,” in Fluorine in Life Sciences: Pharmaceuticals, Medicinal Diagnostics, and Agrochemicals, eds. Haufe & Leroux, Academic Press, 141-180 (2019), which is hereby incorporated by reference in its entirety). This may reflect the restricted number of useful building blocks that are commercially available, such as perfluoroalkyl-substituted anilines or isocyanates (—CF₃ functionalized building blocks are readily available but the corresponding higher-order perfluoroalkyl-analogs are not). The present disclosure describes several synthetic strategies to access a set of perfluoroalkylated building blocks used for systematic SAR analysis. The transferability of this toolkit for modifying the selectivity and in vivo properties of several classical type II KIs was also demonstrated. Several compounds were identified that surprisingly exceeded the activity of APS-6-45 (3) and AD80 (64) (Ung et al., “Redefining the Protein Kinase Conformational Space with Machine Learning,” Cell Chemical Biology 25:916-924.e2 (2018); Sonoshita et al., “A Whole-animal Platform to Advance a Clinical Kinase Inhibitor into New Disease Space,” Nat. Chem. Biol. 14(3):291-298 (2018); Zhao et al., “Exploration of Type II Binding Mode: A Privileged Approach for Kinase Inhibitor Focused Drug Discovery?,” ACS Chem. Biol. 9:1230-1241 (2014), which are hereby incorporated by reference in their entirety), including 12 new compounds that provide unprecedented suppression and >95% rescue of oncogenic Ret^{M955T}-induced lethality in the Drosophila model. Finally, comparative SAR and computational analyses suggest unique complementarity between perfluoroalkyl groups and specific electrostatic pockets within kinase targets, as well as a physicochemical rationalization for the observed pharmacokinetic (PK) and pharmacodynamic (PD) profiles.

Developing approaches for rational or intended polypharmacology is challenging given the complexity of refining small molecule interactions across many biological targets. Some approaches have attempted structure-based predictions, pattern recognition, or machine learning. The present disclosure presents an advancement to a chemical genetic strategy that uses directed libraries to systematically query polypharmacology across the kinome. Compound libraries were developed by functionalizing known drug cores with an array of perfluoroalkyl-substituents at a site to engage the type II subpocket of kinases, a region that is shared among all kinases but is diverse in terms of shape, sequence, and binding properties. An initial series of perfluoroalkylated-analogs of sorafenib (1)

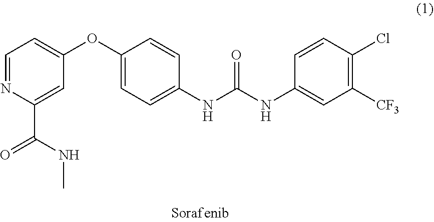 [SMILES: CNC(=O)c1cc(Oc2ccc(NC(=O)Nc3ccc(Cl)c(C(F)(F)F)c3)cc2)ccn1]

and regorafenib (2)

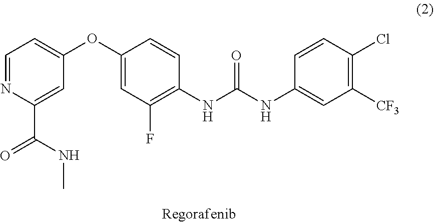 [SMILES: CNC(=O)c1cc(Oc2ccc(NC(=O)Nc3ccc(Cl)c(C(F)(F)F)c3)c(F)c2)ccn1]

demonstrated significant improvements in efficacy relative to the parent drugs and allowed for the identification of a preliminary lead compound, APS-6-45 (3), which performed well in a mouse xenograft assay for medullary thyroid carcinoma (“MTC”). Further studies in a RET-mutant MTC Drosophila model led to the development of a toolkit of perfluoroalkyl-aniline building blocks that could be transferred onto additional type II inhibitor scaffolds for systematic SAR analysis and hyper-optimization of a drug. However, despite their similar size and shape, the hydrocarbon counterparts for a series of regorafenib-based perfluoroalkyl-analogs proved essentially ineffective in vivo, demonstrating the utility of perfluoroalkanes as probe appendages for whole animal studies. RET binding (Kd) and in cell IC₅₀ measurements showed that on target potency did not correlate well with in vivo efficacy. Kinome wide binding data indicated that individual perfluoroalkanes can be used to adjust the profile of a drug core. Finally, cell permeability measurements, as well as structural and molecular properties calculations, revealed that the unique physicochemical properties of perfluoroalkyl-substituted drugs contribute significantly to favorable pharmacokinetic (PK) and pharmacodynamic (PD) profiles. Thus, the structural complementarity of the perfluoroalkylated building blocks allows for a drug core to be incrementally extended in order to accommodate the type II binding pocket of specific kinases. This toolkit and the described strategies should enable the further development of chemical probes to explore in vivo kinase polypharmacology in a rational manner.

### BRIEF DESCRIPTION OF THE DRAWINGS

FIGS. 1A-1C are schematic illustrations showing one embodiment of a chemical toolkit of perfluoroalkanes to engage the ‘CAP’ subpocket of diverse kinases. FIG. 1A shows the Classical Type-2 Inhibitor format consisting of several pharmacophores including a hinge-binder element, linker, and CAP. In the present disclosure, the classical Type-2 CAP was evolved to one that includes various perfluoroalkane substitutions, in both the meta- and para-position. FIG. 1B illustrates chemical structures of clinical compounds (1, 2) and a previously described sorafelog, APS-6-45 (3). FIG. 1C shows the ‘Chemical Toolbox’ that includes caps with systematic alterations of perfluoroalkane groups. In the initial library of analogs cores 1A and 2A were used as the constant hinge-binder and linker elements to which the various CAP groups were incorporated.

FIG. 2 is a schematic illustration showing the possible chemical reaction that may explain the failure of the Ullman-type coupling when employing I-C₄F₉ as a substrate. Presumably, the cuprate (21) that forms in situ is not stable under the reaction conditions and collapses to 22 at a faster rate than cross-coupling occurs. A variety of conditions were attempted but the only products ever isolated were 23 and 24. See also: FIG. 7C.

FIGS. 3A-3E show that the unique target/anti-target profiles and physicochemical properties dictate in vivo efficacy of perfluoroalkylated compounds. The binding profiles for a series of structurally related compounds (FIG. 3A) determined against a panel of 468 kinases; 38 (0% rescue), 43 (85% rescue), and 65 (99% rescue) are shown as examples. The profiling data was filtered down to 330 kinases based on variance. FIG. 3B is a table of the top 25 kinases bound by 4-9-8, 8-82-1, and 8-6-2. On the regorafenib core, 4-9-8 (—CF₃) showed strong binding to wild-type and mutant RET^{M918T} and low overall selectivity, while the larger 8-82-1 (-sC₄F₉) was highly selective across the kinome, and despite not being as potent on RET, showed selectivity for the M918T mutant over wild-type. Swapping the -sC₄F₉ substituent onto the pyrazolopyrimidine core (8-6-2) produced a compound that was highly potent on both wild-type and RET^{M918T}, with an overall selectivity profile in between 4-9-8 and 8-82-1. FIG. 3C is a plot of the difference in binding between 38 and 43 across 330 individual kinases from the filtered data set. Overall the larger substituent showed greater selectivity (-sC4F9 vs —CF₃). FIG. 3D shows molecular stick models showing partial charges for a series of matched Ph-R^{F}/R^{H} model fragments; Ph-sC₄F₉ vs Ph-sC₄H₉ and Ph-nC₄F₉ vs Ph-nC₄H₉ serve as examples. FIG. 3E shows the molecular electrostatic potential (MEP) mapped onto the van der Waals surface of the matched Ph-R^{F}/R^{H} model fragments; MEP maps are shown as a surface/stick overlay, and from a top, left, and front view of the surface. The overall difference between the Ph-R^{F} and Ph-R^{H} MEP maps is as striking as the difference in performance between the R^{F}- and R^{H}-functionalized drugs. The increased electron density of the perfluoroalkyl-substituents appears to be related to favorable PK and PD profiles.

FIG. 4 is a graphic showing that the Sorafenib and Regorafenib Scaffolds demonstrate relatively large tolerance for perfluoroalkane substitutions on the CAP. The rescue activity of the full library of 1A (black) and 2A (red) perfluroalkyl CAP analogs in the fly RET^{M918T} MTC model is shown. In the absence of any compound, RET^{M918T} viability is 0% (adults/pupae×100). Rescue by compounds is quantified as the percent viability of adult flies that emerge from the pupae stage during development. Compounds are ordered based on substitution pattern, including a meta-series (left), 2,5-series (middle), and para-series (right). Compounds 35 and 36 surpassed the activity of previously described analog APS-6-45 (3) Compounds were administered through fly food and assayed for effects on viability against greater than 25 animals each.

FIG. 5 compares representative R^{F} and R^{H} pairs demonstrating the striking activity of perfluoroalkane analogs in vivo. Perfluoroalkane and matched hydrocarbon analogs on both the sorafenib (1A) and regorafenib (2A) cores were tested for effects on fly viability and assayed as described for FIG. 4. The perfluoroalkane analogs demonstrated marked rescue activity relative to the hydrocarbon derivatives; for examples see 35 vs 51 and 36 vs 52.

FIG. 6 shows the activity of perfluoroalkylated CAPs transferred to unique kinase hinge-binder scaffolds. Rescue activity of the full library of pyrazolopyrimidine (blue) and pyrrolopyrimidine (purple) perfluoroalkyl CAP analogs in the fly RET^{M918T} model. In the absence of any compound, RET^{M918T} viability is 0% (adults/pupae×100). Rescue by compounds is quantified as the percent viability of adult flies that emerge from the pupae stage during development. Compounds are ordered based on substitution patterns, including a meta-series (left), 2,5-series (middle), and para-series (right). Thirteen compounds in these series surpass the activity of previously described analog AD80 (64). Compounds were administered through fly food and assayed for effects on viability against greater than 25 animals each.

FIGS. 7A-7D show strategies to synthesize various perfluoroalkane containing type II KIs. FIG. 7A shows the reaction scheme where the urea-linker was formed in the final step of the synthetic sequence by reacting the sorafenib (4) and regorafenib (5) core building blocks with either a commercially available isocyanate (R¹═H, F; R², R³═H, —CF₃) or an acyl imidazole intermediate that was generated in situ by reacting N,N′-carbonyldiimidazole with an in house synthesized perfluoroalkyl-aniline building block (R¹═H, F; R², R³═H, —C₂F₅, -nC₃F₇, -iC₃F₇, -nC₄F₉, -sC₄F₉). FIG. 7B shows the synthesis of the para-substituted aniline series (6-10) by sodium hydrosulfite mediated radical coupling of aniline with an iodo perfluoroalkane (I-R^{F}) under phase-transfer conditions. TBAH=tetrabutylammonium hydrogensulfate; MTBE=tert-butyl methyl ether. FIG. 7C shows the synthesis of the majority of the meta-(R═H; 11, 13, 15, 17, 19) and 2,5-substituted (R═F; 12, 14, 16, 18, 20) aniline series obtained by Ullmann-type coupling of a nitro-substituted aryl iodide with an iodo perfluoroalkane, followed by reduction of the nitro-group by palladium catalyzed hydrogenation or with iron. Synthesis of 19 and 20 were not achieved by this synthetic sequence due to failure of the copper-mediated coupling (see FIG. 2), under a variety of conditions. FIG. 7D shows the alternative synthetic strategies for 19 and 20 developed from the same starting point (10). Acetylation (10→25), nitration/deacetylation (25→26), diazotization/reduction and hydrogenation provided 19, while diazotization (10→27), rearrangement (27→28), nitration, and hydrogenation provided 20.

FIGS. 8A-8D show the in vitro and cell-based target engagement on wild-type and mutant forms of RET. FIG. 8A is a pair of plots of the in vitro binding of compounds 40 and 50 on the wild-type RET kinase domain. The binding curve for regorafenib is shown for comparison purposes. Assays were completed in duplicate. FIG. 8B is a series of plots of the in vitro binding towards wild-type RET kinase domain for a series of analogs with systematic alterations in size and branching of perfluoroalkane groups, including compounds 38, 40, 36, 42, 43, and 46. These analogs all include the regorafenib-based scaffold (2A). In general, increasing the size of the R^{F}-substituent reduced direct binding on RET^{WT}. FIG. 8C is a plot comparing the in vivo rescue activity against RET^{M918T} flies and direct in vitro binding towards wild-type RET for the indicated compounds. This data suggested that potency on RET^{WT} does not determine rescue activity in the RET^{M918T} fly model. FIG. 8D is a graph of the cell-based target engagement for the indicated compounds against wild-type and M918T RET. IC50 values were determined based on 8-point titration curves of free ligand. Competition assays were completed in technical triplicate against full-length human RET-Nluc expressed in 293T cells, complexed with an active-site fluorescent tracer. This data indicates that most compounds bind with similar potency on wild-type and mutant RET.

FIGS. 9A-9C show the physicochemical comparison of R^{F} and R^{H} Analogs. FIG. 9A is a graph of the MDCK and Caco-2 permeability assays for the indicated compounds. Compounds were assayed at 5 μM and transport (Papp, B-A and Papp, A-B, ×10⁻⁶ cm/s) values were measured to determine influx over efflux ratios. Sorafenib (1), APS-6-45 (3), APS-8-100-2 (40), and APS-9-23-6 (50) all have ratios <2 in MDCK, suggesting that they are unlikely substrates of the P-gp transporter. 1, 3, and 40 have ratios >2 in Caco-2, suggesting that they are potential substrates of certain transporters. In Caco2, 50 showed a ratio <1, suggesting that this compound is unlikely a substrate of active transport. FIG. 9B shows images of the matched pairs of R^{F}/R^{H}-analogs showing a striking difference in appearance when mixed in PBS buffer. A few compounds from the regorafenib series are shown as general examples (35 vs 51, 41 vs 53, 44 vs 55, and 45 vs 57). FIG. 9C shows the bond and molecular dipoles for a series of matched Ph-R^{F}/R^{H} model fragments; Ph-sC₄F₉ vs Ph-sC₄H₉ and Ph-nC₄F₉ vs Ph-nC₄H₉ serve as examples and are shown from a left and right perspective. There are differences in magnitude for bond and molecular dipoles, and R^{F}- and R^{H}-substituents have opposite charge profiles.

FIGS. 10A-10H are a summary of the key structural and physicochemical factors related to favorable PK/PD profiles and in vivo efficacy of perfluoroalkyl-drugs. FIG. 10A shows side-by-side comparisons of MEP maps of the internal faces of the RET, BRAF, and SRC type II binding pockets. The bottom face is comprised of residues along the interface between the N- and C-lobes. The MEP for the entire protein was mapped onto the corresponding individual faces, which essentially provided a cross section of the binding pocket. FIG. 10B shows the structural alignment of RET(B06) (SEQ ID NO:1), BRAF(5HI2) (SEQ ID NO:2), and SRC(3EL8) (SEQ ID NO:3) showing the N- and C-lobe fragments that combine to form the bottom face of the type II pocket; RET(B06) is colored dark purple, SOR is teal, BRAF is forest green, AD57 is bright orange and SRC is slate. Residues associated with individual bottom face fragments are indicated (red lines) in a partial sequence alignment with full length human RET(F8TLS5) (SEQ ID NO:4); the relative locations of the DFG- and HRD-motifs are also shown. FIG. 10C is a graph of the in vitro kinase binding data for a series of related para-substituted regorafenib core analogs against RET, RET(M918T), BRAF, and SRC. Percent inhibition shown represents an average of two experimental replicates using 1 μM drug. FIG. 10D is a graph of the calculated DFG-pocket volumes (Å³) for RET, BRAF, and SRC. FIG. 10E is a graph of the calculated Connolly solvent excluded volumes (Å³) for the Ph-R^{F}/R^{H} matched pair model fragments. FIG. 10F is a graph of the calculated CLogP values for the Ph-R^{F}/R^{H} matched pair model fragments. FIG. 10G is a graph of the calculated molecular dipoles (debye) for the Ph-R^{F}/R^{H} matched pair model fragments. FIG. 10H is a graph of the atomic (H, C, F) and group electronegativity (Pauling, xr) values for select R^{F}(blue)-, R^{F/H} (purple)- and R^{H}(red)-substituents (Huheey, J. E., “The Electronegativity of Groups,” J. Phys. Chem. 69:3284-3291 (1965), which is hereby incorporated by reference in its entirety).

FIG. 11 shows the bond and molecular dipoles for Ph-R^{F}/R^{H} model fragments. The bond and molecular dipoles were calculated for a series of matched pairs of Ph-R^{F} (—CF₃, —C₂F₅, -nC₃F₇, -iC₃F₇, -nC₄F₉, and -sC₄F₉) and Ph-R^{H} (—CH₃, —C₂H₅, -nC₃H₇, -iC₃H₇, -nC₄H₉, and -sC₄H₉) CAP-group fragments. Bond dipoles are colored red and molecular dipoles are blue. Top, bottom, left, and right views are shown.

FIG. 12 shows the atomic numbers and partial charges of the Ph-R^{F}/R^{H} model fragments. The atomic numbers and partial charges are shown for a series of matched pairs of Ph-R^{F} (—CF₃, —C₂F₅, -nC₃F₇, -iC₃F₇, -nC₄F₉, and -sC₄F₉) and Ph-R^{H} (—CH₃, —C₂H₅, -nC₃H₇, - iC₃H₇, -nC₄H₉, and -sC₄H₉) CAP group fragments. Huckel, MMFF94, Lowdin, and Mulliken charges are listed, as well as Lowdin and Mulliken populations. Structures are shown as sticks; hydrogen in white, carbon in gray and fluorine in yellow; partial charges are displayed on a red(δ⁻)-white(0)-blue(δ⁺) color ramp.

FIG. 13 shows the MEPs mapped onto the van der Waals surface of a series of Ph-R^{F} model fragments. The MEP maps are shown for R^{F}=—CF₃, —C₂F₅, -nC₃F₇, -iC₃F₇, -nC₄F₉, and -sC₄F₉. The surface maps are shown from several perspectives, and as an overlay with the stick structure.

FIG. 14 shows side-by-side MEP map comparisons of Ph-R^{F}/R^{H} model fragments. The MEPs are shown mapped onto the van der Waals surface for a series of Ph-R^{F}/R^{H} matched pairs (—CF₃ vs —CH₃; —C₂F₅ vs —C₂H₅; -nC₃F₇ vs -nC₃H₇; -iC₃F₇ vs -iC₃H₇; -nC₄F₉ vs -nC₄H₉; -sC₄F₉ vs -sC₄H₉). The surface maps are shown from top, left, and front perspectives.

FIGS. 15A-15C show the calculated partial charges, dipoles, and MEP maps for model dipeptide Me-Val-Leu-Ac. FIG. 15A shows the basic structure and stick representations of the model dipeptide showing atomic numbers (hydrogen in white, carbon in gray, nitrogen in blue, and oxygen in red) and partial charges (red(δ⁺)-white(0)-blue(δ⁻) color ramp). Huckel, MMFF94, Lowdin, and Mulliken charges are listed, as well as Lowdin and Mulliken populations. FIG. 15B shows the calculated bond and molecular dipoles for the model dipeptide (Me-VL-Ac). Bond dipoles are colored red and molecular dipoles are blue. Front and back views are shown. FIG. 15C shows the calculated (HF 6-311G) MEP map for the model dipeptide (Me-VL-Ac), shown from the front and back. The MEP map is shown as an overlay with the stick structure or as an opaque surface. Areas of positive electrostatic potential are observed at the hydrogen atoms of hydrocarbon side-chains, amide protons (N—H) and α-carbons; these are complimentary to the MEP maps calculated for the Ph-R^{F} CAP-group model fragments.

FIGS. 16A-16B are schematic illustrations showing the physicochemical rationalization for differences in the PK/PD profiles of R^{F}- and R^{H}-compounds. FIG. 16A shows that through hyperconjugation, R^{H}-substituents (e.g., —CH₃) donate electron density into the pi-system of phenyl CAP groups. This can make them more susceptible to oxidative degradation. R^{H}-substituents are only capable of forming van der Waals interactions within a protein binding pocket. LB=Lewis Base. FIG. 16B shows the strong electron withdrawal from R^{F}-substituents decreases pi-system electron density, preventing oxidative metabolism of the phenyl CAP group, while also strengthening pi-stacking interactions. R^{F}_substituents are capable of multipolar interactions at the fluorine atoms and tetrel bonding at the associated carbons.

FIGS. 17A-17E show known crystal structures of BRAF and SRC help to define the type II binding pocket in a RET homology model. FIG. 17A shows the alignment of the ten RET type II (inactive state) homology models available from kinametrix.com. FIG. 17B shows the alignment of sorafenib (SOR)-bound-BRAF (5HI2) and AD57-bound-SRC (3EL8) structures. SOR is colored teal, BRAF is forest green, AD57 is bright orange, and SRC is slate. FIG. 17C shows the alignment of the SOR/BRAF and AD57/SRC structures with RET homology model B06 from kinametrix.com. RET(B06) is colored dark purple. FIG. 17D shows the structures of BRAF and SRC removed from the alignment in FIG. 17C, leaving the corresponding ligands (SOR/AD57) to help define the type II binding pocket of the RET(B06) homology model. The structurally different molecular cores of SOR and AD57 both contain CAP groups with meta-trifluoromethyl-substituents that are directed to a pocket adjacent to the DFG- and HRD-motifs. FIG. 17E shows surface representations of the RET(B06)/SOR/AD57 alignment, showing the hinge (left) and DFG/HRD (middle) sides of the type II binding pocket. Distance of the ligand-pair (SOR/AD57) from the RET(B06) homology model is mapped onto the surface (2-20 angstroms). Expansion (right) of the DFG/HRD side of the type II binding pocket (green outline) revealed that the meta-trifluoromethyl-substituents of SOR and AD57 would bind in a subpocket occupied by ‘greasy’ amino acid side-chain residues; side-chains shown as sticks are within 10 Å of the SOR/AD57 —CF₃ substituents.

FIGS. 18A-18F are MEP maps of wild-type RET. FIG. 18A is an overlay of the RET(B06) homology model and the SOR/AD57 ligand pair, with the N- and C-lobes colored magenta and slate respectively. SOR and AD57 are shown as sticks and are colored teal and bright orange respectively. FIG. 18B is the front and back views of the molecular electrostatic potential mapped onto the surface of the RET(B06) homology model. FIG. 18C shows the electrostatic potential inside the type II binding pocket of the RET(B06) homology model, visualized by opening the structure across the N- and C-lobes, and applying the MEP map for the entire protein to the individual faces. MEP maps of the internal faces of RET(B06) essentially revealed a cross-section of the hydrocarbon-lined pocket directly adjacent to the SOR/AD57 —CF₃ groups. This cross-section displayed a large area of positive electrostatic potential, complementary to the negative electrostatic potential associated with R^{F}_substituents (see FIGS. 13 and 14). Relative to BRAF and SRC, MEP maps of the internal faces, of the RET(B06) type II binding pocket, showed the largest overall area positive electrostatic potential (see FIGS. 20A-20F and 21A-21F). FIG. 18D is the underside view of the bottom face of the RET(B06) type II binding pocket. The bottom face is comprised of residues from the N- and C-lobes, and is shown as a white surface with 50% transparency. FIG. 18E is the overhead view showing the relative orientation of the ligands (SOR/AD57) to the bottom face (white surface) of the type II binding pocket. FIG. 18F shows the MEP of RET(B06) mapped onto the surface representation of the bottom face of the type II binding pocket. The observed area of positive electrostatic potential appeared greater than both BRAF and SRC (see FIGS. 10A-10H, 20A-20F, and 21A-21F).

FIGS. 19A-19F are MEP maps of an in silico generated RET(B06)^{M918T} mutant. FIG. 19A is an overlay of the SOR/AD57 ligand pair and the M918T mutant that was derived in silico from the RET(B06) homology model. The point mutation is shown as a green sphere and was measured to be 16 Å from the para-chloro-substituent of SOR. The N- and C-lobes are colored light magenta and blue respectively. SOR and AD57 are shown as sticks and are colored teal and bright orange respectively. FIG. 19B is the front and back views of the molecular electrostatic potential mapped onto the surface of the RET(B06)^{M918T} homology model mutant. Compared to wild-type RET(B06), a very minor difference in volume and electrostatic potential were observed on the surface of the protein at the M918T site. FIG. 19C shows the electrostatic potential along the faces of the type II binding pocket of the RET(B06)^{M918T} mutant is identical to the wild-type RET(B06) model. This was observed for both potential threonine side-chain rotamers. FIG. 19D is the underside view of the bottom face of the RET(B06)^{M918T} type II binding pocket. The bottom face is comprised of residues from the N- and C-lobes, and is shown as a white surface with 50% transparency. FIG. 19E is the overhead view showing the relative orientation of the ligands (SOR/AD57) to the bottom face (white surface) of the type II binding pocket. FIG. 19F shows the MEP of RET(B06)^{M918T} mapped onto the surface representation of the bottom face of the type II binding pocket. The observed area of positive electrostatic potential is identical to the corresponding wild-type model.

FIGS. 20A-20F are MEP maps of a sorafenib-bound-BRAF structure. FIG. 20A is a schematic representation of the structure of SOR bound to BRAF in the type II conformation (5HI2). N- and C-lobes are shown in red and purple respectively, SOR is shown in teal. FIG. 20B is the front and back views of the molecular electrostatic potential mapped onto the BRAF surface. FIG. 20C shows the electrostatic potential inside the type II binding pocket of sorafenib-bound-BRAF, visualized by opening the structure across the N- and C-lobes. MEP maps of the internal faces of the BRAF type II binding pocket showed a cross-sectional area of positive electrostatic potential that was smaller than RET(B06), but larger than SRC (see FIGS. 18A-18F, 19A-19F, and 21A-21F). FIG. 20D is the underside view of the bottom face of the BRAF type II binding pocket. The bottom face is comprised of residues from the N- and C-lobes, and is shown as a white surface with 50% transparency. FIG. 20E is the overhead view showing the relative orientation of SOR to the bottom face (white surface) of the type II binding pocket. FIG. 20F shows the MEP of BRAF mapped onto the surface representation of the bottom face of the pocket. The observed area of positive electrostatic potential appeared to be less than RET(B06) but greater than SRC (see FIGS. 10A-10H, 18A-18F, 19A-19F, and 21A-21F).

FIGS. 21A-21F are MEP maps of the AD57-bound-BRAF structure. FIG. 21A is a schematic representation of the structure of AD57 bound to SRC in the type II conformation (3EL8). N- and C-lobes are shown in marine and chartreuse respectively, AD57 is shown in bright orange. FIG. 21B is the front and back views of the molecular electrostatic potential mapped onto the SRC surface. FIG. 21C shows the electrostatic potential inside the type II binding pocket of AD57-bound-SRC, visualized by opening the structure across the N- and C-lobes. MEP maps of the internal faces of the SRC type II binding pocket showed a cross-sectional area of positive electrostatic potential that was smaller than both RET and BRAF (see FIGS. 10A-10H, 18A-18F, 19A-19F, and 20A-20F). FIG. 21D is the underside view of the bottom face of the SRC type II binding pocket. The bottom face is comprised of residues from the N- and C-lobes, and is shown as a white surface with 50% transparency. FIG. 21E is the overhead view showing the relative orientation of AD57 to the bottom face (white surface) of the type II binding pocket. FIG. 21F shows the MEP of SRC mapped onto the surface representation of the bottom face of the pocket. The observed area of positive electrostatic potential appeared to be less than both RET and BRAF (see FIGS. 10A-10H, 18A-18F, 19A-19F, and 20A-20F).

FIGS. 22A-22F are tables showing the results of 3D hepatocellular carcinoma (“HCC”) cell line data for certain pyrazolo- and pyrrolopyrimidine compounds disclosed herein at 0.5 μm.

FIGS. 23A-23F are tables showing the results of 3D HCC cell line data for certain pyrazolo- and pyrrolopyrimidine compounds disclosed herein at 0.05 μm.

FIGS. 24A-24F are tables showing the results of 3D HCC cell line data for certain pyrazolo- and pyrrolopyrimidine compounds disclosed herein at 5.0 μm.

FIGS. 25A-25G are tables showing the results of 3D colorectal cancer (“CRC”) organoid line data for certain pyrazolo- and pyrrolopyrimidine compounds disclosed herein at 0.5 μm.

### DETAILED DESCRIPTION

One aspect of the present disclosure relates to a compound of formula (I) having the following structure:

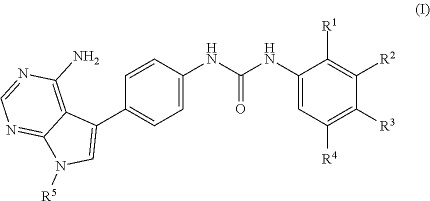

or a stereoisomer, pharmaceutically acceptable salt, oxide, or solvate thereof, wherein
R¹, R², R³, and R⁴ are independently selected from the group consisting of H, F, CF₃, C₂F₅, C₃F₇, C₄F₉, C₅F₁₁, C₆F₁₃, C₇F₁₅, and C₈F₁₇; and
R⁵ is a C₁-C₆ alkyl or a C₃-C₇ cycloalkyl.

As used above, and throughout the description herein, the following terms, unless otherwise indicated, shall be understood to have the following meanings. If not defined otherwise herein, all technical and scientific terms used herein have the same meaning as is commonly understood by one of ordinary skill in the art to which this technology belongs.

The term “alkyl” means an aliphatic hydrocarbon group which may be straight or branched having about 1 to about 6 carbon atoms in the chain (or the number of carbons designated by “Cₙ-Cₙ”, where n is the numerical range of carbon atoms). Branched means that one or more lower alkyl groups such as methyl, ethyl, or propyl are attached to a linear alkyl chain. Exemplary alkyl groups include methyl, ethyl, n-propyl, i-propyl, n-butyl, t-butyl, n-pentyl, and 3-pentyl.

The term “cycloalkyl” means a non-aromatic, saturated or unsaturated, mono- or multi-cyclic ring system of about 3 to about 7 carbon atoms, or of about 5 to about 7 carbon atoms, and which may include at least one double bond. Exemplary cycloalkyl groups include, without limitation, cyclopropyl, cyclobutyl, cyclopentyl, cyclohexyl, cycloheptyl, cyclopropenyl, cyclobutenyl, cyclopentenyl, cyclophenyl, anti-bicyclopropane, and syn-tricyclopropane.

In some embodiments of the compounds of formula (I),
R¹ is H;
R² is H;
R³ is H;
R⁴ is selected from the group consisting of CF₃, C₂F₅, C₃F₇, and C₄F₉; and
R⁵ is a C₃ alkyl. In accordance with these embodiments, exemplary compounds of formula (I) include, but are not limited to,

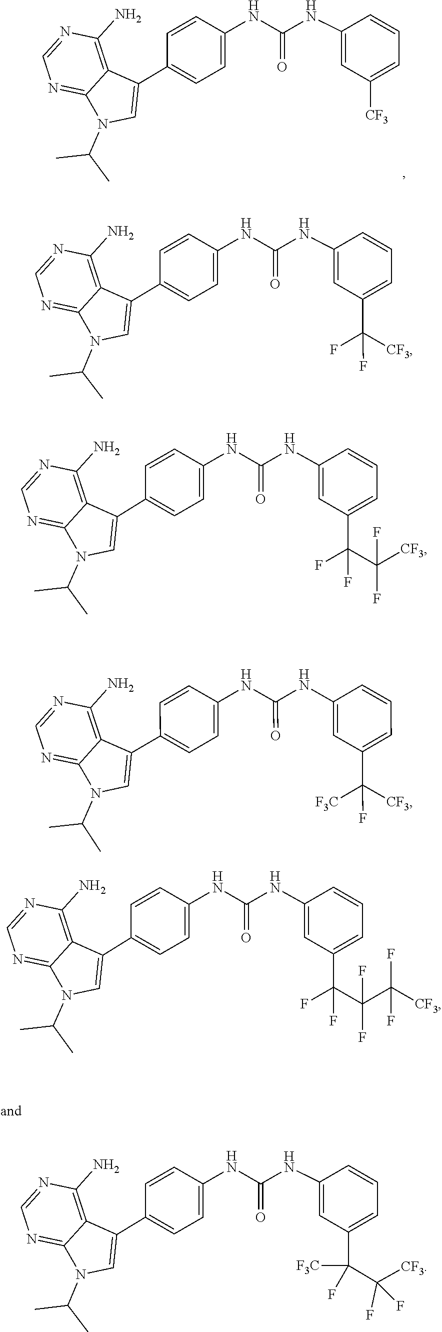

In some embodiments of the compounds of formula (I)
R¹ is F;
R² is H;
R³ is H; and
R⁴ is selected from the group consisting of CF₃, C₂F₅, C₃F₇, and C₄F₉. In accordance with these embodiments, exemplary compounds of formula (I) include, but are not limited to,

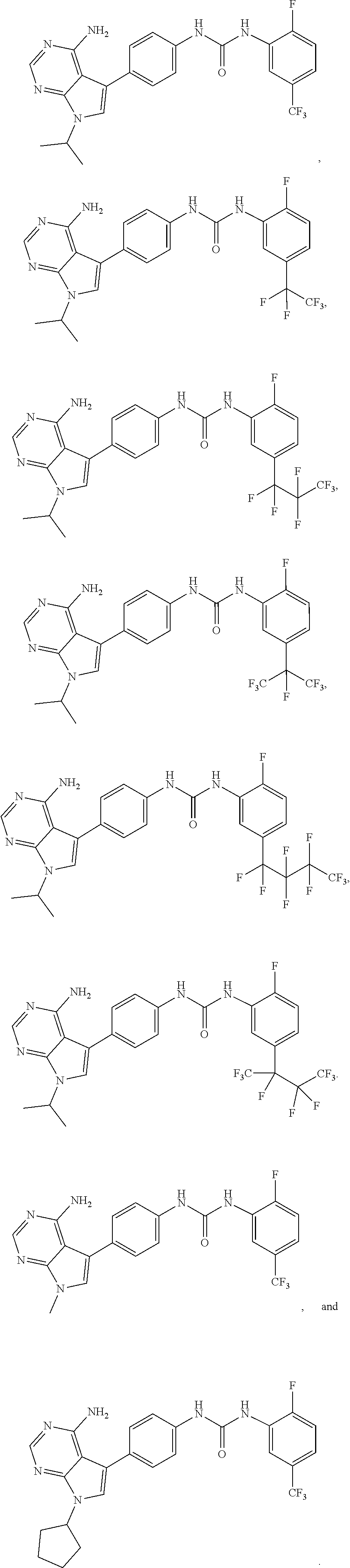

In some embodiments of the compounds of formula (I)
R¹ is H;
R² is H;
R³ is selected from the group consisting of CF₃, C₂F₅, C₃F₇, and C₄F₉;
R⁴ is H; and
R⁵ is a C₃ alkyl. In accordance with these embodiments, exemplary compounds of formula (I) include, but are not limited to,

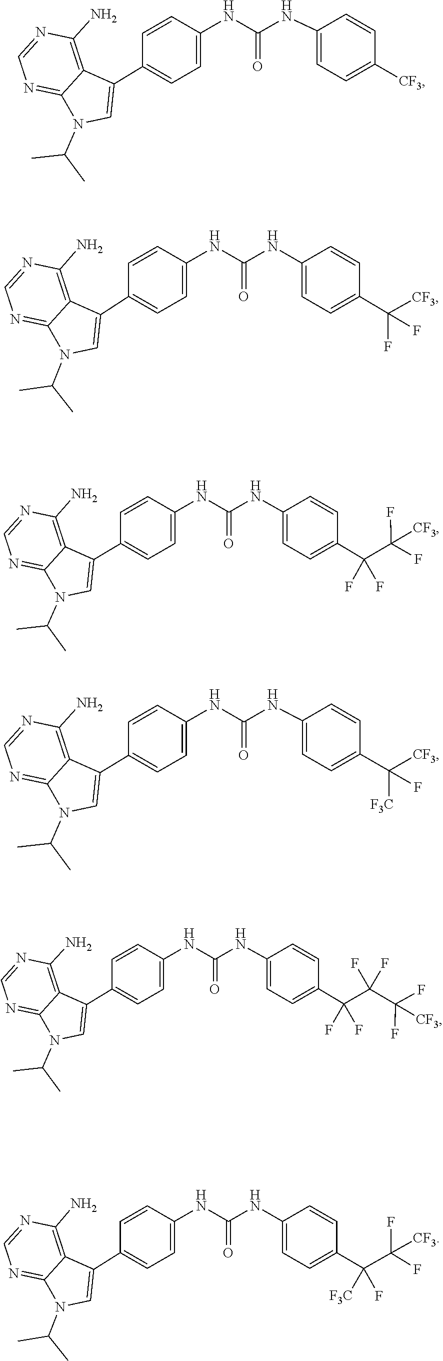 [SMILES: CC(C)n1cc(-c2ccc(NC(=O)Nc3ccc(C(F)(F)F)cc3)cc2)c2c(N)ncnc21 ; CC(C)n1cc(-c2ccc(NC(=O)Nc3ccc(C(F)(F)C(F)(F)F)cc3)cc2)c2c(N)ncnc21 ; CC(C)n1cc(-c2ccc(NC(=O)Nc3ccc(C(F)(F)C(F)(F)C(F)(F)F)cc3)cc2)c2c(N)ncnc21 ; CC(C)n1cc(-c2ccc(NC(=O)Nc3ccc(C(F)(C(F)(F)F)C(F)(F)F)cc3)cc2)c2c(N)ncnc21 ; CC(C)n1cc(-c2ccc(NC(=O)Nc3ccc(C(F)(F)C(F)(F)C(F)(F)C(F)(F)F)cc3)cc2)c2c(N)ncnc21 ; CC(C)n1cc(-c2ccc(NC(=O)Nc3ccc(C(F)(C(F)(F)F)C(F)(F)C(F)(F)F)cc3)cc2)c2c(N)ncnc21]

Another aspect of the present disclosure relates to a compound of formula (II) having the following structure:

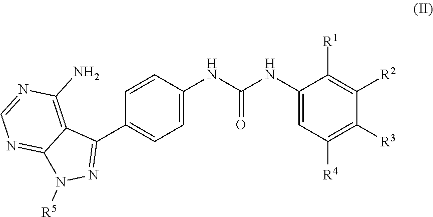

or a stereoisomer, pharmaceutically acceptable salt, oxide, or solvate thereof, wherein
R¹, R², and R³ are independently selected from the group consisting of H, F, CF₃, C₂F₅, C₃F₇, C₄F₉, C₅F₁₁, C₆F₁₃, C₇F₁₅, and C₈F₁₇;
R⁴ is selected from the group consisting of H, F, C₂F₅, C₃F₇, C₄F₉, C₅F₁₁, C₆F₁₃, C₇F₁₅, and C₈F₁₇; and
R⁵ is a C₁-C₆ alkyl or a C₃-C₇ cycloalkyl.

In some embodiments of the compounds of formula (II)
R¹ is H;
R² is H;
R³ is H;
R⁴ is selected from the group consisting of C₂F₅, C₃F₇, and C₄F₉; and
R⁵ is a C₃ alkyl. In accordance with these embodiments, exemplary compounds of formula (II) include, but are not limited to,

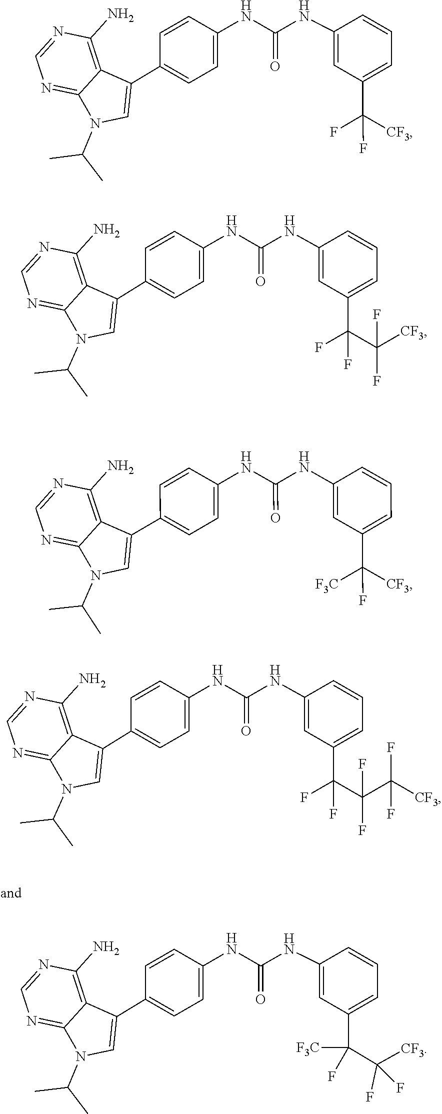 [SMILES: CC(C)n1cc(-c2ccc(NC(=O)Nc3cccc(C(F)(F)C(F)(F)F)c3)cc2)c2c(N)ncnc21 ; CC(C)n1cc(-c2ccc(NC(=O)Nc3cccc(C(F)(F)C(F)(F)C(F)(F)F)c3)cc2)c2c(N)ncnc21 ; CC(C)n1cc(-c2ccc(NC(=O)Nc3cccc(C(F)(C(F)(F)F)C(F)(F)F)c3)cc2)c2c(N)ncnc21 ; CC(C)n1cc(-c2ccc(NC(=O)Nc3cccc(C(F)(F)C(F)(F)C(F)(F)C(F)(F)F)c3)cc2)c2c(N)ncnc21 ; CC(C)n1cc(-c2ccc(NC(=O)Nc3cccc(C(F)(C(F)(F)F)C(F)(F)C(F)(F)F)c3)cc2)c2c(N)ncnc21]

In some embodiments of the compounds of formula (II)
R¹ is F;
R² s H;
R³ is H;
R⁴ is selected from the group consisting of C₂F₅, C₃F₇, and C₄F₉; and
R⁵ is a C₃ alkyl. In accordance with these embodiments, exemplary compounds of formula (II) include, but are not limited to,

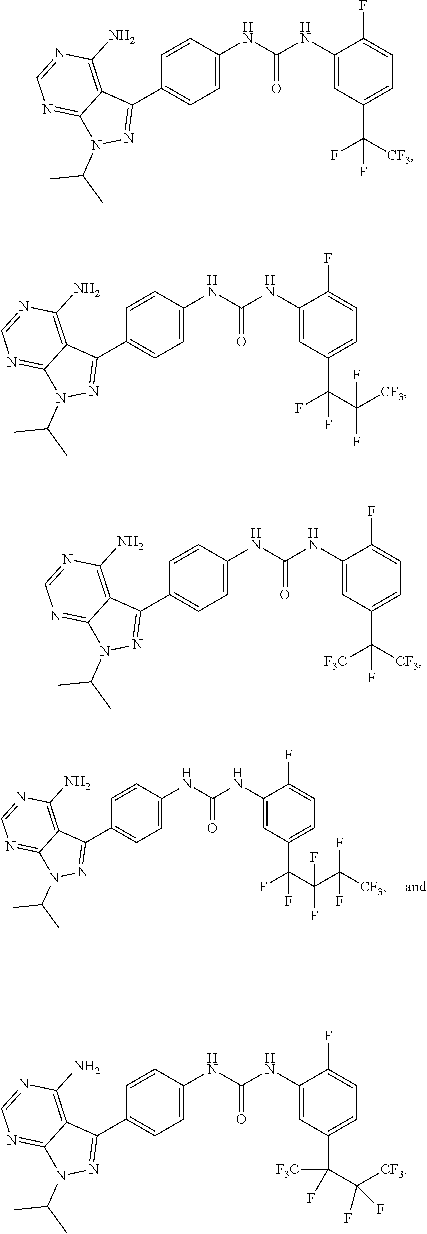 [SMILES: CC(C)n1nc(-c2ccc(NC(=O)Nc3cc(C(F)(F)C(F)(F)F)ccc3F)cc2)c2c(N)ncnc21 ; CC(C)n1nc(-c2ccc(NC(=O)Nc3cc(C(F)(F)C(F)(F)C(F)(F)F)ccc3F)cc2)c2c(N)ncnc21 ; CC(C)n1nc(-c2ccc(NC(=O)Nc3cc(C(F)(C(F)(F)F)C(F)(F)F)ccc3F)cc2)c2c(N)ncnc21 ; CC(C)n1nc(-c2ccc(NC(=O)Nc3cc(C(F)(F)C(F)(F)C(F)(F)C(F)(F)F)ccc3F)cc2)c2c(N)ncnc21 ; CC(C)n1nc(-c2ccc(NC(=O)Nc3cc(C(F)(C(F)(F)F)C(F)(F)C(F)(F)F)ccc3F)cc2)c2c(N)ncnc21]

In some embodiments of the compounds of formula (II)
R¹ is H;
R² is H;
R⁴ is selected from the group consisting of CF₃, C₂F₅, C₃F₇, and C₄F₉;
R⁴ is H; and
R⁵ is a C₃ alkyl. In accordance with these embodiments, exemplary compounds of formula (II) include, but are not limited to,

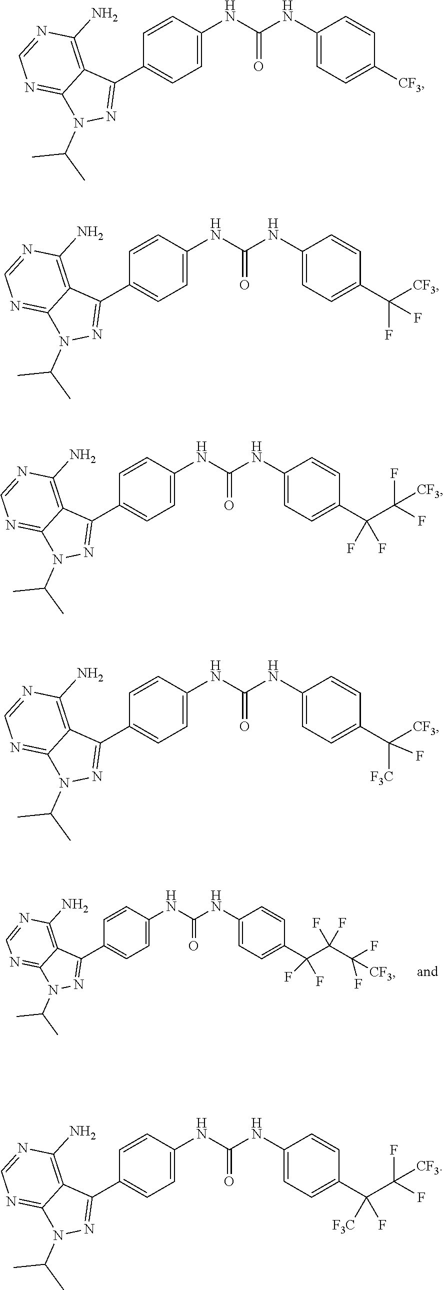 [SMILES: CC(C)n1nc(-c2ccc(NC(=O)Nc3ccc(C(F)(F)F)cc3)cc2)c2c(N)ncnc21 ; CC(C)n1nc(-c2ccc(NC(=O)Nc3ccc(C(F)(F)C(F)(F)F)cc3)cc2)c2c(N)ncnc21 ; CC(C)n1nc(-c2ccc(NC(=O)Nc3ccc(C(F)(F)C(F)(F)C(F)(F)F)cc3)cc2)c2c(N)ncnc21 ; CC(C)n1nc(-c2ccc(NC(=O)Nc3ccc(C(F)(C(F)(F)F)C(F)(F)F)cc3)cc2)c2c(N)ncnc21 ; CC(C)n1nc(-c2ccc(NC(=O)Nc3ccc(C(F)(F)C(F)(F)C(F)(F)C(F)(F)F)cc3)cc2)c2c(N)ncnc21 ; CC(C)n1nc(-c2ccc(NC(=O)Nc3ccc(C(F)(C(F)(F)F)C(F)(F)C(F)(F)F)cc3)cc2)c2c(N)ncnc21]

In some embodiments of the compounds of formula (I) and the compounds of formula (II) described herein, only one of the substituents R¹, R², R³, and R⁴ is a perfluoroalkyl group selected from the group consisting of C₂F₅, C₃F₇, C₄F₉, C₅F₁₁, C₆F₁₃, C₇F₁₅, and C₈F₁₇, and the remaining R¹, R², R³, and R⁴ substituents are selected from H, F, or CF₃. In some embodiments, the remaining R¹, R², R³, and R⁴ substituents are all H. In some embodiments, the one perfluoroalkyl group of the substituents R¹, R², R³, and R⁴ is in the para position. In some embodiments, the one perfluoroalkyl group of the substituents R¹, R², R³, and R⁴ is in the meta position. In some embodiments, R⁵ is a branched C₃ alkyl.

In some embodiments of the compounds of formula (I) and the compounds of formula (II) described herein, only one of the substituents R¹, R², R³, and R⁴ is a perfluoroalkyl group selected from the group consisting of C₃F₇ or C₄F₉, and the remaining R¹, R², R³, and R⁴ substituents are selected from H, F, or CF₃. In some embodiments, the remaining R¹, R², R³, and R⁴ substituents are all H. In some embodiments, the one perfluoroalkyl group of the substituents R¹, R², R³, and R⁴ is in the para position. In some embodiments, the one perfluoroalkyl group of the substituents R¹, R², R³, and R⁴ is in the meta position. In some embodiments, R⁵ is a branched C₃ alkyl.

In some embodiments of the compounds of formula (I) and the compounds of formula (II) described herein, the pyrazolo- and pyrrolopyrimidine compounds are functionalized with a -sC₂F₅ in the meta- or para-position.

In some embodiments of the compounds of formula (I) and the compounds of formula (II) described herein, the pyrazolo- and pyrrolopyrimidine compounds are functionalized with a para-substituted straight chain -nC₃F₇ or branched -iC₃F₇.

In some embodiments of the present disclosure, the compounds of formula (I) and the compounds of formula (II) may be optionally substituted.

The phrases “substituted or unsubstituted” and “optionally substituted” mean a group may (but does not necessarily) have a substituent at each substitutable atom of the group (including more than one substituent on a single atom), and the identity of each substituent is independent of the others.

The term “substituted” means that one or more hydrogen on a designated atom is replaced with a selection from the indicated group, provided that the designated atom's normal valency is not exceeded. “Unsubstituted” atoms bear all of the hydrogen atoms dictated by their valency. When a substituent is oxo (i.e., ═O), then 2 hydrogens on the atom are replaced. Combinations of substituents and/or variables are permissible only if such combinations result in stable compounds. By “stable compound” it is meant a compound that is sufficiently robust to survive isolation to a useful degree of purity from a reaction mixture and formulation into an efficacious therapeutic agent. Examples of substitution include, but are not limited to, substitution with halogens, alkyl groups, perfluoroalkyl groups, alkoxy groups, aryl groups, heteroaryl groups, and heterocycles.

As used herein, the term “halogen” means fluoro, chloro, bromo, or iodo.

The term “alkoxy” means groups of from 1 to 6 carbon atoms of a straight, branched, or cyclic configuration and combinations thereof attached to the parent structure through an oxygen. Examples include methoxy, ethoxy, propoxy, isopropoxy, butoxy, cyclopropyloxy, cyclohexyloxy, and the like. Alkoxy also includes methylenedioxy and ethylenedioxy in which each oxygen atom is bonded to the atom, chain, or ring from which the methylenedioxy or ethylenedioxy group is pendant so as to form a ring. Thus, for example, phenyl substituted by alkoxy may be, for example,

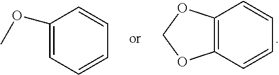

The term “aryl” means an aromatic monocyclic or multi-cyclic (polycyclic) ring system of 6 to about 19 carbon atoms, or of 6 to about 10 carbon atoms, and includes arylalkyl groups. The ring system of the aryl group may be optionally substituted. Representative aryl groups of the present disclosure include, but are not limited to, groups such as phenyl, naphthyl, azulenyl, phenanthrenyl, anthracenyl, fluorenyl, pyrenyl, triphenylenyl, chrysenyl, and naphthacenyl.

The term “heteroaryl” means an aromatic monocyclic or multi-cyclic ring system of about 5 to about 19 ring atoms, or about 5 to about 10 ring atoms, in which one or more of the atoms in the ring system is/are element(s) other than carbon, for example, nitrogen, oxygen, or sulfur. In the case of multi-cyclic ring system, only one of the rings needs to be aromatic for the ring system to be defined as “heteroaryl.” Preferred heteroaryls contain about 5 to 6 ring atoms. The prefix aza, oxa, thia, or thio before heteroaryl means that at least a nitrogen, oxygen, or sulfur atom, respectively, is present as a ring atom. A nitrogen, carbon, or sulfur atom in the heteroaryl ring may be optionally oxidized; the nitrogen may optionally be quaternized. Representative heteroaryls include pyridyl, 2-oxo-pyridinyl, pyrimidinyl, pyridazinyl, pyrazinyl, triazinyl, furanyl, pyrrolyl, thiophenyl, pyrazolyl, imidazolyl, oxazolyl, isoxazolyl, thiazolyl, isothiazolyl, triazolyl, oxadiazolyl, thiadiazolyl, tetrazolyl, indolyl, isoindolyl, benzofuranyl, benzothiophenyl, indolinyl, 2-oxoindolinyl, dihydrobenzofuranyl, dihydrobenzothiophenyl, indazolyl, benzimidazolyl, benzooxazolyl, benzothiazolyl, benzoisoxazolyl, benzoisothiazolyl, benzotriazolyl, benzo[1,3]dioxolyl, quinolinyl, isoquinolinyl, quinazolinyl, cinnolinyl, pthalazinyl, quinoxalinyl, 2,3-dihydro-benzo[1,4]dioxinyl, benzo[1,2,3]triazinyl, benzo[1,2,4]triazinyl, 4H-chromenyl, indolizinyl, quinolizinyl, 6aH-thieno[2,3-d]imidazolyl, 1H-pyrrolo[2,3-b]pyridinyl, imidazo[1,2-a]pyridinyl, pyrazolo[1,5-a]pyridinyl, [1,2,4]triazolo[4,3-a]pyridinyl, [1,2,4]triazolo[1,5-a]pyridinyl, thieno[2,3-b]furanyl, thieno[2,3-b]pyridinyl, thieno[3,2-b]pyridinyl, furo[2,3-b]pyridinyl, furo[3,2-b]pyridinyl, thieno[3,2-d]pyrimidinyl, furo[3,2-d]pyrimidinyl, thieno[2,3-b]pyrazinyl, imidazo[1,2-a]pyrazinyl, 5,6,7,8-tetrahydroimidazo[1,2-a]pyrazinyl, 6,7-dihydro-4H-pyrazolo[5,1-c][1,4]oxazinyl, 2-oxo-2,3-dihydrobenzo[d]oxazolyl, 3,3-dimethyl-2-oxoindolinyl, 2-oxo-2,3-dihydro-1H-pyrrolo[2,3-b]pyridinyl, benzo[c][1,2,5]oxadiazolyl, benzo[c][1,2,5]thiadiazolyl, 3,4-dihydro-2H-benzo[b][1,4]oxazinyl, 5,6,7,8-tetrahydro-[1,2,4]triazolo[4,3-a]pyrazinyl, [1,2,4]triazolo[4,3-a]pyrazinyl, 3-oxo-[1,2,4]triazolo[4,3-a]pyridin-2(3H)-yl, and the like.

As used herein, “heterocycle” refers to a stable 3- to 18-membered ring (radical) of carbon atoms and from one to five heteroatoms selected from nitrogen, oxygen, and sulfur. The heterocycle may be a monocyclic or a polycyclic ring system, which may include fused, bridged, or spiro ring systems; and the nitrogen, carbon, or sulfur atoms in the heterocycle may be optionally oxidized; the nitrogen atom may be optionally quaternized; and the ring may be partially or fully saturated. Examples of such heterocycles include, without limitation, azepinyl, azocanyl, pyranyl dioxanyl, dithianyl, 1,3-dioxolanyl, tetrahydrofuryl, dihydropyrrolidinyl, decahydroisoquinolyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, 2-oxoazepinyl, oxazolidinyl, oxiranyl, piperidinyl, piperazinyl, 4-piperidonyl, pyrrolidinyl, pyrazolidinyl, thiazolidinyl, tetrahydropyranyl, thiamorpholinyl, thiamorpholinyl sulfoxide, and thiamorpholinyl sulfone.

Further heterocycles and heteroaryls are described in Katritzky et al., eds., Comprehensive Heterocyclic Chemistry: The Structure, Reactions, Synthesis and Use of Heterocyclic Compounds, Vol. 1-8, Pergamon Press, N.Y. (1984), which is hereby incorporated by reference in its entirety.

By “compound(s) of the disclosure” and equivalent expressions, it is meant compounds herein described, which expression includes the prodrugs, the pharmaceutically acceptable salts, the oxides, and the solvates, e.g. hydrates, where the context so permits.

Compounds described herein may contain one or more asymmetric centers and may thus give rise to enantiomers, diastereomers, and other stereoisomeric forms. Each chiral center may be defined, in terms of absolute stereochemistry, as (R)- or (S)-. The present disclosure is meant to include all such possible isomers, as well as mixtures thereof, including racemic and optically pure forms. Optically active (R)- and (S)-, (−)- and (+)-, or (D)- and (L)-isomers may be prepared using chiral synthons or chiral reagents, or resolved using conventional techniques. All tautomeric forms are also intended to be included.

In some embodiments of the present disclosure, the compounds of formula (I) and/or of formula (II) may be mixed with a carrier to form a composition. In some embodiments, the carrier is a pharmaceutically-acceptable carrier

The term “pharmaceutically acceptable” means it is, within the scope of sound medical judgment, suitable for use in contact with the cells of humans and lower animals without undue toxicity, irritation, allergic response, and the like, and are commensurate with a reasonable benefit/risk ratio.

As would be understood by a person of ordinary skill in the art, the recitation of “a compound” is intended to include salts, solvates, oxides, and inclusion complexes of that compound as well as any stereoisomeric form, or a mixture of any such forms of that compound in any ratio. Thus, in accordance with some embodiments of the disclosure, a compound as described herein, including in the contexts of pharmaceutical compositions, methods of treatment, and compounds per se, is provided as the salt form.

The term “solvate” refers to a compound in the solid state, where molecules of a suitable solvent are incorporated in the crystal lattice. A suitable solvent for therapeutic administration is physiologically tolerable at the dosage administered. Examples of suitable solvents for therapeutic administration are ethanol and water. When water is the solvent, the solvate is referred to as a hydrate. In general, solvates are formed by dissolving the compound in the appropriate solvent and isolating the solvate by cooling or using an antisolvent. The solvate is typically dried or azeotroped under ambient conditions.

Inclusion complexes are described in Remington, The Science and Practice of Pharmacy, 19th Ed. 1:176-177 (1995), which is hereby incorporated by reference in its entirety. The most commonly employed inclusion complexes are those with cyclodextrins, and all cyclodextrin complexes, natural and synthetic, are specifically encompassed by the present disclosure.

Compounds of the present disclosure may be prepared according to methods of synthesis described in the Examples infra.

A further aspect of the present disclosure relates to a method of treating cancer in a subject. This method includes administering to a subject in need thereof a compound of formula (I) having the following structure:

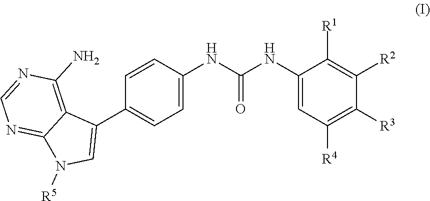

or a stereoisomer, pharmaceutically acceptable salt, oxide, or solvate thereof, wherein
R¹, R², R³, and R⁴ are independently selected from the group consisting of H, F, CF₃, C₂F₅, C₃F₇, C₄F₉, C₅F₁₁, C₆F₁₃, C₇F₁₅, and C₈F₁₇; and
R⁵ is a C₁-C₆ alkyl or a C₃-C₇ cycloalkyl.

Yet another aspect of the present disclosure relates to a method of treating cancer in a subject. This method includes administering to a subject in need thereof a compound of formula (II) having the following structure:

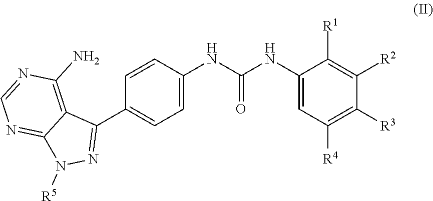

or a stereoisomer, pharmaceutically acceptable salt, oxide, or solvate thereof, wherein
R¹, R², and R³ are independently selected from the group consisting of H, F, CF₃, C₂F₅, C₃F₇, C₄F₉, C₅F₁₁, C₆F₁₃, C₇F₁₅, and C₈F₁₇;
R⁴ is selected from the group consisting of H, F, C₂F₅, C₃F₇, C₄F₉, C₅F₁₁, C₆F₁₃, C₇F₁₅, and C₈F₁₇; and
R⁵ is a C₁-C₆ alkyl or a C₃-C₇ cycloalkyl.

The term “treating” means amelioration or relief from the symptoms and/or effects associated with the disease(s) or disorder(s) described herein.

The term “pharmaceutically acceptable salt” refers to salts prepared from pharmaceutically acceptable non-toxic acids or bases including inorganic acids and bases and organic acids and bases.

In some embodiments of the present disclosure, the cancer is selected from the group consisting of cancer is selected from the group consisting of Acute Lymphoblastic Leukemia (ALL), Acute Myeloid Leukemia (AML), Adrenocortical Carcinoma, Adrenal Cortex Cancer, Anal Cancer, Appendix Cancer, Astrocytomas, Atypical Teratoid/Rhabdoid Tumor, Basal Cell Carcinoma, Extrahepatic Bile Duct Cancer, Bladder Cancer, Bone Cancer, Brain Tumors, Breast Cancer, Bronchial Tumors, Burkitt Lymphoma, Carcinoid Tumor, Cardiac (Heart) Tumors, Cervical Cancer, Cholangiocarcinoma, Chronic Lymphocytic Leukemia (CLL), Chronic Myelogenous Leukemia (CML), Chronic Myeloproliferative Neoplasms, Colorectal Cancer, Cutaneous T-Cell Lymphoma, Ductal Carcinoma In Situ (DCIS), Endometrial Cancer, Ependymoma, Esophageal, Esthesioneuroblastoma, Ewing Sarcoma, Intraocular Melanoma, Retinoblastoma, Malignant Fibrous Histiocytoma of Bone, Osteosarcoma, Gallbladder Cancer, Gastric (Stomach) Cancer, Gastrointestinal Carcinoid Tumor, Gastrointestinal Stromal Tumors (GIST), Gestational Trophoblastic Disease, Gliomas, Hairy Cell Leukemia, Head and Neck Cancer, Hepatocellular (Liver) Cancer, Langerhans Cell Histiocytosis, Hodgkin Lymphoma, Hypopharyngeal Cancer, Intraocular Melanoma, Kaposi Sarcoma, Kidney cancer, Langerhans [symbol] sis, Leukemia, Lung Cancer, Lymphoma, Medullary Thyroid Carcinoma, Melanoma, Intraocular (Eye) Melanoma, Merkel Cell Carcinoma, Malignant Mesothelioma, Metastatic Squamous Neck Cancer with Occult Primary, Multiple Endocrine Neoplasia Syndromes, Multiple Myeloma/Plasma Cell Neoplasms, Myelodysplastic Syndromes, Myelodysplastic/Myeloproliferative Neoplasms, and Chronic Myeloproliferative Neoplasms, Chronic Myelogenous Leukemia (CML), Acute Myeloid Leukemia (AML), Nasopharyngeal Cancer, Neuroblastoma, Non-Hodgkin Lymphoma, Non-Small Cell Lung Cancer, Oral Cancer, Lip and Oral Cavity Cancer, Oropharyngeal Cancer, Ovarian Cancer, Pancreatic Cancer and Pancreatic Neuroendocrine Tumors (Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Plasma Cell Neoplasm/Multiple Myeloma, Primary Central Nervous System (CNS) Lymphoma, Prostate Cancer, Rectal Cancer, Renal Cell (Kidney) Cancer, Retinoblastoma, Rhabdomyosarcoma, Salivary Gland Cancer, Sarcoma, Sezary Syndrome, Small Cell Lung Cancer, Small Intestine Cancer, Soft Tissue Sarcoma, Squamous Cell Carcinoma, Squamous Neck Cancer, Stomach (Gastric) Cancer, T-Cell Lymphoma, Testicular Cancer, Throat Cancer, Thymoma and Thymic Carcinoma, Thyroid Cancer, Transitional Cell Cancer of the Renal Pelvis and Ureter, Urethral Cancer, Uterine Cancer, Endometrial and Uterine Sarcoma, Vaginal Cancer, Vulvar Cancer, Waldenstrom Macroglobulinemia, and Wilms Tumor.

Administering may be carried out orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes. Compounds may be administered alone or with suitable pharmaceutical carriers, and can be in solid or liquid form, such as tablets, capsules, powders, solutions, suspensions, or emulsions.

It will be understood that the specific dose level for any particular patient will depend upon a variety of factors, including the activity of the specific compound employed, the age, body weight, general health, sex, diet, time of administration, route of administration, rate of excretion, drug combination, and the severity of the particular disease undergoing therapy.

While it may be possible for compounds of formula (I), and of formula (II) to be administered as the raw chemical, they may also be administered as a pharmaceutical composition.

Thus, a further aspect of the present disclosure relates to a composition comprising a compound of formula (I) or formula (II) described herein and a carrier.

In accordance with some embodiments of the present disclosure, there is provided a pharmaceutical composition comprising a compound of formula (I) and/or a compound of formula (II) or a pharmaceutically acceptable salt or solvate thereof, together with one or more carriers (e.g., a pharmaceutically acceptable carrier(s)) thereof and optionally one or more other therapeutic ingredients. The carrier(s) must be “acceptable” in the sense of being compatible with the other ingredients of the formulation and not deleterious to the recipient thereof. Furthermore, notwithstanding the statements herein regarding the term “compound” including salts thereof as well, so that independent claims reciting “a compound” will be understood as referring to salts thereof as well, if in an independent claim reference is made to a compound or a pharmaceutically acceptable salt thereof, it will be understood that claims which depend from that independent claim which refer to such a compound also include pharmaceutically acceptable salts of the compound, even if explicit reference is not made to the salts in the dependent claim.

Formulations include those suitable for oral, parenteral (including subcutaneous, intradermal, intramuscular, intravenous, and intraarticular), rectal and topical (including dermal, buccal, sublingual, and intraocular) administration. The most suitable route may depend upon the condition and disorder of the recipient. The formulations may conveniently be presented in unit dosage form and may be prepared by any of the methods well known in the art of pharmacy. Such methods include the step of bringing into association a compound of formula (I) and/or compounds of formula (II) or pharmaceutically acceptable salts or solvates thereof (“active ingredient”) with the carrier, which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations suitable for oral administration may be presented as discrete units such as capsules, cachets, or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary, or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may optionally be coated or scored and may be formulated so as to provide sustained, delayed or controlled release of the active ingredient therein.

The pharmaceutical compositions may include a “pharmaceutically acceptable inert carrier,” and this expression is intended to include one or more inert excipients, which include, for example and without limitation, starches, polyols, granulating agents, microcrystalline cellulose, diluents, lubricants, binders, disintegrating agents, and the like. If desired, tablet dosages of the disclosed compositions may be coated by standard aqueous or nonaqueous techniques. “Pharmaceutically acceptable carrier” also encompasses controlled release means.

Pharmaceutical compositions may also optionally include other therapeutic ingredients, anti-caking agents, preservatives, sweetening agents, colorants, flavors, desiccants, plasticizers, dyes, and the like. Any such optional ingredient must be compatible with the compounds of formula (I) and/or the compounds of formula (II) to insure the stability of the formulation. The composition may contain other additives as needed including, for example, lactose, glucose, fructose, galactose, trehalose, sucrose, maltose, raffinose, maltitol, melezitose, stachyose, lactitol, palatinite, starch, xylitol, mannitol, myoinositol, and the like, and hydrates thereof, and amino acids, for example alanine, glycine and betaine, and peptides and proteins, for example albumen.

Examples of excipients for use as the pharmaceutically acceptable carriers and the pharmaceutically acceptable inert carriers and the aforementioned additional ingredients include, but are not limited to, binders, fillers, disintegrants, lubricants, anti-microbial agents, and coating agents.

Dose ranges for adult humans vary, but may generally be from about 0.005 mg to 10 g/day orally. Tablets or other forms of presentation provided in discrete units may conveniently contain an amount of compound of formula (I) and/or a compound of formula (II) which is effective at such dosage or as a multiple of the same, for instance, units containing 5 mg to 500 mg, or around 10 mg to 200 mg. The precise amount of compound administered to a patient will be the responsibility of the attendant physician. However, the dose employed will depend on a number of factors, including the age and sex of the patient, the precise disorder being treated, and its severity.

A dosage unit (e.g., an oral dosage unit) can include from, for example, 1 to 30 mg, 1 to 40 mg, 1 to 100 mg, 1 to 300 mg, 1 to 500 mg, 2 to 500 mg, 3 to 100 mg, 5 to 20 mg, 5 to 100 mg (e.g., 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 150 mg, 200 mg, 250 mg, 300 mg, 350 mg, 400 mg, 450 mg, 500 mg) of the compounds described herein.

Additional information about pharmaceutical compositions and their formulation is described in Remington: The Science and Practice of Pharmacy, 20^{th} Edition, 2000, which is hereby incorporated by reference in its entirety.

The compounds of the present disclosure can be administered, e.g., by intravenous injection, intramuscular injection, subcutaneous injection, intraperitoneal injection, topical, sublingual, intraarticular (in the joints), intradermal, buccal, ophthalmic (including intraocular), intranasaly (including using a cannula), or by other routes. The compounds can be administered orally, e.g., as a tablet or cachet containing a predetermined amount of the active ingredient, gel, pellet, paste, syrup, bolus, electuary, slurry, capsule, powder, granules, as a solution or a suspension in an aqueous liquid or a non-aqueous liquid, as an oil-in-water liquid emulsion or a water-in-oil liquid emulsion, via a micellar formulation (see, e.g., PCT Publication No. WO 97/11682, which is hereby incorporated by reference in its entirety) via a liposomal formulation (see, e.g., EP Patent No. 736299, PCT Publication No. WO 99/59550, and PCT Publication No. WO 97/13500, which is hereby incorporated by reference in its entirety), via formulations described in PCT Publication No. WO 03/094886 (which is hereby incorporated by reference in its entirety) or in some other form. The agents can also be administered transdermally (i.e., via reservoir-type or matrix-type patches, microneedles, thermal poration, hypodermic needles, iontophoresis, electroporation, ultrasound, or other forms of sonophoresis, jet injection, or a combination of any of the preceding methods (Prausnitz et al. Nature Reviews Drug Discovery 3:115 (2004), which is hereby incorporated by reference in its entirety). The agents can be administered locally.

The compounds can be administered in the form a suppository or by other vaginal or rectal means. The compounds can be administered in a transmembrane formulation as described in PCT Publication No. WO 90/07923, which is hereby incorporated by reference in its entirety. The compounds can be administered non-invasively via the dehydrated particles, such as those described in U.S. Pat. No. 6,485,706, which is hereby incorporated by reference in its entirety. The compounds can be administered in an enteric-coated drug formulation such as those described in PCT Publication No. WO 02/49621, which is hereby incorporated by reference in its entirety. The compounds can be administered intranasaly using formulations such as those described in U.S. Pat. No. 5,179,079, which is hereby incorporated by reference in its entirety. Formulations suitable for parenteral injection are described in PCT Publication No. WO 00/62759, which is hereby incorporated by reference in its entirety. The compounds can be administered using the casein formulation described in U.S. Patent Application Publication No. 2003/0206939 and PCT Publication No. WO 00/06108, which are hereby incorporated by reference in their entirety. The compounds can be administered using the particulate formulations described in U.S. Patent Application Publication No. 20020034536, which is hereby incorporated by reference in its entirety.

The compounds, alone or in combination with other suitable components, can be administered by pulmonary route utilizing several techniques including, but not limited to, intratracheal instillation (delivery of solution into the lungs by syringe), intratracheal delivery of liposomes, insufflation (administration of powder formulation by syringe or any other similar device into the lungs), and aerosol inhalation. Aerosols (e.g., jet or ultrasonic nebulizers, metered-dose inhalers (“MIDIs”), and dry-Powder inhalers (“DPIs”)) can also be used in intranasal applications. Aerosol formulations are stable dispersions or suspensions of solid material and liquid droplets in a gaseous medium and can be placed into pressurized acceptable propellants, such as hydrofluoroalkanes (IFAs, i.e., IFA-134a and IFA-227, or a mixture thereof), dichlorodifluoromethane (or other chlorofluorocarbon propellants such as a mixture of Propellants 11, 12, and/or 114), propane, nitrogen, and the like. Pulmonary formulations may include permeation enhancers such as fatty acids, and saccharides, chelating agents, enzyme inhibitors (e.g., protease inhibitors), adjuvants (e.g., glycocholate, surfactin, span 85, and nafamostat), preservatives (e.g., benzalkonium chloride or chlorobutanol), and ethanol (normally up to 5% but possibly up to 20%, by weight). Ethanol is commonly included in aerosol compositions as it can improve the function of the metering valve and in some cases also improve the stability of the dispersion.

Pulmonary formulations may also include surfactants which include, but are not limited to, bile salts and those described in U.S. Pat. No. 6,524,557 and references therein, which are hereby incorporated by reference in their entirety. The surfactants described in U.S. Pat. No. 6,524,557, e.g., a C₈-C₁₆ fatty acid salt, a bile salt, a phospholipid, or alkyl saccharide are advantageous in that some of them also reportedly enhance absorption of the compound in the formulation.

Also suitable are dry powder formulations comprising a therapeutically effective amount of active compound blended with an appropriate carrier and adapted for use in connection with a dry-powder inhaler. Absorption enhancers that can be added to dry powder formulations include those described in U.S. Pat. No. 6,632,456, which is hereby incorporated by reference in its entirety. PCT Publication No. WO 02/080884, which is hereby incorporated by reference in its entirety, describes new methods for the surface modification of powders. Aerosol formulations may include those described in U.S. Pat. Nos. 5,230,884 and 5,292,499; PCT Publication Nos. WO 017/8694 and 01/78696; and U.S. Patent Application Publication No. 2003/019437, 2003/0165436; and PCT Publication No. WO 96/40089 (which includes vegetable oil), which are hereby incorporated by reference in their entirety. Sustained release formulations suitable for inhalation are described in U.S. Patent Application Publication Nos. 2001/0036481, 2003/0232019, and 2004/0018243 as well as in PCT Publication Nos. WO 01/13891, 02/067902, 03/072080, and 03/079885, which are hereby incorporated by reference in their entirety.

Pulmonary formulations containing microparticles are described in PCT Publication No. WO 03/015750, U.S. Patent Application Publication No. 2003/0008013, and PCT Publication No. WO 00/00176, which are hereby incorporated by reference in their entirety. Pulmonary formulations containing stable glassy state powder are described in U.S. Patent Application Publication No. 2002/0141945 and U.S. Pat. No. 6,309,671, which are hereby incorporated by reference in their entirety. Other aerosol formulations are described in EP Patent No. 1338272, PCT Publication No. WO 90/09781, U.S. Pat. Nos. 5,348,730 and 6,436,367, PCT Publication No. WO 91/04011, and U.S. Pat. Nos. 6,294,153 and 6,290,987, which are hereby incorporated by reference in their entirety, which describe a liposomal based formulation that can be administered via aerosol or other means.

Powder formulations for inhalation are described in U.S. Patent Application Publication No. 2003/0053960 and PCT Publication No. WO 01/60341, which are hereby incorporated by reference in their entirety. The compounds can be administered intranasally as described in U.S. Patent Application Publication No. 2001/0038824, which is hereby incorporated by reference in its entirety.

Solutions of medicament in buffered saline and similar vehicles are commonly employed to generate an aerosol in a nebulizer. Simple nebulizers operate on Bernoulli's principle and employ a stream of air or oxygen to generate the spray particles. More complex nebulizers employ ultrasound to create the spray particles. Both types are well known in the art and are described in standard textbooks of pharmacy such as Sprowls' American Pharmacy and Remington's The Science and Practice of Pharmacy.

Other devices for generating aerosols employ compressed gases, usually hydrofluorocarbons and chlorofluorocarbons, which are mixed with the medicament and any necessary excipients in a pressurized container. These devices are likewise described in standard textbooks such as Sprowls and Remington.

The compounds can be incorporated into a liposome to improve half-life. The compounds can also be conjugated to polyethylene glycol (“PEG”) chains. Methods for pegylation and additional formulations containing PEG-conjugates (i.e., PEG-based hydrogels, PEG modified liposomes) can be found in Harris and Chess, Nature Reviews Drug Discovery 2:214-221, which is hereby incorporated by reference in its entirety, and the references therein. The compounds can be administered via a nanocochleate or cochleate delivery vehicle (BioDelivery Sciences International). The compounds can be delivered transmucosally (i.e., across a mucosal surface such as the vagina, eye, or nose) using formulations such as that described in U.S. Pat. No. 5,204,108, which is hereby incorporated by reference in its entirety. The compounds can be formulated in microcapsules as described in PCT Publication No. WO 88/01165, which is hereby incorporated by reference in its entirety. The compounds can be administered intra-orally using the formulations described in U.S. Patent Application Publication No. 2002/0055496, PCT Publication No. WO 00/47203, and U.S. Pat. No. 6,495,120, which are hereby incorporated by reference in their entirety. The compounds can be delivered using nanoemulsion formulations described in PCT Publication No. WO 01/91728, which is hereby incorporated by reference in its entirety.

The compounds may be delivered directly to a targeted cell/tissue/organ. Additionally, and/or alternatively, the compounds may be administered to a non-targeted area along with one or more agents that facilitate migration of the compounds to (and/or uptake by) a targeted tissue, organ, or cell. As will be apparent to one of ordinary skill in the art, the compound itself can be modified to facilitate its transport to a target tissue, organ, or cell, including its transport across the blood-brain barrier; and/or to facilitate its uptake by a target cell (e.g., its transport across cell membranes).

In vivo administration can be accomplished either via systemic administration to the subject or via targeted administration to affected tissues, organs, and/or cells, as described above. Typically, the compounds of formula (I) and/or compounds of formula (II), will be administered to a patient in a vehicle that delivers the compound(s) to the target cell, tissue, or organ. Typically, the compounds of the present disclosure will be administered as a pharmaceutical formulation, such as those described above.

Contacting (including in vivo administration) can be carried out as frequently as required and for a duration that is suitable to provide the desired effect. For example, contacting can be carried out once or multiple times, and in vivo administration can be carried out with a single sustained-release dosage formulation or with multiple (e.g., daily) doses.

The amount to be administered will, of course, vary depending upon the particular conditions and treatment regimen. The amount/dose required to obtain the desired effect may vary depending on the agent, formulation, cell type, culture conditions (for ex vivo embodiments), the duration for which treatment is desired, and, for in vivo embodiments, the individual to whom the agent is administered.

Effective amounts can be determined empirically by those of skill in the art. For example, this may involve assays in which varying amounts of the compound of the disclosure are administered to cells in culture and the concentration effective for obtaining the desired result is calculated. Determination of effective amounts for in vivo administration may also involve in vitro assays in which varying doses of agent are administered to cells in culture and the concentration of agent effective for achieving the desired result is determined in order to calculate the concentration required in vivo. Effective amounts may also be based on in vivo animal studies.

The compounds can be administered alone or as an active ingredient of a pharmaceutical formulation, such as those described above. The compounds can be administered in a form where the active ingredient is substantially pure.

A further aspect of the present disclosure relates to a method of treating a subject for a diabetes. This method involves administering to a subject in need of treatment for diabetes a compound of formula (I) and/or a compound of formula (II) under conditions effective to treat the subject for diabetes. Diabetes can be divided into two broad types of diseases: type I (T1D) and type II (T2D). The term “diabetes” also refers herein to a group of metabolic diseases in which patients have high blood glucose levels, including type I diabetes (T1D), type II diabetes (T2D), gestational diabetes, congenital diabetes, maturity onset diabetes (MODY), cystic fibrosis-related diabetes, hemochromatosis-related diabetes, drug-induced diabetes (e.g., steroid diabetes), and several forms of monogenic diabetes.

Thus, in some embodiments, the subject to be treated for diabetes, has been diagnosed as having one or more of type I diabetes (T1D), type II diabetes (T2D), gestational diabetes, congenital diabetes, maturity onset diabetes (MODY), cystic fibrosis-related diabetes, hemochromatosis-related diabetes, drug-induced diabetes, or monogenic diabetes. Drug induced diabetes relates to a condition that is caused through the use of drugs that are toxic to beta cells (e.g., steroids, antidepressants, second generation antipsychotics, and immunosuppressive). Exemplary immunosuppressive drugs include, but are not limited to, members of the cortisone family (e.g., prednisone and dexamethasome), rapamycin/sirolimus, everolimus, and calciuneurin inhibitors (e.g., FK-506/tacrolimus).

A further aspect of the present disclosure relates to a method of treating a subject for a neurological disease or disorder. This method involves administering to a subject in need of treatment for a neurological disorder a compound of formula (I) and/or a compound of formula (II) under conditions effective to treat the subject for the condition. Exemplary neurological disorders include, but are not limited to Alzheimer's Disease, Parkinson's Disease, Pick's Disease, Niemann-Pick's Disease, multiple sclerosis, neuropathies, tauopathies and amyoloidosis.

In the methods of the present disclosure involving selecting a subject, the subject is preferably a human subject, but can be any animal, including a laboratory animal in the context of a clinical trial or screening or activity experiment. Thus, as can be readily appreciated by one of ordinary skill in the art, the methods of the present disclosure are particularly suited to administration to any animal, particularly a mammal, and including, but by no means limited to, humans, domestic animals, such as feline (e.g., cats) or canine (e.g., dogs) subjects, farm animals, such as but not limited to bovine (e.g., cows), equine (e.g., horses), caprine (e.g., goats), ovine (e.g., sheep), and porcine (e.g., pigs) subjects, wild animals (whether in the wild or in a zoological garden), research animals, such as mice, rats, rabbits, guinea pigs, goats, sheep, pigs, dogs, cats, horses, cows, camels, llamas, monkeys, zebrafish etc., avian species, such as chickens, turkeys, songbirds, etc., i.e., for veterinary medical use.

Preferences and options for a given aspect, feature, embodiment, or parameter of the technology described herein should, unless the context indicates otherwise, be regarded as having been disclosed in combination with any and all preferences and options for all other aspects, features, embodiments, and parameters of the technology.

The present technology may be further illustrated by reference to the following examples.

### EXAMPLES

The examples below are intended to exemplify the practice of embodiments of the disclosure but are by no means intended to limit the scope thereof.

### Example 1—Materials and Methods

### Materials and Methods

All solvents were purchased from Sigma-Aldrich (St. Louis, MO) and were used as received; anhydrous solvents were used for chemical reactions, and HPLC grade solvents were used for aqueous work-ups, recrystallizations, and chromatography. The palladium metal on solid support, used in hydrogenation reactions, was purchased from Sigma-Aldrich (St. Louis, MO) as 10% w/w on activated carbon (dry basis), with 50% w/w water added (Degussa type); designated in procedures as “5% w/w on activated carbon.” Other reagents were purchased from various vendors and were used as received. Reactions were run as described in the individual procedures using standard double manifold and syringe techniques. Glassware was dried in a 130° C. oven for 12 h prior to use, or was flame-dried. The pH of aqueous solutions was estimated using pH paper. Vacuum filtrations were carried out using a house vacuum line (˜100 torr). In the individual procedures, the phrases “concentration under vacuum” and “concentrated to dryness” mean that solvent was removed on a rotary evaporator using a diaphragm pump (with an automatic vacuum regulator) and remaining traces of volatiles were removed on a high-vacuum (<1 torr) oil pump. Unless specified otherwise, the term “flask” refers to the round-bottomed variety. Reactions were monitored by TLC using EMD silica gel 60 F₂₅₄ (250 μm) glass-backed plates (visualized by UV fluorescence quenching and stained with basic KMnO₄ solution) and by liquid chromatography-tandem mass spectrometry (LC-MS). Analysis by reverse-phase LC-MS was carried out on a Waters Acquity I-Class UPLC system, with a C18 column (2.1×30 mm; 1.7 μm particle size), heated at 50° C., eluted at 0.6 mL/min, and using a 3 min linear gradient method with a mobile phase consisting of water/acetonitrile (0.1% v/v formic acid added to each): 95:5→1:99 (0-2.5 min), then 1:99(2.5-3 min). Sample runs were monitored using alternating positive/negative electrospray ionization (50-1000 amu) and UV detection at 254 nm. Dimensions of plugs, pads, and columns for filtration or flash chromatography are reported as: ((diameter×length) cm). The 5¾ inch pipets (4 mL) used for filtration and micro scale flash chromatography were purchased from Fisher Scientific (Waltham, MA). Automated preparative normal- and reverse-phase chromatography was carried out with an Interchim PuriFlash 450 purification system with a diode array detector (runs were monitored at 220-400 nm). Pre-packed silica gel cartridges (12, 25, and 40 g; 15 μm particle size) were employed for normal-phase (silica gel) chromatography, eluting at 20-30 mL/min. Preparative reverse-phase chromatography was carried out with an Agilent 1260 Infinity using a C18 column (30×100 mm; 5 μm particle size) with a multiwavelength detector, eluting at 40 mL/min with a pressure limit of 200 bar; crude samples were injected with an autosampler, typically in a 90:10 mixture of MeOH/DMSO (1.5 mL/injection). Carbon-decoupled ¹H NMR spectra were recorded at 400 MHz on a Bruker spectrometer and are reported in ppm using the residual solvent signal (dimethylsulfoxide-d6=2.50 ppm) as an internal standard. Data are reported as: {(shift), [(s=singlet, d=doublet, dd=doublet of doublets, ddd=doublet of a doublet of doublets, t=triplet, dt=doublet of triplets, q=quartet, quin=quintet, sext=sextet, sept=septet, m=multiplet, br=broad, ap=apparent), (J=coupling constant in Hz), (integration)]}. Proton-decoupled ¹³C NMR spectra were recorded at 100 MHz on a Bruker spectrometer and are reported in ppm using the residual solvent signal (dimethylsulfoxide-d₆=39.5 ppm) as an internal standard. Proton-decoupled ¹⁹F NMR spectra were recorded at 376 MHz on a Bruker spectrometer and are reported in ppm using added CFCl₃ (0.00 ppm) as an internal standard; compounds with only one signal were integrated relative to a known amount of the internal standard.

### Sequence Alignments

Protein sequence alignments were performed using Pymol (v2.3.4) and JalView (v2.11.1.2). Sequences were obtained from Uniprot (full length human RET), the PDB and KinaMetrix.com.

### Calculation/Visualization of Energy/Geometry Minimizations and Molecular Properties

All calculations related to the Ph-R^{F}/R^{H} CAP-group model fragments were performed using the ChemBio3D Ultra (v12.0) software package; CLogP and Connolly solvent excluded volume were calculated using the standard interface. The GAMESS Interface, included with ChemBio3D Ultra, was used for quantum mechanical calculations. The Hartree-Fock method was implemented for all quantum mechanical calculations, and aside from the basis set, default parameters were applied. Energy/geometry minimizations were performed with a 3-21G basis set, while a 6-311G basis set was used for molecular properties calculations (molecular surfaces, Lowdin charges/populations, Mulliken charges/populations, dipoles, molecular surfaces, electron density and electrostatic potential). The calculation results (e.g. partial charges, bond/molecular dipoles, van der Waals surfaces and MEP maps; contained in the GAMESS.out file) were visualized with Jmol (v14.30.2): an open-source Java viewer for chemical structures in 3D. PyMol (v2.3.4; pymol.org) was used to calculate and visualize protein structures, distance measurements and MEP maps pertaining to the RET(B06) homology model (obtained from KinaMetrix.com), the corresponding in silico derived mutant (M918T), as well as the structures (obtained from the PDB, of BRAF (5H₁₂), and SRC (3EL8). MEP maps were calculated using the APBS Electrostatics plugin included with PyMol. Throughout the disclosure, dipoles and MEP maps are intended only as relative metrics; MEP map scales are shown in arbitrary units (arb. unit). DFG(-out)-pocket volume calculations were performed with POVME v2.0.

### Fly Stocks

Kinase-mutated fly stocks were obtained from Bloomington Drosophila Stock Center (BDSC; Bloomington, IN). The active mutant form of Drosophila Ret (dRet^{955T}) carries an M955T mutation corresponding to the M918T mutation commonly reported for multiple endocrine neoplasia (MEN) type 2B patients12,14,16,34. The ptc-gal4, UAS-GFP;UAS-dRet^{M955T}/SM5_{tub-gal80}-TM6B transgenic flies were prepared according to standard protocols and crossed with non-transgenic w-flies (BDSC) to generate ptc-gal4, UAS-GFP; UAS-dRet^{M955T}(ptc>dRet^{M955T}) flies for chemical screening.

### Example 2—Synthesis of Intermediates

### 4-(Perfluoroethyl)aniline (APS-8-89 (6))

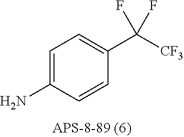 [SMILES: Nc1ccc(C(F)(F)C(F)(F)F)cc1]

A 150 mL sealable heavy-walled vessel was charged with tetrabutylammonium hydrogen sulfate (460 mg, 1.35 mmol), sodium bicarbonate (1.25 mg, 14.9 mmol) and water (35 mL). Sodium hydrosulfite (2.82 g, 16.2 mmol) was added in portions to the stirred solution over 1 min. The solution was diluted with methyl tert-butyl ether (MTBE; 15 mL) and then aniline (1.23 mL, 13.5 mmol) was added in a steady stream by syringe. To the biphasic mixture was added a solution of perfluoroethyl iodide (3.65 g, 14.8 mmol) and MTBE (10 mL) over 5 min by pipet; the solution was generated by bubbling perfluoroethyl iodide gas through MTBE cooled to 0° C. The headspace was purged with Ar, the vessel was sealed, and the reaction was stirred for 11 h protected from light. The biphasic mixture was transferred to a separatory funnel, the layers were separated, and the aqueous phase was extracted with Et₂O (50 mL). The organic extracts were pooled, washed with brine (25 mL), dried (Na₂SO₄), and filtered. The filtrate was concentrated under vacuum to leave a red oil, which was purified by silica gel chromatography (40 g cartridge), eluting at 40 mL/min and using a linear gradient of hexanes/CH₂Cl₂: 100:0→60:40 over 33 column volumes. Obtained 531 mg (19%) of the title compound as a yellow liquid: 1H NMR (400 MHz, DMSO-d₆) δ 7.25 (d, J=8.8 Hz, 2H), 6.66 (d, J=8.8 Hz, 2H), 5.84 (s, 2H); ¹⁹F NMR (376 MHz, DMSO-d₆) δ −83.8 (s, 3F), −111.0 (s, 2F); LC-MS (ESI+) m/z: [M+H]⁺ Calcd for C₈H₇F₅N 212.0; Found 212.1.

### 4-(Perfluoropropan-2-yl)aniline (APS-8-42-1 (8))

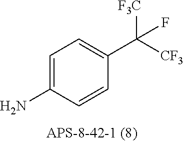 [SMILES: Nc1ccc(C(F)(C(F)(F)F)C(F)(F)F)cc1]

A 40 mL vial was charged with tetrabutylammonium hydrogen sulfate (298 mg, 0.878 mmol), sodium bicarbonate (811 mg, 9.65 mmol), and water (15 mL). Sodium hydrosulfite (1.68 g, 9.65 mmol) was added in portions to the stirred solution over 1 min. The solution was diluted with MTBE (15 mL) and then aniline (800 μL, 11.0 mmol) was added in a steady stream. To the biphasic mixture was added heptafluoro-2-iodopropane (1.37 mL, 9.63 mmol) over 5 min by syringe. The headspace was purged with Ar, the vial was sealed with a screwcap, and the reaction was stirred for 8 h protected from light. The biphasic mixture was transferred to a separatory funnel, the layers were separated, and the aqueous phase was extracted with Et₂O (25 mL). The organic extracts were pooled, washed with brine (25 mL), dried (Na₂SO₄), and filtered. The filtrate was concentrated under vacuum to leave a red oil, which was purified by silica gel chromatography (40 g cartridge), eluting at 40 mL/min and using a linear gradient of hexanes/CH₂Cl₂: 100:0→50:50 over 33 column volumes. Obtained 1.40 g (61%) of the title compound as a yellow liquid: ¹H NMR (400 MHz, DMSO-d₆) δ 7.23 (d, J=8.6 Hz, 2H), 6.68 (d, J=8.3 Hz, 2H), 5.75 (d, J=4.9 Hz, 2H); ¹⁹F NMR (376 MHz, DMSO-d₆) δ −75.0 (d, J=9.2 Hz, 6F), −179.7-−179.5 (m, 1F); LC-MS (ESI+) m/z: [M+H]⁺ Calcd for C₉H₇F₇N 262.0; Found 262.1.

### 4-(Perfluoropropyl)aniline (APS-8-42-2 (7))

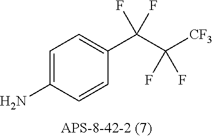 [SMILES: Nc1ccc(C(F)(F)C(F)(F)C(F)(F)F)cc1]

A 40 mL vial was charged with tetrabutylammonium hydrogen sulfate (298 mg, 0.878 mmol), sodium bicarbonate (811 mg, 9.65 mmol) and water (15 mL). Sodium hydrosulfite (1.68 g, 9.65 mmol) was added in portions to the stirred solution over 1 min. The solution was diluted with MTBE (15 mL) and then aniline (800 μL, 11.0 mmol) was added in a steady stream. To the biphasic mixture was added perfluoropropyl iodide (1.39 mL, 9.63 mmol) over 5 min by syringe. The headspace was purged with Ar, the vial was sealed with a screwcap, and the reaction was stirred for 8 h protected from light. The biphasic mixture was transferred to a separatory funnel, the layers were separated, and the aqueous phase was extracted with Et₂O (25 mL). The organic extracts were pooled, washed with brine (25 mL), dried (Na₂SO₄), and filtered. The filtrate was concentrated under vacuum to leave a red oil, which was purified by silica gel chromatography (40 g cartridge), eluting at 40 mL/min and using a linear gradient of hexanes/CH₂Cl₂: 100:0→50:50 over 33 column volumes. Obtained 893 mg (39%) of the title compound as an orange liquid: 1H NMR (400 MHz, DMSO-d₆) δ 7.23 (d, J=8.8 Hz, 2H), 6.66 (d, J=8.8 Hz, 2H), 5.86 (s, 2H); ¹⁹F NMR (376 MHz, DMSO-d₆) δ −79.1-−79.0 (m, 3F), −108.1-−108.0 (m, 2F), −125.7 (s, 2F); LC-MS (ESI+) m/z: [M+H]⁻ Calcd for C₉H₇F₇N 262.0; Found

[symbol]

### 4-(Perfluorobutan-2-yl)aniline (APS-7-81 (10))

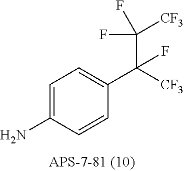 [SMILES: Nc1ccc(C(F)(C(F)(F)F)C(F)(F)C(F)(F)F)cc1]

A 100 mL flask was charged with tetrabutylammonium hydrogen sulfate (373 mg, 1.10 mmol), sodium bicarbonate (1.02 g, 12.1 mmol) and water (25 mL). Sodium hydrosulfite (2.48 g, 12.1 mmol) was added in portions to the stirred solution over 1 min. The solution was diluted with MTBE (25 mL) and then aniline (1.00 mL, 11.0 mmol) was added in a steady stream. To the biphasic mixture was added nonafluoro-2-iodobutane (2.00 mL, 12.1 mmol) over 5 min by syringe. The headspace was purged with Ar and the reaction was stirred under a balloon for 10 h protected from light. The biphasic mixture was transferred to a separatory funnel, the layers were separated, and the aqueous phase was extracted with Et₂O (25 mL). The organic extracts were pooled, washed with brine (25 mL), dried (Na₂SO₄), and filtered. The filtrate was concentrated under vacuum to leave a red oil, which was purified by silica gel chromatography (40 g cartridge), eluting at 40 mL/min and using a linear gradient of hexanes/CH₂Cl₂: 100:0→50:50 over 30 column volumes. Obtained 1.71 g (50%) of the title compound as an orange liquid: 1H NMR (400 MHz, DMSO-d₆) δ 7.21 (d, J=8.3 Hz, 2H), 6.68 (d, J=8.6 Hz, 2H), 5.74 (s, 2H); ¹⁹F NMR (376 MHz, DMSO-d₆) δ −73.7-−73.4 (m, 3F), −78.3 (d, J=11.4 Hz, 3F), −121.0-−120.6 (m, 2F), −181.9-−181.6 (m, 1F); LC-MS (ESI+) m/z: [M+H]⁺ Calcd for C₁₀H₇F₉N 312.0; Found 312.2.

### 4-(Perfluorobutyl)aniline (APS-8-45 (9))

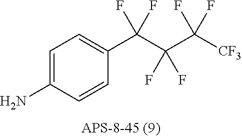 [SMILES: Nc1ccc(C(F)(F)C(F)(F)C(F)(F)C(F)(F)F)cc1]

A 40 mL vial was charged with tetrabutylammonium hydrogen sulfate (298 mg, 0.878 mmol), sodium bicarbonate (811 mg, 9.65 mmol) and water (15 mL). Sodium hydrosulfite (1.68 g, 9.65 mmol) was added in portions to the stirred solution over 1 min. The solution was diluted with MTBE (15 mL) and then aniline (800 μL, 11.0 mmol) was added in a steady stream. To the biphasic mixture was added nonafluoro-1-iodobutane (1.66 mL, 9.65 mmol) over 5 min by syringe. The headspace was purged with Ar, the vial was sealed with a screwcap, and the reaction was stirred for 16 h protected from light. The biphasic mixture was transferred to a separatory funnel, the layers were separated, and the aqueous phase was extracted with Et₂O (25 mL). The organic extracts were pooled, washed with brine (25 mL), dried (Na₂SO₄), and filtered. The filtrate was concentrated under vacuum to leave a red oil, which was purified by silica gel chromatography (40 g cartridge), eluting at 40 mL/min and using a linear gradient of hexanes/CH₂Cl₂: 100:0→50:50 over 33 column volumes. Obtained 846 mg (31%) of the title compound as an orange liquid: ¹H NMR (400 MHz, DMSO-d₆) δ 7.24 (d, J=8.7 Hz, 2H), 6.66 (d, J=8.8 Hz, 2H), 5.86 (s, 2H); ¹⁹F NMR (376 MHz, DMSO-d₆) δ −80.2-−80.0 (m, 3F), −107.4-−107.3 (m, 2F), −122.1-−122.0 (m, 2F), −124.9-−124.7 (m, 2F); LC-MS (ESI+) m/z: [M+H]⁺ Calcd for C₁₀H₇F₉N 312.0; Found 312.1.

### 1-Nitro-3-(perfluoroethyl)benzene (APS-8-94a (11a))

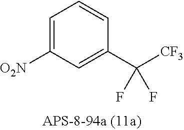 [SMILES: O=[N+]([O-])c1cccc(C(F)(F)C(F)(F)F)c1]

A 75 mL sealable heavy-walled vessel was charged with activated copper powder (3.32 g, 52.2 mmol) dry DMSO (20 mL; deoxygenated by bubbling Ar for 10 min) and 1-iodo-3-nitrobenzene (3.25 g, 13.1 mmol). To the mixture was added a solution of perfluoroethyl iodide (4.81 g, 19.5 mmol) and DMSO (10 mL) in a steady stream by syringe; the solution was generated by bubbling perfluoroethyl iodide gas through DMSO at room temperature. The headspace was purged with Ar, the vessel was sealed, and the reaction was heated at 120° C. for 24 h. The reaction mixture was allowed to cool to room temperature and then was filtered through a pad (3×3 cm) of Celite under vacuum; washed pad with Et₂O (2×20 mL). The combined filtrates were diluted with Et₂O (100 mL), then washed with a 60:40 mixture of saturated NH₄Cl solution/concd NH₄OH solution (3×50 mL), water (2×50 mL) and brine (2×50 mL), dried (MgSO₄), and filtered. Concentration under vacuum gave an orange semi-solid, which was triturated with hexanes (10-15 mL) and filtered to remove the solid precipitate. The filtrate was concentrated to dryness to provide 2.28 g (72%) of the title compound as an orange oil, which was used in the next reaction without further purification (see 11).

### 3-(Perfluoroethyl)aniline (APS-8-94 (11))

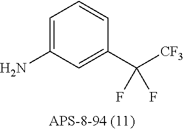 [SMILES: Nc1cccc(C(F)(F)C(F)(F)F)c1]

To a 100 mL flask, containing 1-nitro-3-(perfluoroethyl)benzene (11a; 2.28 g, 9.46 mmol), was added MeOH (50 mL) and iron powder (3.17 g, 56.8 mmol). The mixture was cooled to 0° C., and concentrated HCl (9.5 mL, 116 mmol) was added dropwise by pipet over 2-3 min. The reaction was allowed to warm to room temperature over 3 h, and was stirred for an additional 3 h. The reaction was decanted from the unreacted iron, diluted with water (50 mL) and CH₂Cl₂ (50 mL), and then the pH of the aqueous phase was adjusted to ˜7 with 6 M NaOH (19-20 mL). The mixture was vacuum-filtered through a pad of Celite/sand (50:50), and the filter-cake was washed with CH₂Cl₂ (50 mL). The combined filtrates were transferred to a separatory funnel, the layers were separated, and the aqueous phase was extracted with CH₂Cl₂ (2×50 mL). The organic extracts were pooled, dried (Na₂SO₄), and filtered. The filtrate was concentrated under vacuum to leave an orange liquid, which was purified by silica gel chromatography (40 g cartridge), eluting at 20 mL/min and using a linear gradient of hexanes/CH₂Cl₂: 100:0→50:50 over 35 column volumes. Obtained 1.13 g (57%) of the title compound as a pale-yellow liquid: 1H NMR (400 MHz, DMSO-d₆) δ 7.22 (t, J=8.2 Hz, 1H), 6.78-6.84 (m, 2H), 6.72 (d, J=7.8 Hz, 1H), 5.57 (br s, 2H); ¹⁹F NMR (376 MHz, DMSO-d₆) δ −83.6-−83.5 (m, 3F), −113.3-−113.2 (m, 2F); LC-MS (ESI+) m/z: [M+H]⁺ Calcd for C₈H₇F₅N 212.1; Found 212.1.

### 1-Nitro-3-(perfluoropropan-2-yl)benzene (APS-7-34-1 (15a))

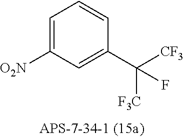 [SMILES: O=[N+]([O-])c1cccc(C(F)(C(F)(F)F)C(F)(F)F)c1]

To a mixture of 1-iodo-3-nitrobenzene 1.50 g, 6.02 mmol), activated copper powder (1.53 g, 24.1 mmol) and dry DMF (17 mL; deoxygenated by bubbling Ar for 10 min) was added heptafluoro-2-iodopropane (1.28 mL, 9.00 mmol) via syringe over 1 min. The mixture was stirred under Ar and heated at 80° C. for 24 h. The reaction mixture was allowed to cool to room temperature and then was filtered through a pad (3×3 cm) of Celite under vacuum; washed pad with Et₂O (2×20 mL). The combined filtrates were diluted with Et₂O (100 mL), then washed with a 60:40 mixture of saturated NH₄Cl solution/concd NH₄OH solution (3×50 mL), water (2×50 mL) and brine (2×50 mL), dried (MgSO₄), and filtered. Concentration under vacuum gave an orange semi-solid, which was purified by silica gel chromatography (40 g cartridge), eluting at 30 mL/min and using a linear gradient of hexanes/CH₂Cl₂: 100:0→75:25 over 28 column volumes. Obtained 1.51 g (81%) of the title compound as a yellow oil, which was used directly in the next step (see 15).

### 3-(Perfluoropropan-2-yl)aniline APS-7-42 (15))

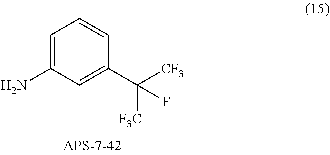 [SMILES: Nc1cccc(C(F)(C(F)(F)F)C(F)(F)F)c1]

A 100 mL flask was charged with 1-nitro-3-(perfluoropropan-2-yl)benzene (15a; [symbol] mol), MeOH (30 mL) and iron powder (1.74 g, 31.2 mmol). The mixture was cooled to 0° C., and concentrated HCl (5.1 mL, 62 mmol) was added dropwise by pipet over 5 min. The reaction was allowed to warm to room temperature over 30 min, and was stirred for an additional 5 h. The reaction was decanted away from unreacted iron powder and diluted with water (75 mL) and CH₂Cl₂ (75 mL), and the pH of the aqueous phase was adjusted to 7-8 with 6 M NaOH solution (˜10.4 mL). The resulting mixture was vacuum-filtered through a pad of sand layered on top of Celite and the filter-cake was washed with CH₂Cl₂ (30 mL). The combined filtrates were transferred to a separatory funnel, diluted with brine (30 mL) and the layers were separated. The aqueous phase was extracted with CH₂Cl₂ (2×30 mL). The organic extracts were pooled, dried (Na₂SO₄), and filtered. The filtrate was concentrated under vacuum to leave an oil, which was purified by silica gel chromatography (40 g cartridge), eluting at 30 mL/min and using a linear gradient of hexanes/CH₂Cl₂: 100:0→80:20 over 23 column volumes. Obtained 960 mg (71%) of the title compound as a yellow liquid: ¹H NMR (400 MHz, DMSO-d₆) δ 7.22 (t, J=7.9 Hz, 1H), 6.83 (s, 1H), 6.77 (ddd, J=8.1, 2.3, 0.9 Hz, 1H), 6.69 (d, J=7.8 Hz, 1H), 5.57 (br s, 2H); ¹⁹F NMR (376 MHz, DMSO-d₆) δ −74.6 (d, J=6.9 Hz, 6F), −181.5-−181.3 (m, 1F); LC-MS (ESI+) m/z: [M+H]⁺ Calcd for C₉H₇F₇N 262.0; Found 262.1.

### 1-Nitro-3-(perfluoropropyl)benzene (APS-7-48 (13a))

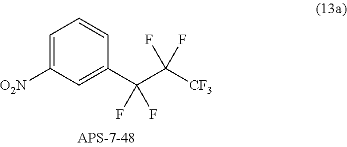 [SMILES: O=[N+]([O-])c1cccc(C(F)(F)C(F)(F)C(F)(F)F)c1]

To a mixture of 1-iodo-3-nitrobenzene (2.00 g, 8.03 mmol), activated copper powder (2.04 g, 32.1 mmol) and dry DMF (20 mL; deoxygenated by bubbling Ar for 10 min) was added perfluoropropyl iodide (2.00 mL, 13.9 mmol) via syringe over 1 min. The mixture was stirred under Ar and heated at 100° C. for 24 h. The reaction mixture was allowed to cool to room temperature and then was filtered through a pad (3×3 cm) of Celite under vacuum; washed pad with Et₂O (2×20 mL). The combined filtrates were diluted with Et₂O (100 mL), then washed with a 60:40 mixture of saturated NH₄Cl solution/concd NH₄OH solution (3×50 mL), water (2×50 mL), and brine (2×50 mL), dried (MgSO₄), and filtered. Concentration under vacuum gave an orange semi-solid, which was triturated with hexanes (5-10 mL) and filtered to remove the solid precipitate. The filtrate was concentrated to dryness to provide 2.29 g (81%) of the title compound as a yellow oil, which was used in the next reaction without further purification (see 13).

### 3-(Perfluoropropyl)aniline (APS-7-49 (13))

To a 100 mL flask containing 1-nitro-3-(perfluoropropyl)benzene (13a; 2.29 g,

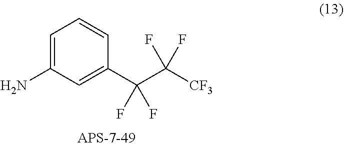 [SMILES: Nc1cccc(C(F)(F)C(F)(F)C(F)(F)F)c1]

7.87 mmol), MeOH (30 mL) and iron powder (2.64 g, 47.3 mmol). The mixture was cooled to 0° C., and concentrated HCl (7.75 mL, 94.6 mmol) was added dropwise by pipet over 5 min. The reaction was allowed to warm to room temperature over 30 min, and was stirred for an additional 3 h. The reaction was decanted away from unreacted iron powder, then diluted with water (75 mL) and CH₂Cl₂ (75 mL), and the pH of the aqueous phase was adjusted to 7-8 with 6 M NaOH solution (˜15.8 mL). The resulting mixture was vacuum-filtered through a pad of sand layered on top of Celite and the filter-cake was washed with CH₂Cl₂ (30 mL). The combined filtrates were transferred to a separatory funnel, diluted with brine (30 mL) and the layers were separated. The aqueous phase was extracted with CH₂Cl₂ (2×30 mL). The organic extracts were pooled, dried (Na₂SO₄), and filtered. The filtrate was concentrated under vacuum to leave an oil, which was purified by silica gel chromatography (40 g cartridge), eluting at 30 mL/min and using a linear gradient of hexanes/CH₂Cl₂: 100:0→80:20 over 23 column volumes. Obtained 1.02 g (50%) of the title compound as a yellow liquid: 1H NMR (400 MHz, DMSO-d₆) δ 7.22 (t, J=7.8 Hz, 1H), 6.78-6.85 (m, 2H), 6.70 (d, J=7.6 Hz, 1H), 5.56 (s, 2H); ¹⁹F NMR (376 MHz, DMSO-d₆) δ −79.3-−79.2 (m, 3F), −110.3-−110.2 (m, 2F), −125.8 (s, 2F); LC-MS (ESI+) m/z: [M+H]⁺ Calcd for C₉H₇F₇N 262.0; Found 262.2.

### N-(4-(Perfluorobutan-2-yl)phenyl)acetamide (APS-8-81 (25))

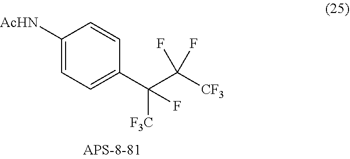 [SMILES: CC(=O)Nc1ccc(C(F)(C(F)(F)F)C(F)(F)C(F)(F)F)cc1]

To a solution of 4-(perfluorobutan-2-yl)aniline (10; 2.42 g, 7.78 mmol) and CH₂Cl₂ (15 mL) was added acetic anhydride (880 μL, 9.31 mmol) and then pyridine (760 μL, 9.40 mmol). The solution was stirred for 9 h, then poured into 0.5 M HCl (50 mL) and extracted with CH₂Cl₂ (3×50 mL). The organic extracts were pooled, dried (MgSO₄), and filtered. Concentration under vacuum gave an off-white solid, which was recrystallized from hexanes/EtOAc (˜30:1, −40 mL) to yield 1.82 g (66%) of the title compound as a white solid. The filtrate was concentrated to dryness and purified by silica gel chromatography (40 g cartridge), eluting at 30 mL/min and using a linear gradient of hexanes/EtOAc: 100:0→50:50 over 24 column volumes. Obtained an additional 644 mg of product (2.46 g in total, 90% overall): 1H NMR (400 MHz, DMSO-d₆) δ 10.29 (s, 1H), 7.81 (d, J=8.6 Hz, 2H), 7.58 (d, J=8.8 Hz, 2H), 2.08 (s, 3H); ¹⁹F NMR (376 MHz, DMSO-d₆) δ −73.3-−73.1 (m, 3F), −78.3 (d, J=11.4 Hz, 3F), −120.8-−120.6 (m, 2F), −182.3-−182.0 (m, 1F); LC-MS (ESI+) m/z: [M+H]⁺ Calcd for C₁₂H₉F₉NO 354.1; Found 354.3.

### 2-Nitro-4-(perfluorobutan-2-yl)aniline (APS-8-91 (26))

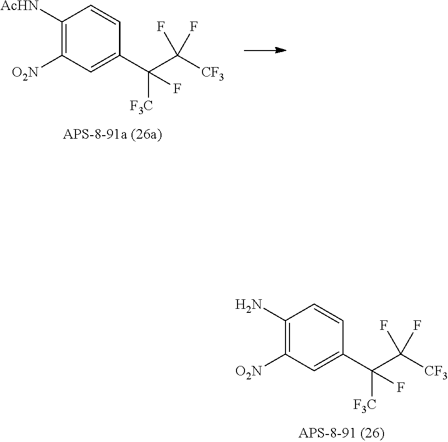 [SMILES: CC(=O)Nc1ccc(C(F)(C(F)(F)F)C(F)(F)C(F)(F)F)cc1[N+](=O)[O-] ; Nc1ccc(C(F)(C(F)(F)F)C(F)(F)C(F)(F)F)cc1[N+](=O)[O-]]

A 100 mL flask was charged with N-(4-(perfluorobutan-2-yl)phenyl)acetamide (25; 2.41 g, 6.82 mmol) and 25 mL coned H₂SO₄ (25 mL), and the solution was cooled to 0° C. A solution of fuming HNO₃ (340 μL, 90% w/w, 7.19 mmol) and concd H₂SO₄ (3 mL) was added down the wall of the flask by pipet over 5 min. The reaction was allowed to warm to room temperature over 2 h and was stirred for an additional 3 h; this generated 26a. Small chunks of ice (˜50 g) were added in portions over 5-10 min. The reaction was allowed to warm to room temperature and stirred for 30 min. The resulting precipitate was collected by vacuum filtration and washed with water (2×5 mL). Air-drying yielded 2.30 g (85%) of the title compound as a yellow solid: 1H NMR (400 MHz, DMSO-d₆) δ 8.15 (d, J=2.2 Hz, 1H), 7.92 (br s, 2H), 7.60 (d, J=9.5 Hz, 1H), 7.23 (dd, J=9.2, 0.6 Hz, 1H); ¹⁹F NMR (376 MHz, DMSO-d₆) δ −73.5-−73.3 (m, 3F), −78.3 (d, J=13.7 Hz, 3F), −120.8-−120.6 (m, 2F), −181.8-−181.3 (m, 1F); LC-MS (ESI+) m/z: [M+H]⁺ Calcd for C₁₀H₆F₉N₂O₂ 357.0; Found 357.0.

### 1-Nitro-3-(perfluorobutan-2-yl)benzene (APS-8-96 (19a))

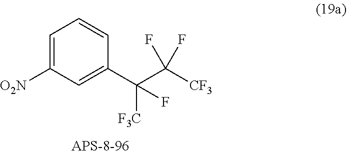 [SMILES: O=[N+]([O-])c1cccc(C(F)(C(F)(F)F)C(F)(F)C(F)(F)F)c1]

A solution of 2-nitro-4-(perfluorobutan-2-yl)aniline (26; 2.20 g, 4.84 mmol), concd H₂SO₄ (900 μL, 16.9 mmol) and EtOH (30 mL) was heated at reflux and sodium nitrite (1.00 g, 14.5 mmol) was added in portions over 1.5 h. The solution was heated at reflux for an additional 4 h and then allowed to cool to room temperature. The solution was poured into crushed ice and was extracted with Et₂O (3×75 mL). The organic extracts were pooled, washed with a saturated NaHCO₃ (75 mL), water (75 mL) and brine (75 mL), dried (MgSO₄), and filtered. Concentration under vacuum gave an orange oil, which was used in the next reaction without further purification (see 19).

### 3-(Perfluorobutan-2-yl)aniline (APS-8-97 (19))

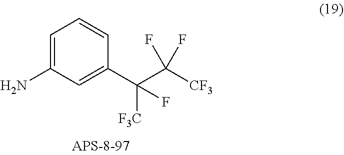 [SMILES: Nc1cccc(C(F)(C(F)(F)F)C(F)(F)C(F)(F)F)c1]

To a 100 mL flask, containing 1-nitro-3-(perfluorobutan-2-yl)benzene (19a; ˜4.84 mmol from previous reaction), was added palladium (310 mg, 5% w/w on activated carbon, 0.15 mmol, 3 mol %) and MeOH (20 mL). A 3-way inlet adapter was attached, the flask was evacuated using a house vacuum line and then back-filled with H₂ from a balloon. The mixture was stirred under a balloon of H₂ for 6 h and then vacuum filtered through a pad of Celite (3×3 cm); washed pad with MeOH (2×5 mL). The combined filtrates were concentrated under vacuum to leave an oil, which was purified by silica gel chromatography (40 g cartridge), eluting at 30 mL/min and using a linear gradient of hexanes/CH₂Cl₂: 100:0→50:50 over 30 column volumes. Obtained 1.36 g (90% over two steps) of the title compound as an orange liquid: ¹H NMR (400 MHz, DMSO-d₆) δ 7.21 (t, J=7.9 Hz, 1H), 6.82 (s, 1H), 6.76 (ddd, J=8.2, 2.2, 0.9 Hz, 1H), 6.68 (d, J=7.8 Hz, 1H), 5.56 (s, 2H); ¹⁹F NMR (376 MHz, DMSO-d₆) δ −73.1-−72.9 (s, 3F), −78.4 (d, J=13.7 Hz, 3F), −120.7-−120.5 (m, 2F), −182.2 (s, 1F); LC-MS (ESI+) m/z: [M+H]⁺ Calcd for C₁₀H₇F₉N 312.0; Found 312.1.

### 1-Nitro-3-(perfluorobutyl)benzene (APS-7-34-2 (17a))

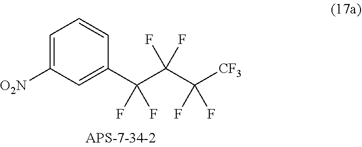 [SMILES: O=[N+]([O-])c1cccc(C(F)(F)C(F)(F)C(F)(F)C(F)(F)F)c1]

To a mixture of 1-iodo-3-nitrobenzene (1.50 g, 6.02 mmol), activated copper powder (1.53 g, 24.1 mmol) and dry DMF (17 mL; deoxygenated by bubbling Ar for 10 min) was added perfluorobutyl iodide (1.55 mL, 9.01 mmol) via syringe over 1 min. The mixture was stirred under Ar and heated at 100° C. for 24 h. The reaction mixture was allowed to cool to room temperature and then was filtered through a pad (3×3 cm) of Celite under vacuum; washed pad with Et₂O (2×20 mL). The combined filtrates were diluted with Et₂O (100 mL), then washed with a 60:40 mixture of saturated NH₄Cl solution/concd NH₄OH solution (3×50 mL), water (2×50 mL) and brine (2×50 mL), dried (MgSO₄), and filtered. Concentration under vacuum gave an orange semi-solid, which was triturated with hexanes (5-10 mL) and filtered to remove the solid precipitate. The filtrate was concentrated to dryness to provide 1.84 g (84%) of the title compound as a yellow oil, which was used in the next reaction without further purification (see 17).

### 3-(Perfluorobutyl)aniline (APS-7-43 (17))

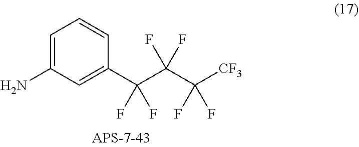 [SMILES: Nc1cccc(C(F)(F)C(F)(F)C(F)(F)C(F)(F)F)c1]

To a 100 mL flask containing 1-nitro-3-(perfluorobutyl)benzene (17a; 1.84 g, 5.39 mmol), MeOH (30 mL) and iron powder (1.81 g, 32.4 mmol). The mixture was cooled to 0° C., and concentrated HCl (5.3 mL, 65 mmol) was added dropwise by pipet over 5 min. The reaction was allowed to warm to room temperature over 30 min, and was stirred for an additional 1 h. The reaction was decanted away from unreacted iron powder, then diluted with water (75 mL) and CH₂Cl₂ (75 mL), and the pH of the aqueous phase was adjusted to 7-8 with 6 M NaOH solution (˜10.8 mL). The resulting mixture was vacuum-filtered through a pad of sand layered on top of Celite and the filter-cake was washed with CH₂Cl₂ (30 mL). The combined filtrates were transferred to a separatory funnel, diluted with brine (30 mL) and the layers were separated. The aqueous phase was extracted with CH₂Cl₂ (2×30 mL). The organic extracts were pooled, dried (Na₂SO₄), and filtered. The filtrate was concentrated under vacuum to leave an oil, which was purified by silica gel chromatography (40 g cartridge), eluting at 30 mL/min and using a linear gradient of hexanes/CH₂Cl₂: 100:0→80:20 over 28 column volumes. Obtained 700 mg (42%) of the title compound as a yellow liquid: ¹H NMR (400 MHz, DMSO-d₆) δ 7.18-7.26 (m, 1H), 6.79-6.85 (m, 2H), 6.71 (d, J=7.8 Hz, 1H), 5.56 (s, 2H); ¹⁹F NMR (376 MHz, DMSO-d₆) δ −80.1 (t, J=9.2 Hz, 3F), −109.6-−109.4 (m, 2F), −122.2-−122.0 (m, 2F), −125.0-−124.8 (m, 2F); LC-MS (ESI+) m/z: [M+H]⁺ Calcd for C₁₀H₇F₉N 312.0; Found 312.2.

### 1-Fluoro-2-nitro-4-(perfluoroethyl)benzene (APS-7-29a (12a))

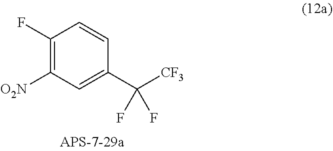 [SMILES: O=[N+]([O-])c1cc(C(F)(F)C(F)(F)F)ccc1F]

A 75 mL sealable heavy-walled vessel was charged with activated copper powder (3.30 g, 51.9 mmol) dry DMSO (20 mL; deoxygenated by bubbling Ar for 10 min) and 1-fluoro-4-iodo-2-nitrobenzene (3.47 g, 13.0 mmol). To the mixture was added a solution of perfluoroethyl iodide (4.80 g, 14.8 mmol) and DMSO (10 mL) in a steady stream by syringe; the solution was generated by bubbling perfluoroethyl iodide gas through DMSO at room temperature. The headspace was purged with Ar, the vessel was sealed, and the reaction was heated at 120° C. for 24 h. The reaction mixture was allowed to cool to room temperature and then was filtered through a pad (3×3 cm) of Celite under vacuum; washed pad with Et₂O (2× [symbol] The combined filtrates were diluted with Et₂O (100 mL), then washed with a 60:40 mixture of saturated NH₄Cl solution/coned NH₄OH solution (3×50 mL), water (2×50 mL) and brine (2×50 mL), dried (MgSO₄), and filtered. Concentration under vacuum gave an orange semi-solid, which was triturated with hexanes (10-15 mL) and filtered to remove the solid precipitate. The filtrate was concentrated to dryness to provide 2.79 g (83%) of the title compound as an orange oil, which was used in the next reaction without further purification (see 12).

### 2-Fluoro-5-(perfluoroethyl)aniline (APS-7-29 (12))

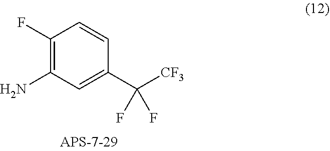 [SMILES: Nc1cc(C(F)(F)C(F)(F)F)ccc1F]

To a 100 mL flask, containing 1-fluoro-2-nitro-4-(perfluoroethyl)benzene (12a; 2.79 g, 10.8 mmol), was added MeOH (50 mL) and iron powder (3.61 g, 64.6 mmol). The mixture was cooled to 0° C., and concentrated HCl (10.6 mL, 129 mmol) was added dropwise by pipet over 2-3 min. The reaction was allowed to warm to room temperature over 3 h, and was stirred for an additional 3 h. The reaction was decanted from the unreacted iron, diluted with water (50 mL) and CH₂Cl₂ (50 mL), and then the pH of the aqueous phase was adjusted to ˜7 with 6 M NaOH (21.5 mL). The mixture was vacuum-filtered through a pad of Celite/sand (50:50), and the filter-cake was washed with CH₂Cl₂ (50 mL). The combined filtrates were transferred to a separatory funnel, the layers were separated, and the aqueous phase was extracted with CH₂Cl₂ (2×50 mL). The organic extracts were pooled, dried (Na₂SO₄), and filtered. The filtrate was concentrated under vacuum to leave an orange liquid, which was purified by silica gel chromatography (40 g cartridge), eluting at 20 mL/min and using a linear gradient of hexanes/CH₂Cl₂: 100:0→60:40 over 23 column volumes. Obtained 2.26 g (76%) of the title compound as a pale-yellow liquid: ¹H NMR (400 MHz, DMSO-d₆) δ 7.21 (dd, J=11.2, 8.6 Hz, 1H), 7.05 (dd, J=8.2, 2.3 Hz, 1H), 6.74-6.81 (m, 1H), 5.65 (br s, 2H); ¹⁹F NMR (376 MHz, DMSO-d₆) δ −83.7-−83.7 (m, 3F), −112.6-−112.5 (m, 2F), −129.2 (s, 1F); LC-MS (ESI+) m/z: [M+H]⁺ Calcd for C₈H₆F₆N 230.0; Found 230.2.

### 1-Fluoro-2-nitro-4-(perfluoropropan-2-yl)benzene (APS-6-36 (16a))

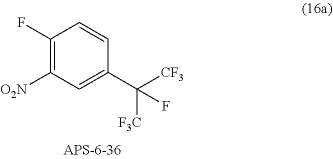 [SMILES: O=[N+]([O-])c1cc(C(F)(C(F)(F)F)C(F)(F)F)ccc1F]

To a mixture of 1-fluoro-4-iodo-2-nitrobenzene (1.25 g, 4.68 mmol), activated copper powder (1.20 g, 18.9 mmol) and dry DMF (13 mL; deoxygenated by bubbling Ar for 10 min) was added heptafluoro-2-iodopropane (1.00 mL, 7.03 mmol) via syringe over 1 min. The mixture was stirred under Ar and heated at 100° C. for 48 h. The reaction mixture was allowed to cool to room temperature and then was filtered through a pad (3×3 cm) of Celite under vacuum; washed pad with Et₂O (2×20 mL). The combined filtrates were diluted with Et₂O (100 mL), then washed with a 60:40 mixture of saturated NH₄Cl solution/concd NH₄OH solution (3×30 mL), water (2×30 mL) and brine (2×30 mL), dried (MgSO₄), and filtered. Concentration under vacuum gave 1.38 g of an orange semi-solid, which was purified by silica gel chromatography (25 g cartridge), eluting at 20 mL/min and using a linear gradient of hexanes/CH₂Cl₂: 100:0→80:20 over 25 column volumes. Obtained 680 mg (47%) of the title compound as a colorless oil: 1H NMR (400 MHz, DMSO-d₆) δ 8.36 (dd, J=6.6, 2.4 Hz, 1H), 8.16 (dt, J=8.7, 3.1 Hz, 1H), 7.86-7.94 (m, 1H); ¹⁹F NMR (376 MHz, DMSO-d₆) δ −74.6 (d, J=6.8 Hz, 6F), −112.9 (s, 1F), −180.1-−179.9 (m, 1F).

### 2-Fluoro-5-(perfluoropropan-2-yl)aniline (A PS-6-39 (16))

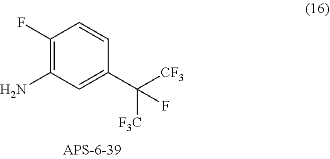 [SMILES: Nc1cc(C(F)(C(F)(F)F)C(F)(F)F)ccc1F]

A 50 mL flask was charged with 1-fluoro-2-nitro-4-(perfluoropropan-2-yl)benzene (16a; 665 mg, 2.15 mmol), MeOH (15 mL) and iron powder (720 mg, 12.9 mmol). The mixture was cooled to 0° C., and concentrated HCl (2.1 mL, 26 mmol) was added dropwise by pipet over 5 min. The reaction was allowed to warm to room temperature over 30 min, and was stirred for an additional 3 h. The reaction was vacuum-filtered through a pad (2×2 cm) of Celite to remove unreacted iron powder and the pad was washed with MeOH (5 mL). The combined filtrates were diluted with water (75 mL) and CH₂Cl₂ (75 mL), and the pH of the aqueous phase was adjusted to 7-8 with 6 M NaOH solution (˜4.3 mL). The resulting mixture was vacuum-filtered through a pad (4×4 cm total size) of sand (3×4 cm) layered on top of Celite (1×4 cm) and the filter-cake was washed with CH₂Cl₂ (30 mL). The combined filtrates were transferred to a separatory funnel, the layers were separated, and the aqueous phase was extracted with CH₂Cl₂ (2×50 mL). The organic extracts were pooled, dried (Na₂SO₄), and filtered. The filtrate was concentrated under vacuum to leave an oil, which was purified by silica gel chromatography (25 g cartridge), eluting at 20 mL/min and using a linear gradient of hexanes/CH₂Cl₂: 100:0→50:50 over 25 column volumes. Obtained 473 mg (79%) of the title

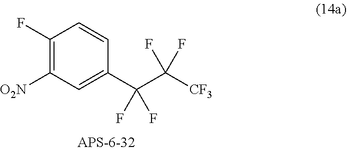 [SMILES: O=[N+]([O-])c1cc(C(F)(F)C(F)(F)C(F)(F)F)ccc1F]

compound as a colorless oil: 1H NMR (400 MHz, DMSO-d₆) δ 7.21 (dd, J=11.2, 8.6 Hz, 1H), 7.07 (dd, J=8.2, 1.8 Hz, 1H), 6.71-6.78 (m, 1H), 5.65 (br s, 2H); ¹⁹F NMR (376 MHz, DMSO-d₆) δ −74.8 (d, J=6.8 Hz, 6F), −130.6 (s, 1F), −180.4-−180.2 (m, 1F); LC-MS (ESI+) m/z: [M+H]⁺ Calcd for C₉H₆F₈N 280.1; Found 280.2.

### 1-Fluoro-2-nitro-4-(perfluoropropyl)benzene (APS-6-32 (14a))

To a mixture of 1-fluoro-4-iodo-2-nitrobenzene (1.25 g, 4.68 mmol), activated copper powder (1.19 g, 18.7 mmol) and dry DMF (13 mL; deoxygenated by bubbling Ar for 10 min) was added heptafluoro-1-iodopropane (1.01 mL, 7.00 mmol) via syringe over 1 min. The mixture was stirred under Ar and heated at 100° C. for 48 h. The reaction mixture was allowed to cool to room temperature and then was filtered through a pad (3×3 cm) of Celite under vacuum; washed pad with Et₂O (2×20 mL). The combined filtrates were diluted with Et₂O (100 mL), then washed with a 60:40 mixture of saturated NH₄Cl solution/concd NH₄OH solution (3×30 mL), water (2×30 mL) and brine (2×30 mL), dried (MgSO₄), and filtered. Concentration under vacuum gave an orange semi-solid, which was purified by silica gel chromatography (40 g cartridge), eluting at 30 mL/min and using a linear gradient of hexanes/CH₂Cl₂: 100:0→50:50 over 35 column volumes. Obtained 860 mg (59%) of the title compound as a colorless oil: ¹H NMR (400 MHz, DMSO-d₆) δ 8.43 (dd, J=6.7, 2.3 Hz, 1H), 8.21 (dt, J=8.7, 3.2 Hz, 1H), 7.90 (dd, J=10.8, 9.0 Hz, 1H).

### 2-Fluoro-5-(perfluoropropyl)aniline (APS-6-35 (14))

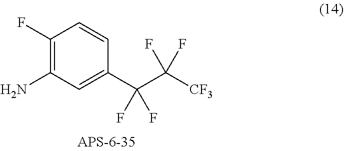 [SMILES: Nc1cc(C(F)(F)C(F)(F)C(F)(F)F)ccc1F]

A 50 mL flask was charged with 1-fluoro-2-nitro-4-(perfluoropropyl)benzene (14a; 850 mg, 2.75 mmol), MeOH (20 mL) and iron powder (768 mg, 13.8 mmol). The mixture was cooled to 0° C., and concentrated HCl (2.25 mL, 27.5 mmol) was added dropwise by pipet over 5 min. The reaction was allowed to warm to room temperature over 1 h, and was stirred for an additional 24 h. The reaction was vacuum-filtered through a pad (2×2 cm) of Celite to remove unreacted iron powder and the pad was washed with MeOH (5 mL). The combined filtrates were diluted with water (75 mL) and CH₂Cl₂ (75 mL), and the pH of the aqueous phase was adjusted to 7-8 with 6 M NaOH solution (˜4.6 mL). The resulting mixture was vacuum-filtered through a pad (4×4 cm total size) of sand (3×4 cm) layered on top of Celite (1×4 cm) and the filter-cake was washed with CH₂Cl₂ (30 mL). The combined filtrates were transferred to a separatory funnel, the layers were separated, and the aqueous phase was extracted with CH₂Cl₂ (2×50 mL). The organic extracts were pooled, dried (Na₂SO₄), and filtered. The filtrate was concentrated under vacuum to leave −750 mg of a yellow oil, which was purified by silica gel chromatography (25 g cartridge), eluting at 20 mL/min and using a linear gradient of hexanes/CH₂Cl₂: 100:0→70:30 over 25 column volumes. Obtained 382 mg (50%) of the title compound as a colorless oil: 1H NMR (400 MHz, DMSO-d₆) δ 7.22 (dd, J=11.2, 8.6 Hz, 1H), 7.04 (dd, J=8.2, 2.3 Hz, 1H), 6.77 (dt, J=8.3, 3.3 Hz, 1H), 5.65 (br s, 2H); ¹⁹F NMR (376 MHz, DMSO-d₆) δ −79.1-−79.1 (m, 3F), −109.6-−109.4 (m, 2F), −125.7 (s, 2F), −129.0 (s, 1F); LC-MS (ESI+) m/z: [M+H]⁺ Calcd for C₉H₆F₈N 280.1; Found 280.2.

### 4-(Perfluorobutan-2-yl)benzenediazonium tetrafluoroborate (APS-8-61 (27))

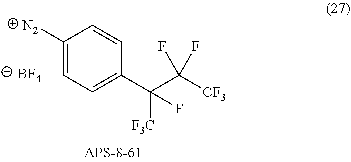

To a solution of 4-(perfluorobutan-2-yl)aniline (10; 5.72 g, 18.4 mmol) and EtOH (60 mL), in a 200 mL flask, was added HBF₄ (4.60 mL, 50% w/w in water, 36.7 mmol) in a steady stream via syringe over 1 min. The solution was stirred for 1 h and then cooled to 0° C. Isopentyl nitrite (5.00 mL, 37.2 mmol) was added dropwise over 20 min via syringe and the reaction was allowed to warm to room temperature overnight. After 14 h the resulting mixture was diluted with Et₂O (120 mL) and allowed to stand at 2° C. for 8 h. The precipitate was collected by vacuum filtration and washed with Et₂O (10 mL). Air-drying gave 4.78 g (63%) of the title compound as a white solid, which was used in the next reaction without further purification.

### 2-Fluoro-5-(perfluorobutan-2-yl)aniline (APS-8-71 (20))

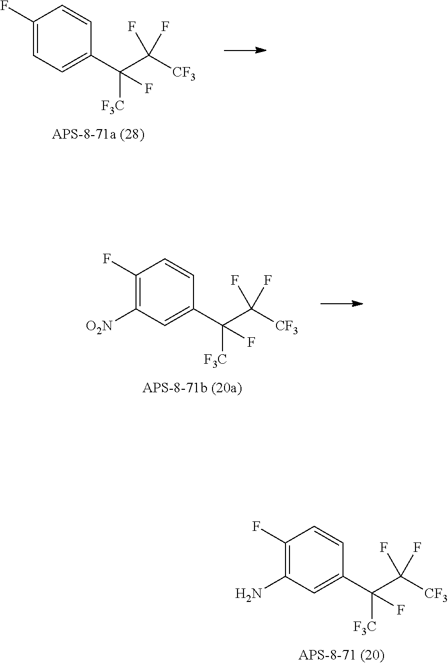 [SMILES: Fc1ccc(C(F)(C(F)(F)F)C(F)(F)C(F)(F)F)cc1 ; O=[N+]([O-])c1cc(C(F)(C(F)(F)F)C(F)(F)C(F)(F)F)ccc1F ; Nc1cc(C(F)(C(F)(F)F)C(F)(F)C(F)(F)F)ccc1F]

A 100 mL flask was equipped with an air-cooled reflux condenser and outlet adapter that was attached to a piece of tygon tubing, which was run into a water bath trap in a 1 L Erlenmeyer flask (to trap the BF₃ generated during the reaction). The flask was charged with 4-(perfluorobutan-2-yl)benzenediazonium tetrafluoroborate (27; 4.75 g, 11.6 mmol) and then was heated at 100° C., at which point the reaction initiated. The temperature was slowly increased to 120° C. over 1 h, which resulted in complete conversion of the solid into a liquid. At this point the evolution of BF₃ had ceased and the flask was allowed to cool to room temperature. The resulting liquid was diluted with pentane (10 mL) and filtered through a plug of Na₂SO₄ in a 4 mL pipet; washed plug with pentane (2 mL). The combined filtrates were concentrated under vacuum to leave 3.51 g of 1-fluoro-4-(perfluorobutan-2-yl)benzene (28) as a clear liquid. To the flask was added concd H₂SO₄ (20 mL) followed by fuming HNO₃ (820 μL, 90% w/w, 17.3 mmol). The mixture was heated at 80° C. for 16 h. After cooling to room temperature, the reaction was poured into 50:50 ice/water (200 mL) and extracted with Et₂O (1×100 mL, 2×50 mL). The organic extracts were pooled, washed with saturated NaHCO₃ (2×50 mL), water (50 mL) and brine (50 mL), dried (MgSO₄), and filtered. Concentration under vacuum gave 3.26 g of 1-fluoro-2-nitro-4-(perfluorobutan-2-yl)benzene (20a) as a yellow oil. To the 200 mL flask containing the oil was added palladium (1.0 g, 5% w/w on activated carbon, 0.47 mmol, 4 mol %) and MeOH (50 mL). A 3-way inlet adapter was attached, the flask was evacuated using a house vacuum line and then back-filled with H₂ from a balloon. The mixture was stirred under a balloon of H₂ for 7 h and then vacuum filtered through a pad of Celite (3×3 cm); washed pad with MeOH (2×10 mL). The combined filtrates were concentrated under vacuum to leave an oil, which was purified by silica gel chromatography (40 g cartridge), eluting at 30 mL/min and using a linear gradient of hexanes/CH₂Cl₂: 100:0→50:50 over 33 column volumes. Obtained 1.92 g (50% over three steps) of the title compound as a yellow liquid: 1H NMR (400 MHz, DMSO-d₆) δ 7.21 (dd, J=11.1, 8.7 Hz, 1H), 7.07 (d, J=8.1 Hz, 1H), 6.69-6.78 (m, 1H), 5.65 (s, 2H); ¹⁹F NMR (376 MHz, DMSO-d₆) δ −73.3-−73.0 (m, 3F), −78.4 (d, J=11.4 Hz, 3F), −120.8-−120.6 (m, 2F), −130.8-−130.5 (m, 1F), −181.3-−181.0 (m, 1F); LC-MS (ESI+) m/z: [M+H]⁺ Calcd for C₁₀H₆F₁₀N 330.0; Found 330.1.

### 1-Fluoro-2-nitro-4-(perfluorobutyl)benzene (APS-6-37 (18a))

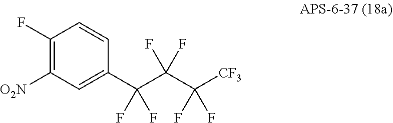 [SMILES: O=[N+]([O-])c1cc(C(F)(F)C(F)(F)C(F)(F)C(F)(F)F)ccc1F]

To a mixture of 1-fluoro-4-iodo-2-nitrobenzene (1.25 g, 4.68 mmol), activated copper powder (1.20 g, 18.9 mmol) and dry DMF (13 mL; deoxygenated by bubbling Ar for 10 min) was added heptafluoro-1-iodobutane (1.21 mL, 7.03 mmol) via syringe over 1 min. The mixture was stirred under Ar and heated at 100° C. for 48 h. The reaction mixture was allowed to cool to room temperature and then was filtered through a pad (3×3 cm) of Celite under vacuum; washed pad with Et₂O (2×20 mL). The combined filtrates were diluted with Et₂O (100 mL), then washed with a 60:40 mixture of saturated NH₄Cl solution/concd NH₄OH solution (3×30 mL), water (2×30 mL) and brine (2×30 mL), dried (MgSO₄), and filtered. Concentration under vacuum gave an orange semi-solid, which was purified by silica gel chromatography (25 g cartridge), eluting at 20 mL/min and using a linear gradient of hexanes/CH₂Cl₂: 100:0→60:40 over 30 column volumes. Obtained 1.07 g (64%) of the title compound as a colorless oil: 1H NMR (400 MHz, DMSO-d₆) δ 8.43 (dd, J=6.7, 2.3 Hz, 1H), 8.21 (dt, J=8.7, 3.2 Hz, 1H), 7.90 (dd, J=10.8, 8.8 Hz, 1H); ¹⁹F NMR (376 MHz, DMSO-d₆) δ −80.0 (t, J=9.2 Hz, 3F), −109.0 (t, J=13.7 Hz, 2F), −111.5 (s, 1F), −121.7-−121.4 (m, 2F), −124.8-−124.6 (m, 2F).

### 2-Fluoro-5-(perfluorobutyl)aniline (APS-6-40 (18))

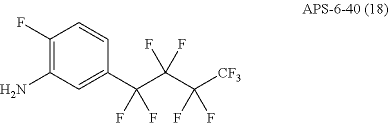 [SMILES: Nc1cc(C(F)(F)C(F)(F)C(F)(F)C(F)(F)F)ccc1F]

A 50 mL flask was charged with 1-fluoro-2-nitro-4-(perfluorobutyl)benzene (18a; 665 mg, 2.15 mmol), MeOH (15 mL) and iron powder (720 mg, 12.9 mmol). The mixture was cooled to 0° C., and concentrated HCl (2.1 mL, 26 mmol) was added dropwise by pipet over 5 min. The reaction was allowed to warm to room temperature over 30 min, and was stirred for an additional 3 h. The reaction was vacuum-filtered through a pad (2×2 cm) of Celite to remove unreacted iron powder and the pad was washed with MeOH (5 mL). The combined filtrates were diluted with water (75 mL) and CH₂Cl₂ (75 mL), and the pH of the aqueous phase was adjusted to 7-8 with 6 M NaOH solution (˜4.3 mL). The resulting mixture was vacuum-filtered through a pad (4×4 cm total size) of sand (3×4 cm) layered on top of Celite (1×4 cm) and the filter-cake was washed with CH₂Cl₂ (30 mL). The combined filtrates were transferred to a separatory funnel, the layers were separated, and the aqueous phase was extracted with CH₂Cl₂ (2×50 mL). The organic extracts were pooled, dried (Na₂SO₄), and filtered. The filtrate was concentrated under vacuum to leave an oil, which was purified by silica gel chromatography (25 g cartridge), eluting at 20 mL/min and using a linear gradient of hexanes/CH₂Cl₂: 100:0→50:50 over 25 column volumes. Obtained 473 mg (79%) of the title compound as a colorless oil: 1H NMR (400 MHz, DMSO-d₆) δ 7.21 (dd, J=11.2, 8.6 Hz, 1H), 7.05 (dd, J=8.2, 2.3 Hz, 1H), 6.77 (dt, J=8.3, 3.3 Hz, 1H), 5.64 (br s, 2H); ¹⁹F NMR (376 MHz, DMSO-d₆) δ −80.1 (t, J=9.2 Hz, 3F), −108.8 (t, J=13.7 Hz, 2F), −122.1 (q, J=9.2 Hz, 2F), −124.9-−124.7 (m, 2F), −129.0 (s, 1F); LC-MS (ESI+) m/z: [M+H]⁺ Calcd for C₁₀H₆F₁₀N 330.0; Found 330.1.

### 4-Fluoro-3-nitrobenzaldehyde (APS-4-48 (66a))

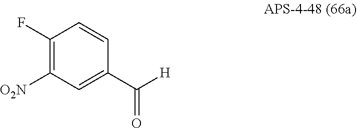 [SMILES: O=Cc1ccc(F)c([N+](=O)[O-])c1]

A 50 mL flask was charged with 4-fluorobenzaldehyde (2.20 mL, 20.5 mmol) and concentrated sulfuric acid (10 mL). The solution was cooled to 0° C. and fuming nitric acid (1.10 mL, 90% w/w in water, 23.3 mmol) was added dropwise over 5 min by pipet. The reaction was stirred for 2.5 h and allowed to warm as the ice-bath melted. The reaction was poured into rapidly stirred ice-water (150 mL), and was stirred for an additional 15 min. The resulting precipitate was collected by vacuum filtration, and the collected solid was washed with water (2×10 mL). Air-drying provided 1.84 g (53%) of the title compound as a white solid: ¹H NMR (400 MHz, DMSO-d₆) δ 10.07 (s, 1H), 8.68 (dd, J=7.5, 2.1 Hz, 1H), 8.33 (ddd, J=8.6, 4.3, 2.2 Hz, 1H), 7.82 (dd, J=11.0, 8.6 Hz, 1H); ¹⁹F NMR (376 MHz, DMSO-d₆) δ −109.6 (s, 1F); LC-MS (ESI+) m/z: [M+H]⁺ Calcd for C₇H₅FNO₃ 170.0; Found 170.1.

### 4-(Difluoromethyl)-1-fluoro-2-nitrobenzene (APS-4-50 (66b))

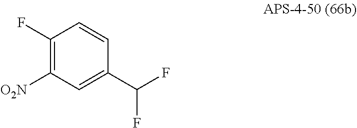 [SMILES: O=[N+]([O-])c1cc(C(F)F)ccc1F]

A flame-dried 50 mL flask, cooled under Ar, was charged with 4-fluoro-3-nitrobenzaldehyde (66a; 715 mg, 4.23 mmol) and CH₂Cl₂ (12 mL). The solution was cooled to −78° C. and (diethylamino)sulfur trifluoride (DAST; 1.12 mL, 8.48 mmol) was added dropwise over 3 min via syringe. The resulting bright-yellow heterogeneous reaction was allowed to stir at −78° C. for 3 h; over this time the reaction became homogeneous and light-red in color. The cooling bath was removed and the solution was allowed to warm to room temperature. The reaction was stirred at room temperature for 16 h and then was quenched with water (10 mL). The pH of the aqueous layer was adjusted to ˜7 with saturated NaHCO₃ solution and the mixture was extracted with CH₂Cl₂ (2×50 mL). The organic extracts were pooled, dried (Na₂SO₄), and filtered. Concentration under vacuum gave 790 mg of an orange oil, which was purified by silica gel chromatography (25 g cartridge), eluting at 20 mL/min and using a linear gradient of hexanes/EtOAc: 100:0→0:100 over 18 column volumes. Obtained 608 mg (75%) of the title compound as a yellow oil: 1H NMR (400 MHz, DMSO-d₆) δ 8.37-8.41 (m, 1H), 8.03-8.08 (m, 1H), 7.73-7.80 (m, 1H), 7.17 (t, J_{HF}=55.3 Hz, 1H); ¹⁹F NMR (376 MHz, DMSO-d₆) δ −109.8 (s, 2F), −114.8 (s, 1F).

### 5-(Difluoromethyl)-2-fluoroaniline (APS-4-53 (66))

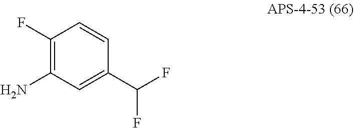 [SMILES: Nc1cc(C(F)F)ccc1F]

A 50 mL flask was charged sequentially with 4-(difluoromethyl)-1-fluoro-2-nitrobenzene (66b; 597 mg, 3.12 mmol), MeOH (15 mL) and iron powder (871 mg, 15.6 mmol). The mixture was stirred at room temperature and an aqueous solution of HCl (8.0 mL, 4.0 M solution in water, 32 mmol) was added dropwise over 1-2 min. After 1 h the reaction was decanted from the unreacted iron, and the remaining solution was diluted with CH₂Cl₂ (50 mL) and water (50 mL). The pH of the aqueous phase was adjusted to ˜7 with 6 N NaOH (˜5.3 mL), then the mixture was transferred to a separatory funnel, the layers were separated, and the aqueous phase was extracted with CH₂Cl₂ (2×30 mL). The organic extracts were pooled, dried (Na₂SO₄), and filtered. Concentration under vacuum gave a yellow oil, which was purified by silica gel chromatography (25 g cartridge), eluting at 20 mL/min and using a linear gradient of hexanes/EtOAc: 100:0→0:100 over 20 column volumes. Obtained 257 mg (51%) of the title compound as a clear colorless oil: ¹H NMR (400 MHz, DMSO-d₆) δ 7.04-7.13 (m, 1H), 6.91-6.97 (m, 1H), 6.86 (t, J_{HF}=56.1 Hz, 1H), 6.66-6.72 (m, 1H), 5.42 (br s, 2H); ¹⁹F NMR (376 MHz, DMSO-d₆) δ −108.0 (s, 2F), −131.5 (s, 1F); LC-MS (ESI+) m/z: [M+H]⁺ Calcd for C₇H₇F₃N 162.1 Found 162.1.

### 4-Chloro-N-methylpicolinamide (APS-4-76 (67))

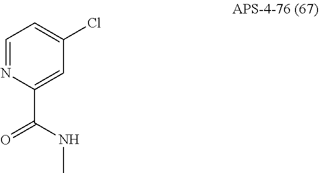

A flame-dried 250 mL flask, cooled under Ar, was charged with 4-chloropicolinic acid (10.0 g, 63.5 mmol), and THE (125 mL). The mixture was cooled to 0° C. and oxalyl chloride (6.70 mL, 79.2 mmol) was added dropwise over 5 min via syringe, followed by DMF (0.1 mL), which was added by syringe in one shot (CAUTION: rapid release of gas). After 30 min the reaction mixture was allowed to warm to room temperature and was stirred under a balloon of Ar for 15 h. The resulting brown solution was concentrated on a rotary-evaporator; a drying tube filled with KOH pellets was used to trap residual HCl. The remaining oil was concentrated to dryness from toluene (3×10 mL) and then was dried further under high vacuum to provide a solid. The crude 4-chloropicolinoyl chloride hydrochloride salt was placed under Ar and THF (50 mL) was added. The dark solution was cooled to 0° C. and methylamine (160 mL, 2.0 M solution in THF, 320 mmol) was added dropwise over 20 min via syringe. After 5 min the reaction was allowed to warm to room temperature and was stirred for 16 h. The reaction mixture was diluted with water (200 mL) and extracted with EtOAc (3×150 mL). The organic extracts were pooled, washed with water (100 mL) and brine (2×100 mL), dried (Na₂SO₄), and filtered. Concentration under vacuum gave ˜11 g of a red-brown oil, which was purified by silica gel chromatography (40 g cartridge), eluting at 30 mL/min and using a linear gradient of hexanes/EtOAc: 100:0→0:100 over 30 column volumes. The appropriate fractions were pooled [symbol] ed to dryness. The remaining clear colorless oil (˜10 g) was dissolved in a mixture of hexanes/CH₂Cl₂ (4:1; 150 mL) and allowed to stand at −20° C. for 12 h. The resulting precipitate was isolated by vacuum filtration, washed with hexanes (2×30 mL) and air-dried to yield 8.90 g (82%) of the title compound as a white solid: ¹H NMR (400 MHz, DMSO-d₆) δ 8.85 (br ap d, J=3.4 Hz, 1H), 8.62 (dd, J=5.3, 0.6 Hz, 1H), 8.01 (dd, J=2.2, 0.6 Hz, 1H), 7.75 (dd, J=5.3, 2.2 Hz, 1H), 2.82 (d, J=4.9 Hz, 3H); ¹³C NMR (100 MHz, DMSO-d₆) δ 163.1, 151.8, 150.0, 144.5, 126.3, 121.8, 26.1; LC-MS (ESI+) m/z: [M+H]⁺ Calcd for C₇H₈ClN₂O 171.0; Found 171.1.

### 4-(4-Aminophenoxy)-N-methylpicolinamide (APS-5-13 (4))

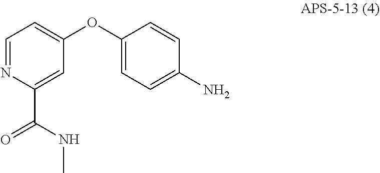 [SMILES: CNC(=O)c1cc(Oc2ccc(N)cc2)ccn1]

A two-necked 100 mL flask (equipped with an inlet adapter and septum) was flame-dried under vacuum and cooled under Ar. The flask was charged with 4-aminophenol (2.09 g, 19.2 mmol) and DMF (30 mL). To the stirred solution was added potassium tert-butoxide (2.14 g, 19.1 mmol) in portions over 1 min. The resulting light-brown mixture was stirred for 2 h, then 4-chloro-N-methylpicolinamide (2.17 g, 12.7 mmol) was added in one portion, and the reaction was heated at 80° C. for 4 h under a balloon of Ar. The reaction was allowed to cool to room temperature and then was poured into stirred ice-water (100 mL). Stirring was continued for 15 min and then the mixture was extracted with EtOAc (1×100 mL and 2×50 mL). The organic extracts were pooled, washed with 1 M KOH (3×50 mL), water (50 mL) and brine (2×50 mL), dried (Na₂SO₄), and filtered. Concentration under vacuum gave 3.23 g of an orange oil, which was purified by silica gel chromatography (40 g cartridge), eluting at 30 mL/min and using a linear gradient of hexanes/EtOAc: 100:0→0:100 over 35 column volumes. Obtained 2.69 g (87%) of the title compound as an off-white solid: ¹H NMR (400 MHz, DMSO-d₆) δ 8.73 (br q, J=4.6 Hz, 1H), 8.45 (d, J=5.6 Hz, 1H), 7.34 (d, J=2.5 Hz, 1H), 7.06 (dd, J=5.5, 2.6 Hz, 1H), 6.86 (d, J=8.8 Hz, 2H), 6.64 (d, J=8.8 Hz, 2H), 5.17 (s, 2H), 2.78 (d, J=4.9 Hz, 3H); ³C NMR (100 MHz, DMSO-d₆) δ 166.8, 163.9, 152.3, 150.1, 146.9, 142.8, 121.6, 114.9, 113.7, 108.3, 26.0; LC-MS (ESI+) m/z: [M+H]⁺ Calcd for C₁₃H₁₄N₃O₂244.1; Found 244.2.

### 4-(4-Amino-3-fluorophenoxy)-N-methylpicolinamide (APS-5-27 (5))

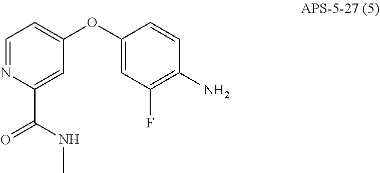 [SMILES: CNC(=O)c1cc(Oc2ccc(N)c(F)c2)ccn1]

An oven-dried two-necked 100 mL flask (equipped with an inlet adapter and septum), under Ar, was charged with 4-amino-3-fluorophenol (1.12 g, 8.81 mmol) and DMF (18 mL). To the stirred solution was added potassium tert-butoxide (978 mg, 8.72 mmol) in portions over 2 min. The resulting dark-purple mixture was stirred for 3 h, then 4-chloro-N-methylpicolinamide (1.06 g, 6.21 mmol) was added in one portion, and the reaction was heated at 90° C. for 10 h under a balloon of Ar. The reaction was allowed to cool to room temperature and then was poured into stirred ice-water (50 mL). Stirring was continued for 15 min and then the mixture was extracted with EtOAc (3×50 mL). The organic extracts were pooled, washed with 1 M KOH (3×50 mL), water (50 mL) and brine (2×50 mL), dried (Na₂SO₄), and filtered. Concentration under vacuum gave a brown solid, which was purified by silica gel chromatography (40 g cartridge), eluting at 30 mL/min and using a linear gradient of hexanes/EtOAc: 100:0→0:100 over 38 column volumes. Obtained 882 mg (54%) of the title compound as a light-brown solid: ¹H NMR (400 MHz, DMSO-d₆) δ 8.74 (br q, J=4.6 Hz, 1H), 8.47 (d, J=5.6 Hz, 1H), 7.35 (d, J=2.5 Hz, 1H), 7.09 (dd, J=5.6, 2.7 Hz, 1H), 7.01 (dd, J=11.9, 2.6 Hz, 1H), 6.81-6.89 (m, 1H), 6.76-6.80 (m, 1H), 5.22 (br s, 2H), 2.78 (d, J=4.9 Hz, 3H); ¹⁹F NMR (376 MHz, DMSO-d₆) δ −130.7 (s, 1F); LC-MS (ESI+) m/z: [M+H]⁺ Calcd for C₁₃H₁₃FN₃O₂ 262.1; Found 262.1.

### 3-Iodo-1-isopropyl-1H-pyrazolo[3,4-d]pyrimidin-4-amine (APS-6-75 (68))

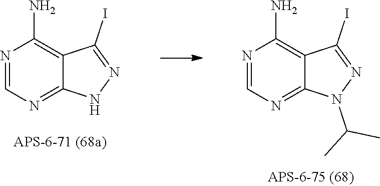

A flame-dried 100 mL flask, cooled under Ar, was charged with 1H-pyrazolo[3,4-d]pyrimidin-4-amine (5.00 g, 37.0 mmol), N-iodosuccinimide (12.5 g, 55.6 mmol) and DMF (40 mL). The mixture was heated at 80° C., under a balloon of Ar, for 22 h. The reaction mixture was allowed to cool to room temperature, diluted with water (40 mL) and stirred for 20 min. The solid was collected by vacuum filtration, washed with water (3×10 mL) and air-dried to provide 3-iodo-1H-pyrazolo[3,4-d]pyrimidin-4-amine (68a; 8.16 g, 31.3 mmol) as an off-white solid. The solid was added to a flame-dried 200 mL flask, under Ar, followed by oven-dried K₂CO₃ (5.19 g, 37.6 mmol) and DMF (60 mL). 2-Bromopropane (3.00 mL, 32.0 mmol) was [symbol] ge in a steady stream and the mixture was heated at 80° C. under a balloon of Ar for 18 h. The reaction was allowed to cool to room temperature, diluted with water (150 mL) and extracted with EtOAc (3×150 mL). The organic extracts were pooled, washed with water (2×100 mL) and brine (2×100 mL), dried (Na₂SO₄), and filtered. Concentration under vacuum gave an orange solid, which was recrystallized from MeOH (˜100 mL). The solid was isolated by vacuum filtration, washed with EtOH (25 mL) and hexanes (2×25 mL), and then air-dried. Obtained 6.13 g (65% over two steps) of the title compound as white needles: ¹H NMR (400 MHz, DMSO-d₆) δ ppm 8.19 (s, 1H), 7.19 (br s, 2H), 4.96 (sept, J=6.7 Hz, 1H), 1.42 (d, J=6.6 Hz, 6H); LC-MS (ESI+) m/z: [M+H]⁺ Calcd for C₈H₁₁IN₅ 304.0, found 304.2.

### 3-(4-Aminophenyl)-1-isopropyl-1H-pyrazolo[3,4-d]pyrimidin-4-amine (APS-6-82 (69))

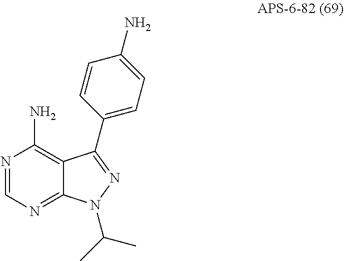 [SMILES: CC(C)n1nc(-c2ccc(N)cc2)c2c(N)ncnc21]

A 40 mL vial was charged with 3-iodo-1-isopropyl-1H-pyrazolo[3,4-d]pyrimidin-4-amine (68; 618 mg, 2.04 mmol), 4-(4,4,5,5-tetramethyl-1,3,2-dioxaborolan-2-yl)aniline (581 mg, 2.65 mmol), Na₂CO₃ (650 mg, 6.13 mmol) and tetrakis(triphenylphosphine)-palladium(0) (118 mg, 0.102 mmol), then 1,4-dioxane (16 mL) and water (4 mL) were added (both solvents were deoxygenated by sparging with Ar for 10 min). The headspace was purged with Ar, the vial was sealed with a screwcap and the reaction mixture was heated at 90° C. for 24 h. After the reaction had cooled to room temperature it was diluted with a mixture (95:5) of CH₂Cl₂/MeOH (15 mL) and water (5 mL), and then vacuum filtered through a pad (3×3 cm) of Celite; the pad was washed with CH₂Cl₂ (2×10 mL). The combined filtrates were transferred to a separatory funnel, diluted with brine (40 mL) and the layers were separated; the aqueous layer was extracted with CH₂Cl₂ (2×50 mL). The organic extracts were pooled, dried (Na₂SO₄), filtered and concentrated to dryness. The remaining semi-solid was purified by silica gel chromatography (25 g cartridge), eluting at 25 mL/min and using a linear gradient of CH₂Cl₂/MeOH: 100:0→90:10 over 30 column volumes. Obtained 520 mg (95%) of the title compound as an off-white solid: 1H NMR (400 MHz, DMSO-d₆) δ ppm 8.19 (s, 1H), 7.31 (d, J=8.6 Hz, 2H), 6.70 (d, J=8.6 Hz, 2H), 5.41 (br s, 2H), 5.01 (sept, J=6.7 Hz, 1H), 1.46 (d, J=6.6 Hz, 6H); LC-MS (ESI+) m/z: [M+H]⁻ Calcd for C₁₄H₁₇N₆ 269.2, found 269.4.

### 5-Iodo-7-isopropyl-7H-pyrrolo[2,3-d]pyrimidin-4-amine (APS-6-67 (70))

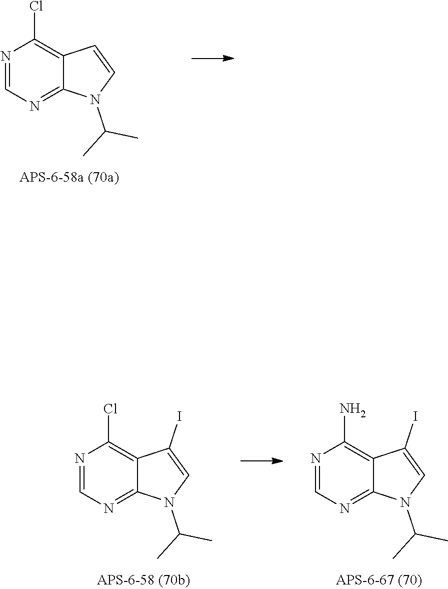

A two-necked 100 mL flask, equipped with inlet and outlet adapters, was charged with 4-chloro-7H-pyrrolo[2,3-d]pyrimidine (2.00 g, 13.0 mmol) and DMF (40 mL), under Ar. The solution was cooled to 0° C. and then NaH (730 mg, 60% w/w in mineral oil, 18.3 mmol) was added in portions over 10 min. The mixture was stirred at 0° C. for 30 min and then at room temperature for 30 min. The mixture was cooled to 0° C. and then 2-bromopropane (1.83 mL, 19.5 mmol) was added dropwise over 3 min. The reaction was allowed to warm to room temperature overnight. After stirring for a total of 18 h, the mixture was poured into water (100 mL) and extracted with EtOAc (3×50 mL). The organic extracts were pooled, washed with brine (3×50 mL), dried (Na₂SO₄), and filtered. Concentration under vacuum gave a solid, which was purified by silica gel chromatography (40 g cartridge), eluting at 30 mL/min and using a linear gradient of hexanes/EtOAc: 100:0→40:60 over 35 column volumes. Obtained 1.85 g (73%) of 4-chloro-7-isopropyl-7H-pyrrolo[2,3-d]pyrimidine (70a) as an off-white solid: LC-MS (ESI+) m/z: [M+H]⁺ Calcd for C₉H₁₁ClN₃ 196.1; Found 196.2.

The solid 70a (1.85 g, 9.46 mmol) was placed in a 100 mL flask along with NaHCO₃ (1.60 g, 19.0 mmol) and MeCN (30 mL), under Ar. To the mixture was added ICl (12.0 mL, 1.0 M solution in CH₂Cl₂, 12 mmol) dropwise over 5 min. The reaction was stirred for 16 h, then was poured into water (150 mL) and extracted with CH₂Cl₂ (3×50 mL). The organic extracts were pooled, dried (Na₂SO₄), filtered and concentrated under vacuum. The remaining solid was purified by silica gel chromatography (40 g cartridge), eluting at 30 mL/min and using a linear gradient of hexanes/EtOAc: 100:0→50:50 over 25 column volumes. Obtained 2.71 g (89%) of 4-chloro-5-iodo-7-isopropyl-7H-pyrrolo[2,3-d]pyrimidine (70b) as a white solid: LC-MS (ESI+) m/z: [M+H]⁺ Calcd for C₉H₁₀ClIN₃ 322.0; Found 322.1.

The solid 70b (1.41 g, 4.39 mmol) was placed in a 48 mL heavy-walled sealable tube along with a 50:50 mixture of 2-PrOH/1,4-dioxane (15 mL). Ammonia gas was bubbled through the mixture for 5 min, then the tube was sealed and the mixture was heated at 130° C. for 18 h. The mixture was cooled to 0° C. before opening the tube, then the contents were transferred to a 100 mL flask and concentrated to dryness. The remaining solid was triturated with a 75:25 mixture of Et₂O/EtOAc (30 mL) and collected by vacuum filtration; washed solid with Et₂O (5 mL). Air-drying provided 1.25 g (95%) of the title compound (70) as a white solid: ¹H NMR (400 MHz, DMSO-d₆) δ 8.08 (s, 1H), 7.57 (s, 1H), 7.28 (br s, 2H), 4.89 (sept, J=6.8 Hz, 1H), 1.40 (d, J=6.8 Hz, 6H); LC-MS (ESI+) m/z: [M+H]⁺ Calcd for C₉H₁₂IN₄ 303.0; Found 303.2.

### 5-Iodo-7-methyl-7H-pyrrolo[2,3-d]pyrimidin-4-amine (APS-4-73)

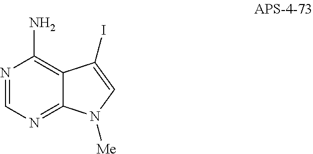 [SMILES: Cn1cc(I)c2c(N)ncnc21]

A 25 mL flask, equipped with an inlet adapter, was charged with 4-chloro-7H-pyrrolo[2,3-d]pyrimidine (500 mg, 3.26 mmol) and DMF (4 mL), under Ar. The solution was cooled to 0° C. and then NaH (183 mg, 60% w/w in mineral oil, 4.58 mmol) was added in portions over 10 min. The mixture was stirred at 0° C. for 15 min and then at room temperature for 30 min. The mixture was cooled to 0° C. and then iodomethane (325 μL, 5.22 mmol) was added dropwise over 5 min. The reaction was allowed to warm to room temperature over 10 min. After stirring for a total of 30 min, the mixture was poured into water (50 mL) and extracted with EtOAc (3×30 mL). The organic extracts were pooled, washed with brine (3×30 mL), dried (Na₂SO₄), and filtered. Concentration under vacuum gave a solid, which was placed under Ar in a 100 mL flask along with NaHCO₃ (411 mg, 4.89 mmol) and MeCN (10 mL). A solution of ICl (4.10 mL, 1.0 M solution in CH₂Cl₂, 4.1 mmol) was added dropwise over 5 min. The dark reaction was stirred for 14 h, then diluted with H₂O (30 mL) and extracted with CH₂Cl₂ (3×30 mL). The organic extracts were pooled, washed with 10% w/v NaHSO₃ (2×30 mL), dried (Na₂SO₄), filtered and concentrated under vacuum. Obtained −800 mg of a brown solid that was triturated with hot EtOAc (˜8 mL) and collected by vacuum filtration to provide 573 mg (60% over 2 steps) of a grey solid. The solid (573 mg, 1.95 mmol) was placed in a 15 mL heavy-walled sealable tube along with a 50:50 mixture of 2-PrOH/1,4-dioxane (8 mL). Ammonia gas was bubbled through the mixture for 5 min, then the tube was sealed and the mixture was heated at 120° C. for 8 h. The mixture was cooled to 0° C. before opening the tube. The contents were allowed to stand at 0° C. for 1 h and the resulting precipitate was collected by vacuum filtration. The collected brown solid was purified by silica gel chromatography (25 g cartridge), eluting at 20 mL/min and using a linear gradient of CH₂Cl₂/MeOH: 100:0→90:10 over 30 column volumes. Obtained 286 mg (53%) of the title compound as a light-orange solid: ¹H NMR (400 MHz, DMSO-d₆) δ 8.10 (s, 1H), 7.42 (s, 1H), 3.67 (s, 3H); LC-MS (ESI+) m/z: [M+H]⁻ Calcd for C₇HgIN₄ 275.0; Found 275.1.

### 7-Cyclopentyl-5-iodo-7H-pyrrolo[2,3-d]pyrimidin-4-amine (APS-4-75)

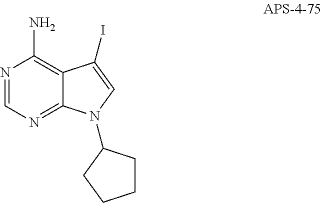 [SMILES: Nc1ncnc2c1c(I)cn2C1CCCC1]

A 25 mL flask, equipped with an inlet adapter, was charged with 4-chloro-7H-pyrrolo[2,3-d]pyrimidine (500 mg, 3.26 mmol) and DMF (4 mL), under Ar. The solution was cooled to 0° C. and then NaH (183 mg, 60% w/w in mineral oil, 4.58 mmol) was added in portions over 10 min. The mixture was stirred at 0° C. for 15 min and then at room temperature for 30 min. The mixture was cooled to 0° C. and then bromocyclopentane (560 μL, 5.22 mmol) was added dropwise over 5 min. The reaction was allowed to warm to room temperature overnight. After stirring for a total of 18 h, the mixture was poured into water (50 mL) and extracted with EtOAc (3×30 mL). The organic extracts were pooled, washed with brine (3×30 mL), dried (Na₂SO₄), and filtered. Concentration under vacuum gave a solid, which was placed under Ar in a 100 mL flask along with NaHCO₃ (411 mg, 4.89 mmol) and MeCN (10 mL). A solution of ICl (4.10 mL, 1.0 M solution in CH₂Cl₂, 4.1 mmol) was added dropwise over 5 min. The dark reaction was stirred for 14 h, then diluted with H₂O (30 mL) and extracted with CH₂Cl₂ (3×30 mL). The organic extracts were pooled, washed with 10% w/v NaHSO₃ (2×30 mL), dried (Na₂SO₄), filtered and concentrated under vacuum. Obtained ˜1 g of a brown solid, which was recrystallized from 2-PrOH (˜10 mL) to provide 700 mg (62% over 2 steps) as silver plates. The solid (700 mg, 2.01 mmol) was placed in a 15 mL heavy-walled sealable tube along with a 70:30 mixture of 2-PrOH/1,4-dioxane (10 mL). Ammonia gas was bubbled through the mixture for 5 min, then the tube was sealed and the mixture was heated at 120° C. for 15 h. The mixture was cooled to 0° C. before opening the tube. The contents were allowed to stand for 1 h and the resulting precipitate was collected by vacuum filtration; washed solid with 2-PrOH. Air-drying provided 475 mg (72%) of the title compound as a white solid: ¹H NMR (400 MHz, DMSO-d₆) δ 8.08 (s, 1H), 7.52 (s, 1H), 4.99 (quin, J=7.6 Hz, 1H), 1.97-2.15 (m, 2H), 1.74-1.92 (m, 4H), 1.65 (br s, 2H); LC-MS (ESI+) m/z: [M+H]⁻ Calcd for C₁₁H₁₄IN₄ 329.0; Found 329.1.

### 5-(4-Aminophenyl)-7-isopropyl-7H-pyrrolo[2,3-d]pyrimidin-4-amine (APS-6-78 (71))

A 40 mL vial was charged with 5-iodo-7-isopropyl-7H-pyrrolo[2,3-d]pyrimidin-

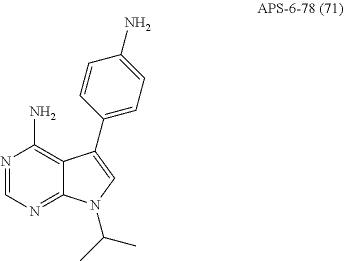 [SMILES: CC(C)n1cc(-c2ccc(N)cc2)c2c(N)ncnc21]

4-amine (70; 1.20 g, 3.97 mmol), 4-(4,4,5,5-tetramethyl-1,3,2-dioxaborolan-2-yl)aniline (1.04 g, 4.75 mmol), Na₂CO₃ (1.26 g, 11.9 mmol) and tetrakis(triphenylphosphine)-palladium(0) (229 mg, 0.198 mmol), then 1,4-dioxane (24 mL) and water (6 mL) were added (both solvents were deoxygenated by sparging with Ar for 10 min). The headspace was purged with Ar, the vial was sealed with a screwcap and the reaction mixture was heated at 90° C. for 18 h. After the reaction had cooled to room temperature it was diluted with a mixture (95:5) of CH₂Cl₂/MeOH (15 mL) and water (5 mL), and then vacuum filtered through a pad (3×3 cm) of Celite; the pad was washed with CH₂Cl₂ (2×10 mL). The combined filtrates were transferred to a separatory funnel, diluted with brine (40 mL) and the layers were separated; the aqueous layer was extracted with CH₂Cl₂ (2×75 mL). The organic extracts were pooled, dried (Na₂SO₄), filtered and concentrated to dryness. The remaining semi-solid was purified by silica gel chromatography (40 g cartridge), eluting at 30 mL/min and using a linear gradient of CH₂Cl₂/MeOH: 100:0→90:10 over 35 column volumes. Obtained 679 mg (64%) of the title compound as an off-white solid: 1H NMR (400 MHz, DMSO-d₆) δ 8.09 (s, 1H), 7.23 (s, 1H), 7.11 (d, J=8.3 Hz, 2H), 6.65 (d, J=8.6 Hz, 2H), 5.16 (s, 2H), 4.94 (sept, J=6.7 Hz, 1H), 1.44 (d, J=6.6 Hz, 6H); LC-MS (ESI+) m/z: [M+H]⁺ Calcd for C₁₅H₁₈N₅ 268.2; Found 268.3.

### 5-(4-Aminophenyl)-7-methyl-7H-pyrrolo[2,3-d]pyrimidin-4-amine (APS-6-88-1)

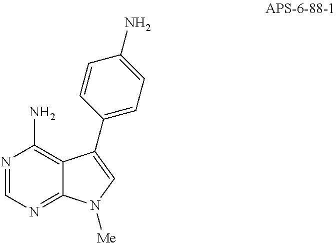 [SMILES: Cn1cc(-c2ccc(N)cc2)c2c(N)ncnc21]

A 20 mL vial was charged with 5-Iodo-7-methyl-7H-pyrrolo[2,3-d]pyrimidin-4-amine (235 mg, 0.857 mmol), 4-(4,4,5,5-tetramethyl-1,3,2-dioxaborolan-2-yl)aniline (244 mg, 1.11 mmol), Na₂CO₃ (273 mg, 2.58 mmol) and tetrakis(triphenylphosphine)-palladium(0) (49.5 mg, 0.043 mmol), then 1,4-dioxane (7.2 mL) and water (1.8 mL) were added (both solvents were deoxygenated by purging with Ar for 10 min). The headspace was purged with Ar, the vial was sealed with a screwcap and the reaction mixture was heated at 90° C. for 21 h. After the reaction had cooled to room temperature it was diluted with a mixture (90:10) of CH₂Cl₂/MeOH (10 mL) and then vacuum filtered through a pad (2.5×2.5 cm) of Celite; the pad was washed with CH₂Cl₂ (2×5 mL). The combined filtrates were transferred to a separatory funnel, diluted with brine (30 mL) and the layers were separated; the aqueous layer was extracted with CH₂Cl₂ (2×30 mL). The organic extracts were pooled, dried (Na₂SO₄), filtered and concentrated to dryness. The remaining semi-solid was purified by silica gel chromatography (25 g cartridge), eluting at 20 mL/min and using a linear gradient of CH₂Cl₂/MeOH: 100:0→90:10 over 35 column volumes. Obtained 67.5 mg (33%) of the title compound as an off-white solid: LC-MS (ESI+) m/z: [M+H]⁺ Calcd for C₁₃H₁₄N₅ 240.3; Found 240.4.

### 5-(4-Aminophenyl)-7-cyclopentyl-7H-pyrrolo[2,3-d]pyrimidin-4-amine (APS-6-88-2)

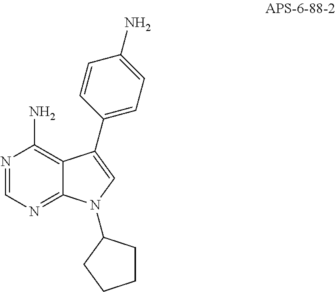 [SMILES: Nc1ccc(-c2cn(C3CCCC3)c3ncnc(N)c23)cc1]

A 20 mL vial was charged with 7-cyclopentyl-5-iodo-7H-pyrrolo[2,3-d]pyrimidin-4-amine (220 mg, 0.670 mmol), 4-(4,4,5,5-tetramethyl-1,3,2-dioxaborolan-2-yl)aniline (191 mg, 0.872 mmol), Na₂CO₃ (213 mg, 2.01 mmol) and tetrakis(triphenylphosphine)-palladium(0) (38.7 mg, 0.034 mmol), then 1,4-dioxane (5.6 mL) and water (1.4 mL) were added (both solvents were deoxygenated by purging with Ar for 10 min). The headspace was purged with Ar, the vial was sealed with a screwcap and the reaction mixture was heated at 90° C. for 21 h. After the reaction had cooled to room temperature it was diluted with a mixture (90:10) of CH₂Cl₂/MeOH (10 mL) and then vacuum filtered through a pad (2.5×2.5 cm) of Celite; the pad was washed with CH₂Cl₂ (2×5 mL). The combined filtrates were transferred to a separatory funnel, diluted with brine (30 mL) and the layers were separated; the aqueous layer was extracted with CH₂Cl₂ (2×30 mL). The organic extracts were pooled, dried (Na₂SO₄), filtered and concentrated to dryness. The remaining semi-solid was purified by silica gel chromatography (25 g cartridge), eluting at 20 mL/min and using a linear gradient of CH₂Cl₂/MeOH: 100:0→90:10 over 35 column volumes. Obtained 83.0 mg (42%) of the title compound as an off-white solid: LC-MS (ESI+) m/z: [M+H]⁺ Calcd for C₁₇H₂₀N₅ 294.4; Found 294.4.

### Example 3—Synthesis of Compounds

### 1-(4-(4-Amino-1-isopropyl-1H-pyrazolo[3,4-d]pyrimidin-3-yl)phenyl)-3-(3-(trifluoromethyl)phenyl)urea (AD57 (92))

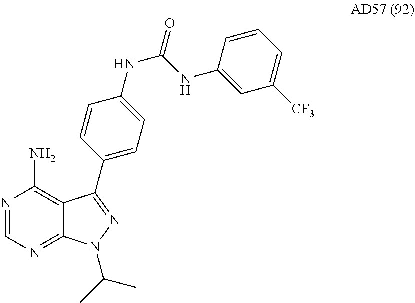 [SMILES: CC(C)n1nc(-c2ccc(NC(=O)Nc3cccc(C(F)(F)F)c3)cc2)c2c(N)ncnc21]

To a −5° C. solution of 69 (150 mg, 0.559 mmol) and a 50:50 mixture of CH₂Cl₂/DMF (2 mL), was added a solution of 1-isocyanato-3-(trifluoromethyl)benzene (80.0 μL, 0.581 mmol) and CH₂Cl₂ (1 mL), down the wall of the flask over 5 min. The solution was allowed to warm to room temperature overnight as the bath melted. After stirring for 12 h, the solution was concentrated to dryness and the remaining material was purified by silica gel chromatography (25 g cartridge), eluting at 25 mL/min and using a linear gradient of CH₂Cl₂/MeOH: 100:0→90:10 over 30 column volumes. Obtained 190 mg (75%) of the title compound as a white solid: ¹H NMR (400 MHz, DMSO-d₆) δ 9.10 (s, 1H), 9.03 (s, 1H), 8.23 (s, 1H), 8.05 (s, 1H), 7.56-7.69 (m, 5H), 7.49-7.56 (m, 1H), 7.33 (d, J=7.6 Hz, 1H), 5.06 (sept, J=6.8 Hz, 1H), 1.49 (d, J=6.6 Hz, 6H); ¹⁹F NMR (376 MHz, DMSO-d₆) δ −60.8 (s, 3F); LC-MS (ESI+) m/z: [M+H]⁺ Calcd for C₂₂H₂₁F₃N₇O 456.2; Found 456.4.

### 1-(4-(4-Amino-1-isopropyl-1H-pyrazolo[3,4-d]pyrimidin-3-yl)phenyl)-3-(3-(perfluoroethyl)phenyl)urea (APS-5-86-1 (93))

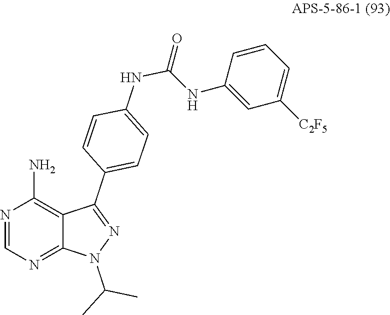

A solution of 3-(perfluoroethyl)aniline (11; 65.3 mg, 0.309 mmol) and CH₂Cl₂ (0.5 mL) was added to a 0° C. solution of CDI (50.2 mg, 0.310 mmol) and CH₂Cl₂ (0.5 mL) dropwise over 1 min. The solution was stirred at 0° C. for 1 h and then at room temperature for 24 h. The acyl imidazole solution was added to a −5° C. solution of 69 (75.5 mg, 0.281 mmol) and a 50:50 mixture of CH₂Cl₂/DMF (1 mL), down the wall of the flask over 5 min. The solution was allowed to warm to room temperature as the bath melted. After stirring for 12 h, the solution was concentrated to dryness and the remaining material was purified by silica gel chromatography (25 g cartridge), eluting at 25 mL/min and using a linear gradient of CH₂Cl₂/MeOH: 100:0→90:10 over 35 column volumes. Obtained 38.3 mg (27%) of the title compound as a white solid: 1H NMR (400 MHz, DMSO-d₆) δ 9.16 (br s, 1H), 9.03 (br s, 1H), 8.23 (s, 1H), 8.03 (br s, 1H), 7.48-7.75 (m, 6H), 7.29 (d, J=7.1 Hz, 1H), 5.06 (sept, J=6.8 Hz, 1H), 1.49 (d, J=6.6 Hz, 6H); ¹⁹F NMR (376 MHz, DMSO-d₆) δ −83.5 (s, 3F), −113.2 (s, 2F); LC-MS (ESI+) m/z: [M+H]⁺ Calcd for C₂₃H₂₁F₅N₇O 506.2; Found 506.7.

### 1-(4-(4-Amino-1-isopropyl-1H-pyrazolo[3,4-d]pyrimidin-3-yl)phenyl)-3-(3-(perfluoropropan-2-yl)phenyl)urea (APS-7-46-1 (94))

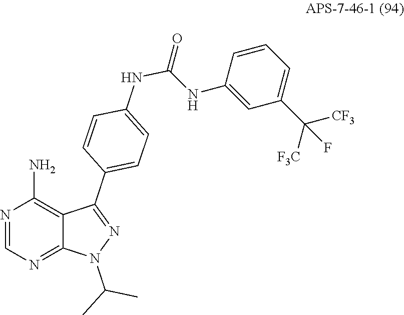 [SMILES: CC(C)n1nc(-c2ccc(NC(=O)Nc3cccc(C(F)(C(F)(F)F)C(F)(F)F)c3)cc2)c2c(N)ncnc21]

A solution of 3-(perfluoropropan-2-yl)aniline (15; 99.5 mg, 0.381 mmol) and CH₂Cl₂ (0.5 mL) was added to a 0° C. solution of CDI (67.5 mg, 0.416 mmol) and CH₂Cl₂ (0.5 mL) dropwise over 1 min. The solution was stirred at 0° C. for 1 h and then at room temperature for 24 h. The acyl imidazole solution was added to a −5° C. solution of 69 (102 mg, 0.380 mmol) and a 50:50 mixture of CH₂Cl₂/DMF (2 mL), down the wall of the flask over 5 min. The solution was allowed to warm to room temperature as the bath melted. After stirring for 12 h, the solution was concentrated to dryness and the remaining material was purified by silica gel chromatography (25 g cartridge), eluting at 25 mL/min and using a linear gradient of CH₂Cl₂/MeOH: 100:0→90:10 over 32 column volumes. Obtained 89.4 mg (42%) of the title compound as a white solid: 1H NMR (400 MHz, DMSO-d₆) δ 9.13 (s, 1H), 8.99 (s, 1H), 8.23 (s, 1H), 8.06 (s, 1H), 7.52-7.68 (m, 6H), 7.26 (d, J=7.6 Hz, 1H), 5.05 (sept, J=6.7 Hz, 1H), 1.49 (d, J=6.6 Hz, 6H); ¹⁹F NMR (376 MHz, DMSO-d₆) δ −74.6 (d, J=9.2 Hz, 6F), −181.2-−181.0 (m, 1F); LC-MS (ESI+) m/z: [M+H]⁻ Calcd for C₂₄H₂₁F₇N₇O 556.2; Found 556.4.

### 1-(4-(4-Amino-1-isopropyl-1H-pyrazolo[3,4-d]pyrimidin-3-yl)phenyl)-3-(3-(perfluoropropyl)phenyl)urea 2,2,2-trifluoroacetate (APS-7-52-1 (95))

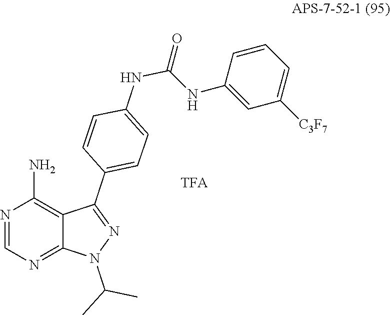

A solution of 3-(perfluoropropyl)aniline (13; 99.2 mg, 0.380 mmol) and CH₂Cl₂ (0.5 mL) was added to a 0° C. solution of CDI (67.5 mg, 0.416 mmol) and CH₂Cl₂ (0.5 mL) dropwise over 1 min. The solution was stirred at 0° C. for 1 h and then at room temperature for 24 h. The acyl imidazole solution was added to a −5° C. solution of 69 (102 mg, 0.380 mmol) and a 50:50 mixture of CH₂Cl₂/DMF (2 mL), down the wall of the flask over 5 min. The solution was allowed to warm to room temperature as the bath melted. After stirring for 12 h, the solution was concentrated to dryness and the remaining material was purified by silica gel chromatography (25 g cartridge), eluting at 25 mL/min and using a linear gradient of CH₂Cl₂/MeOH: 100:0→90:10 over 32 column volumes. Obtained 139 mg (66%) of the title compound as a white solid: 1H NMR (400 MHz, DMSO-d₆) δ 9.45 (s, 1H), 9.35 (s, 1H), 8.42 (s, 1H), 8.04 (s, 1H), 7.51-7.73 (m, 6H), 7.27 (d, J=7.6 Hz, 1H), 5.10 (sept, J=6.6 Hz, 1H), 1.51 (d, J=6.8 Hz, 6H); ¹⁹F NMR (376 MHz, DMSO-d₆) δ −73.9 (s, 3F), −79.0 (t, J=9.2 Hz, 3F), −110.3-−110.0 (m, 2F), −125.7 (s, 2F); LC-MS (ESI+) m/z: [M+H]⁺ Calcd for C₂₄H₂₁F₇N₇O 556.2; Found 556.7.

### 1-(4-(4-Amino-1-isopropyl-1H-pyrazolo[3,4-d]pyrimidin-3-yl)phenyl)-3-(3-(perfluorobutan-2-yl)phenyl)urea 2,2,2-trifluoroacetate (APS-8-56-3 (96))

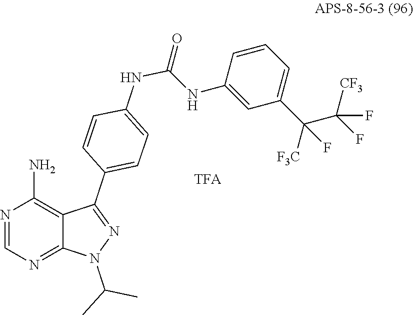 [SMILES: CC(C)n1nc(-c2ccc(NC(=O)Nc3cccc(C(F)(C(F)(F)F)C(F)(F)C(F)(F)F)c3)cc2)c2c(N)ncnc21]

A solution of 3-(perfluorobutan-2-yl)aniline (19; 128 mg, 0.411 mmol) and CH₂Cl₂ (0.5 mL) was added to a 0° C. solution of CDI (70.0 mg, 0.432 mmol) and CH₂Cl₂ (0.5 mL) dropwise over 1 min. The solution was stirred at 0° C. for 1 h and then at room temperature for 24 h. The acyl imidazole solution was added to a −5° C. solution of 69 (110 mg, 0.410 mmol) and a 50:50 mixture of CH₂Cl₂/DMF (2 mL), down the wall of the flask over 5 min. The [symbol] lowed to warm to room temperature as the bath melted. After stirring for 12 h, the solution was concentrated to dryness and the remaining material was purified by silica gel chromatography (25 g cartridge), eluting at 25 mL/min and using a linear gradient of CH₂Cl₂/MeOH: 100:0→90:10 over 30 column volumes. Obtained 154 mg (62%) of the title compound as a white solid: ¹H NMR (400 MHz, DMSO-d₆) δ 9.49 (s, 1H), 9.37 (s, 1H), 8.43 (s, 1H), 8.08 (s, 1H), 7.66-7.72 (m, 2H), 7.58-7.65 (m, 3H), 7.52-7.58 (m, 1H), 7.24 (d, J=7.6 Hz, 1H), 5.10 (sept, J=6.7 Hz, 1H), 1.51 (d, J=6.8 Hz, 6H); ¹⁹F NMR (376 MHz, DMSO-d₆) δ −73.1-−72.8 (m, 3F), −73.9 (s, 3F), −78.5-−78.3 (m, 3F), −120.7-−120.4 (m, 2F), −182.2-−181.8 (m, 1F); LC-MS (ESI+) m/z: [M+H]⁺ Calcd for C₂₅H₂₁F₉N₇O 606.2; Found 606.7.

### 1-(4-(4-Amino-1-isopropyl-1H-pyrazolo[3,4-d]pyrimidin-3-yl)phenyl)-3-(3-(perfluorobutyl)phenyl)urea (APS-7-46-2 (59))

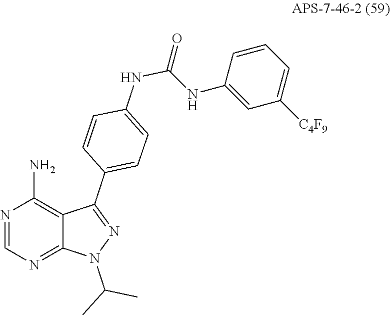

A solution of 3-(perfluorobutyl)aniline (17; 119 mg, 0.382 mmol) and CH₂Cl₂ (0.5 mL) was added to a 0° C. solution of CDI (67.5 mg, 0.416 mmol) and CH₂Cl₂ (0.5 mL) dropwise over 1 min. The solution was stirred at 0° C. for 1 h and then at room temperature for 24 h. The acyl imidazole solution was added to a −5° C. solution of 69 (102 mg, 0.380 mmol) and a 50:50 mixture of CH₂Cl₂/DMF (2 mL), down the wall of the flask over 5 min. The solution was allowed to warm to room temperature as the bath melted. After stirring for 12 h, the solution was concentrated to dryness and the remaining material was purified by silica gel chromatography (25 g cartridge), eluting at 25 mL/min and using a linear gradient of CH₂Cl₂/MeOH: 100:0→90:10 over 32 column volumes. Obtained 65.6 mg (29%) of the title compound as a white solid: ¹H NMR (400 MHz, DMSO-d₆) δ 9.13 (s, 1H), 9.01 (s, 1H), 8.23 (s, 1H), 8.03 (s, 1H), 7.51-7.69 (m, 6H), 7.28 (d, J=7.6 Hz, 1H), 5.05 (sept, J=6.6 Hz, 1H), 1.48 (d, J=6.8 Hz, 6H); ¹⁹F NMR (376 MHz, DMSO-d₆) δ −80.0 (t, J=9.2 Hz, 3F), −109.5-−109.3 (m, 2F), −122.1-−121.9 (m, 2F), −124.9-−124.7 (m, 2F); LC-MS (ESI+) m/z: [M+H]⁺ Calcd for C₂₅H₂₁F₉N₇O 606.2; Found 606.4.

### 1-(4-(4-amino-1-isopropyl-1H-pyrazolo[3,4-d]pyrimidin-3-yl)phenyl)-3-(2-fluoro-5-(trifluoromethyl)phenyl)urea (AD80 (64))

To a −5° C. solution of 69 (403 mg, 1.50 mmol) and a 50:50 mixture of

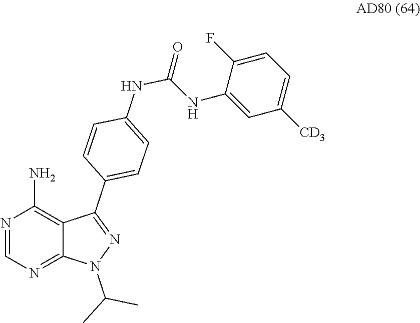 [SMILES: [2H]C([2H])([2H])c1ccc(F)c(NC(=O)Nc2ccc(-c3nn(C(C)C)c4ncnc(N)c34)cc2)c1]

CH₂Cl₂/DMF (6 mL), was added a solution of 2-fluoro-1-isocyanato-5-(trifluoromethyl)benzene (220 μL, 1.52 mmol) and CH₂Cl₂ (1.5 mL), down the wall of the flask over 5 min. The solution was allowed to warm to room temperature overnight as the bath melted. After stirring 12 h, the solution was concentrated to dryness and the remaining material was purified by silica gel chromatography (30 g cartridge), eluting at 30 mL/min and using a linear gradient of CH₂Cl₂/MeOH: 100:0→90:10 over 30 column volumes. Obtained 607 mg (85%) of the title compound as a white solid: ¹H NMR (400 MHz, DMSO-d₆) δ 9.39 (s, 1H), 8.96 (d, J=2.4 Hz, 1H), 8.65 (dd, J=7.3, 2.2 Hz, 1H), 8.23 (s, 1H), 7.63-7.67 (m, 2H), 7.59-7.63 (m, 2H), 7.51 (dd, J=10.3, 8.8 Hz, 1H), 7.37-7.44 (m, 1H), 5.06 (sept, J=6.7 Hz, 1H), 1.49 (d, J=6.6 Hz, 6H); ¹⁹F NMR (376 MHz, DMSO-d₆) δ −60.2 (s, 3F), −123.7 (s, 1F); LC-MS (ESI+) m/z: [M+H]⁺ Calcd for C₂₂H₂₂F₄N₇O 474.2; Found 474.4.

### 1-(4-(4-Amino-1-isopropyl-1H-pyrazolo[3,4-d]pyrimidin-3-yl)phenyl)-3-(2-fluoro-5-(perfluoroethyl)phenyl)urea (APS-5-86-2 (97))

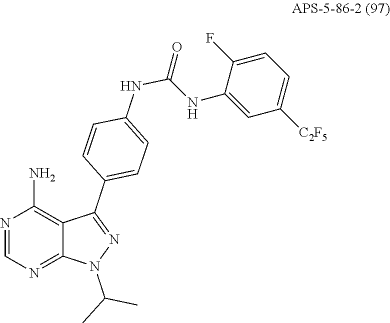

A solution of 2-fluoro-5-(perfluoroethyl)aniline (12; 145 mg, 0.633 mmol) and CH₂Cl₂ (0.5 mL) was added to a 0° C. solution of CDI (103 mg, 0.635 mmol) and CH₂Cl₂ (0.5 mL) dropwise over 1 min. The solution was stirred at 0° C. for 5 min and then at room temperature for 24 h. The acyl imidazole solution was added to a −5° C. solution of 69 (155 mg, 0.578 mmol) and a 50:50 mixture of CH₂Cl₂/DMF (2 mL), down the wall of the flask over 5 min. The solution was allowed to warm to room temperature as the bath melted. After stirring for 12 h, the solution was concentrated to dryness and the remaining material was purified by [symbol] natography (25 g cartridge), eluting at 25 mL/min and using a linear gradient of CH₂Cl₂/MeOH: 100:0→90:10 over 33 column volumes. Obtained 107 mg (35%) of the title compound as a white solid: 1H NMR (400 MHz, DMSO-d₆) δ 9.39 (s, 1H), 8.97 (d, J=2.7 Hz, 1H), 8.64 (dd, J=7.3, 2.0 Hz, 1H), 8.23 (s, 1H), 7.63-7.68 (m, 2H), 7.59-7.63 (m, 2H), 7.54 (dd, J=10.8, 8.8 Hz, 1H), 7.33-7.40 (m, 1H), 5.06 (sept, J=6.8 Hz, 1H), 1.49 (d, J=6.6 Hz, 6H); ¹⁹F NMR (376 MHz, DMSO-d₆) δ −83.6 (s, 3F), −112.5 (br s, 2F), −123.5-−123.3 (m, 1F); LC-MS (ESI+) m/z: [M+H]⁺ Calcd for C₂₃H₂₀F₆N₇O 524.2; Found 524.3.

### 1-(4-(4-Amino-1-isopropyl-1H-pyrazolo[3,4-d]pyrimidin-3-yl)phenyl)-3-(2-fluoro-5-(perfluoropropan-2-yl)phenyl)urea (APS-6-60-2 (98))

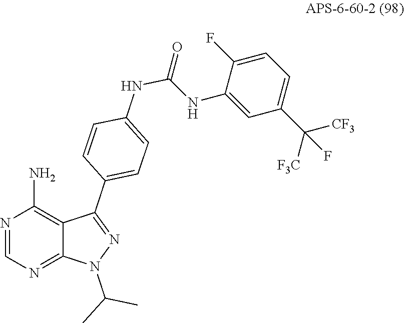 [SMILES: CC(C)n1nc(-c2ccc(NC(=O)Nc3cc(C(F)(C(F)(F)F)C(F)(F)F)ccc3F)cc2)c2c(N)ncnc21]

A solution of 2-fluoro-5-(perfluoropropan-2-yl)aniline (16; 115 mg, 0.412 mmol) and CH₂Cl₂ (0.5 mL) was added to a 0° C. solution of CDI (70.0 mg, 0.432 mmol) and CH₂Cl₂ (0.5 mL) dropwise over 1 min. The solution was stirred at 0° C. for 5 min and then at room temperature for 24 h. The acyl imidazole solution was added to a −5° C. solution of 69 (110 mg, 0.410 mmol) and a 50:50 mixture of CH₂Cl₂/DMF (2 mL), down the wall of the flask over 5 min. The solution was allowed to warm to room temperature as the bath melted. After stirring for 12 h, the solution was concentrated to dryness and the remaining material was purified by silica gel chromatography (25 g cartridge), eluting at 25 mL/min and using a linear gradient of CH₂Cl₂/EtOAc: 100:0→0:100 over 35 column volumes. Obtained 62.1 mg (26%) of the title compound as a white solid: ¹H NMR (400 MHz, DMSO-d₆) δ 9.40 (s, 1H), 8.97 (d, J=2.7 Hz, 1H), 8.67-8.76 (m, 1H), 8.23 (s, 1H), 7.63-7.68 (m, 2H), 7.59-7.63 (m, 2H), 7.55 (dd, J=10.8, 8.8 Hz, 1H), 7.33 (dd, J=7.6, 4.4 Hz, 1H), 6.71 (br s, 2H), 5.06 (sept, J=6.7 Hz, 1H), 1.49 (d, J=6.8 Hz, 6H); ¹⁹F NMR (376 MHz, DMSO-d₆) δ −74.6 (d, J=6.9 Hz, 6F), −124.8 (br s, 1F), −180.1-−179.9 (m, 1F); LC-MS (ESI+) m/z: [M+H]⁻ Calcd for C₂₄H₂₀F₈N₇O 574.2; Found 574.4.

### 1-(4-(4-Amino-1-isopropyl-1H-pyrazolo[3,4-d]pyrimidin-3-yl)phenyl)-3-(2-fluoro-5-(perfluoropropyl)phenyl)urea 2,2,2-trifluoroacetate (APS-6-60-1 (99))

A solution of 2-fluoro-5-(perfluoropropyl)aniline (14; 115 mg, 0.412 mmol) and

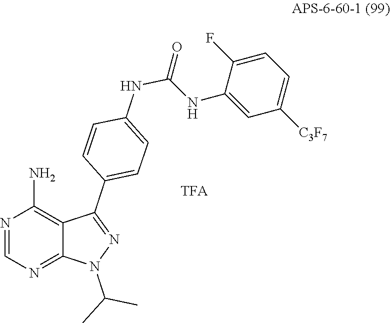

CH₂Cl₂ (0.5 mL) was added to a 0° C. solution of CDI (70.0 mg, 0.432 mmol) and CH₂Cl₂ (0.5 mL) dropwise over 1 min. The solution was stirred at 0° C. for 5 min and then at room temperature for 24 h. The acyl imidazole solution was added to a −5° C. solution of 69 (110 mg, 0.410 mmol) and a 50:50 mixture of CH₂Cl₂/DMF (2 mL), down the wall of the flask over 5 min. The solution was allowed to warm to room temperature as the bath melted. After stirring for 12 h, the solution was concentrated to dryness and the remaining material was purified by reverse-phase chromatography, eluting at 40 mL/min and using a linear gradient of H₂O (with 0.1% v/v TFA)/MeCN (with 0.1% v/v TFA): 90:10→1:99 over 16 minutes. Obtained 119 mg (51%) of the title compound as a white solid: ¹H NMR (400 MHz, DMSO-d₆) δ 9.53 (s, 1H), 9.05 (d, J=2.7 Hz, 1H), 8.63 (dd, J=7.3, 2.0 Hz, 1H), 8.40 (s, 1H), 7.65-7.71 (m, 2H), 7.59-7.65 (m, 2H), 7.55 (dd, J=10.6, 8.7 Hz, 1H), 7.31-7.40 (m, 1H), 5.10 (sept, J=13.4 Hz, 2H), 1.51 (d, J=6.8 Hz, 6H); ¹⁹F NMR (376 MHz, DMSO-d₆) δ −74.0 (s, 3F), −79.0 (t, J=9.2 Hz, 3F), −109.6-−109.4 (m, 2F), −123.1 (br s, 1F), −125.6 (s, 2F); LC-MS (ESI+) m/z: [M+H]⁺ Calcd for C₂₄H₂₀F₈N₇O 574.2; Found 574.4.

### 1-(4-(4-Amino-1-isopropyl-1H-pyrazolo[3,4-d]pyrimidin-3-yl)phenyl)-3-(2-fluoro-5-(perfluorobutan-2-yl)phenyl)urea (APS-8-35-3 (100))

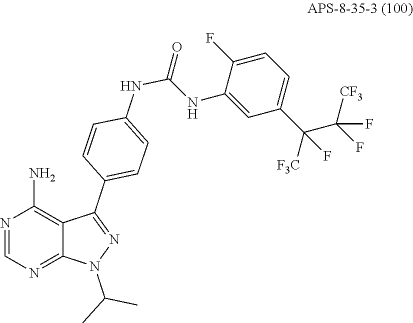 [SMILES: CC(C)n1nc(-c2ccc(NC(=O)Nc3cc(C(F)(C(F)(F)F)C(F)(F)C(F)(F)F)ccc3F)cc2)c2c(N)ncnc21]

A solution of 2-fluoro-5-(perfluorobutan-2-yl)aniline (20; 135 mg, 0.410 mmol) and CH₂Cl₂ (0.5 mL) was added to a 0° C. solution of CDI (70.0 mg, 0.432 mmol) and CH₂Cl₂ (0.5 mL) dropwise over 1 min. The solution was stirred at 0° C. for 5 min and then at room temperature for 24 h. The acyl imidazole solution was added to a −5° C. solution of 69 (110 mg, [symbol] nd a 50:50 mixture of CH₂Cl₂/DMF (2 mL), down the wall of the flask over 5 min. The solution was allowed to warm to room temperature as the bath melted. After stirring for 12 h, the solution was concentrated to dryness and the remaining material was purified by silica gel chromatography (25 g cartridge), eluting at 25 mL/min and using a linear gradient of CH₂Cl₂/EtOAc: 100:0→0:100 over 23 column volumes. Obtained 71.8 mg (28%) of the title compound as a white solid: ¹H NMR (400 MHz, DMSO-d₆) δ 9.40 (s, 1H), 8.97 (d, J=2.9 Hz, 1H), 8.72 (d, J=7.1 Hz, 1H), 8.23 (s, 1H), 7.63-7.67 (m, 2H), 7.59-7.63 (m, 2H), 7.55 (dd, J=11.0, 8.8 Hz, 1H), 7.27-7.36 (m, 1H), 5.06 (sept, J=6.7 Hz, 1H), 1.49 (d, J=6.6 Hz, 6H); ¹⁹F NMR (376 MHz, DMSO-d₆) δ −73.1-−72.9 (m, 3F), −78.3 (d, J=13.7 Hz, 3F), −120.7-−120.3 (m, 2F), −124.9 (br s, 1F), −181.0-−180.7 (m, 1F); LC-MS (ESI+) m/z: [M+H]⁺ Calcd for C₂₅H₂₀F₁₀N₇O 624.2; Found 624.2.

### 1-(4-(4-Amino-1-isopropyl-1H-pyrazolo[3,4-d]pyrimidin-3-yl)phenyl)-3-(2-fluoro-5-(perfluorobutyl)phenyl)urea 2,2,2-trifluoroacetate (APS-6-60-3 (101))

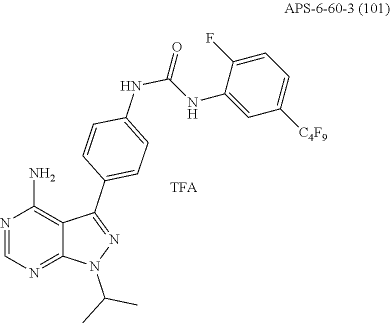

A solution of 2-fluoro-5-(perfluorobutyl)aniline (18; 135 mg, 0.410 mmol) and CH₂Cl₂ (0.5 mL) was added to a 0° C. solution of CDI (70.0 mg, 0.432 mmol) and CH₂Cl₂ (0.5 mL) dropwise over 1 min. The solution was stirred at 0° C. for 5 min and then at room temperature for 24 h. The acyl imidazole solution was added to a −5° C. solution of 69 (110 mg, 0.410 mmol) and a 50:50 mixture of CH₂Cl₂/DMF (2 mL), down the wall of the flask over 5 min. The solution was allowed to warm to room temperature as the bath melted. After stirring for 12 h, the solution was concentrated to dryness and the remaining material was purified by reverse-phase chromatography, eluting at 40 mL/min and using a linear gradient of H₂O (with 0.1% v/v TFA)/MeCN (with 0.1% v/v TFA): 90:10→1:99 over 16 minutes. Obtained 108 mg (42%) of the title compound as a white solid: ¹H NMR (400 MHz, DMSO-d₆) δ 9.49 (s, 1H), 9.03 (d, J=2.9 Hz, 1H), 8.63 (dd, J=7.2, 2.1 Hz, 1H), 8.39 (s, 1H), 7.65-7.71 (m, 2H), 7.59-7.64 (m, 2H), 7.55 (dd, J=10.9, 8.7 Hz, 1H), 7.32-7.41 (m, 1H), 5.09 (sept, J=13.4 Hz, 1H), 1.51 (d, J=6.8 Hz, 6H); ¹⁹F NMR (376 MHz, DMSO-d₆) δ −73.9 (s, 3F), −80.0 (t, J=9.2 Hz, 3F), −108.9-−108.7 (m, 2F), −122.0-−121.8 (m, 2F), −123.1 (br s, 1F), −124.9-−124.6 (m, 2F); LC-MS (ESI+) m/z: [M+H]⁻ Calcd for C₂₅H₂₀F₁₀N₇O 624.2; Found 624.4.

### 1-(4-(4-Amino-1-isopropyl-1H-pyrazolo[3,4-d]pyrimidin-3-yl)phenyl)-3-(4-(trifluoromethyl)phenyl)urea 2,2,2-trifluoroacetate (APS-8-85-1 (102))

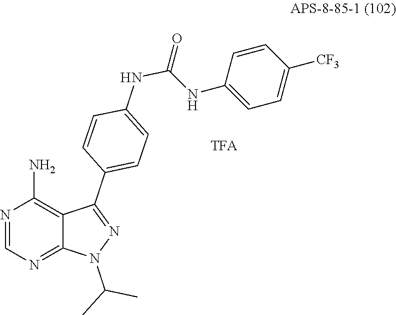 [SMILES: CC(C)n1nc(-c2ccc(NC(=O)Nc3ccc(C(F)(F)F)cc3)cc2)c2c(N)ncnc21]

To a −5° C. solution of 69 (65.0 mg, 0.242 mmol) and a 50:50 mixture of CH₂Cl₂/DMF (1 mL), was added a solution of 1-isocyanato-4-(trifluoromethyl)benzene (38.0 μL, 0.266 mmol) and CH₂Cl₂ (1.5 mL), down the wall of the flask over 5 min. The solution was allowed to warm to room temperature overnight as the bath melted. After stirring 12 h, the solution was concentrated to dryness and the remaining material was purified by reverse-phase chromatography, eluting at 40 mL/min and using a linear gradient of H₂O (with 0.1% v/v TFA)/MeCN (with 0.1% v/v TFA): 90:10→1:99 over 12 minutes. Obtained 50.3 mg (38%) of the title compound as a white solid: 1H NMR (400 MHz, DMSO-d₆) δ 9.41 (s, 1H), 9.31 (s, 1H), 8.41 (s, 1H), 7.57-7.75 (m, 8H), 5.09 (sept, J=6.6 Hz, 1H), 1.51 (d, J=6.8 Hz, 6H); ¹⁹F NMR (376 MHz, DMSO-d₆) δ −59.6 (s, 3F), −73.9 (s, 3F); LC-MS (ESI+) m/z: [M+H]⁺ Calcd for C₂₂H₂₁F₃N₇O 456.2; Found 456.3.

### 1-(4-(4-Amino-1-isopropyl-1H-pyrazolo[3,4-d]pyrimidin-3-yl)phenyl)-3-(4-(perfluoroethyl)phenyl)urea (APS-8-100-3 (60))

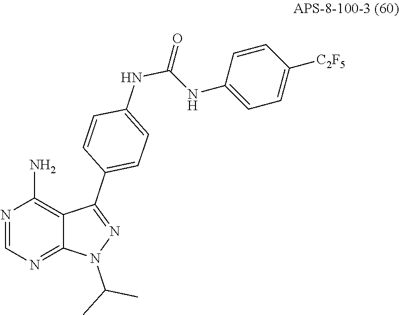

A solution of 4-(perfluoroethyl)aniline (6; 87.0 mg, 0.412 mmol) and CH₂Cl₂ (0.5 mL) was added to a 0° C. solution of CDI (70.0 mg, 0.432 mmol) and CH₂Cl₂ (0.5 mL) dropwise over 1 min. The solution was stirred at 0° C. for 1 h and then at room temperature for 24 h. The acyl imidazole solution was added to a −5° C. solution of 69 (110 mg, 0.410 mmol) and a 50:50 mixture of CH₂Cl₂/DMF (2 mL), down the wall of the flask over 5 min. The solution was allowed to warm to room temperature as the bath melted. After stirring for 12 h, the solution was concentrated to dryness and the remaining material was purified by silica gel chromatoranhy (25 g cartridge), eluting at 25 mL/min and using a linear gradient of CH₂Cl₂/EtOAc: 100:0→0:100 over 30 column volumes. Obtained 72.7 mg (35%) of the title compound as a white solid: 1H NMR (400 MHz, DMSO-d₆) δ 9.18 (s, 1H), 9.04 (s, 1H), 8.24 (s, 1H), 7.69-7.75 (m, 2H), 7.63-7.68 (m, 2H), 7.57-7.63 (m, 4H), 5.05 (sept, J=6.7 Hz, 1H), 1.49 (d, J=6.6 Hz, 6H); ¹⁹F NMR (376 MHz, DMSO-d₆) δ −83.7 (s, 3F), −112.3 (br s, 2F); LC-MS (ESI+) m/z: [M+H]⁺ Calcd for C₂₃H₂₁F₅N₇O 506.2; Found 506.7.

### 1-(4-(4-Amino-1-isopropyl-1H-pyrazolo[3,4-d]pyrimidin-3-yl)phenyl)-3-(4-(perfluoropropan-2-yl)phenyl)urea (APS-8-50-3 (103))

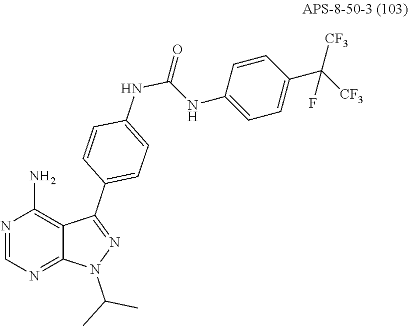 [SMILES: CC(C)n1nc(-c2ccc(NC(=O)Nc3ccc(C(F)(C(F)(F)F)C(F)(F)F)cc3)cc2)c2c(N)ncnc21]

A solution of 4-(perfluoropropan-2-yl)aniline (8; 107 mg, 0.410 mmol) and CH₂Cl₂ (0.5 mL) was added to a 0° C. solution of CDI (70.0 mg, 0.432 mmol) and CH₂Cl₂ (0.5 mL) dropwise over 1 min. The solution was stirred at 0° C. for 1 h and then at room temperature for 24 h. The acyl imidazole solution was added to a −5° C. solution of 69 (110 mg, 0.410 mmol) and a 50:50 mixture of CH₂Cl₂/DMF (2 mL), down the wall of the flask over 5 min. The solution was allowed to warm to room temperature as the bath melted. After stirring for 12 h, the solution was concentrated to dryness and the remaining material was purified by silica gel chromatography (25 g cartridge), eluting at 25 mL/min and using a linear gradient of CH₂Cl₂/MeOH: 100:0→90:10 over 33 column volumes. Obtained 101 mg (44%) of the title compound as a white solid: 1H NMR (400 MHz, DMSO-d₆) δ 9.14 (s, 1H), 9.02 (s, 1H), 8.23 (s, 1H), 7.72 (d, J=8.8 Hz, 2H), 7.62-7.68 (m, 2H), 7.55-7.62 (m, 4H), 5.05 (sept, J=6.7 Hz, 1H), 1.48 (d, J=6.6 Hz, 6H); ¹⁹F NMR (376 MHz, DMSO-d₆) δ −74.8 (d, J=6.9 Hz, 6F), −180.8-−180.6 (m, 1F); LC-MS (ESI+) m/z: [M+H]⁻ Calcd for C₂₄H₂₁F₇N₇O 556.2; Found 556.6.

### 1-(4-(4-Amino-1-isopropyl-1H-pyrazolo[3,4-d]pyrimidin-3-yl)phenyl)-3-(4-(perfluoropropyl)phenyl)urea (APS-8-51-3 (104))

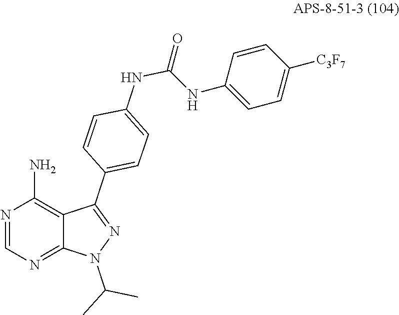

A solution of 4-(perfluoropropyl)aniline (7; 107 mg, 0.410 mmol) and CH₂Cl₂ (0.5 mL) was added to a 0° C. solution of CDI (70.0 mg, 0.432 mmol) and CH₂Cl₂ (0.5 mL) dropwise over 1 min. The solution was stirred at 0° C. for 1 h and then at room temperature for 24 h. The acyl imidazole solution was added to a −5° C. solution of 69 (110 mg, 0.410 mmol) and a 50:50 mixture of CH₂Cl₂/DMF (2 mL), down the wall of the flask over 5 min. The solution was allowed to warm to room temperature as the bath melted. After stirring for 12 h, the solution was concentrated to dryness and the remaining material was purified by silica gel chromatography (25 g cartridge), eluting at 25 mL/min and using a linear gradient of CH₂Cl₂/MeOH: 100:0→90:10 over 35 column volumes. Obtained 97.6 mg (43%) of the title compound as a white solid: ¹H NMR (400 MHz, DMSO-d₆) δ 9.19 (s, 1H), 9.04 (s, 1H), 8.23 (s, 1H), 7.72 (d, J=8.8 Hz, 2H), 7.63-7.68 (m, 2H), 7.56-7.62 (m, 4H), 5.05 (sept, J=6.8 Hz, 1H), 1.49 (d, J=6.6 Hz, 6H); ¹⁹F NMR (376 MHz, DMSO-d₆) δ −79.1-−78.8 (m, 3F), −109.4-−109.1 (m, 2F), −125.6 (s, 2F); LC-MS (ESI+) m/z: [M+H]⁺ Calcd for C₂₄H₂₁F₇N₇O 556.2; Found 556.6.

### 1-(4-(4-Amino-1-isopropyl-1H-pyrazolo[3,4-d]pyrimidin-3-yl)phenyl)-3-(4-(perfluorobutan-2-yl)phenyl)urea (APS-8-6-2 (65))

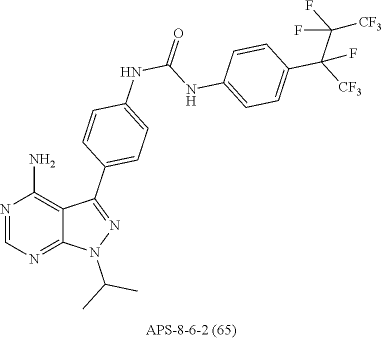 [SMILES: CC(C)n1nc(-c2ccc(NC(=O)Nc3ccc(C(F)(C(F)(F)F)C(F)(F)C(F)(F)F)cc3)cc2)c2c(N)ncnc21]

A solution of 4-(perfluorobutan-2-yl)aniline (10; 128 mg, 0.411 mmol) and CH₂Cl₂ (0.5 mL) was added to a 0° C. solution of CDI (70.0 mg, 0.432 mmol) and CH₂Cl₂ (0.5 mL) dropwise over 1 min. The solution was stirred at 0° C. for 1 h and then at room temperature for 24 h. The acyl imidazole solution was added to a −5° C. solution of 69 (110 mg, 0.410 mmol) and a 50:50 mixture of CH₂Cl₂/DMF (2 mL), down the wall of the flask over 5 min. The solution was allowed to warm to room temperature as the bath melted. After stirring for 12 h, the solution was concentrated to dryness and the remaining material was purified by silica gel chromatography (25 g cartridge), eluting at 25 mL/min and using a linear gradient of CH₂Cl₂/MeOH: 100:0→90:10 over 35 column volumes. Obtained 73.0 mg (29%) of the title compound as a white solid: ¹H NMR (400 MHz, DMSO-d₆) δ 9.15 (s, 1H), 9.02 (s, 1H), 8.23 (s, 1H), 7.73 (d, J=8.6 Hz, 2H), 7.63-7.68 (m, 2H), 7.54-7.62 (m, 4H), 6.74 (br s, 2H), 5.06 (sept, J=6.7 Hz, 1H), 1.49 (d, J=6.6 Hz, 6H); ¹⁹F NMR (376 MHz, DMSO-d₆) δ −73.4-−73.1 (m, 3F), −78.3 (d, J=13.7 Hz, 3F), −120.8-−120.6 (m, 2F), −182.3-−181.9 (m, 1F); LC-MS (ESI+) m/z: [M+H]⁺ Calcd for C₂₅H₂₁F₉N₇O 606.2; Found 606.4.

### 1-(4-(4-Amino-1-isopropyl-1H-pyrazolo[3,4-d]pyrimidin-3-yl)phenyl)-3-(4-(perfluorobutyl)phenyl)urea (APS-8-52-3 (105))

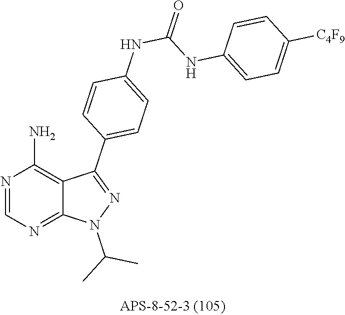

A solution of 4-(perfluorobutyl)aniline (9; 128 mg, 0.411 mmol) and CH₂Cl₂ (0.5 mL) was added to a 0° C. solution of CDI (70.0 mg, 0.432 mmol) and CH₂Cl₂ (0.5 mL) dropwise over 1 min. The solution was stirred at 0° C. for 1 h and then at room temperature for 24 h. The acyl imidazole solution was added to a −5° C. solution of 69 (110 mg, 0.410 mmol) and a 50:50 mixture of CH₂Cl₂/DMF (2 mL), down the wall of the flask over 5 min. The solution was allowed to warm to room temperature as the bath melted. After stirring for 12 h, the solution was concentrated to dryness and the remaining material was purified by silica gel chromatography (25 g cartridge), eluting at 25 mL/min and using a linear gradient of CH₂Cl₂/MeOH: 100:0→90:10 over 35 column volumes. Obtained 45.1 mg (18%) of the title compound as a white solid: ¹H NMR (400 MHz, DMSO-d₆) δ 9.19 (s, 1H), 9.04 (s, 1H), 8.23 (s, 1H), 7.72 (d, J=8.8 Hz, 2H), 7.63-7.68 (m, 2H), 7.57-7.63 (m, 4H), 5.05 (sept, J=6.7 Hz, 1H), 1.49 (d, J=6.6 Hz, 6H); ¹⁹F NMR (376 MHz, DMSO-d₆) δ −80.0 (t, J=9.2 Hz, 3F), −108.6--108.4 (m, 2F), −122.2-−121.8 (m, 2F), −124.9-−124.6 (m, 2F); LC-MS (ESI+) m/z: [M+H]⁺ Calcd for C₂₅H₂₁F₉N₇O 606.2; Found 606.7.

### 1-(4-(4-Amino-7-isopropyl-7H-pyrrolo[2,3-d]pyrimidin-5-yl)phenyl)-3-(3-(trifluoromethyl)phenyl)urea (APS-9-2 (106))

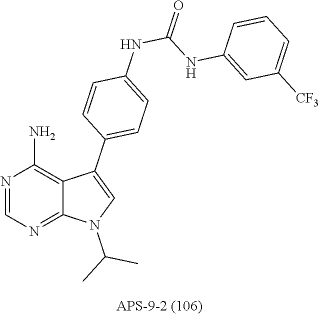 [SMILES: CC(C)n1cc(-c2ccc(NC(=O)Nc3cccc(C(F)(F)F)c3)cc2)c2c(N)ncnc21]

To a −5° C. solution of 71 (100 mg, 0.374 mmol) and CH₂Cl₂ (2 mL), was added a solution of 1-isocyanato-3-(trifluoromethyl)benzene (55.0 μL, 0.399 mmol) and CH₂Cl₂ (0.5 mL), down the wall of the flask over 5 min. The solution was allowed to warm to room temperature overnight as the bath melted and was stirred for a total of 12 h. Purification by silica gel chromatography (25 g cartridge), eluting at 25 mL/min and using a linear gradient of CH₂Cl₂/MeOH: 100:0→90:10 over 30 column volumes, provided 96.1 mg (57%) of the title compound as a white solid: ¹H NMR (400 MHz, DMSO-d₆) δ 9.07 (s, 1H), 8.88 (s, 1H), 8.13 (s, 1H), 8.04 (s, 1H), 7.55-7.62 (m, 3H), 7.49-7.55 (m, 1H), 7.37-7.43 (m, 3H), 7.32 (d, J=7.6 Hz, 1H), 6.04 (br s, 2H), 4.97 (sept, J=6.7 Hz, 1H), 1.46 (d, J=6.6 Hz, 6H); ¹⁹F NMR (376 MHz, DMSO-d₆) δ −60.8 (s, 3F); LC-MS (ESI+) m/z: [M+H]⁺ Calcd for C₂₃H₂₂F₃N₆O 455.2; Found 455.6.

### 1-(4-(4-Amino-7-isopropyl-7H-pyrrolo[2,3-d]pyrimidin-5-yl)phenyl)-3-(3-(perfluoroethyl)phenyl)urea 2,2,2-trifluoroacetate (APS-9-3 (107))

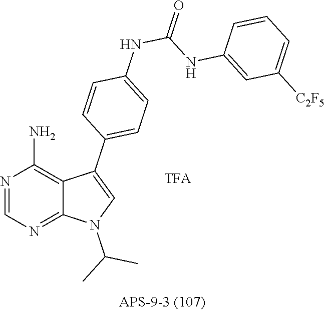

A solution of 3-(perfluoroethyl)aniline (11; 87.0 mg, 0.412 mmol) and CH₂Cl₂ (0.5 mL) was added to a 0° C. solution of CDI (70.0 mg, 0.432 mmol) and CH₂Cl₂ (0.5 mL) dropwise over 1 min. The solution was stirred at 0° C. for 1 h and then at room temperature for 24 h. The acyl imidazole solution was added to a −5° C. solution of 71 (110 mg, 0.411 mmol) and CH₂Cl₂ (2 mL), down the wall of the flask over 5 min. The solution was allowed to warm to room temperature as the bath melted. After 12 h the solution was concentrated to dryness and purified by reverse-phase chromatography, eluting at 40 mL/min and using a linear gradient of [symbol] % v/v TFA)/MeCN (with 0.1% v/v TFA): 90:10→1:99 over 16 min. Obtained 118 mg (57%) of the title compound as a white solid: 1H NMR (400 MHz, DMSO-d₆) δ 9.53 (s, 1H), 9.36 (s, 1H), 8.42 (s, 1H), 8.05 (s, 1H), 7.76 (s, 1H), 7.64 (d, J=8.8 Hz, 3H), 7.55 (t, J=7.9 Hz, 1H), 7.42 (d, J=8.6 Hz, 2H), 7.27 (d, J=7.8 Hz, 1H), 5.03 (sept, J=6.7 Hz, 1H), 1.50 (d, J=6.8 Hz, 6H); ¹⁹F NMR (376 MHz, DMSO-d₆) δ −73.5 (s, 3F), −83.5 (s, 3F), −113.2 (br s, 2F); LC-MS (ESI+) m/z: [M+H]⁺ Calcd for C₂₄H₂₂F₅N₆O 505.2; Found 505.7.

### 1-(4-(4-Amino-7-isopropyl-7H-pyrrolo[2,3-d]pyrimidin-5-yl)phenyl)-3-(3-(perfluoropropan-2-yl)phenyl)urea (APS-7-47-1 (108))

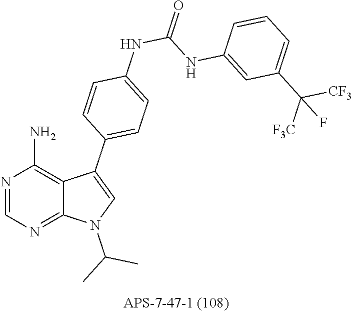 [SMILES: CC(C)n1cc(-c2ccc(NC(=O)Nc3cccc(C(F)(C(F)(F)F)C(F)(F)F)c3)cc2)c2c(N)ncnc21]

A solution of 3-(perfluoropropan-2-yl)aniline (15; 99.5 mg, 0.381 mmol) and CH₂Cl₂ (0.5 mL) was added to a 0° C. solution of CDI (67.5 mg, 0.416 mmol) and CH₂Cl₂ (0.5 mL) dropwise over 1 min. The solution was stirred at 0° C. for 1 h and then at room temperature for 24 h. The acyl imidazole solution was added to a −5° C. solution of 71 (102 mg, 0.382 mmol) and CH₂Cl₂ (2 mL), down the wall of the flask over 5 min. The solution was allowed to warm to room temperature as the bath melted and stirred for a total of 12 h. Purification by silica gel chromatography (25 g cartridge), eluting at 25 mL/min and using a linear gradient of CH₂Cl₂/MeOH: 100:0→90:10 over 32 column volumes, provided 106 mg (50%) of the title compound as a white solid: 1H NMR (400 MHz, DMSO-d₆) δ 9.12 (s, 1H), 8.86 (s, 1H), 8.13 (s, 1H), 8.05 (s, 1H), 7.51-7.62 (m, 4H), 7.36-7.43 (m, 3H), 7.25 (d, J=7.8 Hz, 1H), 5.79-6.27 (m, 2H), 4.96 (sept, J=6.7 Hz, 1H), 1.46 (d, J=6.8 Hz, 6H); ¹⁹F NMR (376 MHz, DMSO-d₆) δ −74.6 (d, J=6.9 Hz, 6F), −181.2-−181.0 (m, 1F); LC-MS (ESI+) m/z: [M+H]⁺ Calcd for C₂₅H₂₂F₇N₆O 555.2; Found 555.4.

### 1-(4-(4-Amino-7-isopropyl-7H-pyrrolo[2,3-d]pyrimidin-5-yl)phenyl)-3-(3-(perfluoropropyl)phenyl)urea 2,2,2-trifluoroacetate (APS-7-52-2 (109))

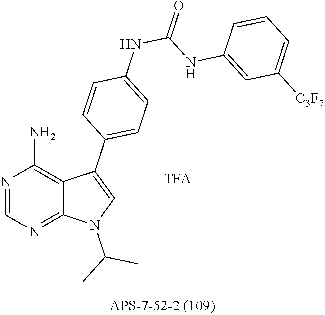

A solution of 3-(perfluoropropyl)aniline (13; 99.2 mg, 0.380 mmol) and CH₂Cl₂ (0.5 mL) was added to a 0° C. solution of CDI (67.5 mg, 0.416 mmol) and CH₂Cl₂ (0.5 mL) dropwise over 1 min. The solution was stirred at 0° C. for 1 h and then at room temperature for 24 h. The acyl imidazole solution was added to a −5° C. solution of 71 (102 mg, 0.382 mmol) and CH₂Cl₂ (2 mL), down the wall of the flask over 5 min. The solution was allowed to warm to room temperature as the bath melted and stirred for a total of 12 h. Purification by silica gel chromatography (25 g cartridge), eluting at 25 mL/min and using a linear gradient of CH₂Cl₂/MeOH: 100:0→90:10 over 30 column volumes, provided 128 mg (61%) of the title compound as a white solid: 1H NMR (400 MHz, DMSO-d₆) δ 9.42 (s, 1H), 9.25 (s, 1H), 8.42 (s, 1H), 8.04 (s, 1H), 7.76 (s, 1H), 7.63 (d, J=8.6 Hz, 3H), 7.51-7.59 (m, 1H), 7.42 (d, J=8.6 Hz, 2H), 7.26 (d, J=7.8 Hz, 1H), 5.03 (quin, J=6.7 Hz, 1H), 1.50 (d, J=6.8 Hz, 6H); ¹⁹F NMR (376 MHz, DMSO-d₆) δ −73.5 (s, 3F), −79.2-−78.9 (m, 3F), −110.3-−110.0 (m, 2F), −125.7 (s, 2F); LC-MS (ESI+) m/z: [M+H]⁺ Calcd for C₂₅H₂₂F₇N₆O 555.2; Found 555.4.

### 1-(4-(4-Amino-7-isopropyl-7H-pyrrolo[2,3-d]pyrimidin-5-yl)phenyl)-3-(3-(perfluorobutan-2-yl)phenyl)urea (APS-8-56-4 (110))

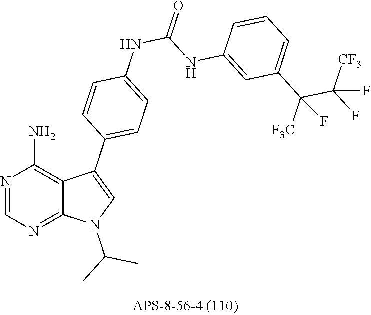 [SMILES: CC(C)n1cc(-c2ccc(NC(=O)Nc3cccc(C(F)(C(F)(F)F)C(F)(F)C(F)(F)F)c3)cc2)c2c(N)ncnc21]

A solution of 3-(perfluorobutan-2-yl)aniline (19; 128 mg, 0.411 mmol) and CH₂Cl₂ (0.5 mL) was added to a 0° C. solution of CDI (70.0 mg, 0.432 mmol) and CH₂Cl₂ (0.5 mL) dropwise over 1 min. The solution was stirred at 0° C. for 1 h and then at room temperature for 24 h. The acyl imidazole solution was added to a −5° C. solution of 71 (110 mg, 0.411 mmol) and CH₂Cl₂ (2 mL), down the wall of the flask over 5 min. The solution was allowed to warm to room temperature as the bath melted and stirred for a total of 12 h. Purification by silica gel chromatography (25 g cartridge), eluting at 25 mL/min and using a linear gradient of CH₂Cl₂/MeOH: 100:0→90:10 over 35 column volumes, provided 135 mg (54%) of the title compound as a white solid: 1H NMR (400 MHz, DMSO-d₆) δ 9.12 (s, 1H), 8.85 (s, 1H), 8.13 (s, 1H), 8.05 (s, 1H), 7.50-7.63 (m, 4H), 7.36-7.44 (m, 3H), 7.24 (d, J=7.1 Hz, 1H), 4.97 (sept, J=6.8 Hz, 1H), 1.46 (d, J=6.6 Hz, 6H); ¹⁹F NMR (376 MHz, DMSO-d₆) δ −73.1-−72.7 (m, 3F), −78.3 (d, J=11.4 Hz, 3F), −120.7-−120.4 (m, 2F), −182.1-−181.9 (m, 1F); LC-MS (ESI+) m/z: [M+H]⁺ Calcd for C₂₆H₂₂F₉N₆O 605.2; Found 605.3.

### 1-(4-(4-Amino-7-isopropyl-7H-pyrrolo[2,3-d]pyrimidin-5-yl)phenyl)-3-(3-(perfluorobutyl)phenyl)urea (APS-7-47-2 (111))

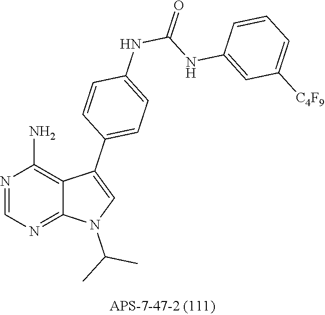

A solution of 3-(perfluorobutyl)aniline (17; 119 mg, 0.382 mmol) and CH₂Cl₂ (0.5 mL) was added to a 0° C. solution of CDI (67.5 mg, 0.432 mmol) and CH₂Cl₂ (0.5 mL) dropwise over 1 min. The solution was stirred at 0° C. for 1 h and then at room temperature for 24 h. The acyl imidazole solution was added to a −5° C. solution of 71 (102 mg, 0.382 mmol) and CH₂Cl₂ (2 mL), down the wall of the flask over 5 min. The solution was allowed to warm to room temperature as the bath melted and stirred for a total of 12 h. Purification by silica gel chromatography (25 g cartridge), eluting at 25 mL/min and using a linear gradient of CH₂Cl₂/MeOH: 100:0→90:10 over 35 column volumes, provided 51.7 mg (22%) of the title compound as a white solid: 1H NMR (400 MHz, DMSO-d₆) δ 9.11 (s, 1H), 8.88 (s, 1H), 8.13 (s, 1H), 8.03 (s, 1H), 7.51-7.66 (m, 4H), 7.35-7.45 (m, 3H), 7.27 (d, J=7.6 Hz, 1H), 6.04 (br s, 2H), 4.96 (sept, J=6.8 Hz, 1H), 1.46 (d, J=6.6 Hz, 6H); ¹⁹F NMR (376 MHz, DMSO-d₆) δ −80.0 (t, J=9.2 Hz, 3F), −109.5-−109.3 (m, 2F), −122.1-−121.8 (m, 2F), −124.9-−124.6 (m, 2F); LC-MS (ESI+) m/z: [M+H]⁻ Calcd for C₂₆H₂₂F₉N₆O 605.2; Found 605.4.

### 1-(4-(4-Amino-7-isopropyl-7H-pyrrolo[2,3-d]pyrimidin-5-yl)phenyl)-3-(2-fluoro-5-(trifluoromethyl)phenyl)urea (APS-6-87 (112))

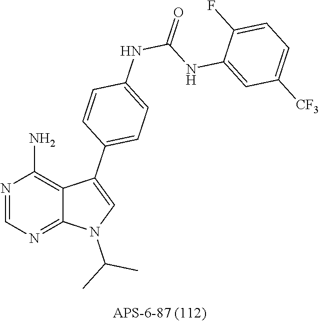 [SMILES: CC(C)n1cc(-c2ccc(NC(=O)Nc3cc(C(F)(F)F)ccc3F)cc2)c2c(N)ncnc21]

To a −5° C. solution of 71 (100 mg, 0.374 mmol) and CH₂Cl₂ (1 mL), was added a solution of 2-fluoro-1-isocyanato-5-(trifluoromethyl)benzene (55.0 μL, 0.380 mmol) and CH₂Cl₂ (1 mL), down the wall of the flask over 5 min. The solution was allowed to warm to room temperature overnight as the bath melted. After stirring 12 h, the resulting precipitate was collected by vacuum filtration; washed solid with CH₂Cl₂. Air-drying provided 111 mg (63%) of the title compound as a white solid: ¹H NMR (400 MHz, DMSO-d₆) δ 9.26 (s, 1H), 8.93 (d, J=2.9 Hz, 1H), 8.64 (dd, J=7.3, 2.2 Hz, 1H), 8.13 (s, 1H), 7.57 (d, J=8.8 Hz, 2H), 7.47-7.54 (m, 1H), 7.36-7.45 (m, 4H), 6.04 (br s, 2H), 4.97 (sept, J=6.7 Hz, 1H), 1.46 (d, J=6.8 Hz, 6H); ¹⁹F NMR (376 MHz, DMSO-d₆) δ −60.2 (s, 3F), −123.8 (s, 1F); LC-MS (ESI+) m/z: [M+H]⁺ Calcd for C₂₃H₂₁F₄N₆O 473.2; Found 473.4.

### 1-(4-(4-Amino-7-isopropyl-7H-pyrrolo[2,3-d]pyrimidin-5-yl)phenyl)-3-(2-fluoro-5-(perfluoroethyl)phenyl)urea (APS-7-38 (113))

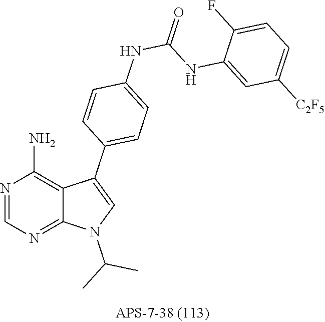

A solution of 2-fluoro-5-(perfluoroethyl)aniline (12; 85.0 mg, 0.375 mmol) and CH₂Cl₂ (0.5 mL) was added to a 0° C. solution of CDI (65.7 mg, 0.405 mmol) and CH₂Cl₂ (0.5 mL) dropwise over 1 min. The solution was stirred at 0° C. for 5 min and then at room temperature for 24 h. The acyl imidazole solution was added to a −5° C. solution of 71 (100 mg, 0.374 mmol) and CH₂Cl₂ (2 mL), down the wall of the flask over 5 min. The solution was allowed to warm to room temperature as the bath melted and stirred for a total of 12 h. Purification by silica gel chromatography (25 g cartridge), eluting at 25 mL/min and using a linear gradient of CH₂Cl₂/MeOH: 100:0→90:10 over 30 column volumes, provided 131 mg (67%) of the title compound as a white solid: 1H NMR (400 MHz, DMSO-d₆) δ 9.28 (s, 1H), 8.95 (d, J=2.9 Hz, 1H), 8.64 (dd, J=7.3, 2.2 Hz, 1H), 8.13 (s, 1H), 7.57 (d, J=8.6 Hz, 3H), 7.39-7.44 (m, 3H), 7.35 (ddd, J=8.5, 4.5, 2.4 Hz, 1H), 6.05 (br s, 2H), 4.97 (sept, J=6.7 Hz, 1H), 1.46 (d, J=6.8 Hz, 6H); ¹⁹F NMR (376 MHz, DMSO-d₆) δ −83.6 (s, 3F), −112.6 (br s, 2F), −123.5 (s, 1F); LC-MS (ESI+) m/z: [M+H]⁺ Calcd for C₂₄H₂₁F₆N₆O 523.2; Found 523.4.

### 1-(4-(4-Amino-7-isopropyl-7H-pyrrolo[2,3-d]pyrimidin-5-yl)phenyl)-3-(2-fluoro-5-(perfluoropropan-2-yl)phenyl)urea (APS-7-25-2 (114))

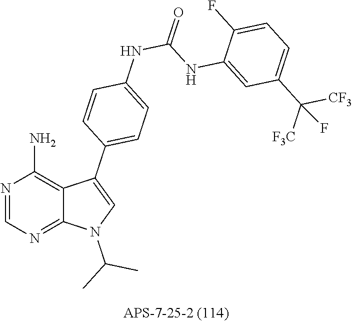 [SMILES: CC(C)n1cc(-c2ccc(NC(=O)Nc3cc(C(F)(C(F)(F)F)C(F)(F)F)ccc3F)cc2)c2c(N)ncnc21]

A solution of 2-fluoro-5-(perfluoropropan-2-yl)aniline (16; 107 mg, 0.383 mmol) and CH₂Cl₂ (0.5 mL) was added to a 0° C. solution of CDI (67.0 mg, 0.413 mmol) and CH₂Cl₂ (0.5 mL) dropwise over 1 min. The solution was stirred at 0° C. for 5 min and then at room temperature for 24 h. The acyl imidazole solution was added to a −5° C. solution of 71 (102 mg, 0.383 mmol) and CH₂Cl₂ (2 mL), down the wall of the flask over 5 min. The solution was allowed to warm to room temperature as the bath melted and stirred for a total of 12 h. Purification by silica gel chromatography (25 g cartridge), eluting at 25 mL/min and using a linear gradient of CH₂Cl₂/MeOH: 100:0→90:10 over 30 column volumes, provided 85.5 mg (39%) of the title compound as a white solid: 1H NMR (400 MHz, DMSO-d₆) δ 9.27 (s, 1H), 8.94 (d, J=2.9 Hz, 1H), 8.71 (dd, J=7.2, 1.8 Hz, 1H), 8.13 (s, 1H), 7.50-7.60 (m, 3H), 7.39-7.44 (m, 3H), 7.27-7.36 (m, 1H), 6.04 (br s, 2H), 4.96 (sept, J=6.8 Hz, 1H), 1.46 (d, J=6.8 Hz, 6H); ¹⁹F NMR (376 MHz, DMSO-d₆) δ −74.7 (d, J=9.2 Hz, 6F), −124.9 (br s, 1F), −180.1--179.9 (m, 1F); LC-MS (ESI+) m/z: [M+H]⁺ Calcd for C₂₅H₂₁F₈N₆O 573.2; Found 573.4.

### 1-(4-(4-Amino-7-isopropyl-7H-pyrrolo[2,3-d]pyrimidin-5-yl)phenyl)-3-(2-fluoro-5-(perfluoropropyl)phenyl)urea (APS-7-25-1 (115))

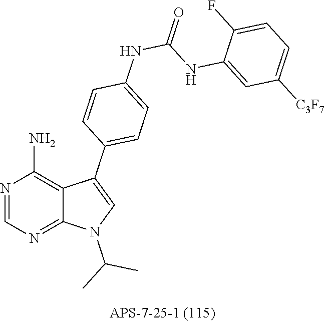

A solution of 2-fluoro-5-(perfluoropropyl)aniline (14; 107 mg, 0.383 mmol) and [symbol] L) was added to a 0° C. solution of CDI (67.0 mg, 0.413 mmol) and CH₂Cl₂ (0.5 mL) dropwise over 1 min. The solution was stirred at 0° C. for 5 min and then at room temperature for 24 h. The acyl imidazole solution was added to a −5° C. solution of 71 (102 mg, 0.383 mmol) and CH₂Cl₂ (2 mL), down the wall of the flask over 5 min. The solution was allowed to warm to room temperature as the bath melted and stirred for a total of 12 h. Purification by silica gel chromatography (25 g cartridge), eluting at 25 mL/min and using a linear gradient of CH₂Cl₂/MeOH: 100:0→90:10 over 30 column volumes, provided 53.0 mg (24%) of the title compound as a white solid: 1H NMR (400 MHz, DMSO-d₆) δ 9.27 (s, 1H), 8.95 (d, J=2.9 Hz, 1H), 8.63 (dd, J=7.3, 2.2 Hz, 1H), 8.13 (s, 1H), 7.50-7.60 (m, 3H), 7.38-7.44 (m, 3H), 7.31-7.37 (m, 1H), 6.04 (br s, 2H), 4.96 (sept, J=6.8 Hz, 1H), 1.46 (d, J=6.8 Hz, 6H); ¹⁹F NMR (376 MHz, DMSO-d₆) δ −79.0 (t, J=9.2 Hz, 3F), −109.6-−109.4 (m, 2F), −123.4 (br. s., 1F), −125.6 (s, 2F); LC-MS (ESI+) m/z: [M+H]⁺ Calcd for C₂₅H₂₁F₈N₆O 573.2; Found 573.4.

### 1-(4-(4-Amino-7-isopropyl-7H-pyrrolo[2,3-d]pyrimidin-5-yl)phenyl)-3-(2-fluoro-5-(perfluorobutan-2-yl)phenyl)urea 2,2,2-trifluoroacetate (APS-8-35-4 (116))

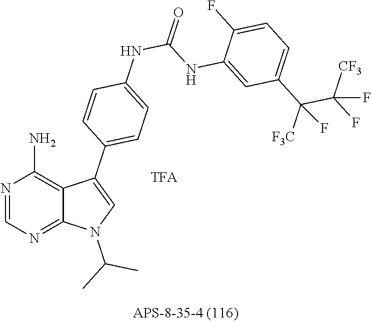 [SMILES: CC(C)n1cc(-c2ccc(NC(=O)Nc3cc(C(F)(C(F)(F)F)C(F)(F)C(F)(F)F)ccc3F)cc2)c2c(N)ncnc21]

A solution of 2-fluoro-5-(perfluorobutan-2-yl)aniline (20; 135 mg, 0.410 mmol) and CH₂Cl₂ (0.5 mL) was added to a 0° C. solution of CDI (70.0 mg, 0.432 mmol) and CH₂Cl₂ (0.5 mL) dropwise over 1 min. The solution was stirred at 0° C. for 5 min and then at room temperature for 24 h. The acyl imidazole solution was added to a −5° C. solution of 71 (110 mg, 0.411 mmol) and CH₂Cl₂ (2 mL), down the wall of the flask over 5 min. The solution was allowed to warm to room temperature as the bath melted and stirred for a total of 12 h. Purification by silica gel chromatography (25 g cartridge), eluting at 25 mL/min and using a linear gradient of CH₂Cl₂/MeOH: 100:0→90:10 over 30 column volumes, provided 53.0 mg (24%) of the title compound as a white solid: ¹H NMR (400 MHz, DMSO-d₆) δ 9.47 (s, 1H), 9.06 (d, J=2.4 Hz, 1H), 8.69 (d, J=6.8 Hz, 1H), 8.43 (s, 1H), 7.77 (s, 1H), 7.61 (d, J=8.8 Hz, 2H), 7.54 (dd, J=10.8, 8.8 Hz, 1H), 7.44 (d, J=8.6 Hz, 2H), 7.31 (dd, J=4.8, 4.0 Hz, 1H), 5.04 (sept, J=6.7 Hz, 1H), 1.50 (d, J=6.6 Hz, 6H); ¹⁹F NMR (376 MHz, DMSO-d₆) δ −73.2-−72.9 (m, 3F), −73.6 (s, 3F), −78.3 (d, J=11.4 Hz, 3F), −120.9-−120.3 (m, 2F), −124.7 (br s, 1F), −181.2-−180.7 (m, 1F); LC-MS (ESI+) m/z: [M+H]⁺ Calcd for C₂₆H₂₁F₁₀N₆O 623.2; Found 623.3.

### 1-(4-(4-amino-7-isopropyl-7H-pyrrolo[2,3-d]pyrimidin-5-yl)phenyl)-3-(2-fluoro-5-(perfluorobutyl)phenyl)urea (APS-7-25-3 (117))

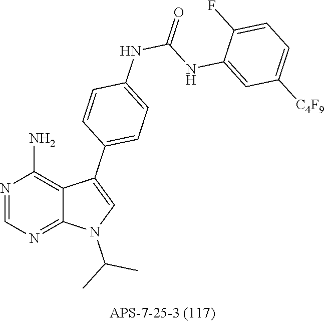

A solution of 2-fluoro-5-(perfluorobutyl)aniline (18; 126 mg, 0.383 mmol) and CH₂Cl₂ (0.5 mL) was added to a 0° C. solution of CDI (67.0 mg, 0.413 mmol) and CH₂Cl₂ (0.5 mL) dropwise over 1 min. The solution was stirred at 0° C. for 5 min and then at room temperature for 24 h. The acyl imidazole solution was added to a −5° C. solution of 71 (102 mg, 0.383 mmol) and CH₂Cl₂ (2 mL), down the wall of the flask over 5 min. The solution was allowed to warm to room temperature as the bath melted and stirred for a total of 12 h. Purification by silica gel chromatography (25 g cartridge), eluting at 25 mL/min and using a linear gradient of CH₂Cl₂/MeOH: 100:0→90:10 over 30 column volumes, provided 101 mg (42%) of the title compound as a white solid: ¹H NMR (400 MHz, DMSO-d₆) δ 9.27 (s, 1H), 8.95 (d, J=2.9 Hz, 1H), 8.63 (dd, J=7.2, 2.1 Hz, 1H), 8.13 (s, 1H), 7.50-7.59 (m, 3H), 7.38-7.44 (m, 3H), 7.31-7.38 (m, 1H), 6.04 (br s, 2H), 4.96 (sept, J=6.6 Hz, 1H), 1.46 (d, J=6.8 Hz, 6H); ¹⁹F NMR (376 MHz, DMSO-d₆) δ −80.0 (t, J=9.2 Hz, 3F), −108.9-−108.6 (m, 2F), −122.0-−121.7 (m, 2F), −123.3 (br s, 1F), −124.9-−124.6 (m, 2F); LC-MS (ESI+) m/z: [M+H]⁺ Calcd for C₂₆H₂₁F₁₀N₆O 623.2; Found 623.5.

### 1-(4-(4-Amino-7-isopropyl-7H-pyrrolo[2,3-d]pyrimidin-5-yl)phenyl)-3-(4-(trifluoromethyl)phenyl)urea (APS-8-85-2 (118))

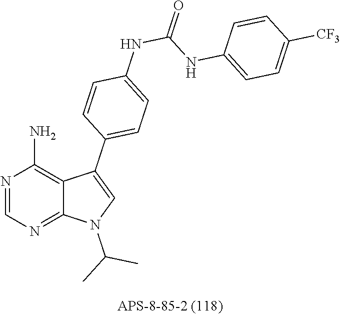 [SMILES: CC(C)n1cc(-c2ccc(NC(=O)Nc3ccc(C(F)(F)F)cc3)cc2)c2c(N)ncnc21]

To a −5° C. solution of 71 (65.0 mg, 0.243 mmol) and CH₂Cl₂ (1 mL), was added a solution of 1-isocyanato-4-(trifluoromethyl)benzene (38.0 μL, 0.266 mmol) and CH₂Cl₂ (1 mL), down the wall of the flask over 5 min. The solution was allowed to warm to room temperature overnight as the bath melted. After stirring 12 h, the resulting precipitate was collected by vacuum filtration; washed solid with CH₂Cl₂. Air-drying provided 38.5 mg (35%) of the title compound as a white solid: 1H NMR (400 MHz, DMSO-d₆) δ 9.13 (s, 1H), 8.90 (s, 1H), 8.13 (s, 1H), 7.61-7.71 (m, 4H), 7.57 (d, J=8.6 Hz, 2H), 7.38-7.43 (m, 3H), 6.04 (br s, 2H), 4.96 (sept, J=6.7 Hz, 1H), 1.46 (d, J=6.6 Hz, 6H); ¹⁹F NMR (376 MHz, DMSO-d₆) δ −59.6 (s, 3F); LC-MS (ESI+) m/z: [M+H]⁺ Calcd for C₂₃H₂₂F₃N₆O 455.2; Found 455.3.

### 1-(4-(4-Amino-7-isopropyl-7H-pyrrolo[2,3-d]pyrimidin-5-yl)phenyl)-3-(4-(perfluoroethyl)phenyl)urea (APS-8-100-4 (61))

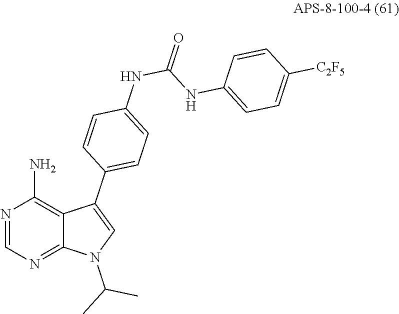

A solution of 4-(perfluoroethyl)aniline (6; 87.0 mg, 0.412 mmol) and CH₂Cl₂ (0.5 mL) was added to a 0° C. solution of CDI (70.0 mg, 0.432 mmol) and CH₂Cl₂ (0.5 mL) dropwise over 1 min. The solution was stirred at 0° C. for 5 min and then at room temperature for 24 h. The acyl imidazole solution was added to a −5° C. solution of 71 (110 mg, 0.411 mmol) and CH₂Cl₂ (2 mL), down the wall of the flask over 5 min. The solution was allowed to warm to room temperature as the bath melted and stirred for a total of 12 h. The resulting precipitate was collected by vacuum filtration; washed solid with CH₂Cl₂. Air-drying provided 47.0 mg (23%) of the title compound as a white solid: ¹H NMR (400 MHz, DMSO-d₆) δ 9.16 (s, 1H), 8.91 (s, 1H), 8.12 (s, 1H), 7.68-7.74 (m, 2H), 7.54-7.63 (m, 4H), 7.38-7.43 (m, 3H), 4.96 (quin, J=6.6 Hz, 1H), 1.46 (d, J=6.6 Hz, 6H); ¹⁹F NMR (376 MHz, DMSO-d₆) δ −83.7 (s, 3F), −112.2 (br s, 2F); LC-MS (ESI+) m/z: [M+H]⁺ Calcd for C₂₄H₂₂F₅N₆O 505.2; Found 505.2.

### 1-(4-(4-Amino-7-isopropyl-7H-pyrrolo[2,3-d]pyrimidin-5-yl)phenyl)-3-(4-(perfluoropropan-2-yl)phenyl)urea (APS-8-50-4 (119))

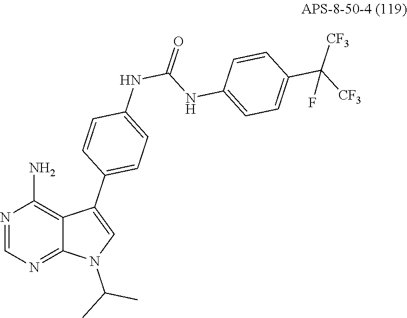 [SMILES: CC(C)n1cc(-c2ccc(NC(=O)Nc3ccc(C(F)(C(F)(F)F)C(F)(F)F)cc3)cc2)c2c(N)ncnc21]

A solution of 4-(perfluoropropan-2-yl)aniline (8; 107 mg, 0.410 mmol) and CH₂Cl₂ (0.5 mL) was added to a 0° C. solution of CDI (70.0 mg, 0.432 mmol) and CH₂Cl₂ (0.5 mL) dropwise over 1 min. The solution was stirred at 0° C. for 1 h and then at room temperature for 24 h. The acyl imidazole solution was added to a −5° C. solution of 71 (110 mg, 0.411 mmol) and CH₂Cl₂ (2 mL), down the wall of the flask over 5 min. The solution was allowed to warm to room temperature as the bath melted and stirred for a total of 12 h. Purification by silica gel chromatography (25 g cartridge), eluting at 25 mL/min and using a linear gradient of CH₂Cl₂/MeOH: 100:0→90:10 over 30 column volumes, provided 112 mg (49%) of the title compound as a white solid: ¹H NMR (400 MHz, DMSO-d₆) δ 9.12 (s, 1H), 8.89 (s, 1H), 8.12 (s, 1H), 7.71 (d, J=8.6 Hz, 2H), 7.54-7.61 (m, 4H), 7.37-7.43 (m, 3H), 6.03 (br s, 2H), 4.96 (sept, J=6.5 Hz, 1H), 1.46 (d, J=6.6 Hz, 6H); ¹⁹F NMR (376 MHz, DMSO-d₆) δ −74.8 (d, J=6.9 Hz, 6F), −180.7-−180.6 (m, 1F); LC-MS (ESI+) m/z: [M+H]⁺ Calcd for C₂₅H₂₂F₇N₆O 555.2; Found 555.3.

### 1-(4-(4-Amino-7-isopropyl-7H-pyrrolo[2,3-d]pyrimidin-5-yl)phenyl)-3-(4-(perfluoropropyl)phenyl)urea 2,2,2-trifluoroacetate (APS-8-51-4 (62))

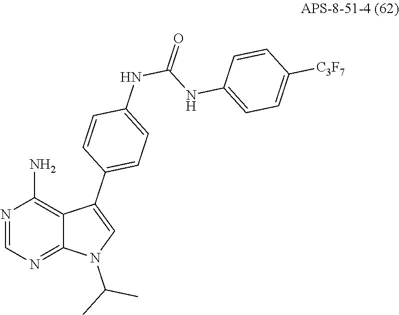

A solution of 4-(perfluoropropyl)aniline (7; 107 mg, 0.410 mmol) and CH₂Cl₂ (0.5 mL) was added to a 0° C. solution of CDI (70.0 mg, 0.432 mmol) and CH₂Cl₂ (0.5 mL) dropwise over 1 min. The solution was stirred at 0° C. for 1 h and then at room temperature for 24 h. The acyl imidazole solution was added to a −5° C. solution of 71 (110 mg, 0.411 mmol) and CH₂Cl₂ (2 mL), down the wall of the flask over 5 min. The solution was allowed to warm to room temperature as the bath melted and stirred for a total of 12 h. Purification by reverse-phase chromatography, eluting at 40 mL/min and using a linear gradient of H₂O (with 0.1% v/v TFA)/MeCN (with 0.1% v/v TFA): 90:10→1:99 over 20 min, provided 94.7 mg (42%) of the title compound as a white solid: 1H NMR (400 MHz, DMSO-d₆) δ 9.56 (s, 1H), 9.36 (s, 1H), 8.42 (s, 1H), 7.69-7.80 (m, 3H), 7.54-7.68 (m, 4H), 7.43 (d, J=8.6 Hz, 2H), 5.03 (sept, J=6.5 Hz, 1H), 1.50 (d, J=6.8 Hz, 6H); ¹⁹F NMR (376 MHz, DMSO-d₆) δ −73.5 (br s, 3F), −79.0 (t, J=9.2 Hz, 3F), −109.4-−109.1 (m, 2F), −125.7 (br s, 2F); LC-MS (ESI+) m/z: [M+H]⁺ Calcd for C₂₅H₂₂F₇N₆O 555.2; Found 555.7.

### 1-(4-(4-Amino-7-isopropyl-7H-pyrrolo[2,3-d]pyrimidin-5-yl)phenyl)-3-(4-(perfluorobutan-2-yl)phenyl)urea 2,2,2-trifluoroacetate (APS-8-82-2 (63))

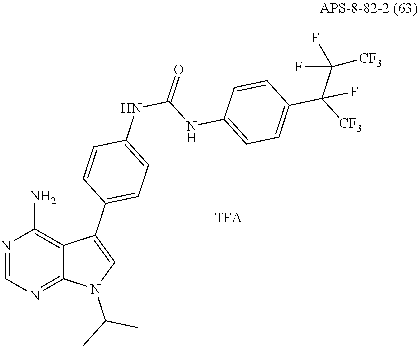 [SMILES: CC(C)n1cc(-c2ccc(NC(=O)Nc3ccc(C(F)(C(F)(F)F)C(F)(F)C(F)(F)F)cc3)cc2)c2c(N)ncnc21]

A solution of 4-(perfluorobutan-2-yl)aniline (10; 107 mg, 0.410 mmol) and CH₂Cl₂ (0.5 mL) was added to a 0° C. solution of CDI (70.0 mg, 0.432 mmol) and CH₂Cl₂ (0.5 mL) dropwise over 1 min. The solution was stirred at 0° C. for 1 h and then at room temperature for 24 h. The acyl imidazole solution was added to a −5° C. solution of 71 (110 mg, 0.411 mmol) and CH₂Cl₂ (2 mL), down the wall of the flask over 5 min. The solution was allowed to warm to room temperature as the bath melted. After 12 h the solution was concentrated to dryness and purified by reverse-phase chromatography, eluting at 40 mL/min and using a linear gradient of H₂O (with 0.1% v/v TFA)/MeCN (with 0.1% v/v TFA): 90:10→1:99 over 20 min. Obtained 164 mg (57%) of the title compound as a white solid: 1H NMR (400 MHz, DMSO-d₆) δ 9.53 (s, 1H), 9.36 (s, 1H), 8.43 (s, 1H), 7.70-7.79 (m, 3H), 7.64 (d, J=8.6 Hz, 2H), 7.57 (d, J=8.6 Hz, 2H), 7.43 (d, J=8.8 Hz, 2H), 5.03 (sept, J=6.5 Hz, 1H), 1.50 (d, J=6.6 Hz, 6H); ¹⁹F NMR (376 MHz, DMSO-d₆) δ −73.4-−73.2 (m, 3F), −73.6 (s, 3F), −78.3 (d, J=11.4 Hz, 3F), −120.8-−120.6 (m, 2F), −182.1 (br s, 1F); LC-MS (ESI+) m/z: [M+H]⁺ Calcd for C₂₆H₂₂F₉N₆O 605.2; Found 605.5.

### 1-(4-(4-Amino-7-isopropyl-7H-pyrrolo[2,3-d]pyrimidin-5-yl)phenyl)-3-(4-(perfluorobutyl)phenyl)urea (APS-8-52-4 (120))

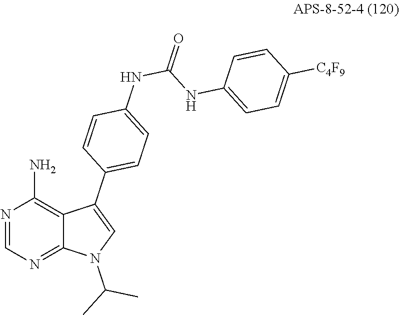

A solution of 4-(perfluorobutyl)aniline (9; 128 mg, 0.411 mmol) and CH₂Cl₂ (0.5 mL) was added to a 0° C. solution of CDI (70.0 mg, 0.432 mmol) and CH₂Cl₂ (0.5 mL) dropwise over 1 min. The solution was stirred at 0° C. for 1 h and then at room temperature for 24 h. The acyl imidazole solution was added to a −5° C. solution of 71 (110 mg, 0.411 mmol) and CH₂Cl₂ (2 mL), down the wall of the flask over 5 min. The solution was allowed to warm to room temperature as the bath melted and stirred for a total of 12 h. Purification by silica gel chromatography (25 g cartridge), eluting at 25 mL/min and using a linear gradient of CH₂Cl₂/MeOH: 100:0→90:10 over 40 column volumes, provided 80.4 mg (32%) of the title compound as a white solid: ¹H NMR (400 MHz, DMSO-d₆) δ 9.17 (s, 1H), 8.92 (s, 1H), 8.12 (s, 1H), 7.71 (d, J=8.8 Hz, 2H), 7.54-7.62 (m, 4H), 7.38-7.43 (m, 3H), 4.96 (quin, J=6.7 Hz, 1H), 1.46 (d, J=6.8 Hz, 6H); ¹⁹F NMR (376 MHz, DMSO-d₆) δ −80.1-−79.9 (m, 3F), −108.6-−108.3 (m, 2F), −122.1-−121.8 (m, 2F), −124.9-−124.6 (m, 2F); LC-MS (ESI+) m/z: [M+H]⁺ Calcd for C₂₆H₂₂F₉N₆O 605.2; Found 605.7.

### 1-(4-(4-Amino-7-methyl-7H-pyrrolo[2,3-d]pyrimidin-5-yl)phenyl)-3-(2-fluoro-5-(trifluoromethyl)phenyl)urea (APS-6-94)

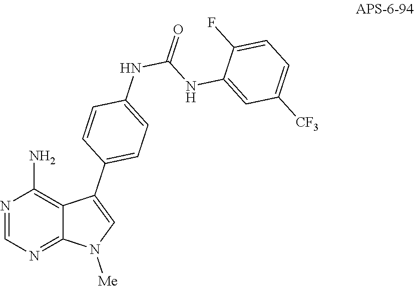 [SMILES: Cn1cc(-c2ccc(NC(=O)Nc3cc(C(F)(F)F)ccc3F)cc2)c2c(N)ncnc21]

To a −5° C. solution of 5-(4-aminophenyl)-7-methyl-7H-pyrrolo[2,3-d]pyrimidin-4-amine (61.0 mg, 0.255 mmol) and CH₂Cl₂ (1 mL), in an 8 mL vial, was added 1-fluoro-2-isocyanato-4-(trifluoromethyl)benzene 37.5 μL, 0.259 mmol) dropwise over 5 min. The reaction mixture was allowed to warm to room temperature over 2 h and was stirred for a total of 20 h. The product was isolated by vacuum filtration; washed solid with CH₂Cl₂. Air-drying yielded 53.8 mg (48%) of the title compound as a white solid: ¹H NMR (400 MHz, DMSO-d₆) δ 9.27 (s, 1H), 8.93 (d, J=2.4 Hz, 1H), 8.64 (dd, J=7.3, 2.2 Hz, 1H), 8.15 (s, 1H), 7.57 (d, J=8.6 Hz, 2H), 7.47-7.54 (m, 1H), 7.40 (d, J=8.3 Hz, 3H), 7.27 (s, 1H), 3.74 (s, 3H); LC-MS (ESI+) m/z: [M+H]⁺ Calcd for C₂₁H₁₇F₄N₆O 445.4; Found 445.4.

### 1-(4-(4-Amino-7-cyclopentyl-7H-pyrrolo[2,3-d]pyrimidin-5-yl)phenyl)-3-(2-fluoro-5-(trifluoromethyl)phenyl)urea (APS-6-91)

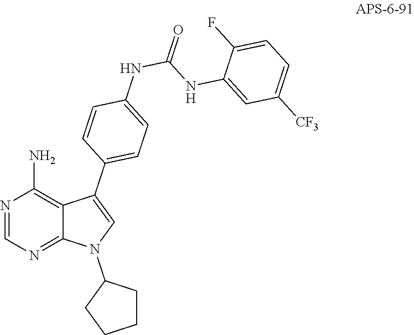 [SMILES: Nc1ncnc2c1c(-c1ccc(NC(=O)Nc3cc(C(F)(F)F)ccc3F)cc1)cn2C1CCCC1]

To a −5° C. solution of 5-(4-aminophenyl)-7-cyclopentyl-7H-pyrrolo[2,3-d]pyrimidin-4-amine (73.8 mg, 0.252 mmol) and CH₂Cl₂ (1 mL), in an 8 mL vial, was added 1-fluoro-2-isocyanato-4-(trifluoromethyl)benzene 37.0 μL, 0.256 mmol) dropwise over 5 min. The reaction mixture was allowed to warm to room temperature over 2 h and was stirred for a total of 20 h. The product was isolated by vacuum filtration; washed solid with CH₂Cl₂. Air-drying yielded 63.6 mg (51%) of the title compound as a white solid: ¹H NMR (400 MHz, DMSO-d₆) δ 9.30 (s, 1H), 8.94 (d, J=2.4 Hz, 1H), 8.64 (dd, J=7.1, 1.7 Hz, 1H), 8.13 (s, 1H), 7.57 (d, J=8.6 Hz, 2H), 7.46-7.54 (m, 1H), 7.34-7.45 (m, 4H), 5.08 (quin, J=7.5 Hz, 1H), 2.03-2.18 (m, 2H), 1.78-1.99 (m, 4H), 1.69 (d, J=6.6 Hz, 2H); LC-MS (ESI+) m/z: [M+H]⁺ Calcd for C₂₅H₂₃F₄N₆O 499.5; Found 499.6.

### Example 4—In Vitro Kinase Percent Inhibition Measurements

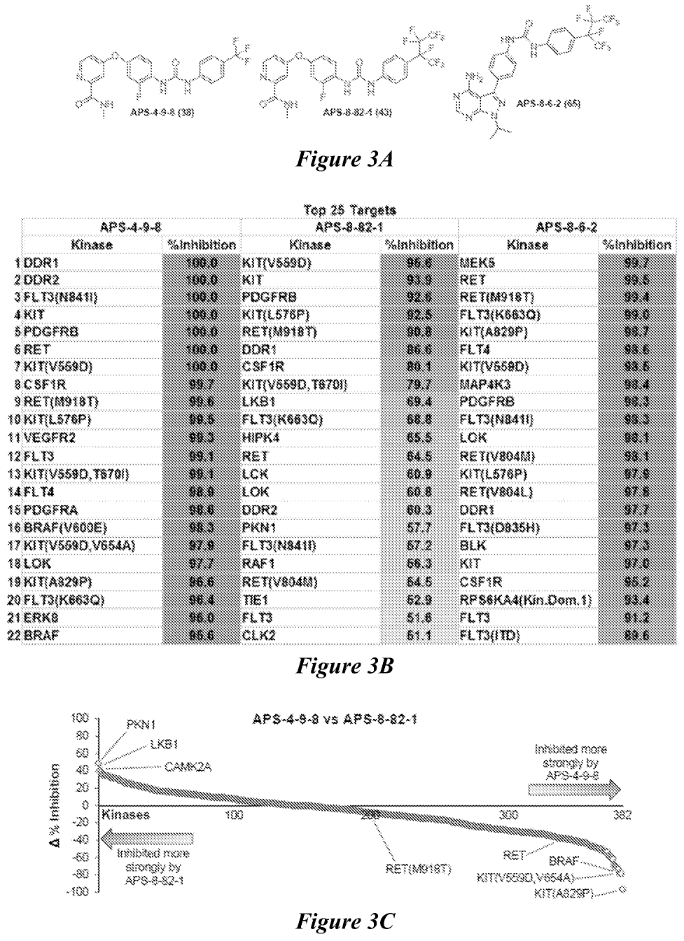

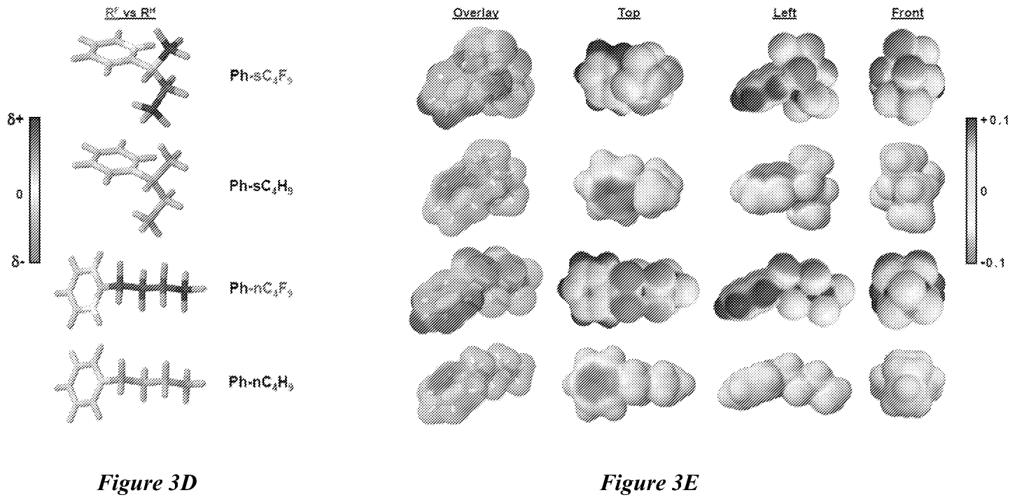

Compounds were assayed against a panel of 468 purified human kinases (DiscoverX) to measure percentage inhibition values and derive kinome profiles (FIGS. 3B-3C). All compounds were screened at 1 μM, using two experimental replicates to determine percent inhibition values. Detailed procedures for kinase reactions and assay formats are described at http://www.discoverx.com. Raw data sets for individual compounds were filtered based on the variance of experimental replicates (cutoff=average variance+standard deviation).

### Example 5-Kd Measurements

Wild-type RET Kd values were determined by DiscoverX (Fremont, CA) using a bead based competition assay (KINOMEscan). In brief, kinases are expressed on phage and immobilized on beads via active site-directed ligands. Test compounds are premixed with kinases and assayed for the ability to compete for immobilized ligands. Binding constants are calculated with a standard dose-response curve and the Hill equation, with Hill slope set to −1. The method has been used extensively to characterize KI-binding data (Davis et al., “Comprehensive Analysis of Kinase Inhibitor Selectivity,” Nat. Biotechnol. 29:1046-1051 (2011), which is hereby incorporated by reference in its entirety). For each compound-RET pair, an 11-point series ranging from 30,000 to 0.5 nM, in three-fold dilutions, was used to derive Kd values. Kd values are the average of two experimental replicates.

### Example 6—Fly Rescue Assay

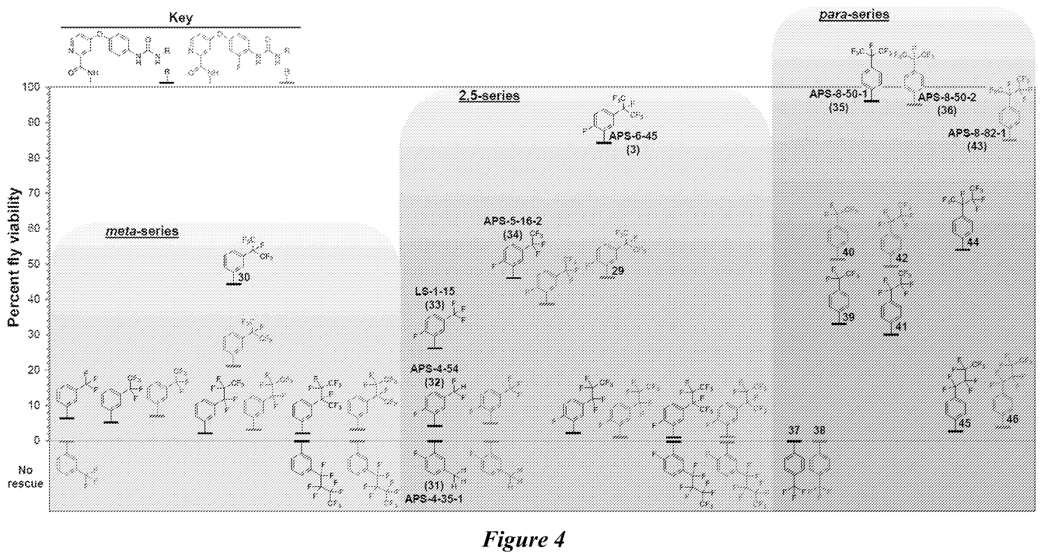

All drugs were synthesized in-house. All compounds were dissolved in DMSO (Sigma-Aldrich; St. Louis, MO) and mixed with semi-defined fly medium (BDSC) to make drug food (0.1% final DMSO concentration). For drug screening, ptc>dRet^{M955T} embryos were raised until adulthood on drug food for 13 d at 25° C. The number of empty pupal cases (i.e., adults) [symbol] that of total pupal cases (P) to determine % viability. The small-molecule screening data is summarized in FIGS. 4, 5, and 6. Fly randomization was used throughout the assays.

### Example 7—Permeability Assays

Caco-2 and MDCK permeability assays to assess transport in both directions, apical to basolateral (A-B) and basolateral to apical (B-A), across a cell monolayer were conducted at Quintara Discovery. The compounds were tested at a final concentration of 5 microM, with HBSS, pH 7.4 on apical side and HBSS, pH 7.4 on basolateral side. Transport was allowed to proceed for 1 hour at 37° C., with compound detection performed by LC-MS/MS. Assays were conducted twice. Papp, B-A and Papp, A-B, ×10⁻⁶ cm/s values were determined at Quintara.

### Example 8—Nanobret Cell Based Target Engagement Assays

Measurements of inhibitor IC₅₀ values against RET (WT and M918T) were carried out according to manufacturer's instructions as provided by Promega for the in-cell kinase assay, and as previously reported (Vasta et al., “Quantitative, Wide-Spectrum Kinase Profiling in Live Cells for Assessing the Effect of Cellular ATP on Target Engagement,” Cell Chem Biol 25:206-214.e11 (2018), which is hereby incorporated by reference in its entirety).

### Results of Examples 1-8

A chemical tool box for perfluoroalkylated type II KI CAP groups

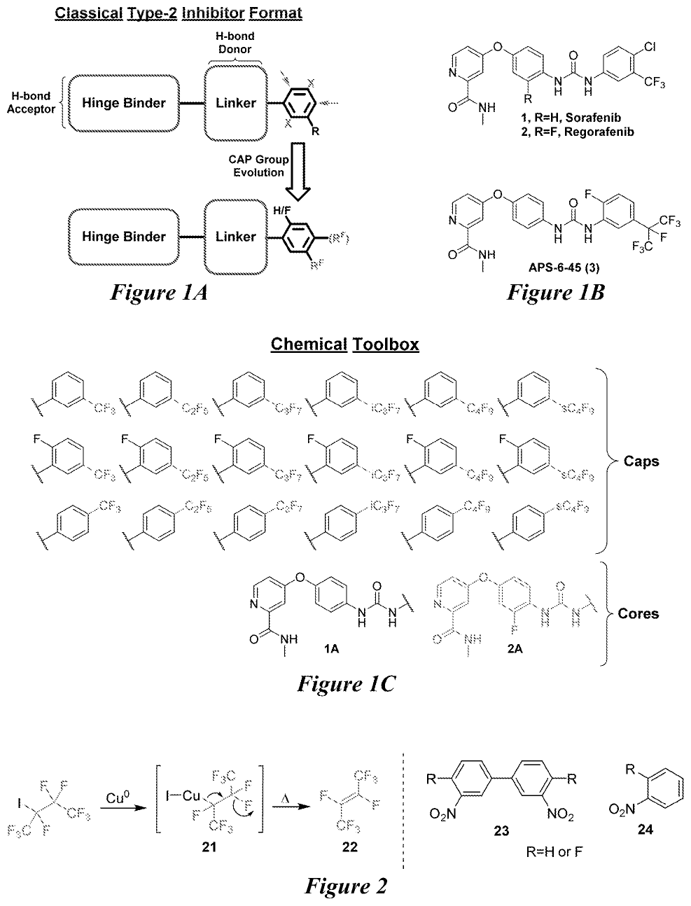

A chemical toolbox (FIG. 1C) was envisioned comprising of 18 perfluoroalkyl-substituted phenyl CAP-groups; meta-substituted, with and without a 2-fluoro-substituent, as well as para-substituted building blocks. Within this toolkit linear and branched perfluoroalkyl-groups ranging in size from —CF₃ to —C₄F₉ were also envisioned. Generation of the requisite aniline building blocks allowed for facile formation of specific embodiments of the core subunits corresponding to sorafenib (1A) and regorafenib (2A), and hence these were used as starting points to examine the effectiveness of perfluoroalkyl-substituted CAP-groups within a type II KI format.

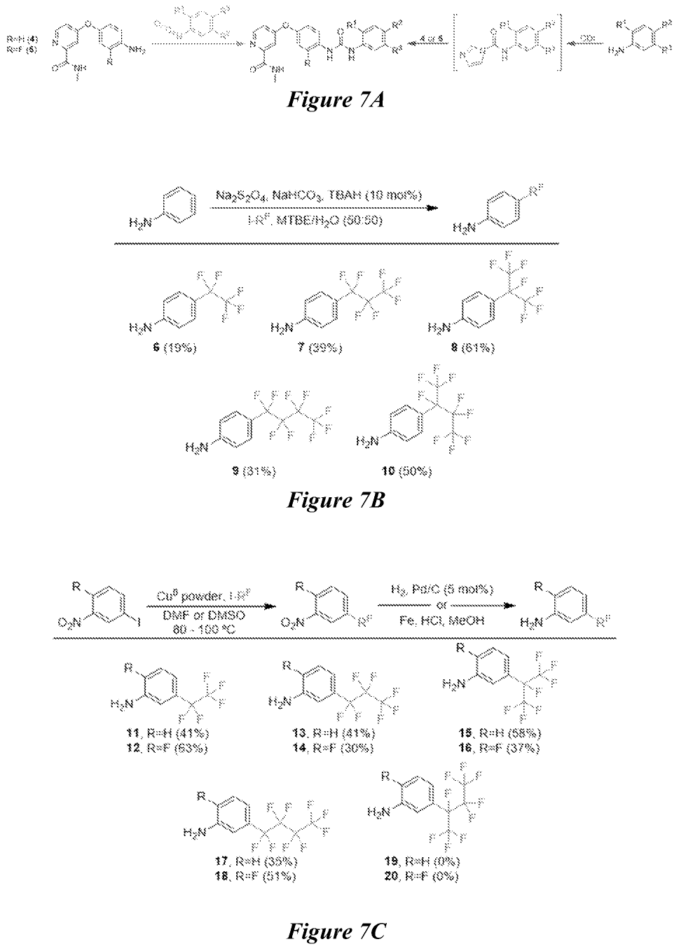

The sorafenib and regorafenib core aniline building blocks (4 and 5; FIG. 7A) could be adorned with a urea-linked CAP group by reaction with an appropriately functionalized commercially available phenylisocyanate (red arrow) or an in situ formed acyl-imidazole (blue arrows); 4 and 5 were synthesized from simpler components. The para-substituted series of perfluoroalkyl-aniline building blocks (6-10) were accessed in reasonable yields from aniline and [symbol] iodo perfluoroalkane, by employing sodium hydrosulfite under phase-transfer conditions (FIG. 7B) (Barata-Vallejo et al., “Perfluoroalkylation Reactions of (Hetero)arenes,” RSC Adv. 5:62498-62518 (2015), which is hereby incorporated by reference in its entirety). The majority of the meta-substituted series could be accessed via a two-step sequence, which began with a copper-mediated coupling between a commercially available nitro aryl iodide and an iodo perfluoroalkane (FIG. 7C) (Mcloughlin & Thrower, “A Route to Fluoroalkyl-substituted Aromatic Compounds Involving Fluoroalkylcopper Intermediates,” Tetrahedron 25:5921-5940 (1969), which is hereby incorporated by reference in its entirety). The resulting nitro aryl perfluoroalkane intermediates were reduced with catalytic Pd/C (R═H) or an excess of iron powder (R═F) to provide aniline building blocks 11-18 in good overall yield. Initial efforts to synthesize anilines 19 and 20 were unsuccessful due to failure of the pivotal copper-mediated coupling reaction when employing nonafluoro-2-iodobutane, under a variety of conditions. Presumably the in situ formed perfluoroalkyl-cuprate (21; or the related organocopper(I) species) is not stable under the reaction conditions and collapses to alkene 22 (FIG. 2). This unproductive consumption of nonafluoro-2-iodobutane ultimately resulted in biaryl 23 and reduction product 24 as the only identifiable components in crude reaction mixtures.

In order to access 19 and 20, alternative synthetic strategies were developed starting from a common intermediate (10; FIG. 7D). Thus, aniline 10 was acylated under standard conditions to provide 25. Nitration of 25 with fuming nitric acid occurred readily at room temperature, and quenching the reaction with water caused rapid cleavage of the acetate to provide 26. Reductive removal of the amine with sodium nitrite, and finally palladium-catalyzed nitro-group reduction gave 19. Alternatively, diazotization of 10 with isoamyl nitrite generated 27, which when heated at 110-120° C. rearranged to 28. Nitration of 28, followed by nitro-group reduction provided 20.

In vivo SAR of an initial perfluoroalkylated KI library

The initial libraries of perfluoroalkyl-analogs of sorafenib and regorafenib were screened in the Drosophila MTC model ptc>Ret^{M955T} (FIG. 4). In this model, compounds are assessed based on their ability to suppress oncogenic Ret^{M955T}-induced tumors, which can be quantified as ‘rescue’ from lethality. This is a whole animal assay in which hits must suppress oncogenic Ret^{M955T} but not normal development processes of the fly (Dar et al., “Chemical Genetic Discovery of Targets and Anti-targets for Cancer Polypharmacology,” Nature 486:80-84 (2012), which is hereby incorporated by reference in its entirety). The baseline viability of ptc>Ret¹⁹⁵⁵T flies was calibrated to zero, which creates a high bar for rescue but also reduces false-positive hits. Hundreds of flies are tested per condition (typical n values are greater than [symbol] unds are administered through fly food.

The sorafenib- and regorafenib-based perfluoroalkyl-analogs, could be separated into three groups: 1) meta-series; 2) 2,5-series; and 3) para-series. As depicted in the chemical toolbox (FIG. 1C), the meta- and para-series include a systematic progression from —CF₃ through to —C₄F₉, including branched and straight chain derivatives. The 2,5-series possess a 2-F-substitution in addition to perfluoroalkyl-groups at the 5 position. In both the meta- and 2,5-series, it was found that fly rescue peaked with -iC₃F₇, but dropped off sharply with -nC₃F₇, -nC₄F₉, and -sC₄F₉. Further, in both series the sorafenib-based scaffold proved optimal

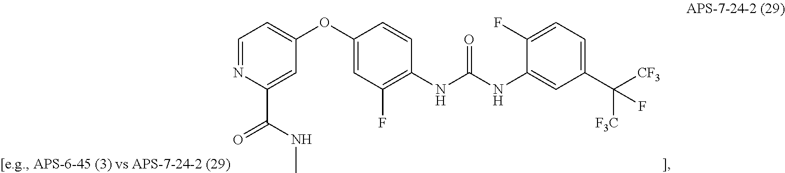 [SMILES: CNC(=O)c1cc(Oc2ccc(NC(=O)Nc3cc(C(F)(C(F)(F)F)C(F)(F)F)ccc3F)c(F)c2)ccn1]

and 2-F-substitution of the CAP group was also generally favored

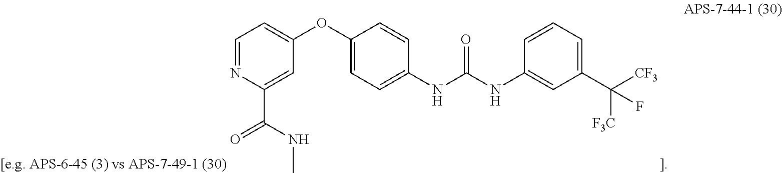 [SMILES: CNC(=O)c1cc(Oc2ccc(NC(=O)Nc3cccc(C(F)(C(F)(F)F)C(F)(F)F)c3)cc2)ccn1]

Within the 2,5-series, it was observed that fly rescue increased as the meta-substituent evolved:

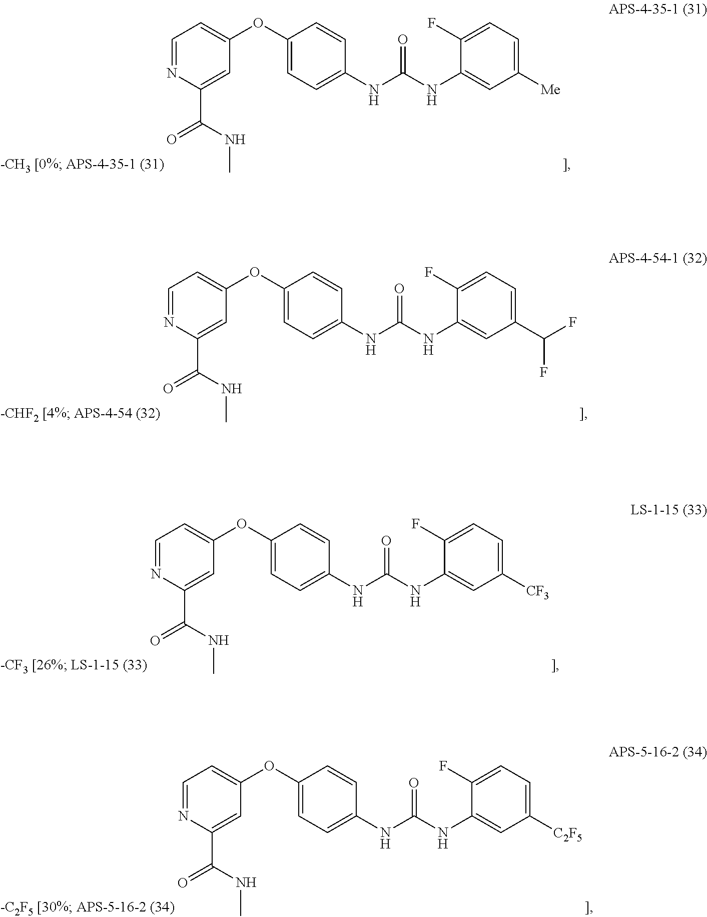

and -iC₃F₇[84%; APS-6-45 (3)]. The SAR among these analogs, and the discovery of APS-6-45 (3), suggested the potential for further optimization/expansion of the prototypical meta-CF₃ group that is commonly found within type II kinase inhibitors (Sonoshita et al., “A Whole-animal Platform to Advance a Clinical Kinase Inhibitor into New Disease Space,” Nat. Chem. Biol. 14(3):291-298 (2018); Zhao et al., “Exploration of Type II Binding Mode: A Privileged Approach for Kinase Inhibitor Focused Drug Discovery?,” ACS Chem. Biol. 9:1230-1241 (2014), which are hereby incorporated by reference in their entirety).

The para-substituted derivatives provided the highest activity compounds, including two analogs

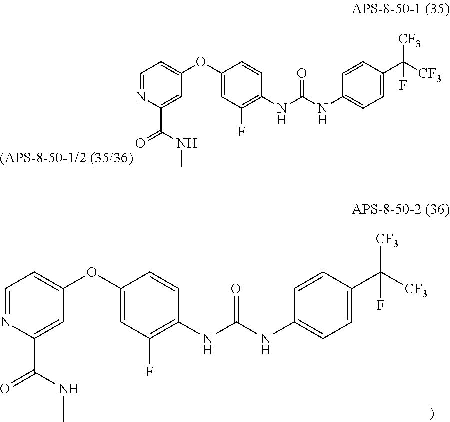 [SMILES: CNC(=O)c1cc(Oc2ccc(NC(=O)Nc3ccc(C(F)(C(F)(F)F)C(F)(F)F)cc3)c(F)c2)ccn1 ; CNC(=O)c1cc(Oc2ccc(NC(=O)Nc3ccc(C(F)(C(F)(F)F)C(F)(F)F)cc3)c(F)c2)ccn1]

that provided ≥95% rescue of fly viability (FIG. 4). Indeed, the para-substituted perfluoroalkyl series was by far the most potent overall, despite the fact that the para-CF₃ analogs

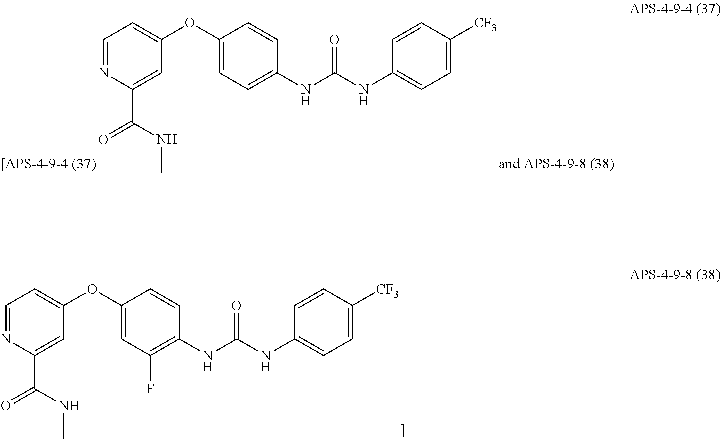 [SMILES: CNC(=O)c1cc(Oc2ccc(NC(=O)Nc3ccc(C(F)(F)F)cc3)cc2)ccn1 ; CNC(=O)c1cc(Oc2ccc(NC(=O)Nc3ccc(C(F)(F)F)cc3)c(F)c2)ccn1]

showed no activity. Both

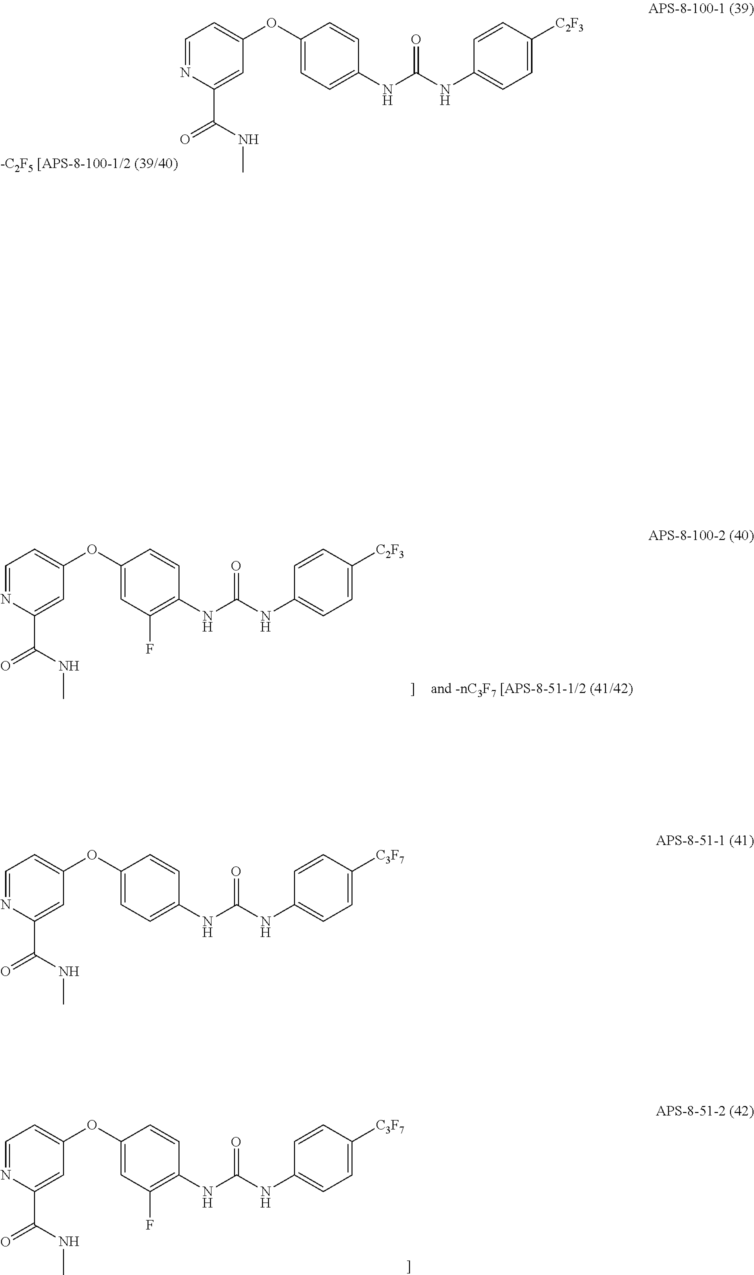

analogs showed rescue ranging from 20-50% with the regorafenib-core displaying slightly better overall activity among the para-series. The -iC₃F₇ analogs [APS-8-50-1/2 (35/36)] showed ≥95% rescue, and 85% rescue was achieved with

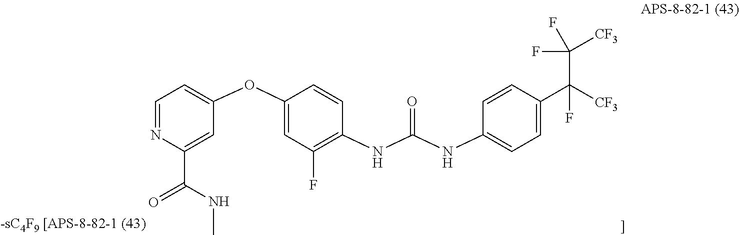 [SMILES: CNC(=O)c1cc(Oc2ccc(NC(=O)Nc3ccc(C(F)(C(F)(F)F)C(F)(F)C(F)(F)F)cc3)c(F)c2)ccn1]

on the regorafenib-core; with 55% rescue observed on the sorafenib-core

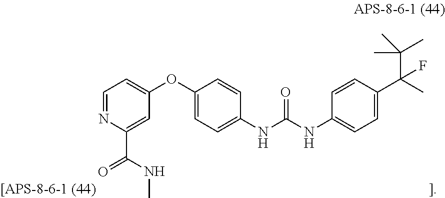 [SMILES: CNC(=O)c1cc(Oc2ccc(NC(=O)Nc3ccc(C(C)(F)C(C)(C)C)cc3)cc2)ccn1]

The branched -sC₄F₉ appeared to represent the upper limit of steric tolerance among the para-series, as there was a steep drop off in activity (1-3%) with the linear -nC₄F₉ analogs

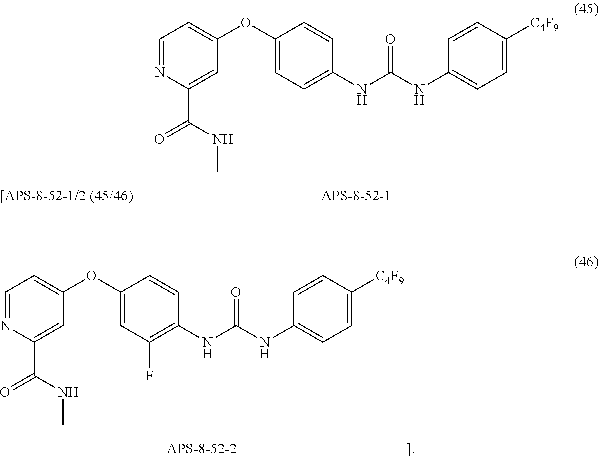

R^{F} vs R^{H} analogs reveal the unique in vivo properties of perfluoroalkanes

Having found several relatively large perfluoroalkylated analogs with strong in vivo activity, the next step was to determine to what extent is the performance of the analogs a unique feature of perfluoroalkanes versus hydrocarbon substituents (—R^{H}) on the CAP-group [APS-4-35-1 (31) vs LS-1-15 (33) provided an initial example; FIG. 4]. To assess this question, matched hydrocarbon pairs were synthesized and tested

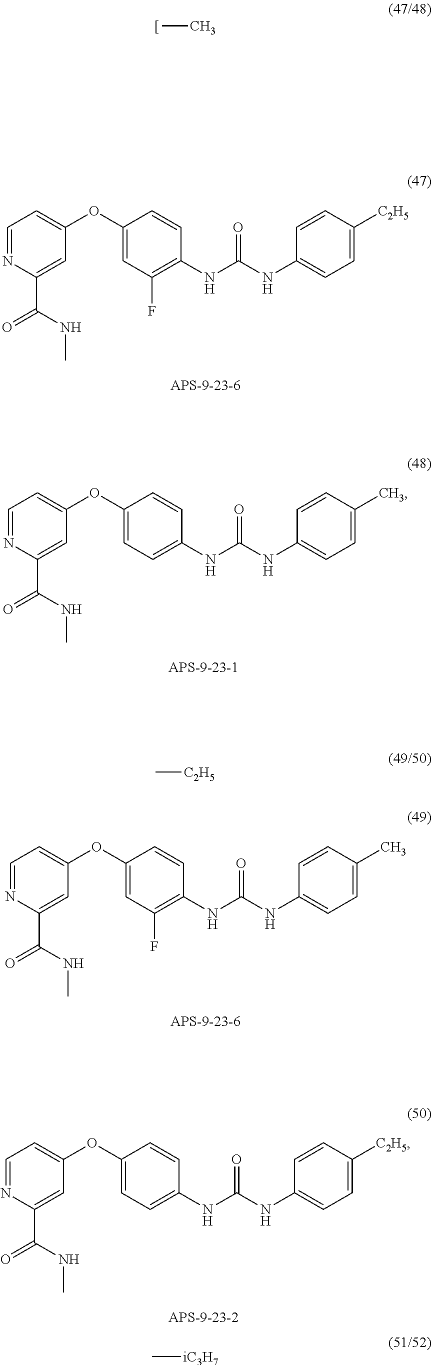

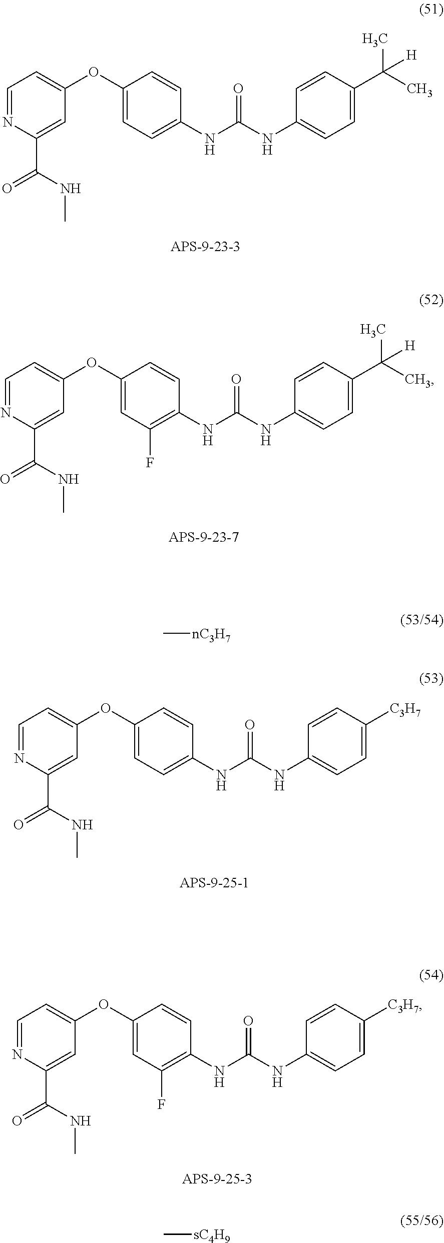

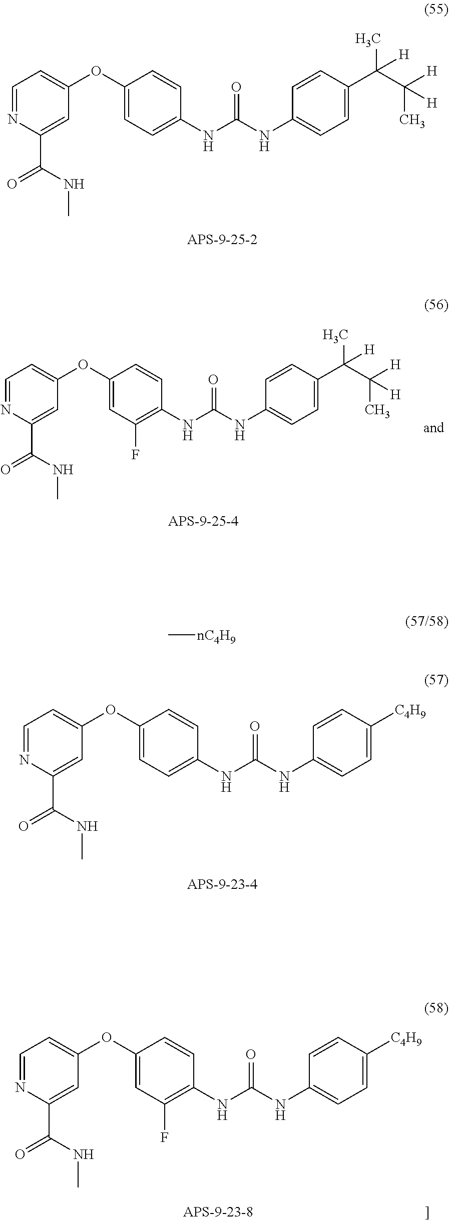

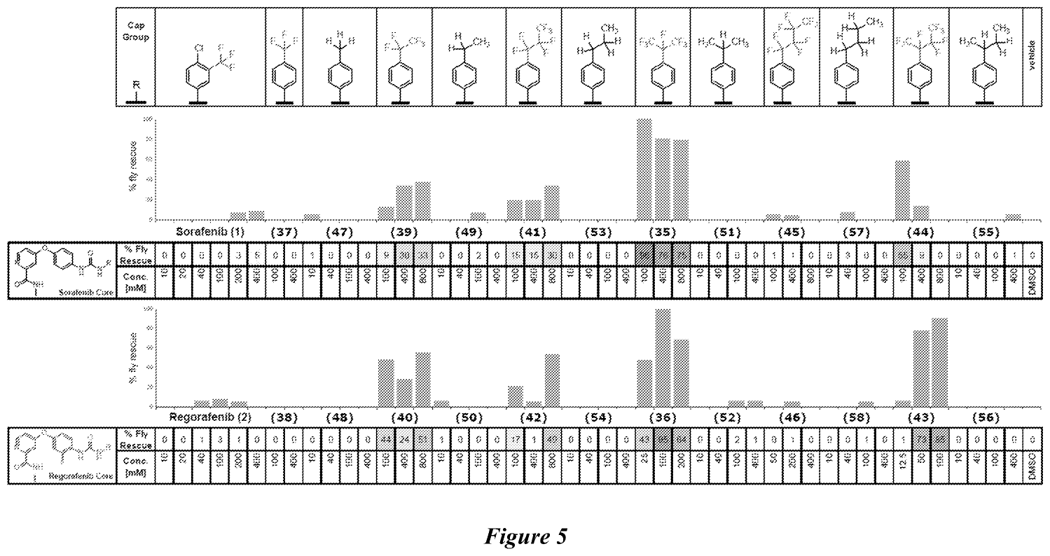

of the perfluoroalkyl-substituted compounds [—CF₃ (37/38), —C₂F₅ (39/40), -iC₃F₇ (35/36), -nC₃F₇ (41/42), -sC₄F₉ (44/43) and -nC₄F₉ (45/46)] within the para-series (FIG. 5). Strikingly, none of the hydrocarbon analogs showed rescue >3% in the MTC model. For example, hydrocarbon compounds APS-9-23-3/7 (51/52; -iC₃H₇) demonstrated 0 and 2% rescue whereas the matched perfluoroalkanes APS-8-50-1/2 (35/36; -iC₃F₇) provided 96% and 95% rescue at optimal doses. For the majority of pairs tested, the hydrocarbon containing analogs provided markedly reduced or no rescue relative to any of the perfluoroalkane containing compounds; —CF₃ vs —CH₃, and -nC₄F₉ vs -nC₄H₉ are exceptions, but with ≤3% rescue.
Systematic alterations in direct target engagement among perfluoroalkyl-analogs

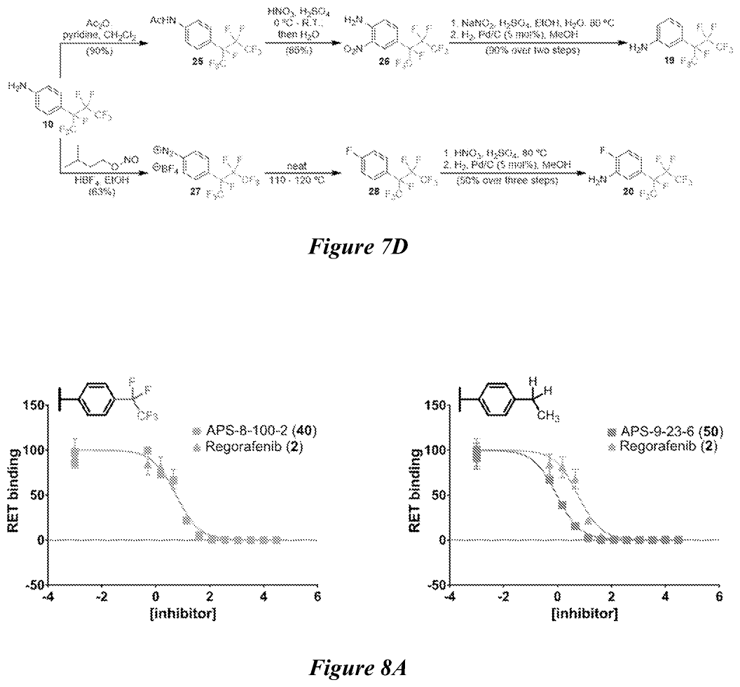

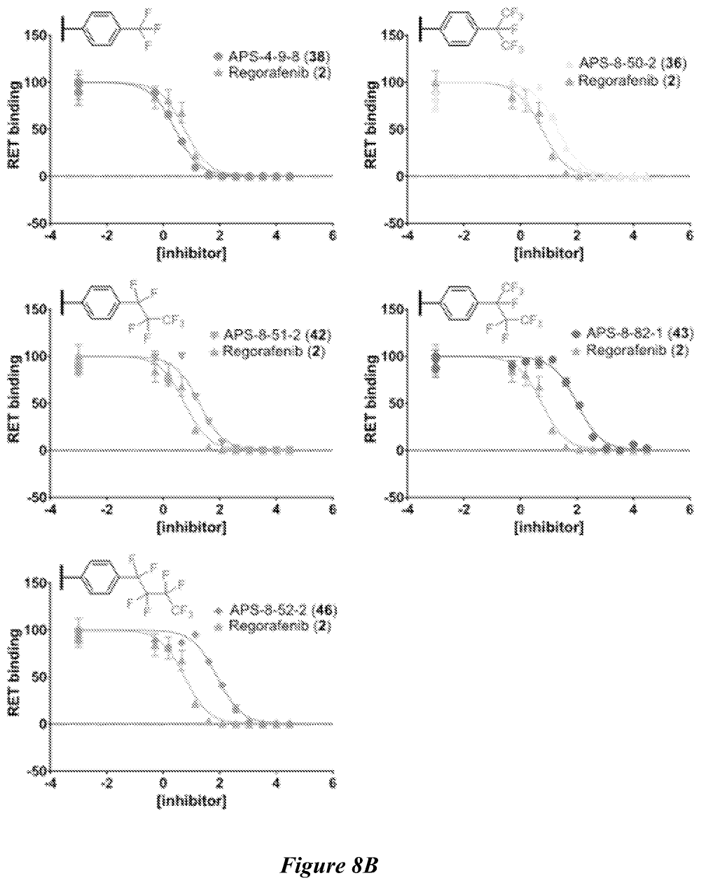

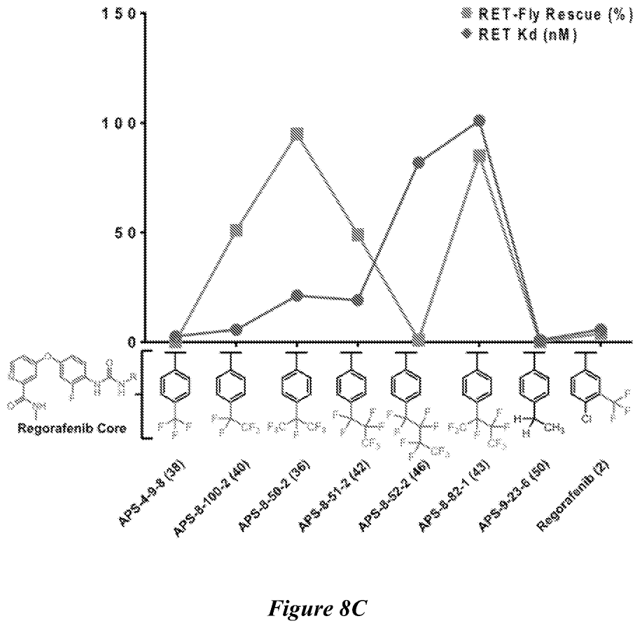

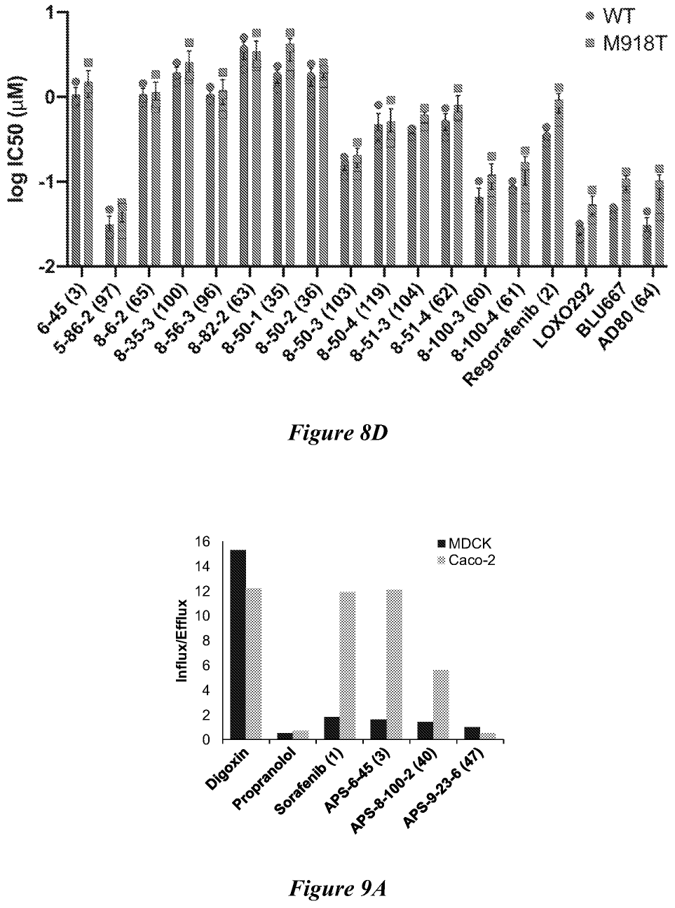

To assess if the differences in activity between R^{F}- and R^{H}-analogs were related to alterations in target engagement, representative analogs for binding on wild-type RET were compared. For this K_{D} values of compounds 40 (para-C₂F₅) and 50 (para-C₂H₅) were determined against recombinant-human, wild-type RET kinase domain. Assays were completed using the bead-based competition format developed by Ambit/DiscoverX and binding curves were plotted relative to regorafenib (2; FIG. 8A). The K_{D} of regorafenib towards RET was determined to be 6.4 nM, which is in close agreement to published values (Wilhelm et al., “Regorafenib (BAY 73-4506): A New Oral Multikinase Inhibitor of Angiogenic, Stromal and Oncogenic Receptor Tyrosine Kinases with Potent Preclinical Antitumor Activity,” Int. J. Cancer 129:245-255 (2011), which is hereby incorporated by reference in its entirety). Moreover, similar K_{D} values of 6.2 and 1 nM were measured for APS-8-100-2 (40) and APS-9-23-6 (50) respectively, against recombinant RET (FIG. 8A), suggesting that the hydrocarbon-containing compounds retain potent in vitro kinase binding activity. This initial experiment suggested that the large difference in fly rescue between 40 (51%) and 50 (1%) does not directly relate to wild-type RET binding (FIGS. 4 and 5).

To further examine how changes within a representative series of CAP-group analogs would impact binding on RET K_{D} values were determined for the rest of the regorafenib-based para-substituted perfluoroalkyl-series (FIG. 8B). Measured affinities towards wild-type RET for the —CF₃ [2.8 nM; APS-4-9-8 (38)] and —C₂F₅[6.2 nM; APS-8-100-2 (40)] analogs were found to be similar to that observed for regorafenib. However, a decreased affinity was measured for the -iC₃F₇[27 nM; APS-8-50-2 (36)], -nC₃F₇[25 nM; APS-8-51-2 (42)], -sC₄F₉[110 nM; APS-8-82-1 (43)], and -nC₄F₉[100 nM; APS-8-52-2 (46)] analogs. Overall, the regorafenib-based, para-substituted, perfluoroalkyl-series demonstrated a progressive decrease in binding affinity as the perfluoroalkyl-group was increased in size. Further, APS-8-82-1 (43), a highly effective compound based on in vivo rescue (85%), demonstrated the weakest binding towards wild-type RET with regard to Kd (110 nM on wild- [symbol] se domain). The divergence between in vitro and in vivo measurements was particularly notable for compounds APS-4-9-8 (38), APS-8-100-2 (40), APS-8-50-2 (36), and APS-8-82-1 (43) (FIG. 8C). Moreover, as best exemplified by comparison of APS-8-50-2 (36; -iC₃F₇)/APS-8-51-2 (42; -nC₃F₇), and APS-8-82-1 (43; -sC₄F₉)/APS-8-52-2 (46; -nC₄F₉), the in vivo model demonstrated strong preferences for branched over linear —C₃F₇ and —C₄F₉; the differences were particularly striking given the similar in vitro Kd measurements observed for these analogs. Thus, a possible (anti-) correlation was observed between improvements in rescue within the in vivo Ret^{M955T}-driven tumor model and decreases in direct binding affinity towards recombinant wild-type RET as measured across the regorafenib-based para-substituted perfluoroalkyl-series.

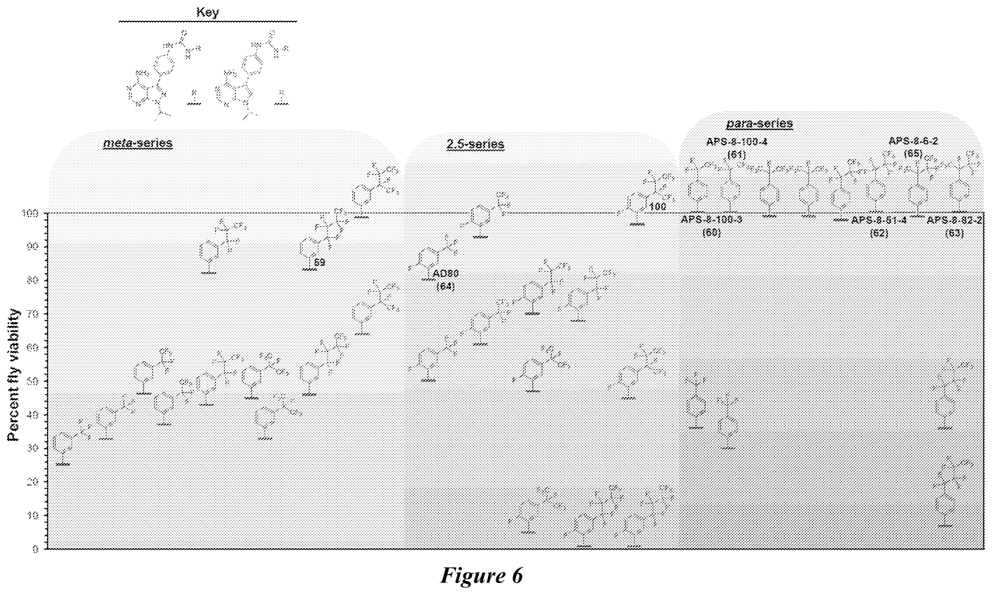

With few exceptions, the results of in vivo rescue assays in Ret^{M955T} flies differed substantially from in vitro binding assays on recombinant RET, possibly suggesting that the unique properties of the perfluoroalkylated CAP-groups can engender a very high level of biological activity. To better ascertain target engagement within an in vivo context, a cellular binding assay termed nanoBRET was utilized (Vasta et al., “Quantitative, Wide-Spectrum Kinase Profiling in Live Cells for Assessing the Effect of Cellular ATP on Target Engagement,” Cell Chem. Biol. 25:206-214.e11 (2018), which is hereby incorporated by reference in its entirety). In this assay, a bioluminescence resonance energy transfer (BRET) signal is generated between nanoLuciferase tagged RET (expressed in HEK 293T cells) and an active-site based fluorescent tracer. Competition experiments with individual drugs result in general loss of BRET signal, which can be transformed into cellular IC50 values against RET (Robers et al., “Quantitative, Real-Time Measurements of Intracellular Target Engagement Using Energy Transfer,” Methods Mol. Biol. 1888:45-71 (2019), which is hereby incorporated by reference in its entirety). Using this assay, in vivo binding was determined for a series of compounds against mutant (M918T) and wild-type RET (FIG. 8D). The compound series included the top twelve performing perfluoroalkyl-drugs in the MTC Drosophila assay (all showed 95-100% rescue), previously identified AD80 (64) and close structural analog 5-86-2 (97), initial lead perfluoroalkyl-drug APS-6-45 (3), and clinical compounds regorafenib (2), LOX0292, and BLU667. For all perfluoroalkyl-drugs examined, the nanoBRET assay did not show a significant difference in cellular IC₅₀ values between WT and mutant (M918T) RET. Similar to the Kd measurements, IC₅₀ values correlated well with the size of the perfluoroalkyl-substituent, but not in vivo fly rescue. Among the pyrazolo- and pyrrolopyrimidine series (FIG. 6), the highest IC₅₀ values were observed for compounds functionalized with a -sC₄F₉ in the meta-[8-56-3 (96), 8-35-3 (100)] or para-position [8-6-2 (65), 8-82-2 (63)], followed bypara-substituted straight chain -nC₃F₇[8-51-3 (104), 8-51-4 (62)] or branched -iC₃F₇[8-50-3 (103), 8-50-4 (119)]; —C₂F₅[8-100-3 (60), 8-100-4 (61)] displayed the lowest IC₅₀ values among the para-substituted compounds tested. On the pyrazolopyrimidine core slightly lower IC50s were observed for meta-compared to para-substituted compounds (compare 60 with 64 and 97), however, this was not observed for the sorafenib core [compare 6-45 (3) with 8-50-1 (35)]. AD80 (64; 80% rescue) displayed a slightly lower IC50 value for wild-type RET over mutant, which was not observed for the closely related APS-5-86-2 (97; 92% rescue), but may contribute to the difference in fly rescue. Interestingly, the three clinical compounds, regorafenib (2), Loxo292 and Blu667, also demonstrated slightly lower IC50 values towards wild-type RET over the M918T mutant.

Physicochemical assessment of RF analogs by absorption and transport

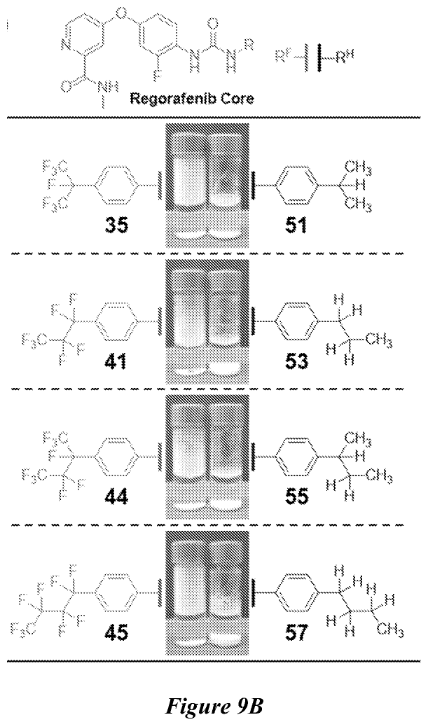

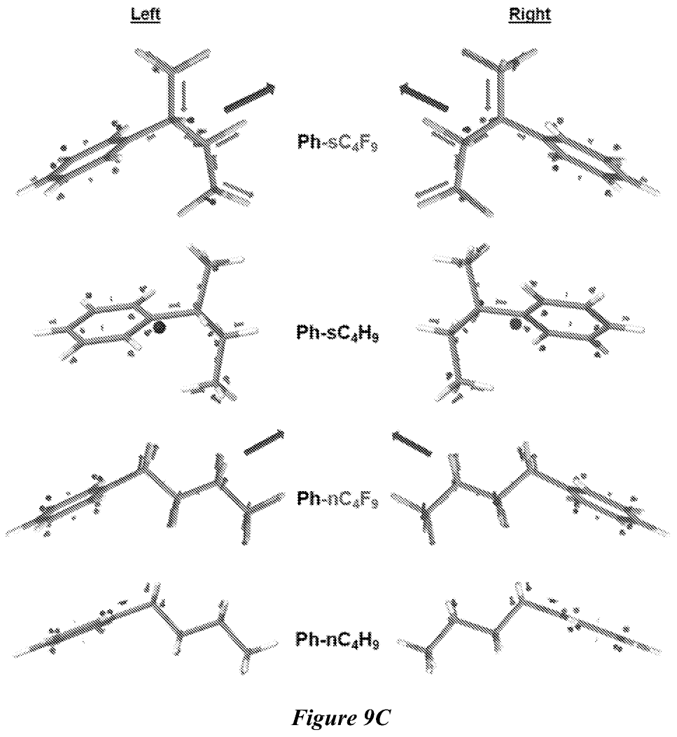

Clear biological differences between the fluorocarbon and hydrocarbon analogs were apparent as assessed by membrane permeability. In both MDCK and Caco-2 permeability assays the hydrocarbon analog APS-9-23-6 (50; —C₂H₅) displayed much faster rates of influx and efflux compared to the fluorocarbon counterpart APS-8-100-2 (40; —C₂F₅), which is indicative of passive diffusion for 50. In both assays the influx/efflux ratio for APS-9-23-6 (50) was <1, indicating that the hydrocarbon analog clears from cells relatively quickly. In contrast, APS-8-100-2 (40) displayed an influx/efflux ratio of ≥1, which suggests that the fluorocarbon analog can accumulate in cells over time (FIG. 9A).

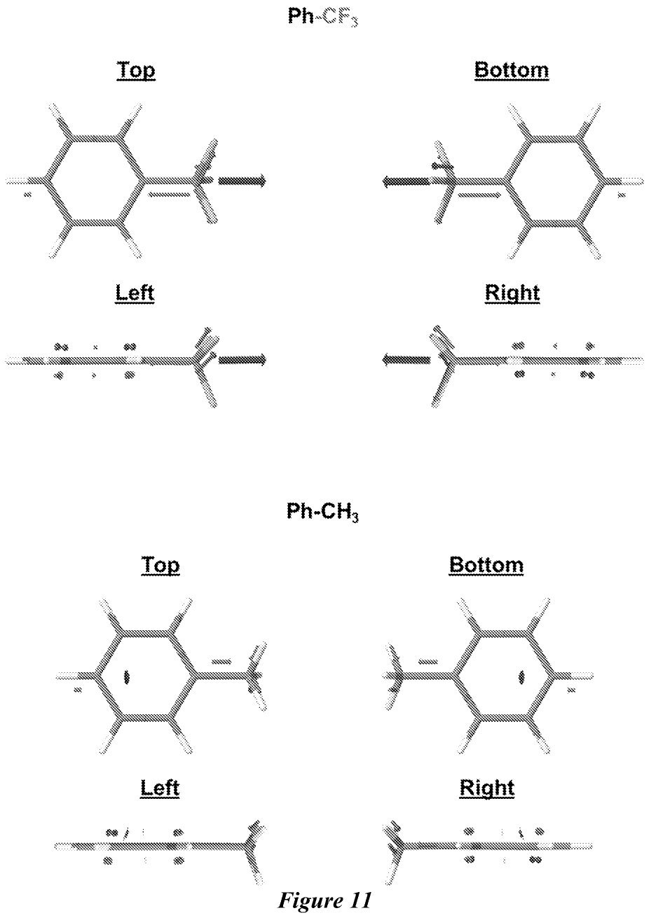

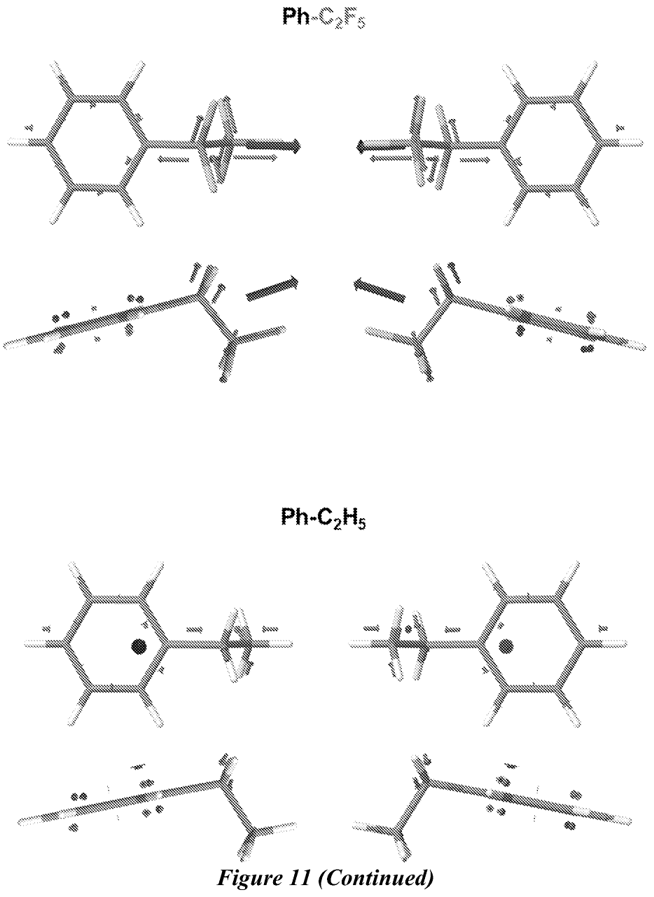

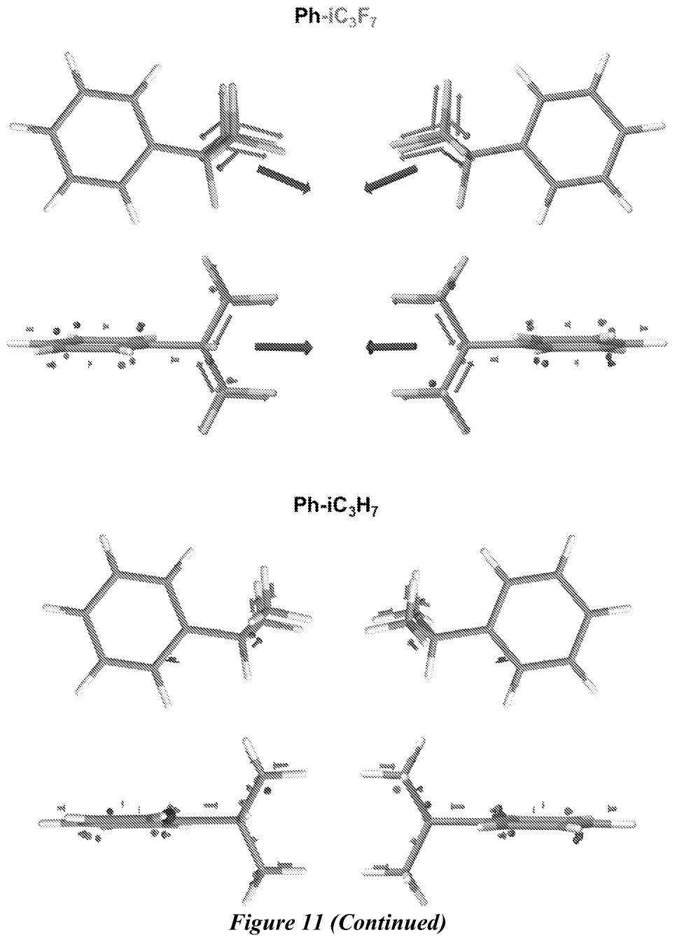

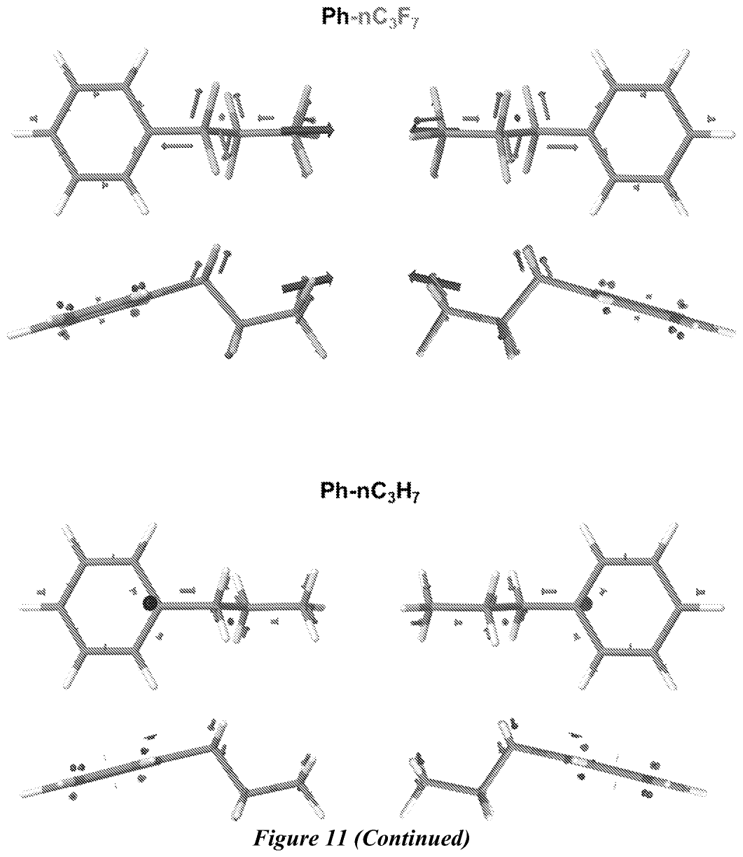

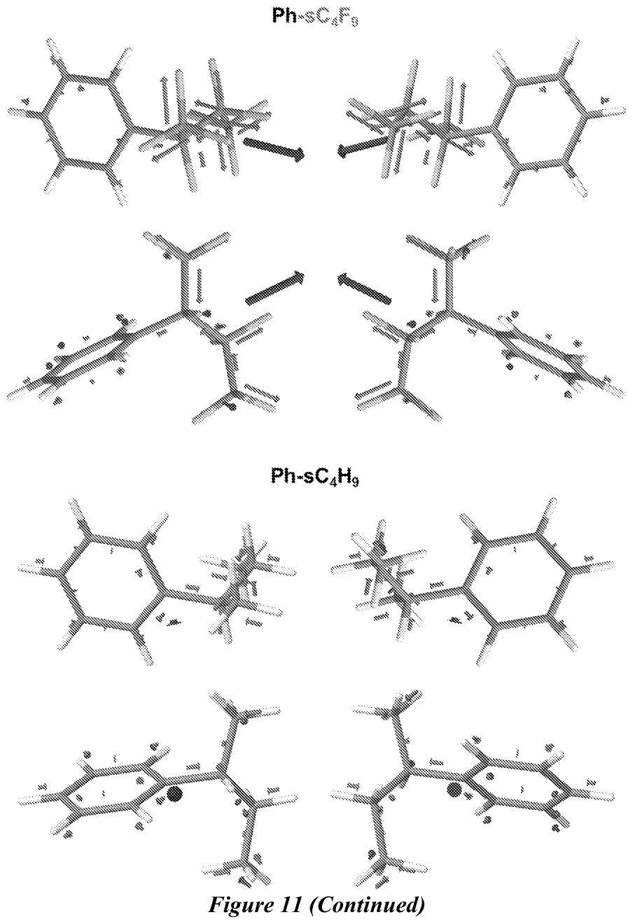

Further, under saturated conditions the perfluoroalkylated compounds formed a distinctive film, whereas the hydrocarbon analogs appeared as large aggregates (FIG. 9B). Bond and molecular dipoles were calculated for a series of CAP-group fragments (Ph-R^{F} vs Ph-R^{H}), which indicated that the difference in appearance might be related to differences in physicochemical properties (FIGS. 9C, 10A-10H, and 11). The bond dipoles along the carbon chain of the perfluoroalkane-substituents are typically larger in magnitude but opposite in sign (e.g. C^{δ+}→F^{δ-} vs C^{δ-}←H^{δ+} and Ph←R^{F} vs Ph→R^{H}) to the hydrocarbon-substituents; R^{F}/R^{H} phenyl ring bond dipoles did not show much variability. The molecular dipoles observed among the phenyl perfluoroalkyl-series (Ph-R^{F}→) are antiparallel to the bond dipoles between the phenyl-ring and the perfluoroalkyl-substituents (Ph←R^{F}). The molecular dipoles for the phenyl hydrocarbon-series are much smaller in magnitude and are perpendicular (↑Ph-R^{H}) to the bond dipoles between the phenyl-ring and the hydrocarbon-substituents (Ph→R^{H}). The magnitude and orientation of the Ph-R^{F} molecular dipoles, as well as the observed formation of aqueous-films, suggest that the perfluoroalkane-drugs could have a propensity to form colloids and micelles (Doak et al., “Colloid Formation by Drugs in Simulated Intestinal Fluid,” J. Med. Chem. [symbol] (2010); Hasegawa, T., “Physicochemical Nature of Perfluoroalkyl Compounds Induced by Fluorine,” Chem. Rec. 17:903-917 (2017), which are hereby incorporated by reference in their entirety).

Transferability of R^{F} CAP-groups to distinct hinge-binding scaffolds

To determine if the perfluoroalkyl tool kit could provide informative SAR and improved analogs on distinct type II scaffolds, a focused library of perfluoroalkyl-analogs was synthesized using pyrazolopyrimidine- and pyrrolopyrimidine-based hinge-binders (FIG. 6). Functionalization of these cores with perfluoroalkyl-substituted cap-groups showed even greater tolerance for modifications and enhanced in vivo potency relative to the sorafenib and regorafenib scaffolds. Overall the pyrazolopyrimidine core proved to be slightly more active than the pyrrolopyrimidine, and para-substituted analogs were generally the most effective.

Several differences between the sorafenib/regorafenib-based analogs and the pyrazolo/pyrrolopyrimidine-series emerged. For example, within the pyrazolopyrimidine- and pyrrolopyrimidine-based analogs the branched -sC₄F₉ provided the highest activity analogs (63 and 65, >99% average rescue) across all compound series, whereas -iC₃F₇ was optimal among the sorafenib/regorafenib-based analogs. Moreover, in contrast to the regorafenib/sorafenib series, several linear -nC₄F₉ analogs showed activity, including APS-7-46-2 (59), a meta-nC₄F₉ analog that generated over 80% rescue. Four compounds that provided 100% rescue were also identified, APS-8-100-3/4 (60/61), APS-8-51-4 (62) and APS-8-82-2 (63). These analogs were markedly more active than any of the previously reported compounds, including AD80 (64). Thus, the perfluoroalkylated toolbox appears transferable across KI scaffolds, and with it was identified several compounds with unprecedented activity within ptc>Ret^{M155T} flies.

### Polypharmacology and Electrostatics

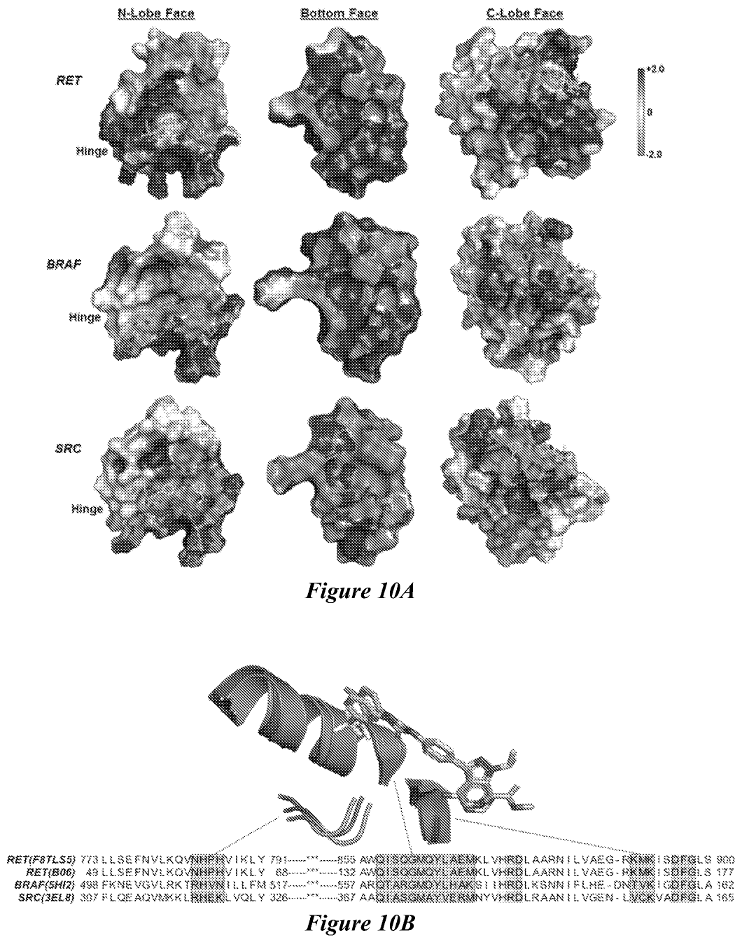

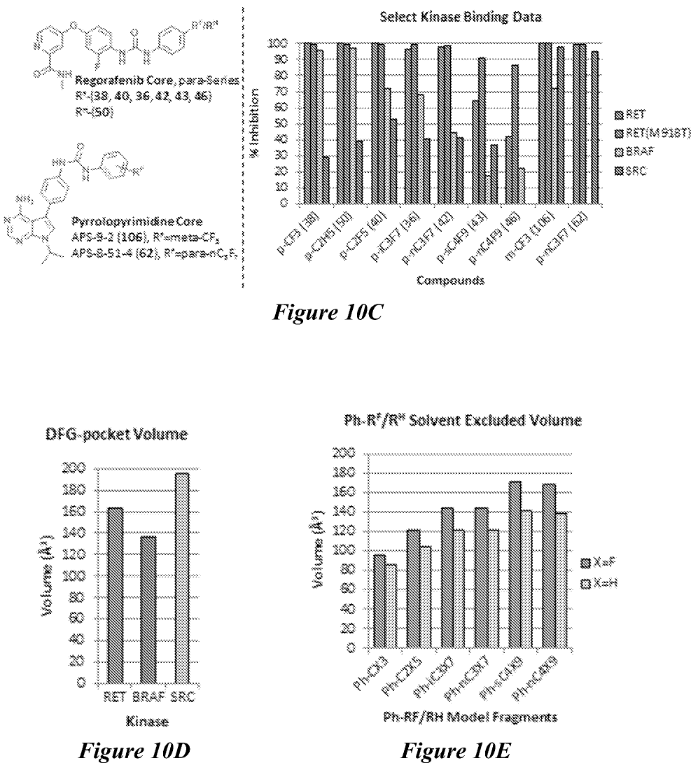

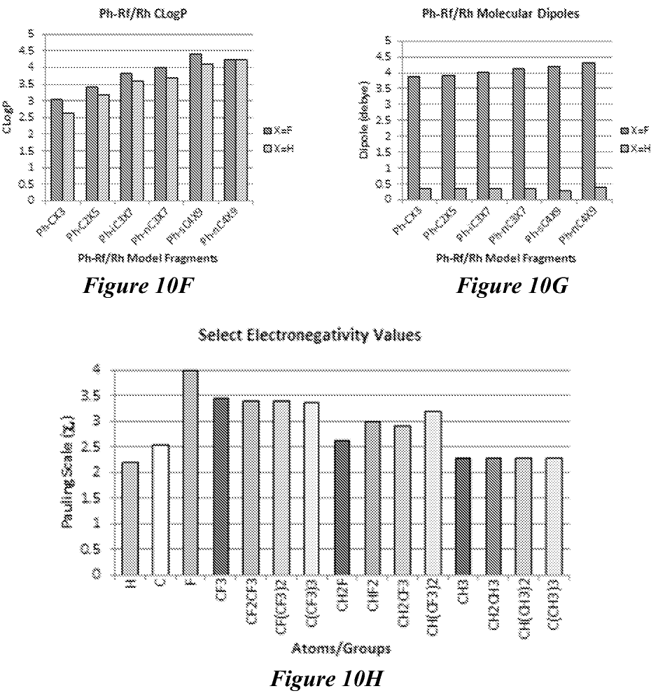

Across the four drug cores examined, the SAR trends among the perfluoroalkylated compounds would be consistent with varying size and shape complementarity between analogs, and the available volume of the DFG pocket of direct kinase targets (Hanson et al., “What Makes a Kinase Promiscuous for Inhibitors?,” Cell Chem. Biol. 26:390-399.e5 (2019), which is hereby incorporated by reference in its entirety). Overall perfluoroalkyl-substituents seemed to be well suited with regard to tuning protarget/antitarget profiles. Support for this possibility was found when the binding profiles of a series of structurally related compounds were compared against a panel of 468 kinases (FIGS. 3A-3C and 10A-10H). On the regorafenib-core, APS-4-9-8 (38; —CF₃) displayed similar potency on wild-type and mutant RET, showed strong binding to a large group of kinases, but provided no fly rescue; while APS-8-82-1 (43; -sC₄F₉) gave 85% rescue, was selective for mutant RET^{M918T}, and bound to only 7 kinases with >80% efficacy (FIGS. 3B and 3C). Pyrazolopyrimidine-based compound APS-8-6-2 (65; -sC₄F₉) proved to be a generally promiscuous KI, but provided one of the highest rescues in the MTC fly assay and was more potent than 43 on wild-type and mutant RET (FIG. 8B).

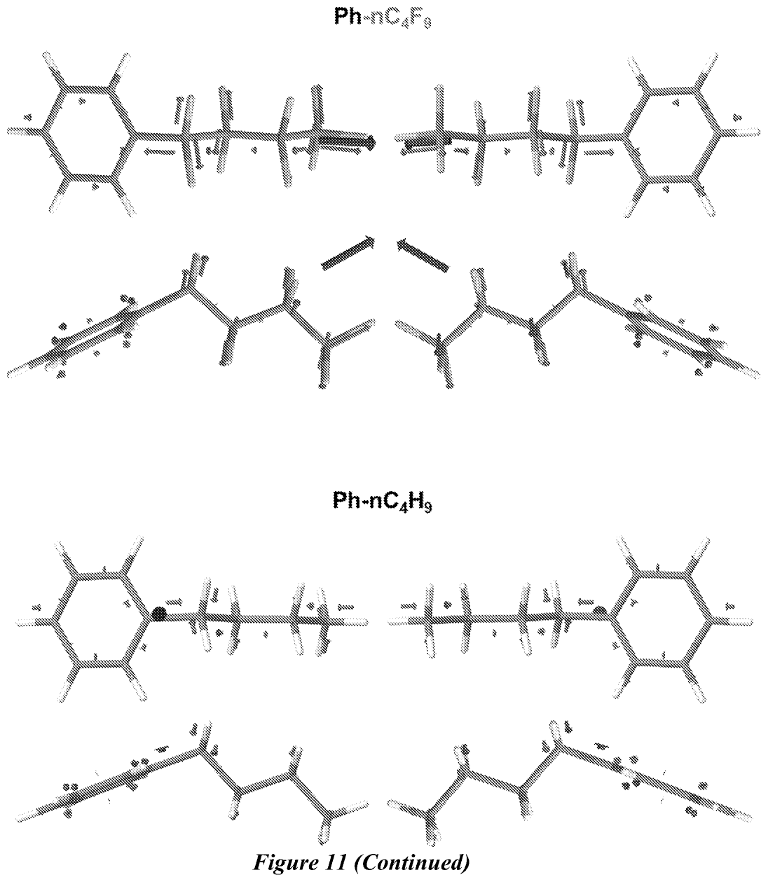

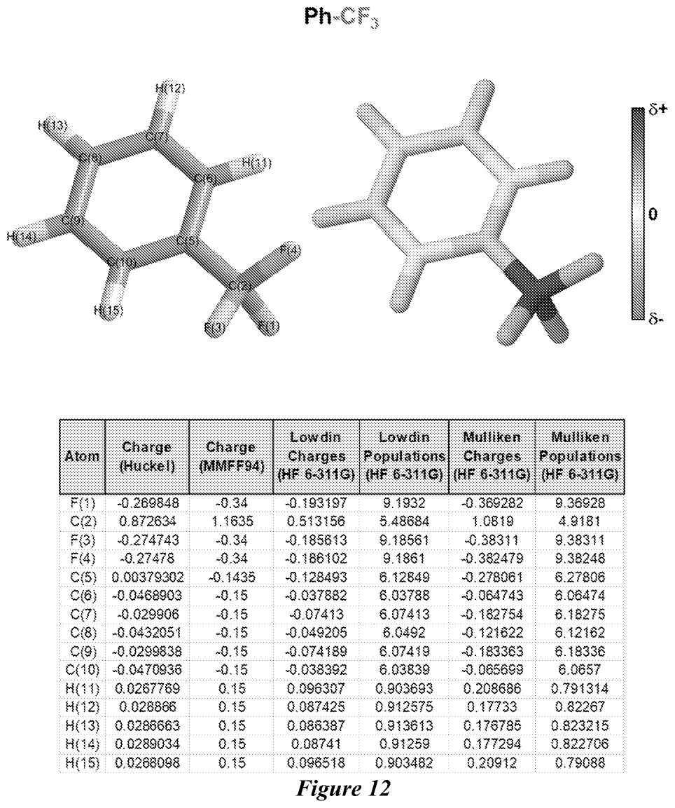

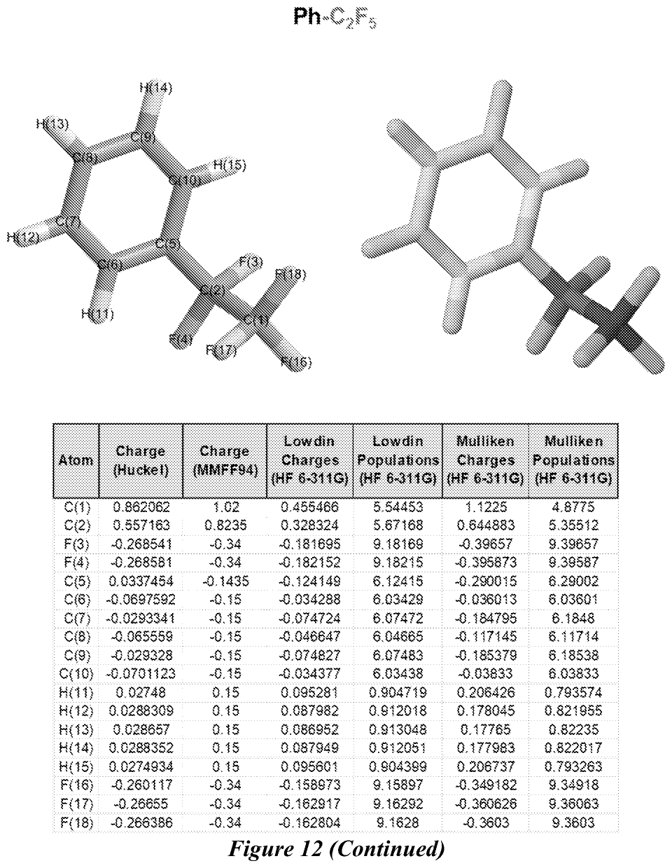

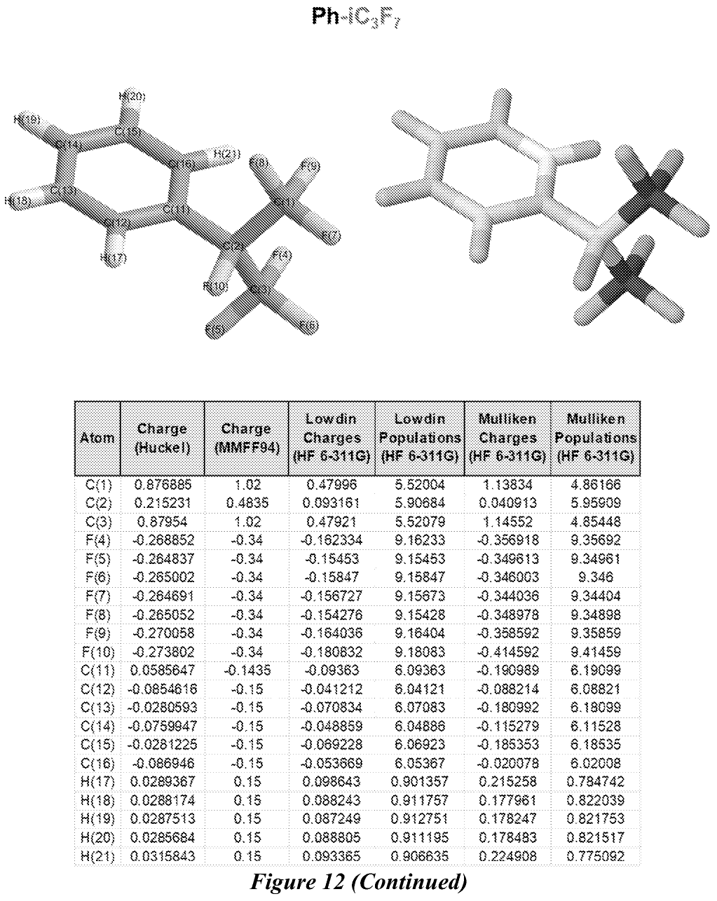

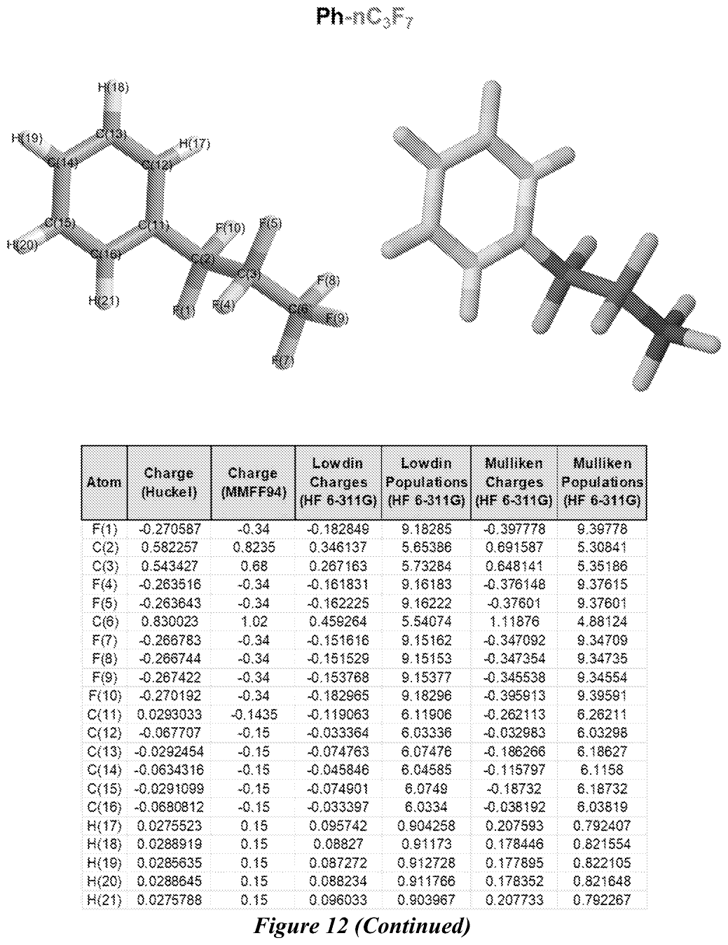

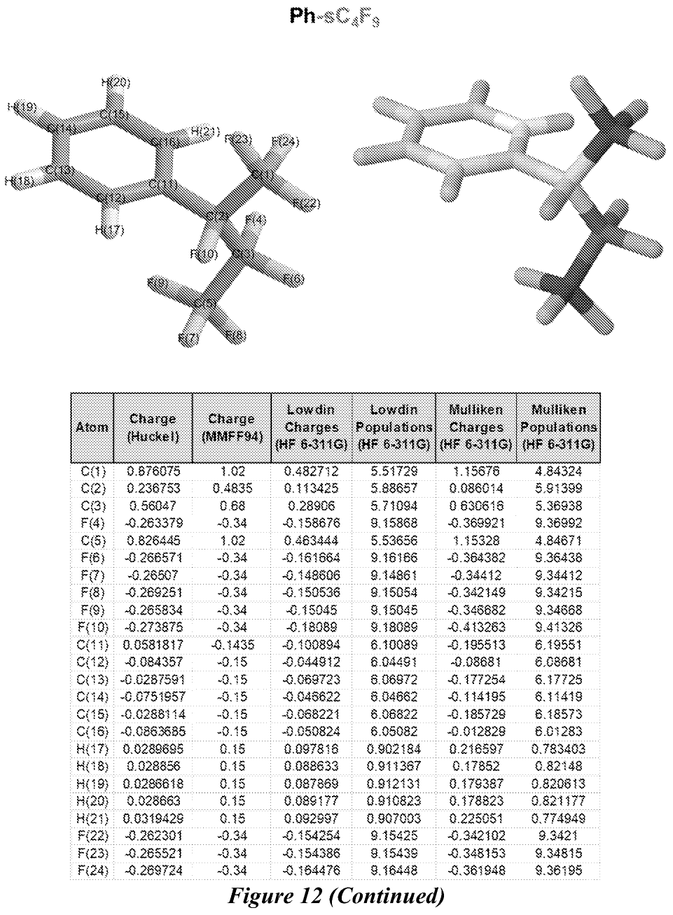

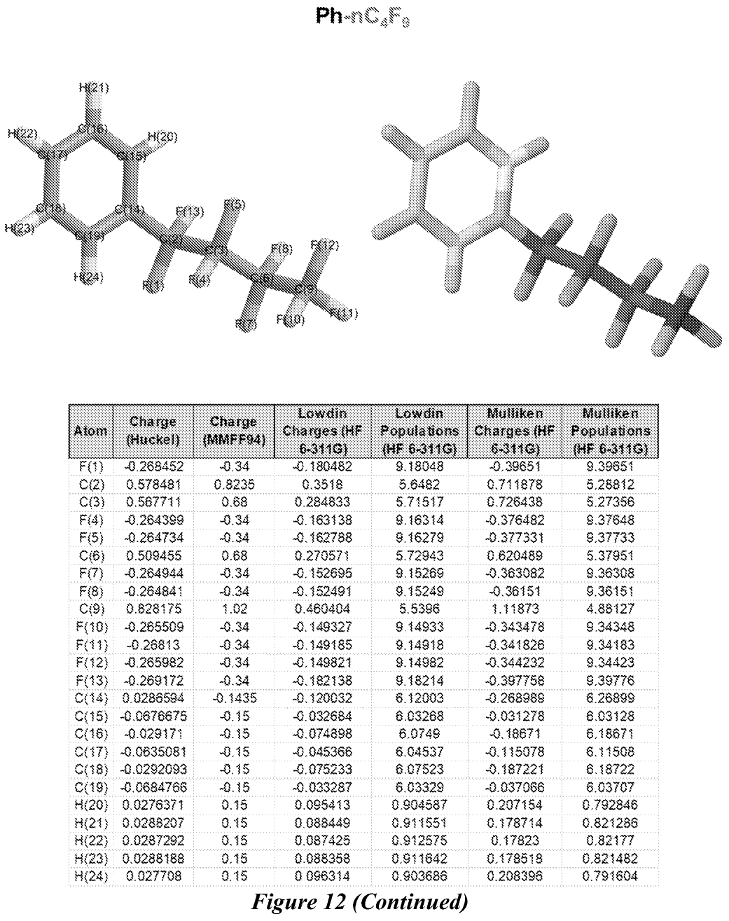

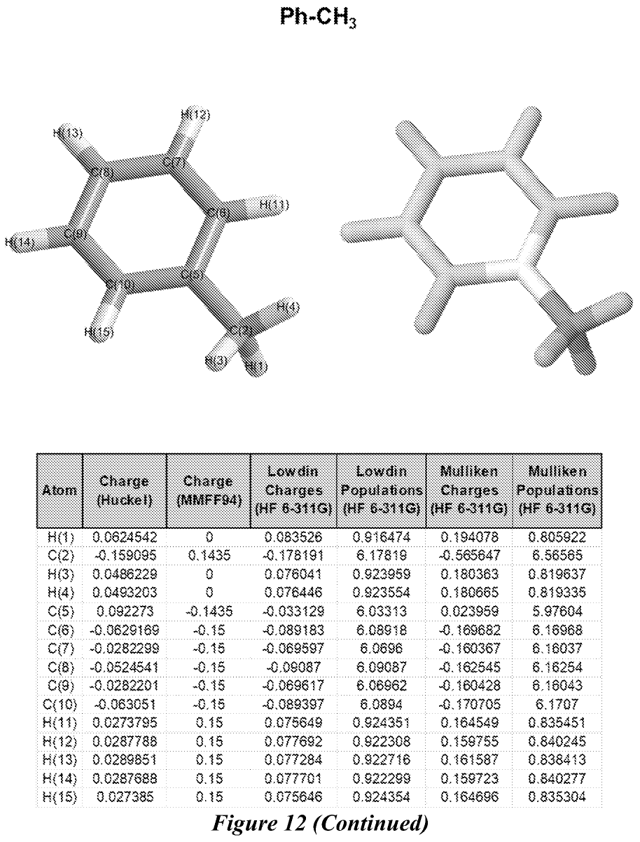

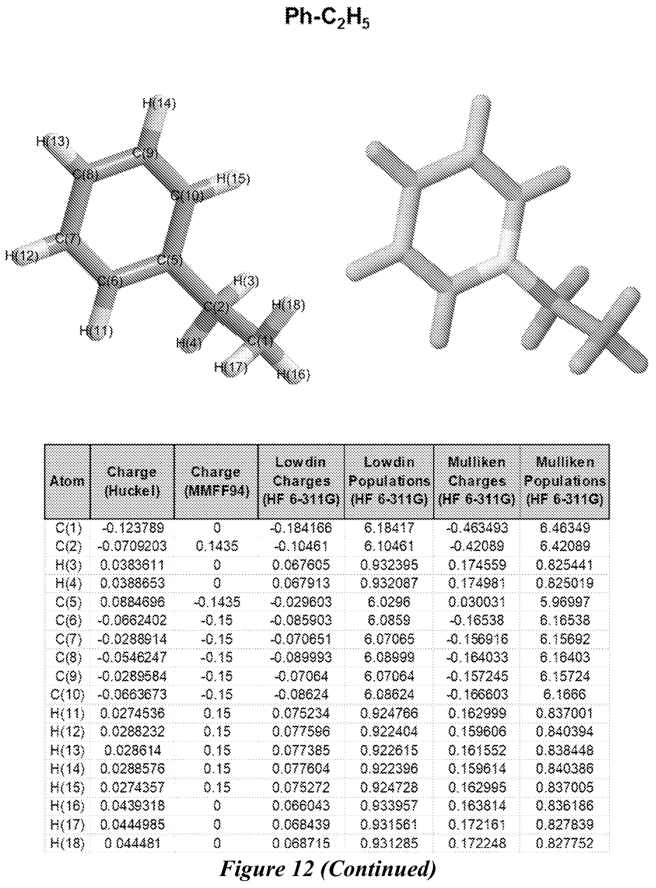

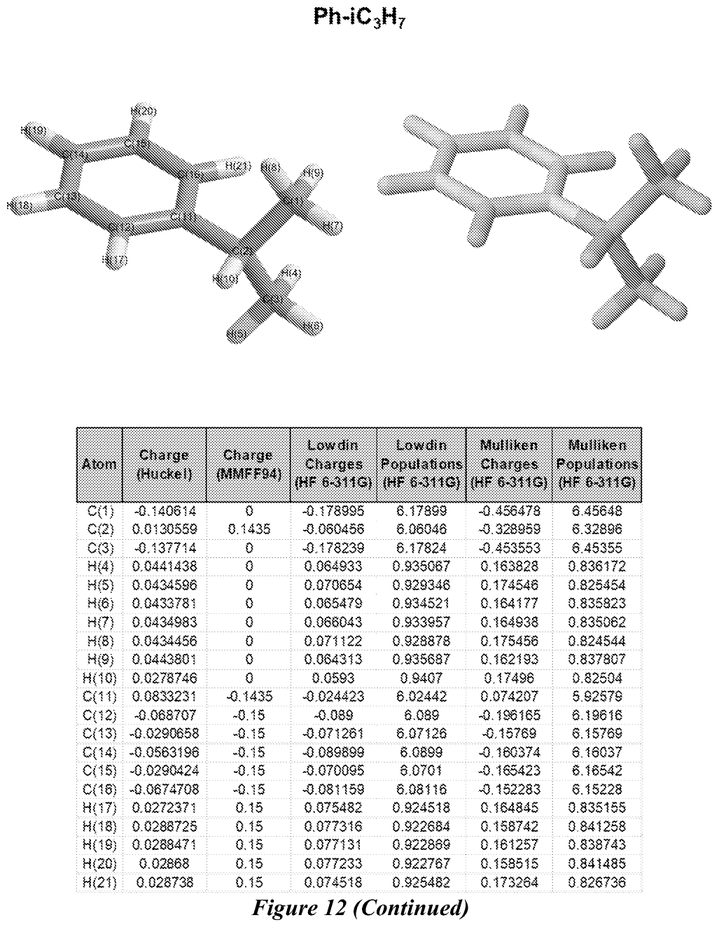

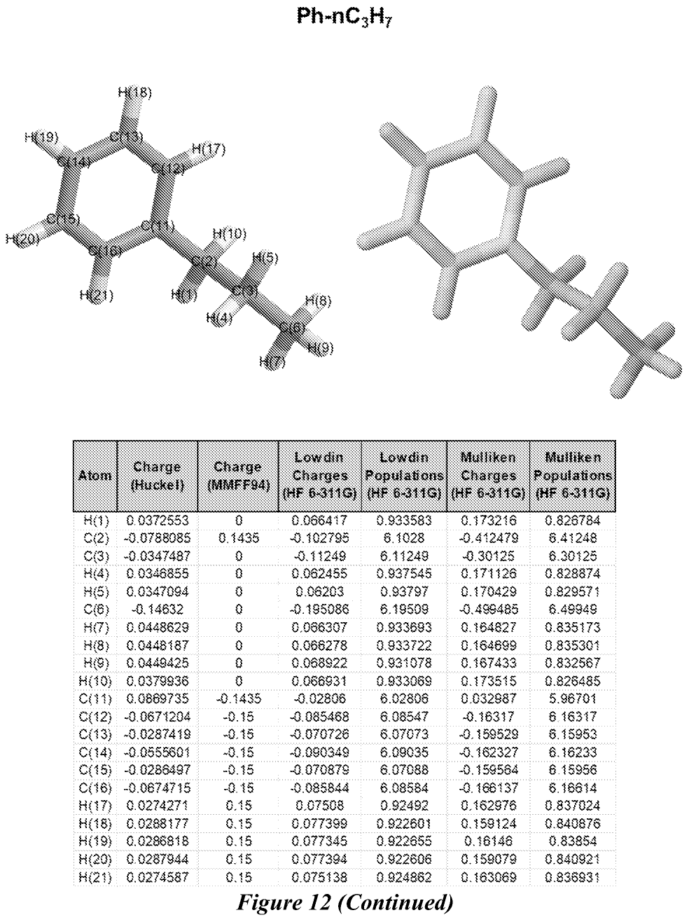

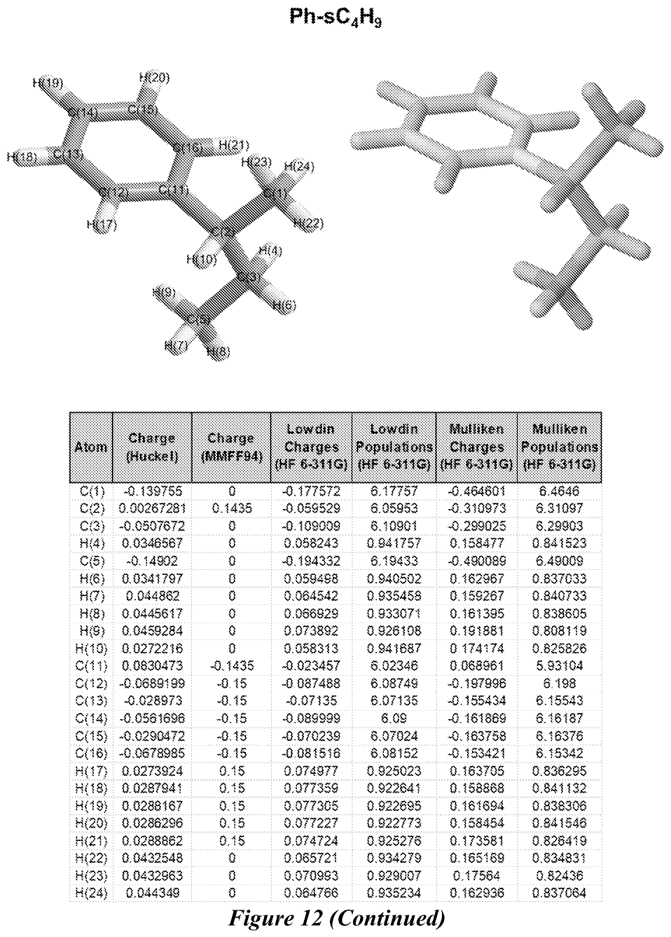

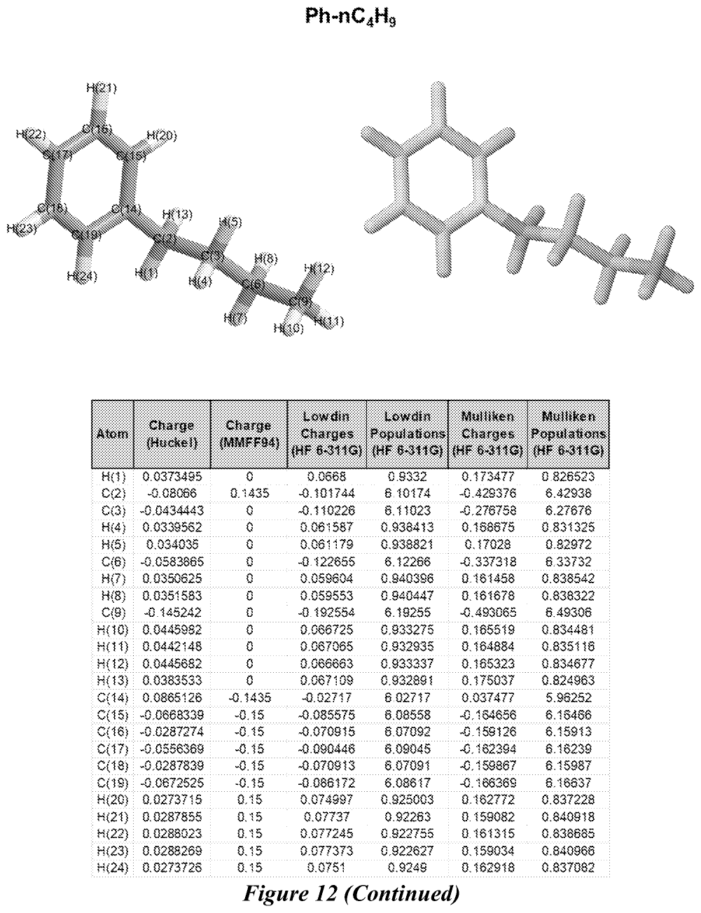

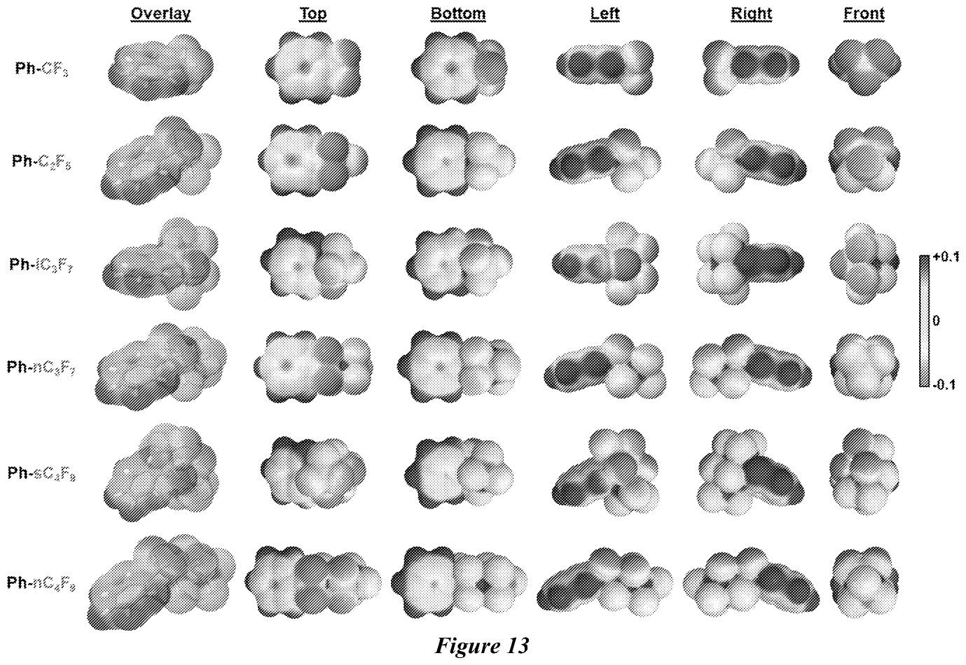

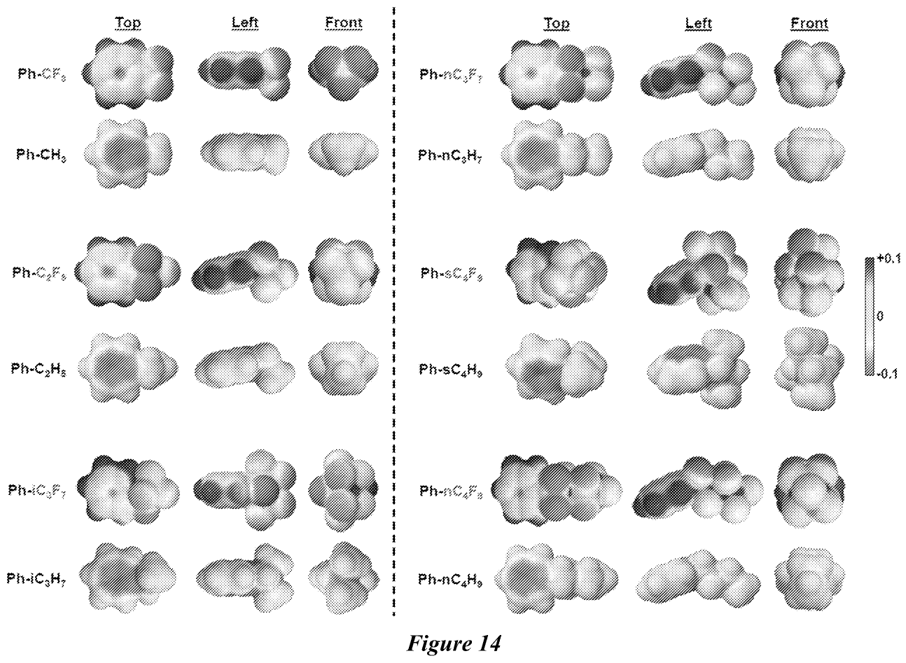

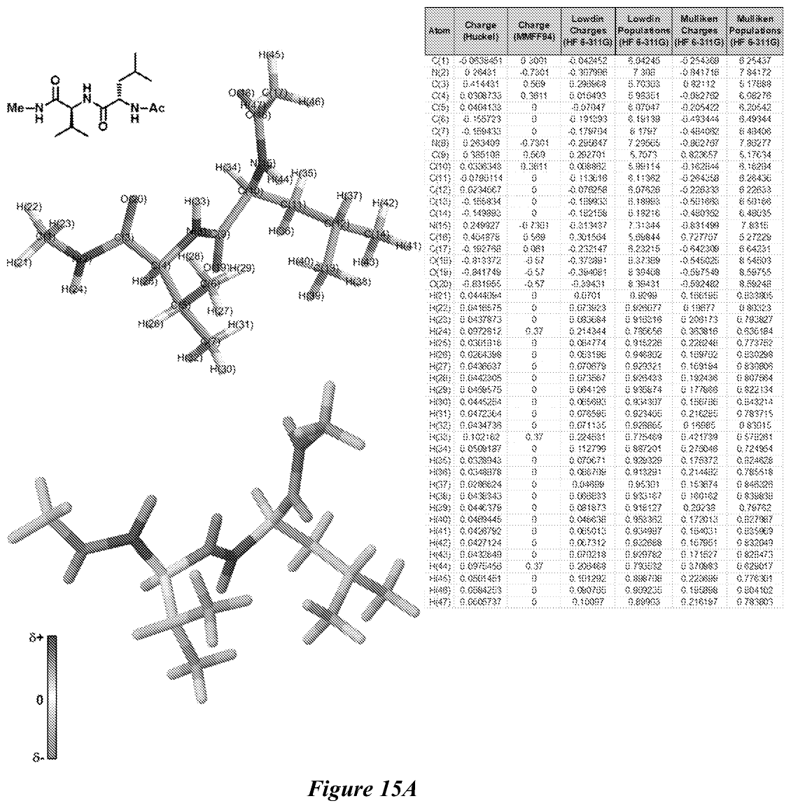

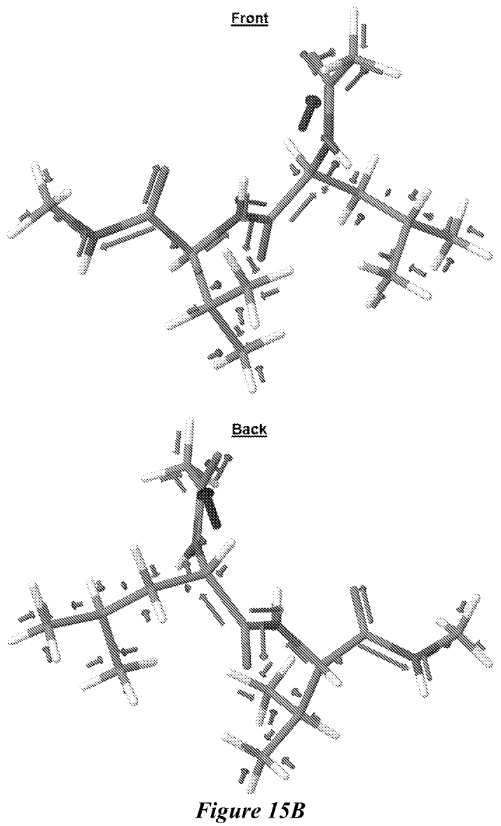

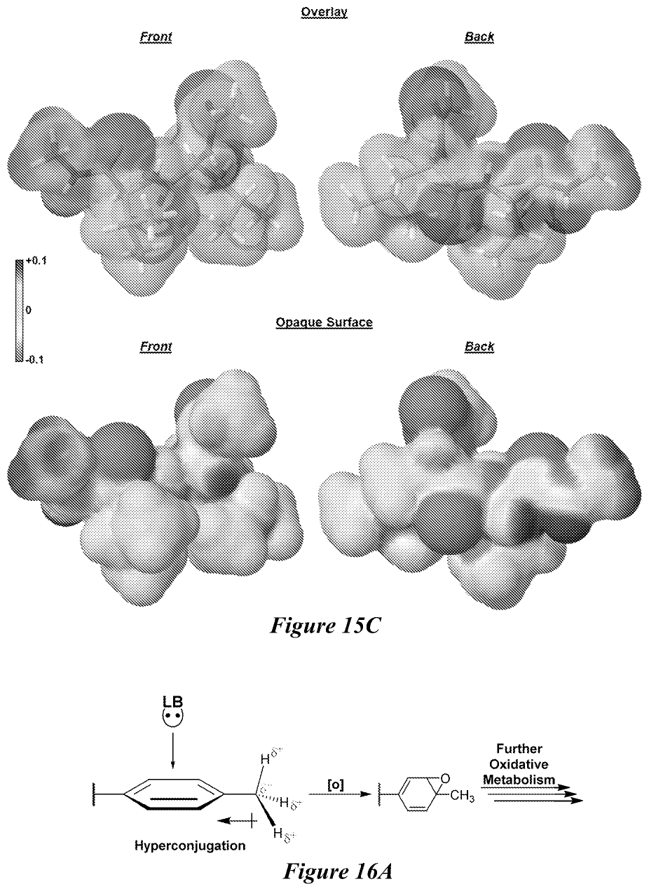

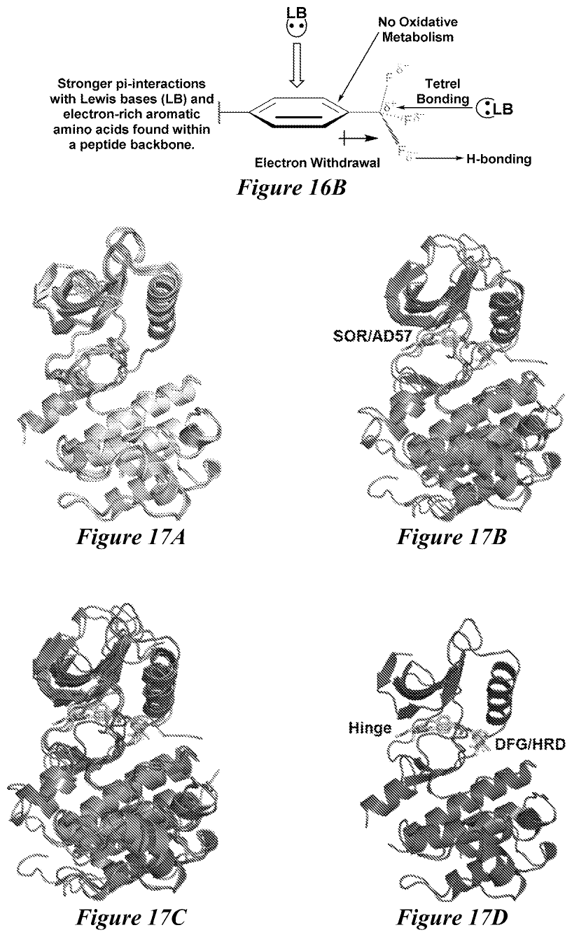

Specific molecular properties were calculated for the individual Ph-R^{F}/R^{H} CAP-group model fragments (e.g. partial charges, dipoles, molecular surfaces and electrostatic potential). Overall, the fluorocarbon- and hydrocarbon-substituents displayed opposite charge profiles, with partial negative charges at the fluorine atoms along the perfluoroalkyl chains, and partial positive charges at the hydrogen atoms along the hydrocarbon chains (FIGS. 3D and 12). These charge differences were visualized by mapping the molecular electrostatic potential (MEP) onto the van der Waals surface of the individual CAP-group fragments (FIG. 3E, FIG. 13, and FIG. 14). MEP maps of the Ph-R^{F} fragments showed increased electron density along the perfluoroalkyl-substituent as well as an electron deficient pi-system; the reverse was observed for the Ph-R^{H} fragments. Similar calculations performed on a model dipeptide (Me-Val-Leu-Ac) revealed partial charges, dipoles and MEP maps that appear to line up favorably with the perfluoroalkyl-substituents (FIGS. 15A-15C). The molecular properties calculations, combined with experimental data, implied that the unique physicochemical properties of perfluoroalkyl-substituted drugs are directly related to their favorable PK and PD profiles (FIGS. 16A-16B and FIGS. 10A-10H).

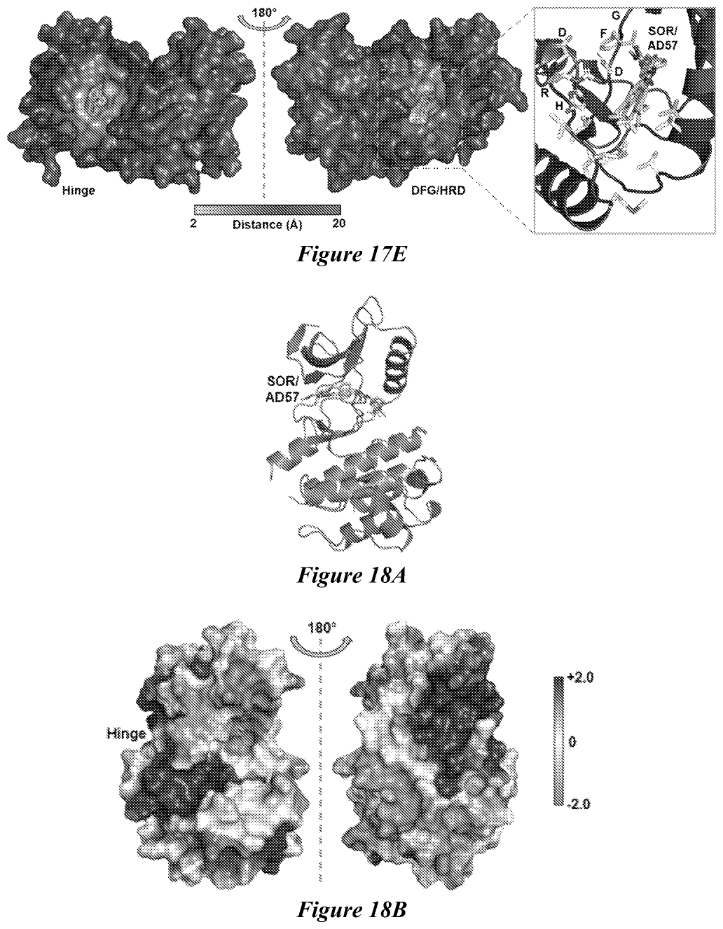

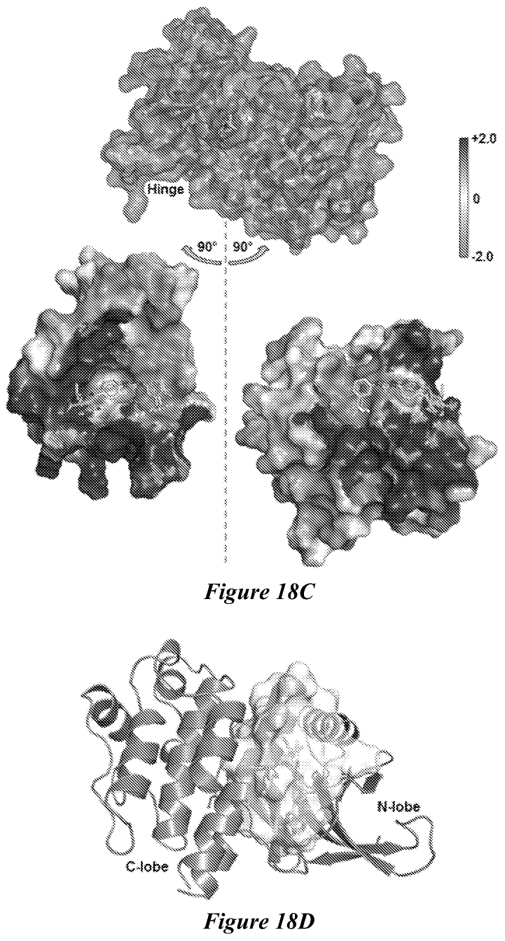

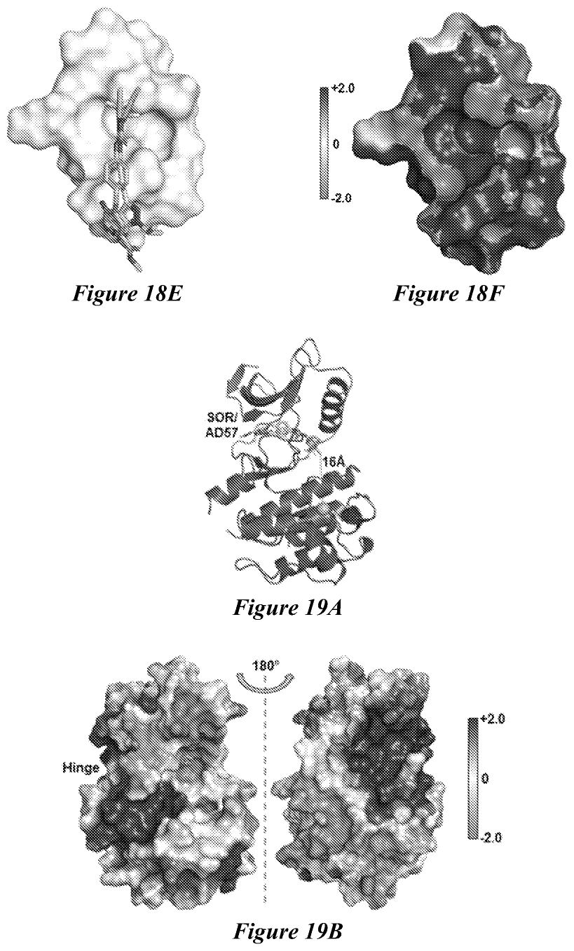

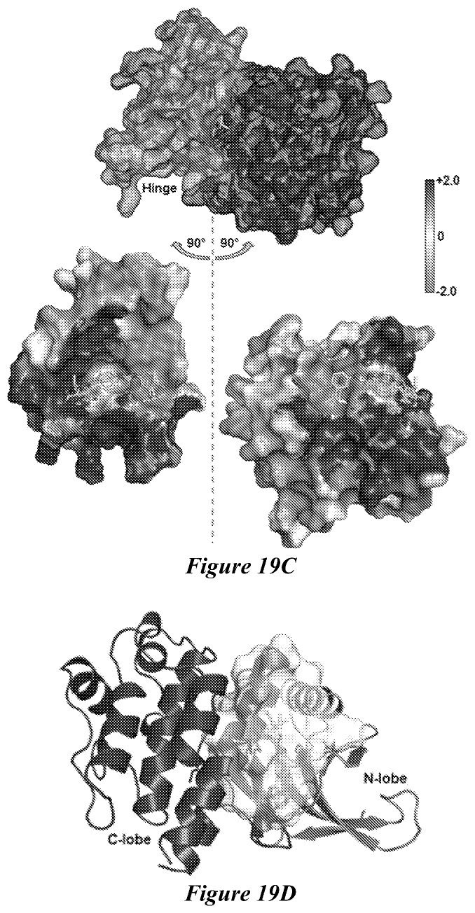

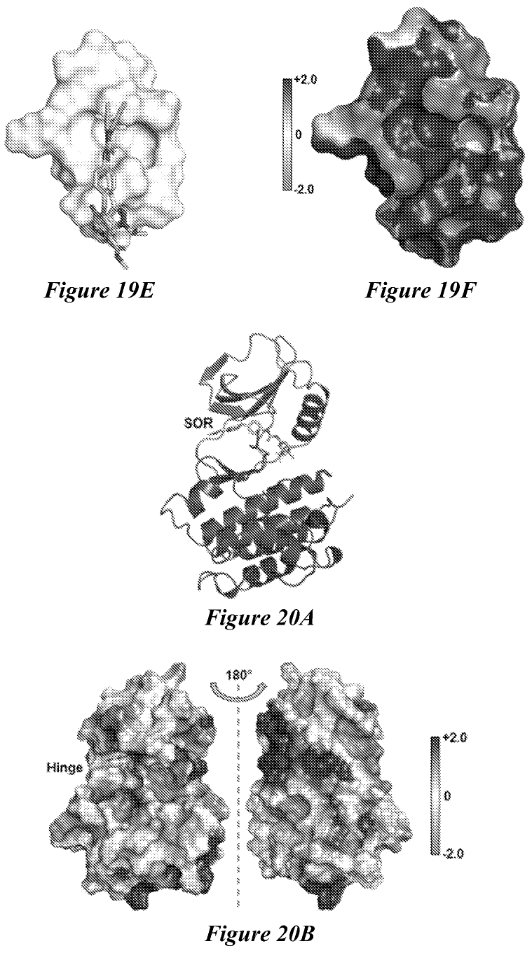

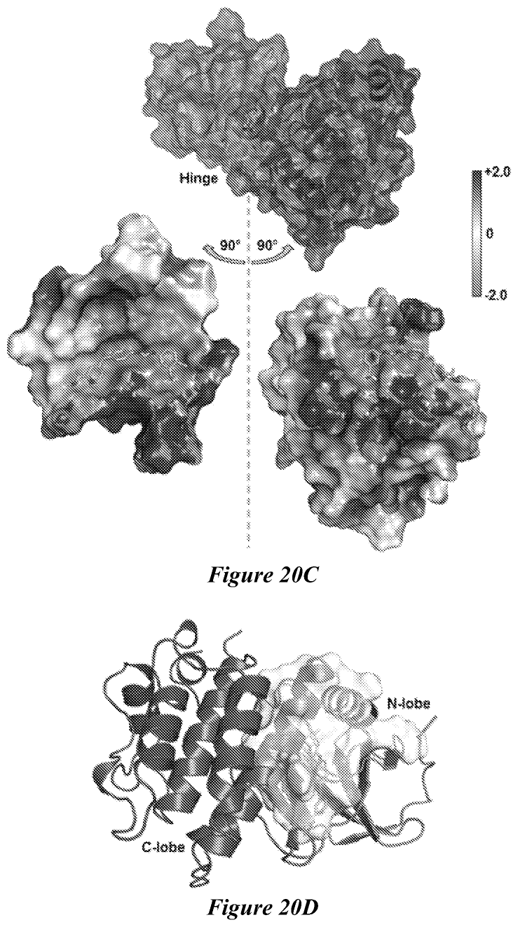

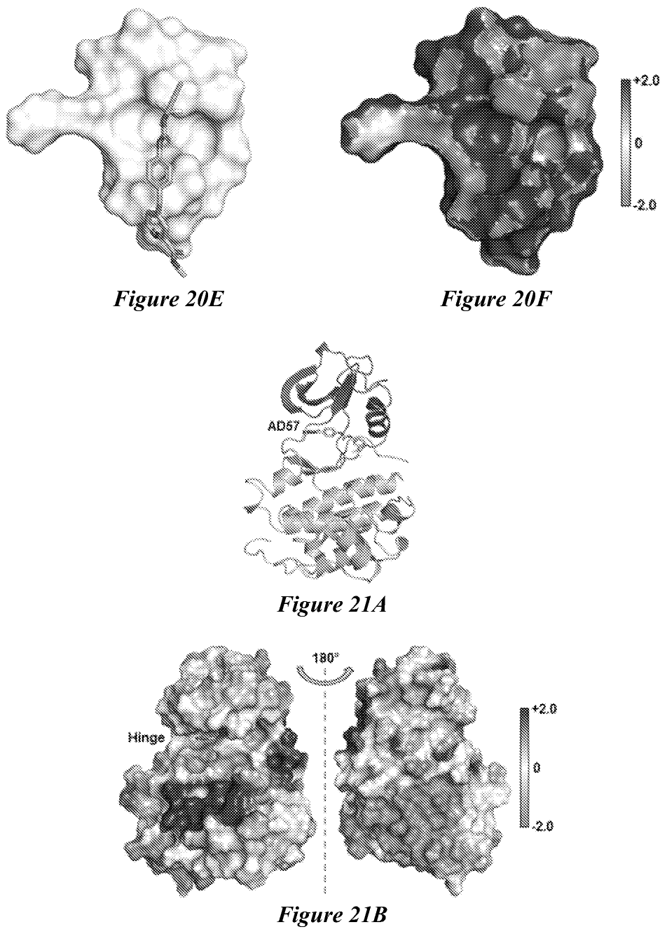

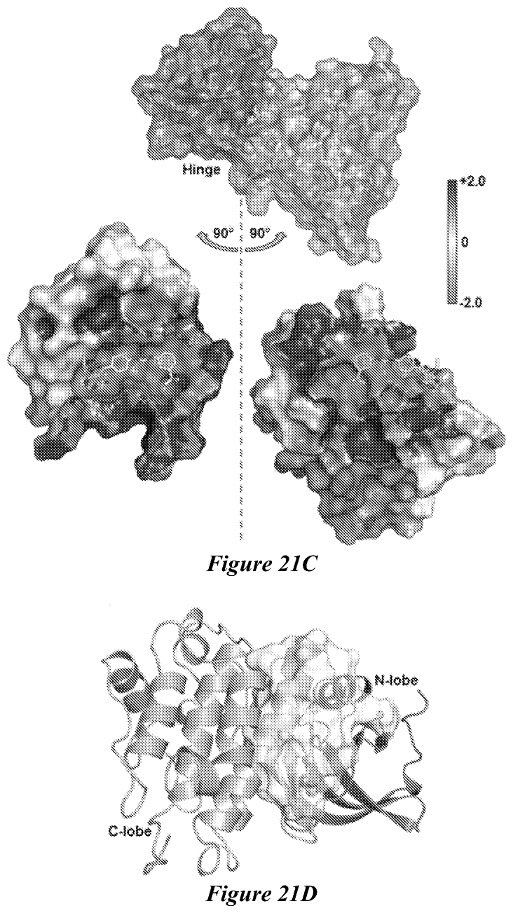

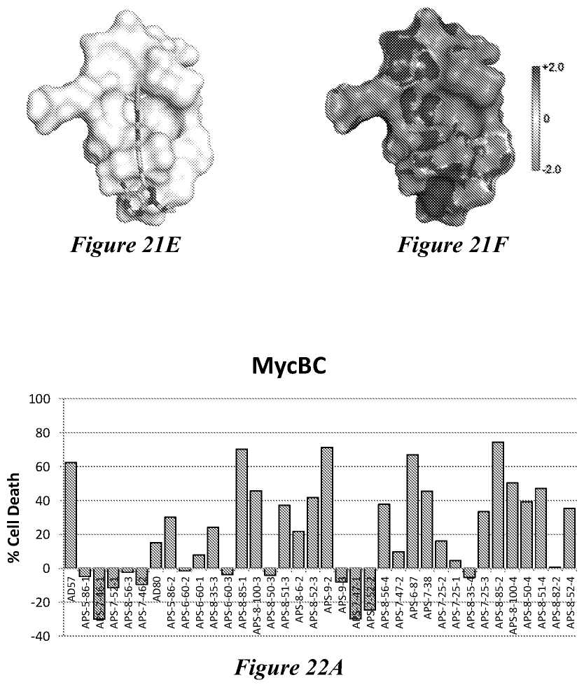

A wider perspective was obtained by examining whole kinase structures. Crystal structures of sorafenib (SOR)-bound BRAF(5HI2) and AD57-bound SRC(3EL8) were aligned with a RET type-II homology model (B06) obtained from KinaMetrix.com (there are no known crystal structures of RET in the type-II conformation). BRAF and SRC were removed from the overlay leaving the ligands (SOR and AD57) to help define the size, shape and amino acid residues associated with a possible RET type-II binding pocket (FIGS. 17A-17E). The MEP map of RET(B06) was calculated, and the structure was opened across the N- and C-lobes in order to examine the individual faces of the binding pocket (FIG. 18A-18C). Positive electrostatic potential was observed in a portion of the binding pocket adjacent to the HRD- and DFG-motifs, which primarily contains amino acid residues with hydrocarbon side chains. The bottom face of the type II pocket, comprised of residues along the interface of the N- and C-lobes, also showed a significant area of positive electrostatic potential (FIGS. 18D-18F). The relative position of the overlaid ligands (SOR and AD57) indicated that the perfluoroalkyl-substituents, of examined drugs, would likely reside in the observed region of positive electrostatic potential, which could contribute to their binding affinity and on-target residence time. MEP map calculations were also performed on a mutant RET^{M918T} structure that was generated by performing in silico mutagenesis on the RET(B06) type II homology model (FIGS. 19A-19F). The internal faces of the RET(B06)^{M918T} structure revealed MEP maps (bottom, N- and C-lobe faces) that were identical to those observed for wild-type. The exterior of the in silico derived RET(B06)^{M918T} mutant displayed very slight differences in molecular surface volume and electrostatic potential compared to wild-type; external differences were observed for both possible threonine rotamers as well as wild-type RET(B06). The distance between the 4-chloro-substituent of SOR and the M918T point mutation was measured to be 16 Å, which appears to be far enough away to prevent observable changes to the internal faces of the type II binding pocket. The BRAF and SRC structures were analyzed in a similar manner. Both showed a concentration of positive electrostatic potential in the same general region of the type II binding pocket that was observed for the homology models of wild-type and mutant (M918T) RET (FIGS. 20A-20F and 21A-21F).

### Discussion of Results

This disclosure presents a chemical toolbox of perfluoroalkyl-substituted CAP-groups to discover novel type II KIs.

The present disclosure demonstrates the exceptional in vivo activity of several analogs of novel type II KIs [e.g. APS-8-50-1 (35; -iC₃F₇), APS-8-50-2 (36; -iC₃F₇), APS-8-100-3 (60; —C₂F₅), APS-8-100-4 (61; —C₂F₅), APS-8-51-4 (62; -nC₃F₇), APS-8-6-2 (65; -sC₄F₉), and APS-8-82-2 (63; -sC₄F₉)] in ptc>Ret^{M955T} flies. These new compounds surpass the in vivo activity of any previous KIs, including AD80 (64) and APS-6-45 (3). The transferability of the CAP-group substituents in the context of several type II KI formats were demonstrated using distinct hinge-binding elements, suggesting that perfluoroalkanes could potentially be broadly applied to KI development and drug discovery. Lastly, it was shown that target potency is not always the driving force of in vivo efficacy in fly models, as there are several additional factors, including distinct polypharmacology, transport, permeability, and basic physicochemical properties that appear to differentiate the new perfluoroalkylated compounds reported here from previously developed KIs. Below are discussed several unique aspects of perfluoroalkyl-groups that may render them particularly well suited for optimizing both structure-activity as well as structure-property relationships (ie. SAR and SPR) of compounds intended for in vivo applications.

The complementary nature of perfluoroalkane building blocks allows for the construction of drug-core analog-series that are best described as directed libraries. Incremental increases in size and electron density that result from gradually extending the perfluoroalkyl-chain allow for systematic-interrogation of disease models and hyper-optimization of specific drug cores. This is a strategy that seems well suited for kinase polypharmacology, as progressively building out a drug core allows for tuning of a pro-target/anti-target profile (Sonoshita et al., “A Whole-animal Platform to Advance a Clinical Kinase Inhibitor into New Disease Space,” Nat. Chem. Biol. 14(3):291-298 (2018), which is hereby incorporated by reference in its entirety). Not only does perfluoroalkylation of a drug-core typically provide analogs with novel structure, it appears to lead to increases in potency, cellular uptake and half-life. Molecular size and shape are inherently important with regard to selectivity and target engagement, as a binding pocket must be accommodated appropriately to optimize such criteria. In this regard, it appears that the electronic properties of perfluoroalkanes contribute greatly to their effectiveness. The polyfluorinated-motif essentially provides a bouquet of partial negative charges that are available to participate in a variety of multi-polar interactions with the partial positive charges along a polypeptide chain (FIG. 3D, FIG. 12, and FIGS. 15A-15C). Further, the overall inductive effect of perfluoroalkanes on a drug scaffold can strengthen the interactions of neighboring groups with the functional groups in a protein binding pocket; specifically by generating electron deficient pi-systems (Ph-R^{F}) and increasing the hydrogen bond donating ability of acidic protons (H—X—R—R^{F}; X═O, NR, S; R=alkyl, aryl, R^{F}). Finally, the unique physicochemical properties of perfluoroalkylated drugs appear to result in favorable PK and PD profiles (FIGS. 16A-16B and FIGS. 10A-10H).

Comparison of the sorafenib/regorafenib para-series perfluoroalkane-analogs with their hydrocarbon-isosteres was instrumental in determining the underlying factors that contribute to the overall efficacy of perfluoroalkyl-substituents. The almost complete lack of rescue provided by the hydrocarbon analogs, in the MTC fly model, could not be explained based on in vitro RET binding measurements (Kd; FIGS. 8A-8C). Among the compounds tested, APS-9-23-6 (50; —C₂H₅) was not effective in the MTC model (1% rescue), but showed strong binding affinity (Kd) for RET. All of the perfluoroalkyl-compounds that were effective in the MTC model showed decent binding to RET, but there was no direct correlation between Kd and fly-rescue. Cellular nanoBRET IC₅₀ measurements also did not appear to be directly associated with fly rescue. Further, none of the drugs tested in the nanoBRET assay showed a significant preference for mutant RET^{M918T} over wild-type. Therefore, it is postulated that differences in fly rescue across the perfluoroalkyl-libraries are most closely related to the protarget/antitarget profiles and the associated residence time of individual analogs instead of activity against a single target, including the Ret^{M955T} driver within the fly models. Among the drugs examined, the observed correlation between general RET (WT or mutant) binding (Kd and IC₅₀) and the size of the perfluoroalkyl-substituent is inconsequential with regard to in vivo efficacy, particularly considering the ineffectiveness of similarly sized hydrocarbon analogs (F is ˜0.27 Å larger than H) (Bondi, A., “Van Der Waals Volumes and Radii,” J. Phys. Chem. 68:441-451 (1964), which is hereby incorporated by reference in its entirety).

Cell permeability assays showed that the perfluoroalkyl-compounds (and sorafenib) tended to accumulate in cells while their hydrocarbon analogs cleared rather quickly. The observed influx/efflux ratios in MDCK and Caco-2 cells implied active transport for perfluoroalkyl-compounds 3 (APS-6-45) and 40 (APS-8-100-2) and passive transport for hydrocarbon analog 50 (APS-9-23-6). Based on the relatively high electron density of the perfluoroalkyl-substituents, the organic anion transporters (OATs) seem potentially relevant since they transport their substrates through a positively charged central pore. More specifically, OATs are involved in thyroid hormone transport as well as the uptake and clearance of albumin-bound amphipathic organic compounds (Niemi et al., “Organic Anion Transporting Polypeptide 1B1: A Genetically Polymorphic Transporter of Major Importance for Hepatic Drug Uptake,” Pharmacol. Rev. 63:157-181 (2011), which is hereby incorporated by reference in its entirety). Sorafenib is known to be >99% protein-bound in blood plasma (Neul et al., “Impact of Membrane Drug Transporters on Resistance to Small-Molecule Tyrosine Kinase Inhibitors,” Trends Pharmacol. Sci. 37:904-932 (2016), which is hereby incorporated by reference in its entirety), which potentially represents the other part of a mechanism that could provide perfluoroalkylated-drugs with a route to the OATs and a pathway into the tumor.

As the electronic properties of the perfluoroalkyl-drugs began to emerge as a driving force, R^{F}/R^{H} differences at a more basic level were studied. The difference in appearance between PBS-suspensions of perfluoroalkyl- and hydrocarbon-drugs was dramatic. The hydrocarbon-drugs appeared mostly as amorphous precipitates at the bottom of the PBS, while the perfluoroalkyl-drugs combined with the liquid to form ordered films that were less dense than buffer alone. Considering their relatively low water solubility, this type of behavior might be important with regard to perfluoroalkyl-drug efficacy.

To further understand the physicochemical properties, the partial charges, dipoles and molecular electrostatic potential (MEP) maps were examined for a series of Ph-R^{F}/R^{H} CAP-group fragments (FIGS. 9A-9C, FIGS. 3A-3E, FIG. 13, FIG. 14, FIG. 11, and FIG. 12). The R^{F}/R^{H} differences were again dramatic, but somewhat expected considering relevant electronegativity values; H (2.20) vs C (2.55) vs F (3.98) (Huheey, J. E., “The Electronegativity of Groups,” J. Phys. Chem. 69:3284-3291 (1965), which is hereby incorporated by reference in its entirety). The dipoles for the perfluoroalkyl-substituents are larger in magnitude but opposite in sign to the hydrocarbon-substituents. Overall, there is a build-up of partial negative charge at the fluorine atoms with the local maximum occurring adjacent to the benzylic position due to additional electron withdrawal from the phenyl ring. The hydrocarbon CAP-group fragments have substituents with partial positive charges at the hydrogen atoms and a concentration of electron density across the pi-system of the phenyl ring. The MEP maps provided a very enlightening visual summary of the calculations as well as insight into the pharmacodynamics of the perfluoroalkyl-drugs.

At the peptide level, partial charges, dipoles and MEP maps of greasy amino acid side chains line up well with the perfluoroalkyl-substituents (FIGS. 9A-9C, FIGS. 3A-3E, FIG. 13, FIG. 14, and FIGS. 15A-15C). Hydrogen-bonds between individual fluorine atoms and backbone N—H protons are implied as well as an electrostatic attraction between the perfluoroalkyl-substituents and hydrocarbon side-chains of amino acid residues. This is in contrast to hydrocarbon-substituents, which interact with the hydrocarbon side-chains of amino acid residues exclusively through van der Waals forces. At the whole protein level, MEP maps of wild-type and mutant RET(B06) homology models, as well as crystal structures of BRAF and SRC, revealed similar areas of positive electrostatic potential in the region of the binding pocket adjacent to the DFG/HRD motifs, where the perfluoroalkyl-substituents would bind (FIGS. 18A-18F, FIGS. 19A-19F, FIGS. 20A-20F, and FIGS. 21A-21F). Qualitatively, RET displayed the largest region of positive potential, BRAF was slightly less and SRC showed the smallest area (FIGS. 10A-10B). Differences in the area of positive electrostatic potential, across RET, BRAF and SRC appeared to correlate with the kinome profiling data for a series of regorafenib-based analogs; the para-substituted analogs showed the strongest binding to RET, followed by BRAF and finally SRC. However, the volume and orientation of the DFG-pocket relative to an inhibitor CAP-group always remains a related factor (FIGS. 10C-10H).

At least within the realm of kinases, several factors appear to contribute to the observed efficacy of perfluoroalkyl-substituted drugs. Size, shape and electrostatic complementarity seem to dictate direct engagement of a target of interest, as well as the pro-/anti-target profile of individual analogs. Further, the unique physicochemical properties of perfluoroalkyl-substituted drugs appear to be responsible for favorable PK and PD profiles.

### Example 9—Hepatocellular Carcinoma (HCC) Assay

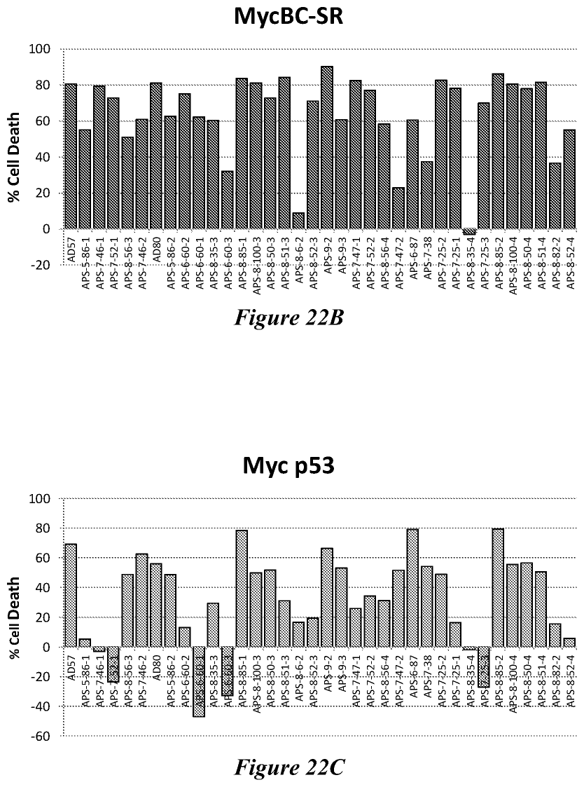

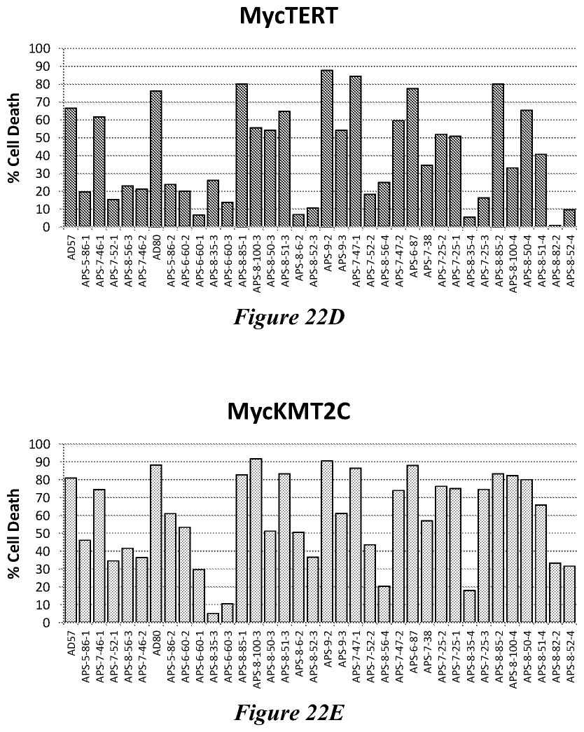

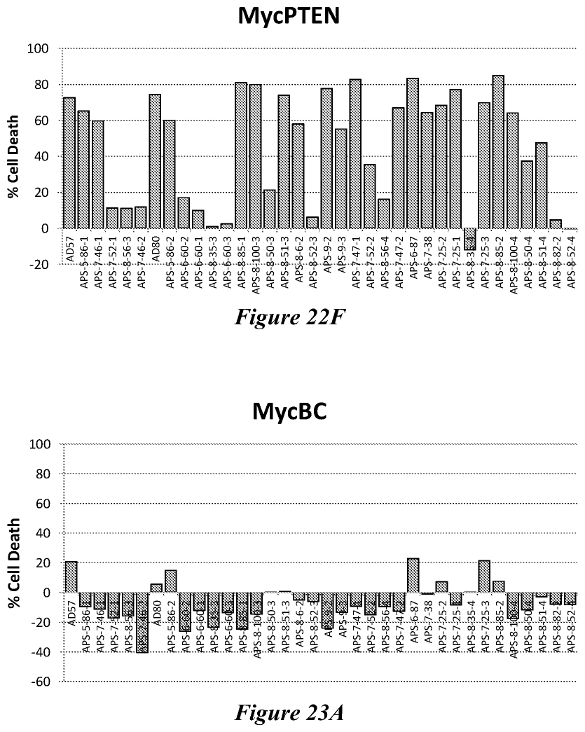

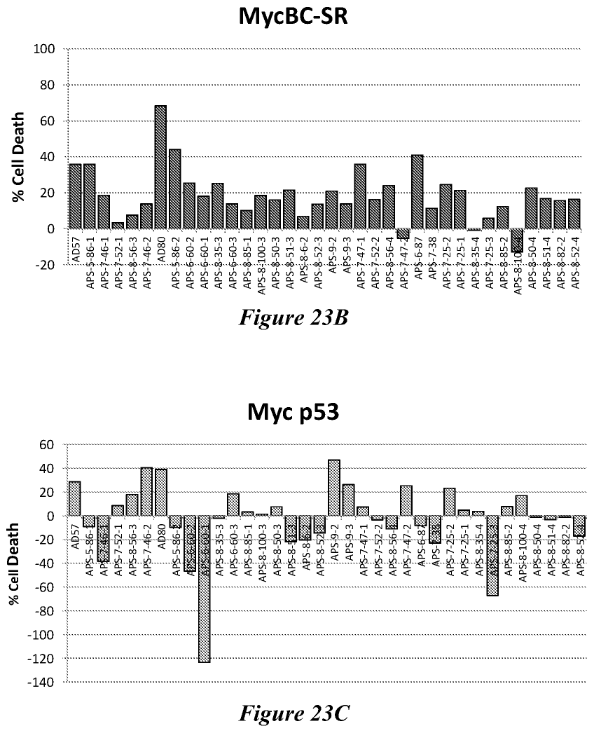

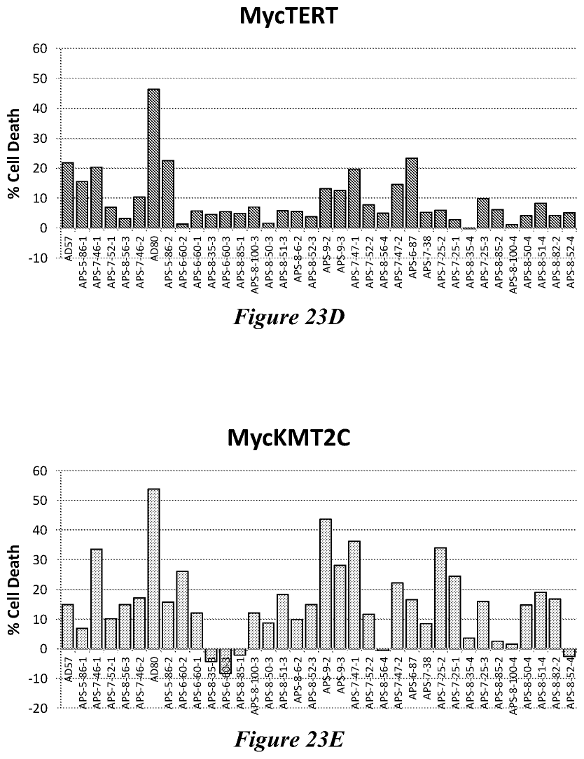

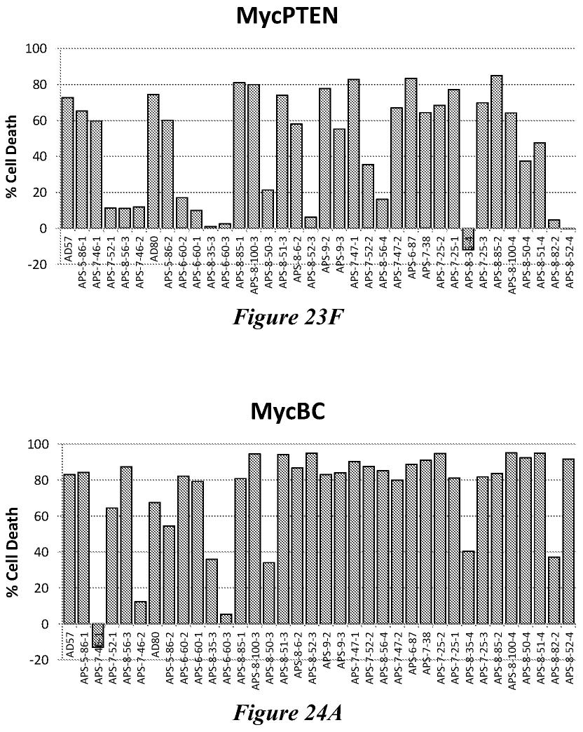

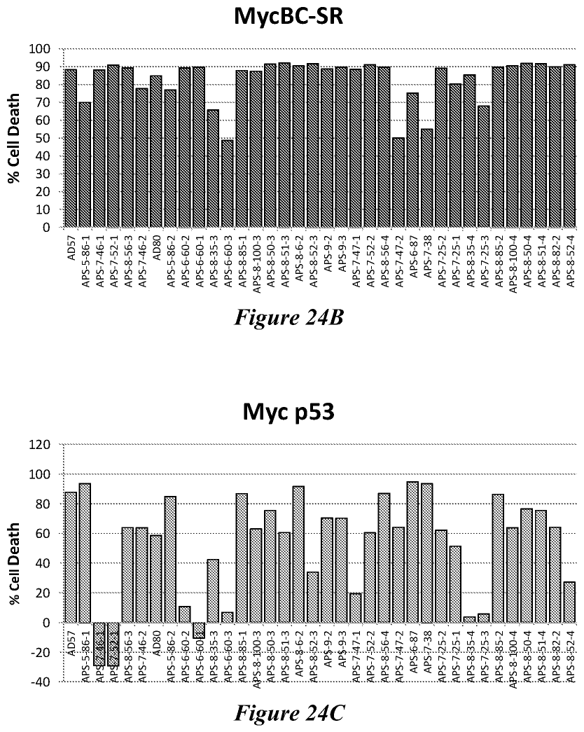

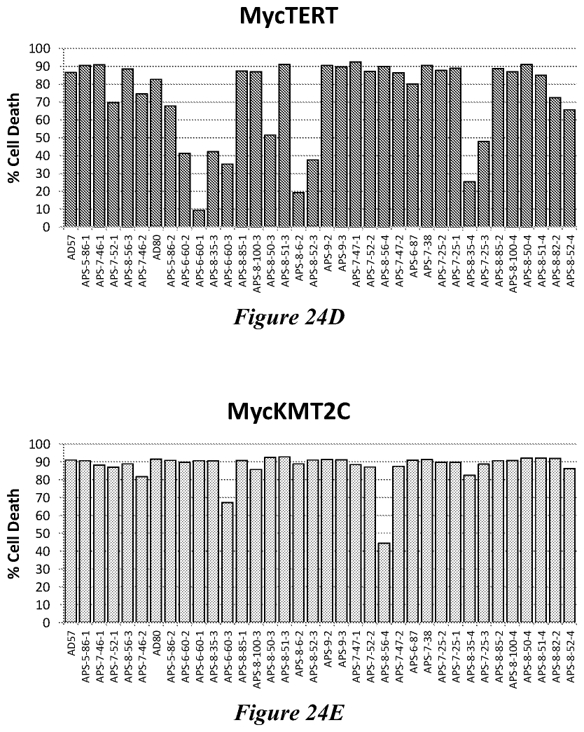

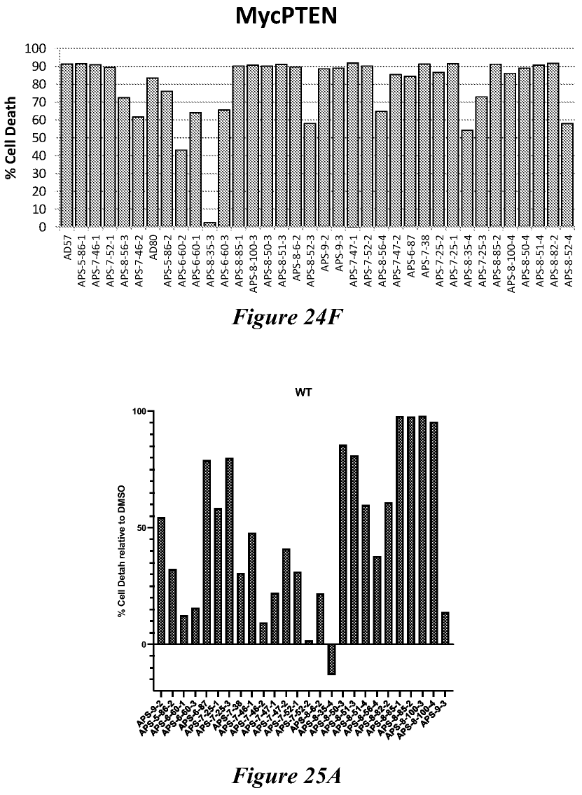

For assays in HCC tumor organoids, 96 well plates were first coated with a 50:50 solution of basal media:Matrigel (35 mL/well), which polymerized for 15 minutes at 37° C. Tumor organoids were taken out of Matrigel, broken, and washed. Tumor organoids were seeded at 1,000 cells per well and were treated the following day with single doses of drugs in technical triplicate. Final DMSO concentrations were kept below 0.5%. Cell viability was measured three days post drug administration with either luminescence from CellTiter-Glo or absorbance at 560 and 590 nm from resazurin. Viability data was analyzed by normalizing individual drug-treated well values to DMSO-treated wells (BC=B-Catenin; SR=sorafenib resistant). Results of 3D HCC cell line data for pyrazolo- and pyrrolopyrimidine compounds at 0.5 μm (FIGS. 22A-F), 0.05 μm (FIGS. 23A-F), and 5.0 μm (FIGS. 24A-F) were obtained. These data indicate several compounds with preferential inhibition of cell proliferation in BC, SR cells. In the BC model, at 5.0 μM, the top ten compounds were: APS-8-100-4, APS-8-52-3, APS-8-51-4, APS-7-25-2, APS-8-100-3, APS-8-51-3, APS-8-50-4, APS-8-52-4, and APS-7-38. In the SR model, at 5.0 μM, the top ten compounds were: APS-8-51-3, APS-8-50-4, APS-8-51-4, APS-8-52-3, APS-8-50-3, APS-7-52-2, APS-8-52-4, APS-7-52-1, and APS-8-100-4.

### Example 10—Colorectal Carcinoma (CRC) Assay

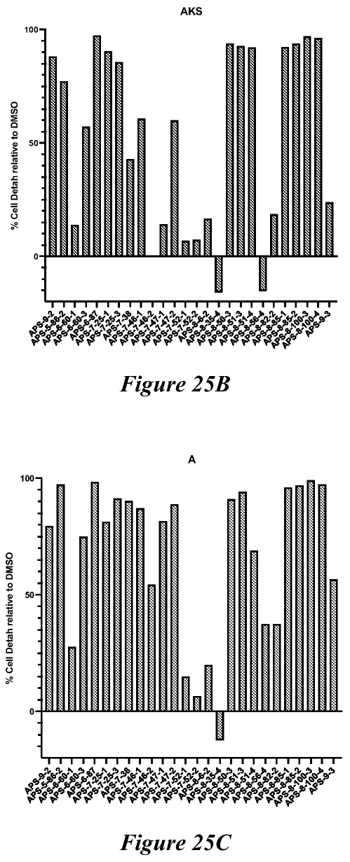

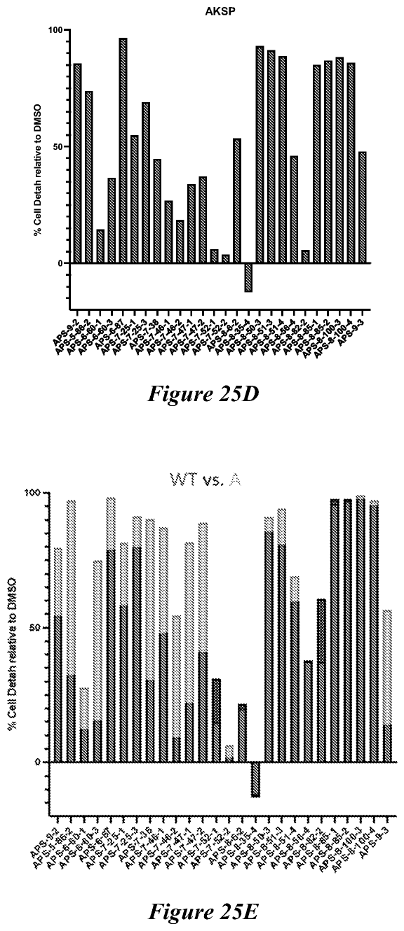

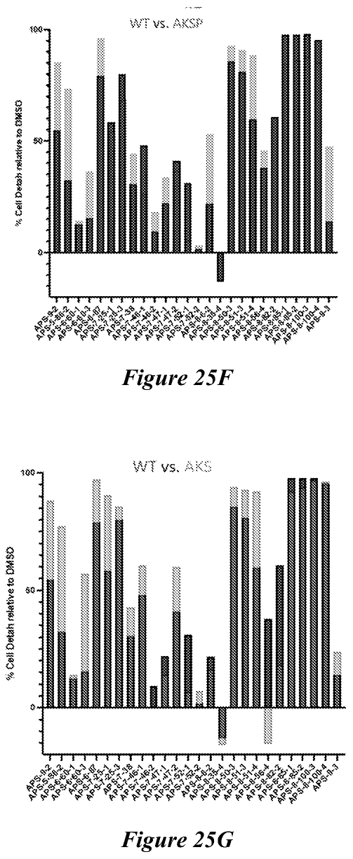

To model different stages of colorectal cancer (CRC), intestinal organoids were derived from genetically engineered mice with the following genotypes Apc^{flx/flx} (A) and Apc^{flx/flx} Kra^{LSL-G12D} Smad4^{flx/flx} (AKS) and transformed ex vivo by addition of 100 nM 4-OHT for 24 hours. Once the organoids acquired a spheroid morphology characteristic of CRC Tumoroids, trp53 was knocked out in AKS tumoroids using CRISPR-CAS9 technology to generate the AKSP line. CRC tumoroid lines as well as wild type intestinal organoids were seeded onto Matrigel in 96 well plates in triplicates 16 hours before being treated with the indicated compounds at a final concentration of 0.5 μM. 72 hours later, cell viability was assessed using Titerglo® assay. Results of 3D CRC organoid line data for pyrazolo- and pyrrolopyrimidine compounds at 0.5 μm were obtained (FIGS. 25A-G). These data indicate several compounds with preferential inhibition of cell proliferation in mutant CRC models over wild-type counterparts, indicative of wide therapeutic index. In the AKSP model, at 5.0 μM, the top ten compounds were: APS-8-35-3, APS-8-35-4, APS-6-87, APS-9-3, AD80, APS-8-85-2, APS-8-51-3, APS-9-2, and APS-8-56-4.

Although preferred embodiments have been depicted and described in detail herein, it will be apparent to those skilled in the relevant art that various modifications, additions, substitutions, and the like can be made without departing from the spirit of the disclosure and these are therefore considered to be within the scope of the disclosure as defined in the claims which follow.

What is claimed:

1. A compound of formula (I) having the following structure: 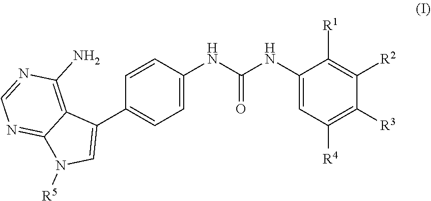
or a stereoisomer, pharmaceutically acceptable salt, oxide, or solvate thereof, wherein
R¹, R², R³, and R⁴ are independently selected from the group consisting of H, F, CF₃, C₂F₅, C₃F₇, C₄F₉, C₅F₁₁, CF₁₃, C₇F₁₅, and C₈F₁₇; and
R⁵ is a C₁-C₆ alkyl or a C₃-C₇ cycloalkyl.

2. The compound of claim 1, wherein
R¹ is H;
R² is H;
R³ is H;
R⁴ is selected from the group consisting of CF₃, C₂F₅, C₃F₇, and C₄F₉; and
R⁵ is a C₃ alkyl.

3. The compound of claim 2, wherein the compound is selected from the group consisting of:

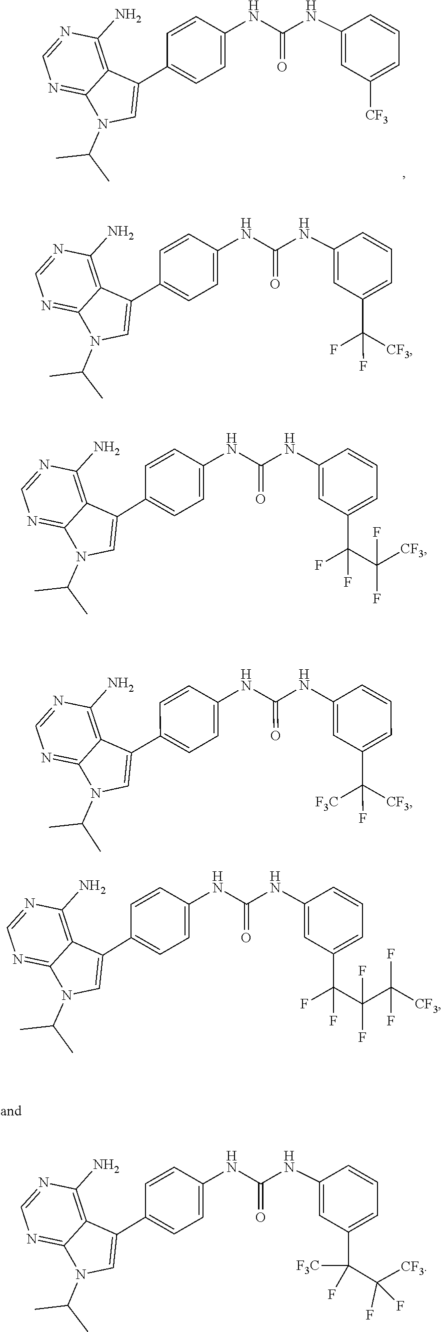

4. The compound of claim 1, wherein
R¹ is F;
R² is H;
R³ is H; and
R⁴ is selected from the group consisting of CF₃, C₂F₅, C₃F₇, and C₄F₉.

5. The compound of claim 4, wherein the compound is selected from the group consisting of:

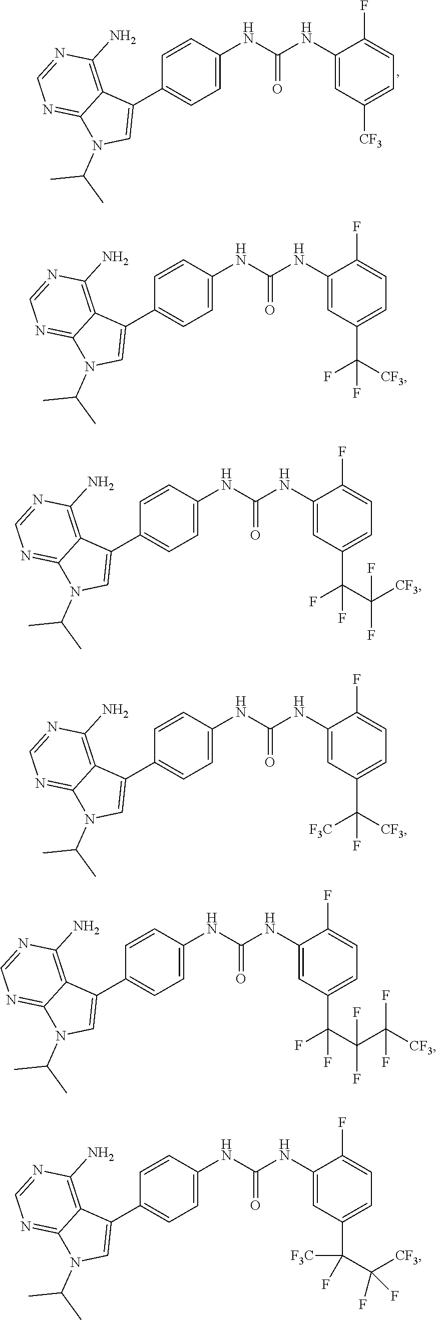

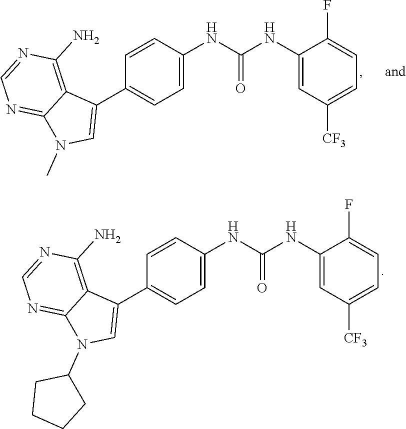 [SMILES: Cn1cc(-c2ccc(NC(=O)Nc3cc(C(F)(F)F)ccc3F)cc2)c2c(N)ncnc21 ; Nc1ncnc2c1c(-c1ccc(NC(=O)Nc3cc(C(F)(F)F)ccc3F)cc1)cn2C1CCCC1]

6. The compound of claim 1, wherein
R¹ is H;
R² is H;
R³ is selected from the group consisting of CF₃, C₂F₅, C₃F₇, and C₄F₉;
R⁴ is H; and
R⁵ is a C₃ alkyl.

7. The compound of claim 6, wherein the compound is selected from the group consisting of:

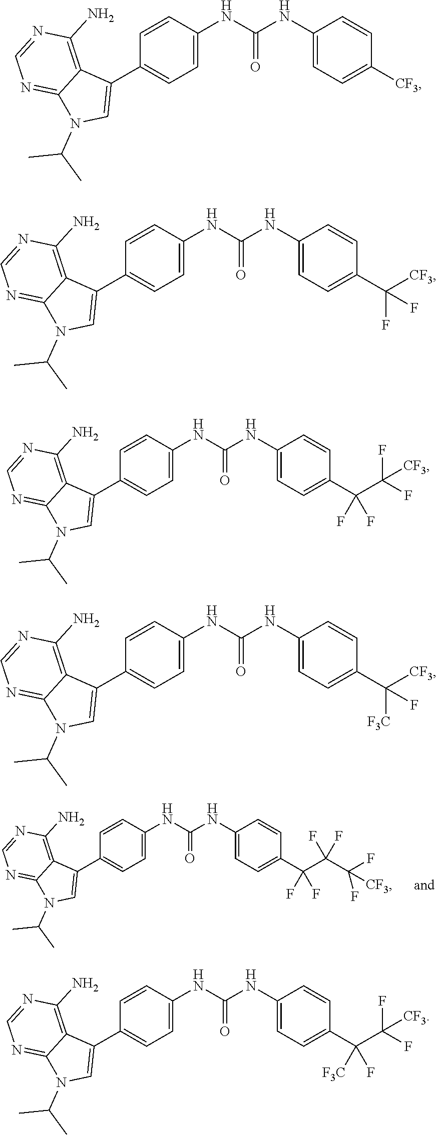

8. A compounds of formula (II) having the following structure: 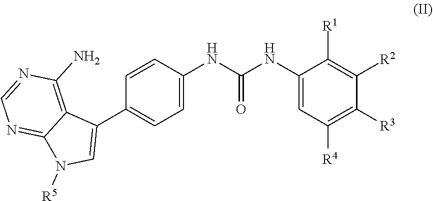
or a stereoisomer, pharmaceutically acceptable salt, oxide, or solvate thereof, wherein
R¹, R², and R³ are independently selected from the group consisting of H, F, CF₃, C₂F₅, C₃F, C₄F₉, C₅F₁₁, C₆F₁₃, C₇F₁₅ and C₈F₁₇;
R⁴ is selected from the group consisting of H, F, C₂F₅, C₃F₇, C₄F₉, C₅F₁₁, C₆F₁₃, C₇F₁₅, and C₈F₁₇; and
R⁵ is a C₁-C₆ alkyl or a C₃-C₇ cycloalkyl.

9. The compound of claim 8, wherein
R¹ is H;
R² is H;
R³ is H;
R⁴ is selected from the group consisting of C₂F₅, C₃F₇, and C₄F₉; and
R⁵ is a C₃ alkyl.

10. The compound of claim 9, wherein the compound is selected from the group consisting of.

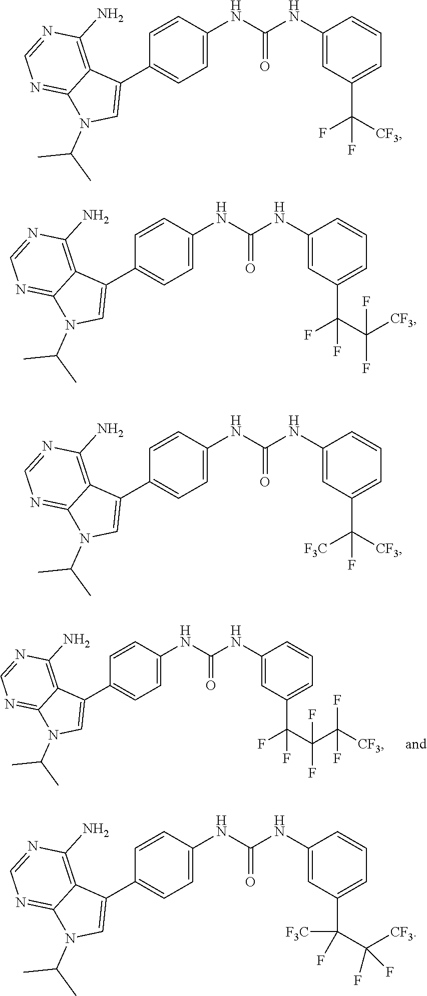

11. The compound of claim 8, wherein
R¹ is F;
R² is H;
R³ is H;
R⁴ is selected from the group consisting of C₂F₅, C₃F₇, and C₄F₉; and
R⁵ is a C₃ alkyl.

12. The compound of claim 11, wherein the compound is selected from the group consisting of.

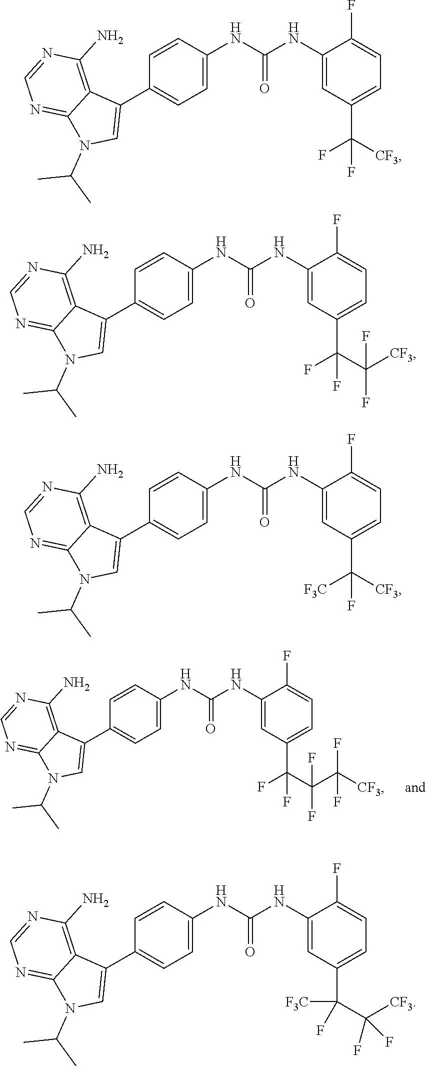

13. The compound of claim 8, wherein
R¹ is H;
R² is H;
R³ is selected from the group consisting of CF₃, C₂F₅, C₃F₇, and C₄F₉;
R⁴ is H; and
R⁵ is a C₃ alkyl.

14. The compound of claim 13, wherein the compound is selected from the group consisting of:

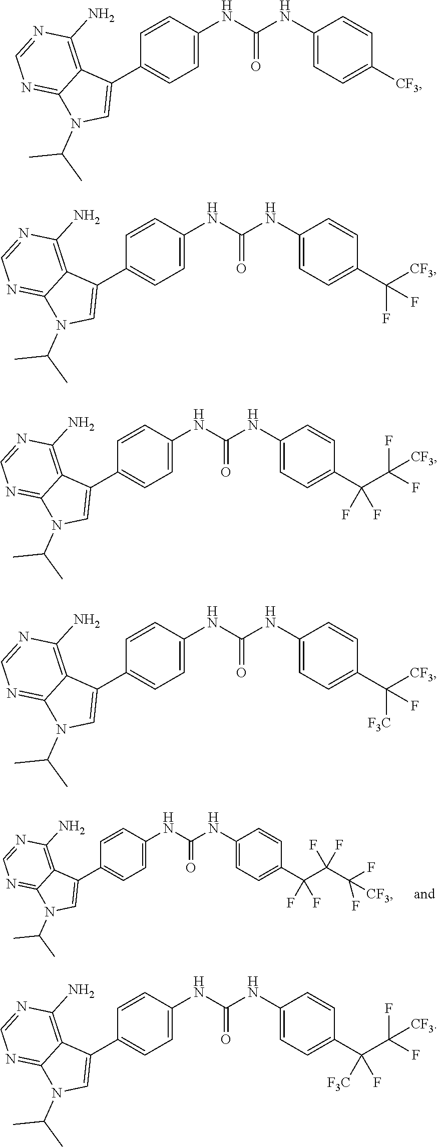

15. A composition comprising:
the compound according to any one of claims 1-14; and
a carrier.

16. A composition according to claim 15, wherein the carrier is a pharmaceutically-acceptable carrier.

17. A method of treating cancer in a subject, said method comprising:
administering to a subject in need thereof a compound of formula (I) having the following structure: 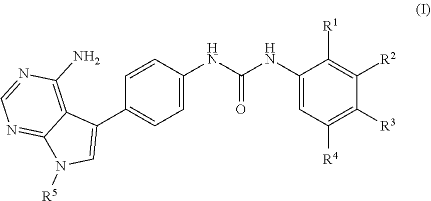
or a stereoisomer, pharmaceutically acceptable salt, oxide, or solvate thereof, wherein
R¹, R², R³, and R⁴ are independently selected from the group consisting of H, F, CF₃, C₂F₅, C₃F₇, C₄F₉, C₅F₁₁, C₆F₁₃, C₇F₁₅, and C₈F₁₇; and
R⁵ is a C₁-C₆ alkyl or a C₃-C₇ cycloalkyl.

18. The method of claim 17, wherein
R¹ is H;
R² is H;
R³ is H;
R⁴ is selected from the group consisting of CF₃, C₂F₅, C₃F₇, and C₄F₉; and
R⁵ is a C₃ alkyl.

19. The method of claim 18, wherein the compound is selected from the group consisting of:

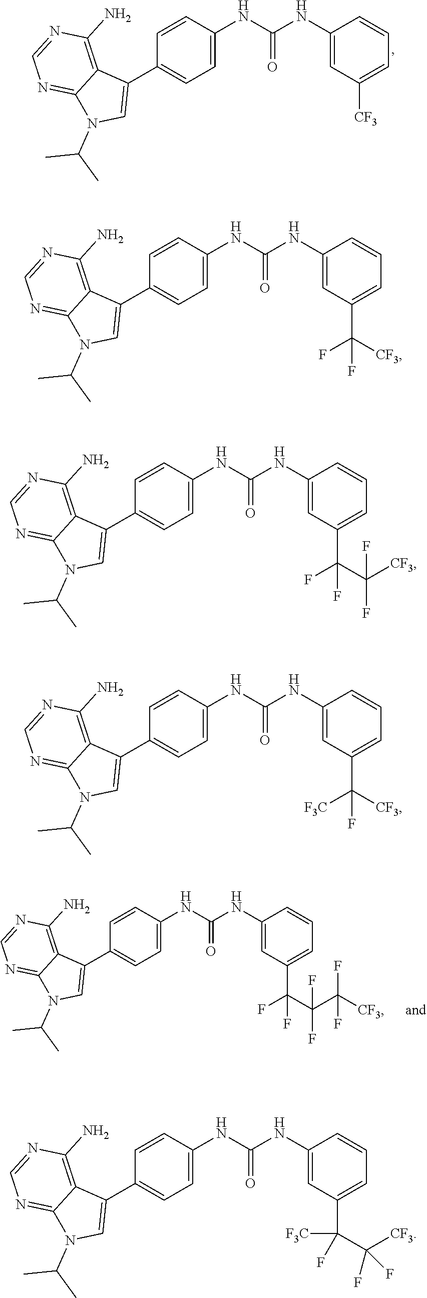

20. The method of claim 17, wherein
R¹ is F;
R² is H;
R³ is H; and
R⁴ is selected from the group consisting of CF₃, C₂F₅, C₃F₇, and C₄F₉.

21. The method of claim 20, wherein the compound is selected from the group consisting of:

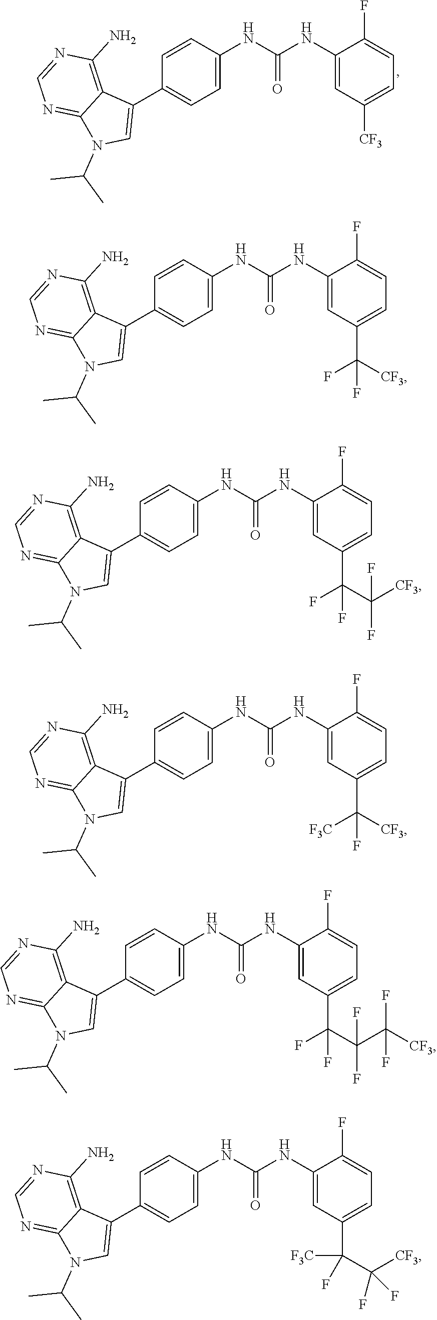

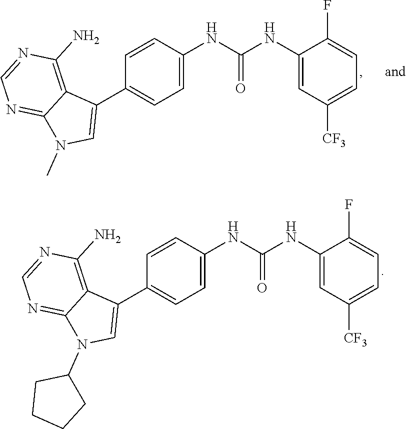 [SMILES: Cn1cc(-c2ccc(NC(=O)Nc3cc(C(F)(F)F)ccc3F)cc2)c2c(N)ncnc21 ; Nc1ncnc2c1c(-c1ccc(NC(=O)Nc3cc(C(F)(F)F)ccc3F)cc1)cn2C1CCCC1]

22. The method of claim 17, wherein
R¹ is H;
R² s H;
R³ is selected from the group consisting of CF₃, C₂F₅, C₃F₇, and C₄F₉;
R⁴ is H; and
R⁵ is a C₃ alkyl.

23. The method of claim 22, wherein the compound is selected from the group consisting of:

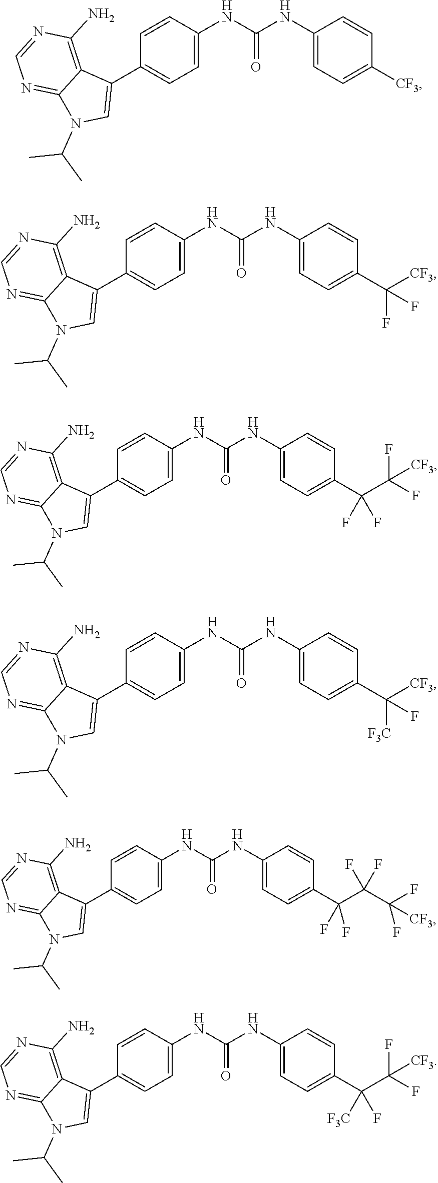

24. A method of treating cancer in a subject, said method comprising:
administering to a subject in need thereof a compound of formula (II) having the following structure: 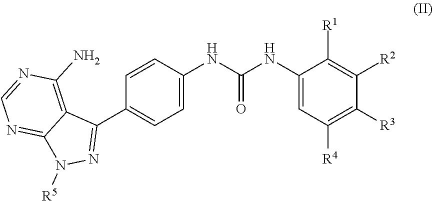
or a stereoisomer, pharmaceutically acceptable salt, oxide, or solvate thereof, wherein
R¹, R², and R³ are independently selected from the group consisting of H, F, CF₃, C₂F₅, C₃F₇, C₄F₉, C₅F₁₁, C₆F₁₃, C₇F₁₅, and C₈F₁₇;
R⁴ is selected from the group consisting of H, F, C₂F₅, C₃F₇, C₄F₉, C₅F₁₁, C₆F₁₃, C₇F₁₅, and C₈F₁₇; and
R⁵ is a C₁-C₆ alkyl or a C₃-C₇ cycloalkyl.

25. The method of claim 24, wherein
R¹ is H;
R² s H;
R³ is H;
R⁴ is selected from the group consisting of C₂F₅, C₃F₇, and C₄F₉; and
R⁵ is a C₃ alkyl.

26. The method of claim 25, wherein the compound is selected from the group consisting of:

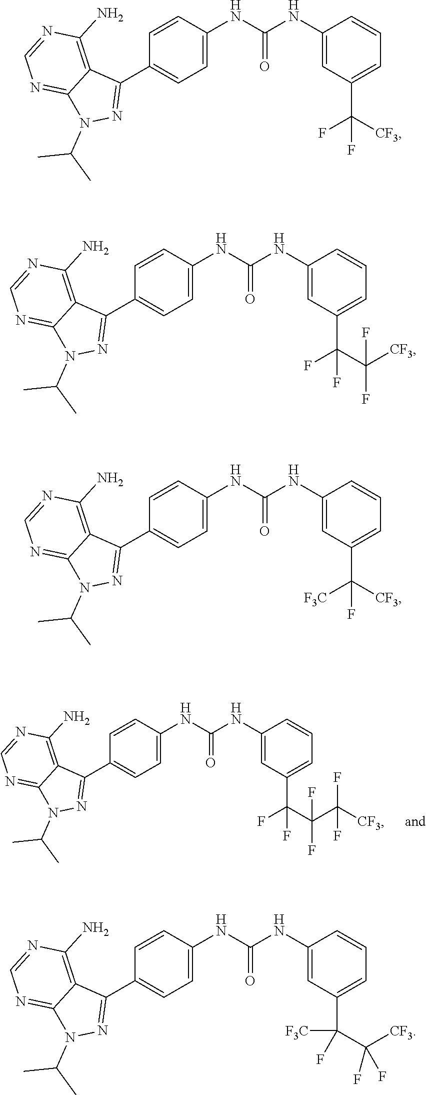 [SMILES: CC(C)n1nc(-c2ccc(NC(=O)Nc3cccc(C(F)(F)C(F)(F)F)c3)cc2)c2c(N)ncnc21 ; CC(C)n1nc(-c2ccc(NC(=O)Nc3cccc(C(F)(F)C(F)(F)C(F)(F)F)c3)cc2)c2c(N)ncnc21 ; CC(C)n1nc(-c2ccc(NC(=O)Nc3cccc(C(F)(C(F)(F)F)C(F)(F)F)c3)cc2)c2c(N)ncnc21 ; CC(C)n1nc(-c2ccc(NC(=O)Nc3cccc(C(F)(F)C(F)(F)C(F)(F)C(F)(F)F)c3)cc2)c2c(N)ncnc21 ; "and" ; CC(C)n1nc(-c2ccc(NC(=O)Nc3cccc(C(F)(C(F)(F)F)C(F)(F)C(F)(F)F)c3)cc2)c2c(N)ncnc21]

27. The method of claim 24, wherein
R¹ is F;
R² is H;
R³ is H;
R⁴ is selected from the group consisting of C₂F₅, C₃F₇, and C₄F₉; and
R⁵ is a C₃ alkyl.

28. The method of claim 27, wherein the compound is selected from the group consisting of:

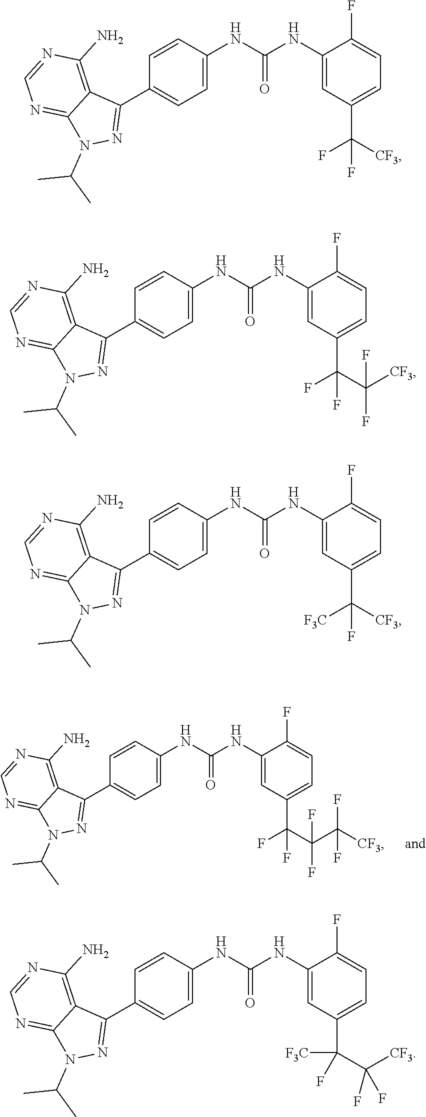 [SMILES: CC(C)n1nc(-c2ccc(NC(=O)Nc3cc(C(F)(F)C(F)(F)F)ccc3F)cc2)c2c(N)ncnc21 ; CC(C)n1nc(-c2ccc(NC(=O)Nc3cc(C(F)(F)C(F)(F)C(F)(F)F)ccc3F)cc2)c2c(N)ncnc21 ; CC(C)n1nc(-c2ccc(NC(=O)Nc3cc(C(F)(C(F)(F)F)C(F)(F)F)ccc3F)cc2)c2c(N)ncnc21 ; CC(C)n1nc(-c2ccc(NC(=O)Nc3cc(C(F)(F)C(F)(F)C(F)(F)C(F)(F)F)ccc3F)cc2)c2c(N)ncnc21 ; "and" ; CC(C)n1nc(-c2ccc(NC(=O)Nc3cc(C(F)(C(F)(F)F)C(F)(F)C(F)(F)F)ccc3F)cc2)c2c(N)ncnc21]

29. The method of claim 24, wherein
R¹ is H;
R² is H;
R³ is selected from the group consisting of CF₃, C₂F₅, C₃F₇, and C₄F₉;
R⁴ is H; and
R⁵ is a C₃ alkyl.

30. The method of claim 29, wherein the compound is selected from the group consisting of:

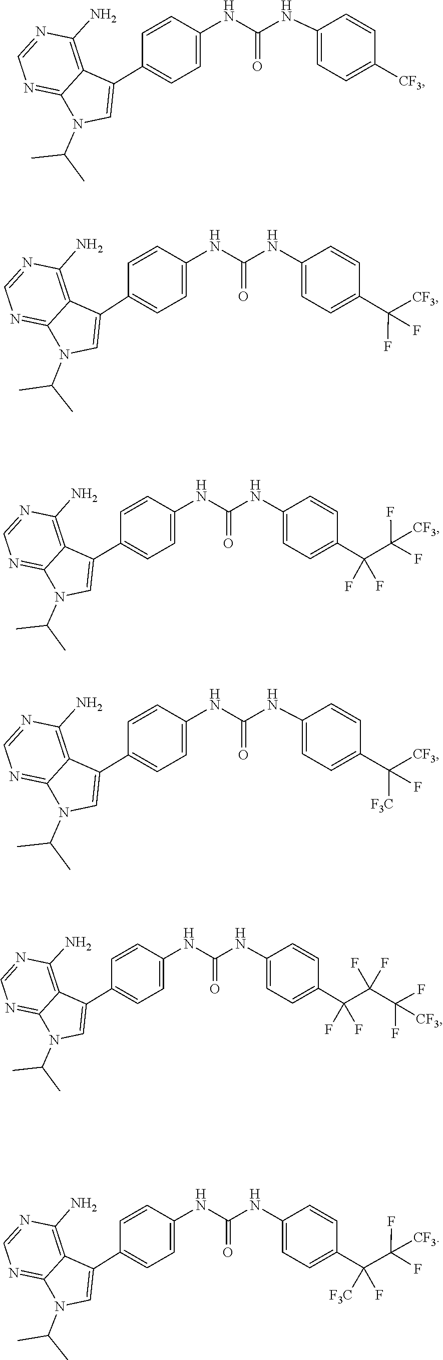 [SMILES: CC(C)n1cc(-c2ccc(NC(=O)Nc3ccc(C(F)(F)F)cc3)cc2)c2c(N)ncnc21 ; CC(C)n1cc(-c2ccc(NC(=O)Nc3ccc(C(F)(F)C(F)(F)F)cc3)cc2)c2c(N)ncnc21 ; CC(C)n1cc(-c2ccc(NC(=O)Nc3ccc(C(F)(F)C(F)(F)C(F)(F)F)cc3)cc2)c2c(N)ncnc21 ; CC(C)n1cc(-c2ccc(NC(=O)Nc3ccc(C(F)(C(F)(F)F)C(F)(F)F)cc3)cc2)c2c(N)ncnc21 ; CC(C)n1cc(-c2ccc(NC(=O)Nc3ccc(C(F)(F)C(F)(F)C(F)(F)C(F)(F)F)cc3)cc2)c2c(N)ncnc21 ; CC(C)n1cc(-c2ccc(NC(=O)Nc3ccc(C(F)(C(F)(F)F)C(F)(F)C(F)(F)F)cc3)cc2)c2c(N)ncnc21]

31. The method according to any one of claims 17-30, wherein said administering is carried out orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes.

32. The method according to any one of claims 17-31, wherein the subject is a mammalian subject.

33. The method according to any one of claims 17-32, wherein the subject is a human subject.

34. The method according to any one of claims 17-33, wherein the cancer is selected from the group consisting of Acute Lymphoblastic Leukemia (ALL), Acute Myeloid Leukemia (AML), Adrenocortical Carcinoma, Adrenal Cortex Cancer, Anal Cancer, Appendix Cancer, Astrocytomas, Atypical Teratoid/Rhabdoid Tumor, Basal Cell Carcinoma, Extrahepatic Bile Duct Cancer, Bladder Cancer, Bone Cancer, Brain Tumors, Breast Cancer, Bronchial Tumors, Burkitt Lymphoma, Carcinoid Tumor, Cardiac (Heart) Tumors, Cervical Cancer, Cholangiocarcinoma, Chronic Lymphocytic Leukemia (CLL), Chronic Myelogenous Leukemia (CML), Chronic Myeloproliferative Neoplasms, Colorectal Cancer, Cutaneous T-Cell Lymphoma, Ductal Carcinoma In Situ (DCIS), Endometrial Cancer, Ependymoma, Esophageal, Esthesioneuroblastoma, Ewing Sarcoma, Intraocular Melanoma, Retinoblastoma, Malignant Fibrous Histiocytoma of Bone, Osteosarcoma, Gallbladder Cancer, Gastric (Stomach) Cancer, Gastrointestinal Carcinoid Tumor, Gastrointestinal Stromal Tumors (GIST), Gestational Trophoblastic Disease, Gliomas, Hairy Cell Leukemia, Head and Neck Cancer, Hepatocellular (Liver) Cancer, Langerhans Cell Histiocytosis, Hodgkin Lymphoma, Hypopharyngeal Cancer, Intraocular Melanoma, Kaposi Sarcoma, Kidney cancer, Langerhans Cell Histiocytosis, Leukemia, Lung Cancer, Lymphoma, Medullary Thyroid Carcinoma, Melanoma, Intraocular [symbol] na, Merkel Cell Carcinoma, Malignant Mesothelioma, Metastatic Squamous Neck Cancer with Occult Primary, Multiple Endocrine Neoplasia Syndromes, Multiple Myeloma/Plasma Cell Neoplasms, Myelodysplastic Syndromes, Myelodysplastic/Myeloproliferative Neoplasms, and Chronic Myeloproliferative Neoplasms, Chronic Myelogenous Leukemia (CML), Acute Myeloid Leukemia (AML), Nasopharyngeal Cancer, Neuroblastoma, Non-Hodgkin Lymphoma, Non-Small Cell Lung Cancer, Oral Cancer, Lip and Oral Cavity Cancer, Oropharyngeal Cancer, Ovarian Cancer, Pancreatic Cancer and Pancreatic Neuroendocrine Tumors (Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Plasma Cell Neoplasm/Multiple Myeloma, Primary Central Nervous System (CNS) Lymphoma, Prostate Cancer, Rectal Cancer, Renal Cell (Kidney) Cancer, Retinoblastoma, Rhabdomyosarcoma, Salivary Gland Cancer, Sarcoma, Sezary Syndrome, Small Cell Lung Cancer, Small Intestine Cancer, Soft Tissue Sarcoma, Squamous Cell Carcinoma, Squamous Neck Cancer, Stomach (Gastric) Cancer, T-Cell Lymphoma, Testicular Cancer, Throat Cancer, Thymoma and Thymic Carcinoma, Thyroid Cancer, Transitional Cell Cancer of the Renal Pelvis and Ureter, Urethral Cancer, Uterine Cancer, Endometrial and Uterine Sarcoma, Vaginal Cancer, Vulvar Cancer, Waldenstrom Macroglobulinemia, and Wilms Tumor.
